(19) United States
(12) Patent Application Publication
(10) Pub. No.: US 2021/0380692 A1
(43) Pub. Date: Dec. 9, 2021

(54) ANTI-TCR ANTIBODY MOLECULES AND USES THEREOF

(71) Applicant: Marengo Therapeutics, Inc., Cambridge, MA (US)

(72) Inventors: Andreas Loew, Boston, MA (US); Seng-Lai Tan, Sudbury, MA (US); Jonathan Hsu, Waltham, MA (US); Brian Edward Vash, Cambridge, MA (US)

(21) Appl. No.: 17/402,322

(22) Filed: Aug. 13, 2021

Related U.S. Application Data
(63) Continuation of application No. PCT/US2020/019319, filed on Feb. 21, 2020.
(60) Provisional application No. 62/808,784, filed on Feb. 21, 2019.
(60) Provisional application No. 62/956,871, filed on Jan. 3, 2020.

(51) Int. Cl. C07K 16/28; G01N 33/68; A61K 35/17; C12N 5/0783

(52) U.S. Cl. CPC C07K 16/2809; G01N 33/6893; A61K 35/17; C12N 5/0636; G01N 2800/26; C07K 2317/24; C07K 2317/74; G01N 2333/7051; C07K 2317/75

(57) ABSTRACT

The disclosure provides antibody molecules that bind to TCR Vβ regions and multispecific molecules comprising said antibody molecules. Additionally, disclosed are nucleic acids encoding the same, methods of producing the aforesaid molecules, pharmaceutical compositions comprising aforesaid molecules, and methods of treating an infectious disease using the aforesaid molecules.

### RELATED APPLICATIONS

This application is a continuation of International Application No. PCT/US2020/019319, filed on Feb. 21, 2020, which claims the benefit of U.S. Provisional Application 62/808,784 filed on Feb. 21, 2019 and U.S. Provisional Application 62/956,871 filed on Jan. 3, 2020, the entire contents of each of which are hereby incorporated by reference.

### SEQUENCE LISTING

The instant application contains a Sequence Listing which has been submitted electronically in ASCII format and is hereby incorporated by reference in its entirety. Said ASCII copy, created on Feb. 17, 2020, is named 53676-733.301 SL.txt and is 1,080,059 bytes in size.

### BACKGROUND

T cell mediated antigen recognition depends on the interaction of the T-cell receptor (TCR) with the antigen-major histocompatibility complex (MHC). The heterodimeric TCRs consist of a combination of α and β chains (αβ TCR) expressed by the majority of T cells, or γδ chains (γδ TCR) present only in about 1-5% of the T cells. A highly diverse TCR repertoire is a fundamental property of an effective immune system. However, it is now understood that the immune repertoire can change greatly with the onset and progression of infectious diseases. Thus, there exists a need in the art for improved strategies for treating infectious diseases by harnessing differences in the immune repertoire.

### SUMMARY OF THE INVENTION

Disclosed herein are, inter alfa, methods of using antibodies directed to the variable chain of the beta subunit of TCR (TCRβV) which bind and, e.g., activate, T cells, e.g., a subset of T cells, to treat infectious diseases. Generally, the invention features a method of expanding, e.g., increasing the number of, a T cell population comprising a TCRβV molecule (e.g., as described herein), the method comprising: contacting the T cell population with an antibody molecule, e.g., humanized antibody molecule, which binds, e.g., specifically binds, to a T cell receptor beta variable chain (TCRβV) region (e.g., an anti-TCRβV antibody molecule), thereby expanding the T cell population. In some embodiments, the T cell population is obtained from or comprised in a subject, e.g., a subject having an infectious disease (e.g., as described herein). Without wishing to be bound by theory, in some embodiments, the TCRβV clonotype bound by the antibody molecule does not have to be the particular TCRβV clonotype that is over-represented, e.g., that shows a higher level or activity, in the subject having the infectious disease.

### Enumerated Embodiments

1. A method of expanding, e.g., increasing the number of, a T cell population comprising a TCRβV molecule (e.g., as described herein), the method comprising: contacting the T cell population with an antibody molecule, e.g., humanized antibody molecule, which binds, e.g., specifically binds, to a T cell receptor beta variable chain (TCRβV) region, thereby expanding the T cell population, wherein the T cell population is obtained from or comprised in a subject having an infectious disease.

2. A method of treating a subject having an infectious disease, the method comprising administering an effective amount of an anti-TCRβV antibody molecule (e.g., a TCRβV agonist) to the subject, thereby treating the infectious disease.

3. A method of evaluating, e.g., identifying the level or activity of a TCRβV molecule in a subject having an infectious disease, the method comprising acquiring a status for the TCRβV molecule in the subject; wherein the level or activity of the TCRβV molecule is higher (e.g., at least about 1.1, 1.2, 1.3, 1.4, 1.5, 2, 3, 4, 5, 6, 7, 8, 9, 10, 20, 30, 40, 50, 60, 70, 80, 90, 100, 200, 300, 400, 500, 600, 700, 800, 900, 1000, 5000, 10,000, or 100,000-fold higher) relative to the level or activity of the TCRβV molecule in a healthy subject (e.g., a subject that does not have the infectious disease).

4. A method of treating a subject having an infectious disease, the method comprising:
(i) acquiring a status for the TCRβV molecule in the subject; and
(ii) administering an effective amount of an anti-TCRβV antibody molecule (e.g., a TCRβV agonist) to the subject, thereby treating the infectious disease;
wherein the level or activity of the TCRβV molecule is higher (e.g., at least about 1.1, 1.2, 1.3, 1.4, 1.5, 2, 3, 4, 5, 6, 7, 8, 9, 10, 20, 30, 40, 50, 60, 70, 80, 90, 100, 200, 300, 400, 500, 600, 700, 800, 900, 1000, 5000, 10,000, or 100,000-fold higher) relative to the level or activity of the TCRβV molecule in a healthy subject (e.g., a subject that does not have the infectious disease).

5. A method of evaluating a subject for the presence of an infectious disease, the method comprising:
(i) acquiring a status for one or more TCRβV molecules in a biological sample from the subject and in a biological sample from a healthy subject (e.g., a subject that does not have the infectious disease); and
(ii) determining whether one or more of the TCRβV molecules exhibits an elevated level or activity in the subject relative to the healthy subject; wherein an elevated level or activity in the subject relative to in the healthy subject is indicative of the presence of the infectious disease.

6. A method of treating a subject having an infectious disease, the method comprising:
(i) acquiring a status for one or more TCRβV molecules in a biological sample from the subject and in a biological sample from a healthy subject (e.g., a subject that does not have the infectious disease);
(ii) determining whether one or more of the TCRβV molecules exhibits an elevated level or activity in the subject relative to the healthy subject; and
(iii) if an elevated level or activity in the subject relative to in the healthy subject is determined, administering an effective amount of an anti-TCRβV antibody molecule (e.g., a TCRβV agonist) to the subject.

7. The method of any of the preceding embodiments, wherein the status is indicative of the subject having the infectious disease or a symptom thereof.

8. The method of any of the preceding embodiments, wherein the status is indicative of responsiveness to a therapy, e.g., a TCRβV molecule.

9. The method of any of the preceding embodiments, wherein the status is determined, e.g., measured, by an assay described herein.

10. The method of any of the preceding embodiments, wherein the acquiring comprises: isolating a biological sample from the subject, contacting the biological sample with an anti-TCRβV antibody molecule (e.g., the same anti-TCRβV antibody molecule or a different anti-TCRβV antibody molecule), and determining a level of T cell expansion in the biological sample, e.g., relative to the level of T cell expansion in a biological sample obtained from a healthy subject (e.g., a subject that does not have the infectious disease).

11. The method of embodiment 10, further comprising administering expanded T cells from the biological sample to the subject.

12. The method of any of the preceding embodiments, wherein the acquiring comprises: isolating a biological sample from the subject, contacting the biological sample with an anti-TCRβV antibody molecule (e.g., the same anti-TCRβV antibody molecule or a different anti-TCRβV antibody molecule), and determining a level of T cell function (e.g., cytotoxic activity) in the biological sample, e.g., relative to the level of T cell expansion in a biological sample obtained from a healthy subject (e.g., a subject that does not have the infectious disease).

13. A method of identifying one or more TCRβV molecules associated with a disease, the method comprising:
(i) acquiring a status for a plurality of TCRβV molecules in a biological sample from a first subject having the disease and in a biological sample from a second subject not having the disease; and
(ii) determining whether one or more of the TCRβV molecules exhibits an elevated level or activity in the first subject relative to the second subject; thereby identifying one or more TCRβV molecules associated with the disease.

14. The method of any of the preceding embodiments, wherein the infectious disease is selected from Epstein-Barr virus (EBV), influenza, human immunodeficiency virus (HIV), simian immunodeficiency virus (SIV), tuberculosis, malaria, or human cytomegalovirus (HCMV).

15. The method of any of the preceding embodiments, wherein the TCRβV is selected from TCRβV V5-6, TCRβV V6-5, TCRβV V7, TCRβV V9, TCRβV V10, TCRβV V12 (e.g., TCRβV V12-4), TCRβV V13, TCRβV V14, TCRβV V19, TCRβV V23-1, or a subfamily member thereof (e.g., as listed in Table 1 or Table 2).

16. The method of any of the preceding embodiments, wherein the anti-TCRβV antibody molecule induces expansion, e.g., increasing the number of, a T cell population comprising a TCRβV molecule (e.g., the TCRβV bound by the anti-TCRβV antibody molecule).

17. The method of embodiment 16, wherein the T cell population comprises CD4 T cells, CD8 T cells, or CD3 T cells.

18. The method of embodiment 16, wherein the T cell population derived from peripheral blood.

19. The method of any of the preceding embodiments, wherein the anti-TCRβV antibody molecule comprises: (1) a heavy chain complementarity determining region (HC CDR1), a HC CDR2 and/or a HC CDR3 of SEQ ID NO: 3, SEQ ID NO: 4, and/or SEQ ID NO: 5; and/or (2) a light chain complementarity determining region 1 (LC CDR1), a LC CDR2, and/or a LC CDR3 of SEQ ID NO: 6, SEQ ID NO: 7, and/or SEQ ID NO: 8.

20. The method of any of the preceding embodiments, wherein the anti-TCRβV antibody molecule comprises: (1) a heavy chain complementarity determining region (HC CDR1), a HC CDR2 and/or a HC CDR3 of SEQ ID NO: 45, SEQ ID NO: 46, and/or SEQ ID NO: 47; and/or (2) a light chain complementarity determining region 1 (LC CDR1), a LC CDR2, and/or a LC CDR3 of SEQ ID NO: 51, SEQ ID NO: 52, and/or SEQ ID NO: 53.

21. The method of any of the preceding embodiments, wherein the anti-TCRβV antibody molecule comprises: (1) a heavy chain complementarity determining region (HC CDR1), a HC CDR2 and/or a HC CDR3 of SEQ ID NO: 48, SEQ ID NO: 49, and/or SEQ ID NO: 50; and/or (2) a light chain complementarity determining region 1 (LC CDR1), a LC CDR2, and/or a LC CDR3 of SEQ ID NO: 54, SEQ ID NO: 55, and/or SEQ ID NO: 56.

22. The method of any of the preceding embodiments, wherein the anti-TCRβV antibody molecule comprises: (1) a heavy chain complementarity determining region (HC CDR1), a HC CDR2 and/or a HC CDR3 of SEQ ID NO: 17, SEQ ID NO: 18, and/or SEQ ID NO: 19; and/or (2) a light chain complementarity determining region 1 (LC CDR1), a LC CDR2, and/or a LC CDR3 of SEQ ID NO: 20, SEQ ID NO: 21, and/or SEQ ID NO: 22.

23. The method of any of the preceding embodiments, wherein the anti-TCRβV antibody molecule comprises: (1) a heavy chain complementarity determining region (HC CDR1), a HC CDR2 and/or a HC CDR3 of SEQ ID NO: 57, SEQ ID NO: 58, and/or SEQ ID NO: 59; and/or (2) a light chain complementarity determining region 1 (LC CDR1), a LC CDR2, and/or a LC CDR3 of SEQ ID NO: 63, SEQ ID NO: 64, and/or SEQ ID NO: 65.

24. The method of any of the preceding embodiments, wherein the anti-TCRβV antibody molecule comprises: (1) a heavy chain complementarity determining region (HC CDR1), a HC CDR2 and/or a HC CDR3 of SEQ ID NO: 60, SEQ ID NO: 61, and/or SEQ ID NO: 62; and/or (2) a light chain complementarity determining region 1 (LC CDR1), a LC CDR2, and/or a LC CDR3 of SEQ ID NO: 66, SEQ ID NO: 67, and/or SEQ ID NO: 68.

25. The method of any of the preceding embodiments, wherein the anti-TCRβV antibody molecule comprises a VH having at least 85% (e.g., at least 85%, 90%, 95%, 96%, 97%, 98%, 99%, or 100%) sequence identity to SEQ ID NO: 9.

26. The method of any of the preceding embodiments, wherein the anti-TCRβV antibody molecule comprises a VL having at least 85% (e.g., at least 85%, 90%, 95%, 96%, 97%, 98%, 99%, or 100%) sequence identity to SEQ ID NO: 10.

27. The method of any of the preceding embodiments, wherein the anti-TCRβV antibody molecule comprises a VH having at least 85% (e.g., at least 85%, 90%, 95%, 96%, 97%, 98%, 99%, or 100%) sequence identity to SEQ ID NO: 9 and a VL having at least X % sequence identity to SEQ ID NO: 10.

28. The method of any of the preceding embodiments, wherein the anti-TCRβV antibody molecule comprises a heavy chain having at least 85% (e.g., at least 85%, 90%, 95%, 96%, 97%, 98%, 99%, or 100%) sequence identity to SEQ ID NO: 69.

29. The method of any of the preceding embodiments, wherein the anti-TCRβV antibody molecule comprises a heavy chain having at least 85% (e.g., at least 85%, 90%, 95%, 96%, 97%, 98%, 99%, or 100%) sequence identity to SEQ ID NO: 70.

30. The method of any of the preceding embodiments, wherein the anti-TCRβV antibody molecule comprises a heavy chain having at least 85% (e.g., at least 85%, 90%, 95%, 96%, 97%, 98%, 99%, or 100%) sequence identity to SEQ ID NO: 71.

31. The method of any of the preceding embodiments, wherein the anti-TCRβV antibody molecule comprises a light chain having at least 85% (e.g., at least 85%, 90%, 95%, 96%, 97%, 98%, 99%, or 100%) sequence identity to SEQ ID NO: 72.

32. The method of any of the preceding embodiments, wherein the anti-TCRβV antibody molecule comprises a heavy chain having at least 85% (e.g., at least 85%, 90%, 95%, 96%, 97%, 98%, 99%, or 100%) sequence identity to SEQ ID NO: 69 and a light chain having at least 85% (e.g., at least 85%, 90%, 95%, 96%, 97%, 98%, 99%, or 100%) sequence identity to SEQ ID NO: 72.

33. The method of any of the preceding embodiments, wherein the anti-TCRβV antibody molecule comprises a heavy chain having at least 85% (e.g., at least 85%, 90%, 95%, 96%, 97%, 98%, 99%, or 100%) sequence identity to SEQ ID NO: 70 and a light chain having at least 85% (e.g., at least 85%, 90%, 95%, 96%, 97%, 98%, 99%, or 100%) sequence identity to SEQ ID NO: 72.

34. The method of any of the preceding embodiments, wherein the anti-TCRβV antibody molecule comprises a heavy chain having at least 85% (e.g., at least 85%, 90%, 95%, 96%, 97%, 98%, 99%, or 100%) sequence identity to SEQ ID NO: 71 and a light chain having at least 85% (e.g., at least 85%, 90%, 95%, 96%, 97%, 98%, 99%, or 100%) sequence identity to SEQ ID NO: 72.

35. The method of any of the preceding embodiments, wherein the anti-TCRβV antibody molecule is co-expressed with an IgJ chain (e.g., an IgJ chain comprising at least 85%, 90%, 95%, 96%, 97%, 98%, 99%, or 100% sequence identity to SEQ ID NO: 76).

36. The method of any of the preceding embodiments, wherein the anti-TCRβV antibody molecule comprises a heavy chain having at least 85% (e.g., at least 85%, 90%, 95%, 96%, 97%, 98%, 99%, or 100%) sequence identity to SEQ ID NO: 69 and a light chain having at least 85% (e.g., at least 85%, 90%, 95%, 96%, 97%, 98%, 99%, or 100%) sequence identity to SEQ ID NO: 72; and wherein the anti-TCRβV antibody molecule is co-expressed with an IgJ chain (e.g., an IgJ chain comprising at least 85%, 90%, 95%, 96%, 97%, 98%, 99%, or 100% sequence identity to SEQ ID NO: 76).

37. The method of any of the preceding embodiments, wherein the anti-TCRβV antibody molecule comprises a VH having at least 85% (e.g., at least 85%, 90%, 95%, 96%, 97%, 98%, 99%, or 100%) sequence identity to SEQ ID NO: 15.

38. The method of any of the preceding embodiments, wherein the anti-TCRβV antibody molecule comprises a VL having at least 85% (e.g., at least 85%, 90%, 95%, 96%, 97%, 98%, 99%, or 100%) sequence identity to SEQ ID NO: 16.

39. The method of any of the preceding embodiments, wherein the anti-TCRβV antibody molecule comprises a VH having at least 85% (e.g., at least 85%, 90%, 95%, 96%, 97%, 98%, 99%, or 100%) sequence identity to SEQ ID NO: 23.

40. The method of any of the preceding embodiments, wherein the anti-TCRβV antibody molecule comprises a VH having at least 85% (e.g., at least 85%, 90%, 95%, 96%, 97%, 98%, 99%, or 100%) sequence identity to SEQ ID NO: 24.

41. The method of any of the preceding embodiments, wherein the anti-TCRβV antibody molecule comprises a VH having at least 85% (e.g., at least 85%, 90%, 95%, 96%, 97%, 98%, 99%, or 100%) sequence identity to SEQ ID NO: 25.

42. The method of any of the preceding embodiments, wherein the anti-TCRβV antibody molecule comprises a VL having at least 85% (e.g., at least 85%, 90%, 95%, 96%, 97%, 98%, 99%, or 100%) sequence identity to SEQ ID NO: 26.

43. The method of any of the preceding embodiments, wherein the anti-TCRβV antibody molecule comprises a VL having at least 85% (e.g., at least 85%, 90%, 95%, 96%, 97%, 98%, 99%, or 100%) sequence identity to SEQ ID NO: 27.

44. The method of any of the preceding embodiments, wherein the anti-TCRβV antibody molecule comprises a VL having at least 85% (e.g., at least 85%, 90%, 95%, 96%, 97%, 98%, 99%, or 100%) sequence identity to SEQ ID NO: 28.

45. The method of any of the preceding embodiments, wherein the anti-TCRβV antibody molecule comprises a VL having at least 85% (e.g., at least 85%, 90%, 95%, 96%, 97%, 98%, 99%, or 100%) sequence identity to SEQ ID NO: 29.

46. The method of any of the preceding embodiments, wherein the anti-TCRβV antibody molecule comprises a VL having at least 85% (e.g., at least 85%, 90%, 95%, 96%, 97%, 98%, 99%, or 100%) sequence identity to SEQ ID NO: 30.

47. The method of any of the preceding embodiments, wherein the anti-TCRβV antibody molecule comprises a VH amino acid sequence as listed in Table 3 or Table 4, and/or a VL amino acid sequence as listed in Table 3 or Table 4.

48. The method of any of the preceding embodiments, wherein the anti-TCRβV antibody molecule selectively or preferentially expands αβ T cels over y6 T cells.

49. The method of any of the preceding embodiments, wherein the anti-TCRβV antibody molecule does not induce cytokine release syndrome (CRS).

50. The method of any of the preceding embodiments, wherein binding of the anti-TCRβV antibody molecule to the TCRβV region results in one, two, three, four, five, six, seven, eight, nine, ten or more (e.g., all) of the following:
(i) reduced level, e.g., expression level, and/or activity of IL-1β;
(ii) reduced level, e.g., expression level, and/or activity of IL-6;
(iii) reduced level, e.g., expression level, and/or activity of TNFα;
(iv) increased level, e.g., expression level, and/or activity of IL-2;
(v) a delay, e.g., at least 1, 2, 3, 4, 5, 6, 7, 8, 9, 10 or more hours delay, in increased level, e.g., expression level, and/or activity of IL-2;
(vi) a delay, e.g., at least 1, 2, 3, 4, 5, 6, 7, 8, 9, 10 hours delay, in increased level, e.g., expression level, and/or activity of IFNγ;
(vii) reduced T cell proliferation kinetics; or
(viii) reduced cytokine storm, e.g., cytokine release syndrome (CRS), e.g., as measured by an assay of Example 3;
(ix) cell killing, e.g., target cell killing,
(x) increased level, e.g., expression level, and/or activity of IL-15; or
(xi) increased Natural Killer (NK) cell proliferation, e.g., expansion, compared to an antibody that binds to: a CD3 molecule, e.g., CD3 epsilon (CD3e) molecule; or a TCR alpha (TCRα) molecule,
wherein the anti-TCRβV antibody molecule:
(1) does not bind to TCRβ V12, TCRβ V5-5*01 or TCRβ V5-1*01;
(2) binds to TCRβ V12 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of the murine mAb Antibody B; and/or
(3) binds to TCRβ V5-5*01 TCRβ V5-1*01 or with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of murine Antibody C.

51. The method of any of the preceding embodiments, wherein binding of the anti-TCRβV antibody molecule to the TCRβV region results in expansion, e.g., at least about 1.1-10 fold expansion (e.g., at least about 1.1, 1.2, 1.3, 1.4, 1.5, 2, 3, 4, 5, 6, 7, 8, 9, or 10 fold expansion), of a population of memory T cells, e.g., T effector memory (T_{EM}) cells, e.g., T_{EM} cells expressing CD45RA (T_{EMRA}) cells, wherein the anti-TCRβV antibody molecule:
(1) does not bind to TCRβ V12, TCRβ V5-5*01 or TCRβ V5-1*01;
(2) binds to TCRβ V12 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of the murine mAb Antibody B; and/or
(3) binds to TCRβ V5-5*01 TCRβ V5-1*01or with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of murine Antibody C.

52. The method of any of the preceding embodiments, wherein binding of the anti-TCRβV antibody molecule to a TCRβV region results in a reduction of at least 2, 5, 10, 20, 50, 100, or 200 fold, or at least 2-200 fold (e.g., 5-150, 10-100, 20-50 fold) in the expression level and or activity of IL-1β as measured by an assay of Example 3.

53. The method of any of the preceding embodiments, wherein binding of the anti-TCRβV antibody molecule to a TCRβV region results in a reduction of at least 2, 5, 10, 20, 50, 100, 200, 300, 400, 500, 600, 700, 800, 900, or 1000 fold, or at least 2-1000 fold (e.g., 5-900, 10-800, 20-700, 50-600, 100-500, or 200-400 fold) in the expression level and or activity of IL-6 as measured by an assay of Example 3.

54. The method of any of the preceding embodiments, wherein binding of the anti-TCRβV antibody molecule to a TCRβV region results in a reduction of at least 2, 5, 10, 20, 50, 100, 200, 300, 400, 500, 600, 700, 800, 900, 1000, or 2000 fold, or at least 2-2000 fold (e.g., 5-1000, 10-900, 20-800, 50-700, 100-600, 200-500, or 300-400 fold) in the expression level and or activity of TNFα as measured by an assay of Example 3.

55. The method of any of the preceding embodiments, wherein binding of the anti-TCRβV antibody molecule to a TCRβV region results in an increase of at least 2, 5, 10, 20, 50, 100, 200, 300, 400, 500, 600, 700, 800, 900, 1000, or 2000 fold, or at least 2-2000 fold (e.g., 5-1000, 10-900, 20-800, 50-700, 100-600, 200-500, or 300-400 fold) in the expression level and or activity of IL-2 as measured by an assay of Example 3.

56. The method of any of the preceding embodiments, wherein the anti-TCRβV antibody molecule binds to one or more (e.g., all) of the following TCRβV subfamilies:
(i) TCRβ V6 subfamily comprising, e.g., TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01;
(ii) TCRβ V10 subfamily comprising, e.g., TCRβ V10-1*01, TCRβ V10-1*02, TCRβ V10-3*01 or TCRβ V10-2*01;
(iii) TCRβ V12 subfamily comprising, e.g., TCRβ V12-4*01, TCRβ V12-3*01, or TCRβ V12-5*01;
(iv) TCRβ V5 subfamily comprising, e.g., TCRβ V5-5*01, TCRβ V5-6*01, TCRβ V5-4*01, TCRβ V5-8*01, or TCRβ V5-1*01;
(v) TCRβ V7 subfamily comprising, e.g., TCRβ V7-7*01, TCRβ V7-6*01, TCRβ V7-8*02, TCRβ V7-4*01, TCRβ V7-2*02, TCRβ V7-2*03, TCRβ V7-2*01, TCRβ V7-3*01, TCRβ V7-9*03, or TCRβ V7-9*01;
(vi) TCRβ V11 subfamily comprising, e.g., TCRβ V11-1*01, TCRβ V11-2*01 or TCRβ V11-3*01;
(vii) TCRβ V14 subfamily comprising, e.g., TCRβ V14*01;
(viii) TCRβ V16 subfamily comprising, e.g., TCRβ V16*01;
(ix) TCRβ V18 subfamily comprising, e.g., TCRβ V18*01;
(x) TCRβ V9 subfamily comprising, e.g., TCRβ V9*01 or TCRβ V9*02;
(xi) TCRβ V13 subfamily comprising, e.g., TCRβ V13*01;
(xii) TCRβ V4 subfamily comprising, e.g., TCRβ V4-2*01, TCRβ V4-3*01, or TCRβ V4-1*01;
(xiii) TCRβ V3 subfamily comprising, e.g., TCRβ V3-1*01;
(xiv) TCRβ V2 subfamily comprising, e.g., TCRβ V2*01;
(xv) TCRβ V15 subfamily comprising, e.g., TCRβ V15*01;
(xvi) TCRβ V30 subfamily comprising, e.g., TCRβ V30*01, or TCRβ V30*02;
(xvii) TCRβ V19 subfamily comprising, e.g., TCRβ V19*01, or TCRβ V19*02;
(xviii) TCRβ V27 subfamily comprising, e.g., TCRβ V27*01;
(xix) TCRβ V28 subfamily comprising, e.g., TCRβ V28*01;
(xx) TCRβ V24 subfamily comprising, e.g., TCRβ V24-1*01;
(xxi) TCRβ V20 subfamily comprising, e.g., TCRβ V20-1*01, or TCRβ V20-1*02;
(xxii) TCRβ V25 subfamily comprising, e.g., TCRβ V25-1*01;
(xxiii) TCRβ V29 subfamily comprising, e.g., TCRβ V29-1*01; or
(xxiv) TCRβ V23 subfamily comprising, e.g., TCRβ V23-1.

57. The method of any of the preceding embodiments, wherein the anti-TCRβV antibody molecule binds to one or more (e.g., all) of the following TCRβV subfamilies:
(i) TCRβ V6 subfamily comprising, e.g., TCRβ V6-5*01;
(ii) TCRβ V10 subfamily comprising, e.g., TCRβ V10-1*01, TCRβ V10-1*02, TCRβ V10-3*01 or TCRβ V10-2*01;
(iii) TCRβ V12 subfamily comprising, e.g., TCRβ V12-4*01, TCRβ V12-3*01, or TCRβ V12-5*01;
(iv) TCRβ V5 subfamily comprising, e.g., TCRβ V5-6*01;
(v) TCRβ V7 subfamily comprising, e.g., TCRβ V7-7*01, TCRβ V7-6*01, TCRβ V7-8*02, TCRβ V7-4*01, TCRβ V7-2*02, TCRβ V7-2*03, TCRβ V7-2*01, TCRβ V7-3*01, TCRβ V7-9*03, or TCRβ V7-9*01;
(vi) TCRβ V14 subfamily comprising, e.g., TCRβ V14*01;
(vii) TCRβ V9 subfamily comprising, e.g., TCRβ V9*01 or TCRβ V9*02;
(viii) TCRβ V13 subfamily comprising, e.g., TCRβ V13*01;
(ix) TCRβ V19 subfamily comprising, e.g., TCRβ V19*01, or TCRβ V19*02; or
(x) TCRβ V23 subfamily comprising, e.g., TCRβ V23-1.

58. The method of any of the preceding embodiments, wherein the infectious disease is SIV and the anti-TCRβV antibody molecule binds to the TCRβ V6 subfamily, e.g., comprising TCRβ V6-5*01.

59. The method of embodiment 58, wherein the subject has a higher, e.g., increased, level or activity of a TCRβ V6 subfamily, e.g., comprising TCRβ V6-5*01.

60. The method of any of the preceding embodiments, wherein the infectious disease is HCMV and the anti-TCRβV antibody molecule binds to the TCRβ V6 subfamily, e.g., comprising TCRβ V6-5*01.

61. The method of embodiment 60, wherein the subject has a higher, e.g., increased, level or activity of a TCRβ V6 subfamily, e.g., comprising TCRβ V6-5*01.

62. The method of any of embodiments 58-61, wherein the anti-TCRβV antibody molecule comprises:
(1) a heavy chain complementarity determining region (HC CDR1), a HC CDR2 and/or a HC CDR3 of SEQ ID NO: 3, SEQ ID NO: 4, and/or SEQ ID NO: 5; and/or
(2) a light chain complementarity determining region 1 (LC CDR1), a LC CDR2, and/or a LC CDR3 of SEQ ID NO: 6, SEQ ID NO: 7, and/or SEQ ID NO: 8.

63. The method of any of embodiments 58-61, wherein the anti-TCRβV antibody molecule comprises:
(1) a heavy chain complementarity determining region (HC CDR1), a HC CDR2 and/or a HC CDR3 of SEQ ID NO: 45, SEQ ID NO: 46, and/or SEQ ID NO: 47; and/or
(2) a light chain complementarity determining region 1 (LC CDR1), a LC CDR2, and/or a LC CDR3 of SEQ ID NO: 51, SEQ ID NO: 52, and/or SEQ ID NO: 53.

64. The method of any of embodiments 58-61, wherein the anti-TCRβV antibody molecule comprises:
(1) a heavy chain complementarity determining region (HC CDR1), a HC CDR2 and/or a HC CDR3 of SEQ ID NO: 48, SEQ ID NO: 49, and/or SEQ ID NO: 50; and/or
(2) a light chain complementarity determining region 1 (LC CDR1), a LC CDR2, and/or a LC CDR3 of SEQ ID NO: 54, SEQ ID NO: 55, and/or SEQ ID NO: 56.

65. The method of any of embodiments 58-64, wherein the anti-TCRβV antibody molecule comprises a VH having at least 85% (e.g., at least 85%, 90%, 95%, 96%, 97%, 98%, 99%, or 100%) sequence identity to SEQ ID NO: 9.

66. The method of any of embodiments 58-65, wherein the anti-TCRβV antibody molecule comprises a VL having at least 85% (e.g., at least 85%, 90%, 95%, 96%, 97%, 98%, 99%, or 100%) sequence identity to SEQ ID NO: 10.

67. The method of any of embodiments 58-64, wherein the anti-TCRβV antibody molecule comprises a VH having at least 85% (e.g., at least 85%, 90%, 95%, 96%, 97%, 98%, 99%, or 100%) sequence identity to SEQ ID NO: 9 and a VL having at least X % sequence identity to SEQ ID NO: 10.

68. The method of any of embodiments 58-67, wherein the anti-TCRβV antibody molecule comprises a heavy chain having at least 85% (e.g., at least 85%, 90%, 95%, 96%, 97%, 98%, 99%, or 100%) sequence identity to SEQ ID NO: 69 and a light chain having at least 85% (e.g., at least 85%, 90%, 95%, 96%, 97%, 98%, 99%, or 100%) sequence identity to SEQ ID NO: 72.

69. The method of any of the preceding embodiments, wherein the infectious disease is EBV and the anti-TCRβV antibody molecule binds to the TCRβ V10 subfamily, e.g., comprising TCRβ V10-1*01, TCRβ V10-1*02, TCRβ V10-3*01 or TCRβ V10-2*01.

70. The method of embodiment 69, wherein the antigen is BZLF1(52-64).

71. The method of embodiment 69 or 70, wherein the MHC restriction is HLA-B*3508.

72. The method of any of embodiments 69-71, wherein the subject has a higher, e.g., increased, level or activity of a TCRβ V10 subfamily, e.g., comprising TCRβ V10-1*01, TCRβ V10-1*02, TCRβ V10-3*01 or TCRβ V10-2*01.

73. The method of any of the preceding embodiments, wherein the infectious disease is malaria and the anti-TCRβV antibody molecule binds to the TCRβ V12 subfamily, e.g., comprising TCRβ V12-4*01, TCRβ V12-3*01, or TCRβ V12-5*01.

74. The method of embodiment 73, wherein the subject has a higher, e.g., increased, level or activity of a TCRβ V12 subfamily, e.g., comprising TCRβ V12-4*01, TCRβ V12-3*01, or TCRβ V12-5*01.

75. The method of any of the preceding embodiments, wherein the infectious disease is tuberculosis and the anti-TCRβV antibody molecule binds to the TCRβ V12 subfamily, e.g., comprising TCRβ V12-4*01, TCRβ V12-3*01, or TCRβ V12-5*01.

76. The method of embodiment 75, wherein the subject has a higher, e.g., increased, level or activity of a TCRβ V12 subfamily, e.g., comprising TCRβ V12-4*01, TCRβ V12-3*01, or TCRβ V12-5*01.

77. The method of any of the preceding embodiments, wherein the infectious disease is HCMV and the anti-TCRβV antibody molecule binds to the TCRβ V12 subfamily, e.g., comprising TCRβ V12-4*01.

78. The method of embodiment 77, wherein the subject has a higher, e.g., increased, level or activity of a TCRβ V12 subfamily, e.g., comprising TCRβ V12-4*01.

79. The method of any of embodiments 73-78, wherein the anti-TCRβV antibody molecule comprises:
(1) a heavy chain complementarity determining region (HC CDR1), a HC CDR2 and/or a HC CDR3 of SEQ ID NO: 17, SEQ ID NO: 18, and/or SEQ ID NO: 19; and/or
(2) a light chain complementarity determining region 1 (LC CDR1), a LC CDR2, and/or a LC CDR3 of SEQ ID NO: 20, SEQ ID NO: 21, and/or SEQ ID NO: 22.

80. The method of any of embodiments 73-78, wherein the anti-TCRβV antibody molecule comprises:
(1) a heavy chain complementarity determining region (HC CDR1), a HC CDR2 and/or a HC CDR3 of SEQ ID NO: 57, SEQ ID NO: 58, and/or SEQ ID NO: 59; and/or
(2) a light chain complementarity determining region 1 (LC CDR1), a LC CDR2, and/or a LC CDR3 of SEQ ID NO: 63, SEQ ID NO: 64, and/or SEQ ID NO: 65.

81. The method of any of embodiments 73-78, wherein the anti-TCRβV antibody molecule comprises:
(1) a heavy chain complementarity determining region (HC CDR1), a HC CDR2 and/or a HC CDR3 of SEQ ID NO: 60, SEQ ID NO: 61, and/or SEQ ID NO: 62; and/or
(2) a light chain complementarity determining region 1 (LC CDR1), a LC CDR2, and/or a LC CDR3 of SEQ ID NO: 66, SEQ ID NO: 67, and/or SEQ ID NO: 68.

82. The method of any of embodiments 73-81, wherein the anti-TCRβV antibody molecule comprises a VH having at least 85% (e.g., at least 85%, 90%, 95%, 96%, 97%, 98%, 99%, or 100%) sequence identity to SEQ ID NO: 15.

83. The method of any of embodiments 73-82, wherein the anti-TCRβV antibody molecule comprises a VL having at least 85% (e.g., at least 85%, 90%, 95%, 96%, 97%, 98%, 99%, or 100%) sequence identity to SEQ ID NO: 16,
optionally wherein the anti-TCRβV antibody molecule comprises a VH having at least 85% (e.g., at least 85%, 90%, 95%, 96%, 97%, 98%, 99%, or 100%) sequence identity to SEQ ID NO: 15 and a VL having at least 85% (e.g., at least 85%, 90%, 95%, 96%, 97%, 98%, 99%, or 100%) sequence identity to SEQ ID NO: 16.

84. The method of any of embodiments 73-81, wherein the anti-TCRβV antibody molecule comprises a VH having at least 85% (e.g., at least 85%, 90%, 95%, 96%, 97%, 98%, 99%, or 100%) sequence identity to SEQ ID NO: 23.

85. The method of any of embodiments 73-81, wherein the anti-TCRβV antibody molecule comprises a VH having at least 85% (e.g., at least 85%, 90%, 95%, 96%, 97%, 98%, 99%, or 100%) sequence identity to SEQ ID NO: 24.

86. The method of any of embodiments 73-81, wherein the anti-TCRβV antibody molecule comprises a VH having at least 85% (e.g., at least 85%, 90%, 95%, 96%, 97%, 98%, 99%, or 100%) sequence identity to SEQ ID NO: 25.

87. The method of any of embodiments 73-86, wherein the anti-TCRβV antibody molecule comprises a VL having at least 85% (e.g., at least 85%, 90%, 95%, 96%, 97%, 98%, 99%, or 100%) sequence identity to SEQ ID NO: 26.

88. The method of any of embodiments 73-86, wherein the anti-TCRβV antibody molecule comprises a VL having at least 85% (e.g., at least 85%, 90%, 95%, 96%, 97%, 98%, 99%, or 100%) sequence identity to SEQ ID NO: 27.

89. The method of any of embodiments 73-86, wherein the anti-TCRβV antibody molecule comprises a VL having at least 85% (e.g., at least 85%, 90%, 95%, 96%, 97%, 98%, 99%, or 100%) sequence identity to SEQ ID NO: 28.

90. The method of any of embodiments 73-86, wherein the anti-TCRβV antibody molecule comprises a VL having at least 85% (e.g., at least 85%, 90%, 95%, 96%, 97%, 98%, 99%, or 100%) sequence identity to SEQ ID NO: 29.

91. The method of any of embodiments 73-86, wherein the anti-TCRβV antibody molecule comprises a VL having at least 85% (e.g., at least 85%, 90%, 95%, 96%, 97%, 98%, 99%, or 100%) sequence identity to SEQ ID NO: 30.

92. The method of any of the preceding embodiments, wherein the infectious disease is HIV and the anti-TCRβV antibody molecule binds to the TCRβ V5 subfamily, e.g., comprising TCRβ V5-6*01.

93. The method of embodiment 92, wherein the antigen is Gag p17 (77-85).

94. The method of embodiment 92 or 93, wherein the MHC restriction is HLA-B*0801

95. The method of any of embodiments 92-94, wherein the subject has a higher, e.g., increased, level or activity of a TCRβ V5 subfamily, e.g., comprising TCRβ V5-6*01.

96. The method of any of the preceding embodiments, wherein the infectious disease is EBV and the anti-TCRβV antibody molecule binds to the TCRβ V7 subfamily, e.g., comprising TCRβ V7-7*01, TCRβ V7-6*01, TCRβ V7-8*02, TCRβ V7-4*01, TCRβ V7-2*02, TCRβ V7-2*03, TCRβ V7-2*01, TCRβ V7-3*01, TCRβ V7-9*03, or TCRβ V7-9*01.

97. The method of embodiment 96, wherein the antigen is EBNA3(339-347).

98. The method of embodiment 96 or 97, wherein the MHC restriction is HLA-B*0801.

99. The method of any of embodiments 96-98, wherein the subject has a higher, e.g., increased, level or activity of a TCRβ V7 subfamily, e.g., comprising TCRβ V7-7*01, TCRβ V7-6*01, TCRβ V7-8*02, TCRβ V7-4*01, TCRβ V7-2*02, TCRβ V7-2*03, TCRβ V7-2*01, TCRβ V7-3*01, TCRβ V7-9*03, or TCRβ V7-9*01.

100. The method of any of the preceding embodiments, wherein the infectious disease is SIV and the anti-TCRβV antibody molecule binds to the TCRβ V14 subfamily, e.g., comprising TCRβ V14*01.

101. The method of embodiment 100, wherein the subject has a higher, e.g., increased, level or activity of a TCRβ V14 subfamily, e.g., comprising TCRβ V14*01.

102. The method of any of the preceding embodiments, wherein the infectious disease is EBV and the anti-TCRβV antibody molecule binds to the TCRβ V9 subfamily, e.g., comprising TCRβ V9*01 or TCRβ V9*02.

103. The method of embodiment 102, wherein the antigen is EBNA1(407-417).

104. The method of embodiment 102 or 103, wherein the MHC restriction is HLA-B*3508 or HLA-B*3501.

105. The method of any of embodiments 102-104, wherein the subject has a higher, e.g., increased, level or activity of a TCRβ V9 subfamily, e.g., comprising TCRβ V9*01 or

106. The method of any of the preceding embodiments, wherein the infectious disease is SIV and the anti-TCRβ V antibody molecule binds to the TCRβ V13 subfamily, e.g., comprising TCRβ V13*01.

107. The method of embodiment 106, wherein the subject has a higher, e.g., increased, level or activity of a TCRβ V13 subfamily, e.g., comprising TCRβ V13*01.

108. The method of any of the preceding embodiments, wherein the infectious disease is influenza and the anti-TCRβ V antibody molecule binds to the TCRβ V19 subfamily, e.g., comprising TCRβ V19*01, or TCRβ V19*02.

109. The method of embodiment 108, wherein the antigen is Matrix protein (58-66).

110. The method of embodiment 108 or 109, wherein the MHC restriction is HLA-A2.

111. The method of any of embodiments 108-110, wherein the subject has a higher, e.g., increased, level or activity of a TCRβ V19 subfamily, e.g., comprising TCRβ V19*01, or TCRβ V19*02.

112. The method of any of the preceding embodiments, wherein the infectious disease is HIV and the anti-TCRβV antibody molecule binds to the TCRβ V19 subfamily, e.g., comprising TCRβ V19*01, or TCRβ V19*02.

113. The method of embodiment 112, wherein the subject has a higher, e.g., increased, level or activity of a TCRβ V19 subfamily, e.g., comprising TCRβ V19*01, or TCRβ V19*02.

114. The method of any of the preceding embodiments, wherein the infectious disease is HIV and the anti-TCRβV antibody molecule binds to the TCRβ V23 subfamily, e.g., comprising TCRβ V23-1.

115. The method of embodiment 114, wherein the subject has a higher, e.g., increased, level or activity of a TCRβ V23 subfamily, e.g., comprising TCRβ V23-1.

116. The method of any of the preceding embodiments, wherein the anti-TCRβ V antibody molecule:
(i) binds specifically to an epitope on TCRβ V, e.g., the same or similar epitope as the epitope recognized by an anti-TCRβ V antibody molecule as described herein, e.g., a second anti-TCRβ V antibody molecule;
(ii) shows the same or similar binding affinity or specificity, or both, as an anti-TCRβ V antibody molecule as described herein, e.g., a second anti-TCRβ V antibody molecule;
(iii) inhibits, e.g., competitively inhibits, the binding of an anti-TCRβV antibody molecule as described herein, e.g., a second anti-TCRβV antibody molecule;
(iv) binds the same or an overlapping epitope with an anti-TCRβV antibody molecule as described herein, e.g., a second anti-TCRβV antibody molecule; or
(v) competes for binding, and/or binds the same epitope, with an anti-TCRβV antibody molecule as described herein, e.g., a second anti-TCRβV antibody molecule,

117. The method of any of the preceding embodiments, wherein the anti-TCRβV antibody molecule comprises an antigen binding domain comprising:
(i) a heavy chain complementarity determining region 1 (HC CDR1), a heavy chain complementarity determining region 2 (HC CDR2) and/or a heavy chain complementarity determining region 3 (HC CDR3) of SEQ ID NO: 1 or SEQ ID NO: 9; or
(ii) a light chain complementarity determining region 1 (LC CDR1), a light chain complementarity determining region 2 (LC CDR2), and/or a light chain complementarity determining region 3 (LC CDR3) of SEQ ID NO: 2, SEQ ID NO: 10, or SEQ ID NO: 11.

118. The method of any of the preceding embodiments, wherein the anti-TCRβV antibody molecule comprises an antigen binding domain comprising a light chain variable region (VL) comprising one, two or all (e.g., three) of a LC CDR1, a LC CDR2 and a LC CDR3 of SEQ ID NO: 2, SEQ ID NO: 10, or SEQ ID NO: 11.

119. The method of any of the preceding embodiments, wherein the anti-TCRβV antibody molecule comprises an antigen binding domain comprising a heavy chain variable region (VH) comprising one, two or all (e.g., three) of a HC CDR1, a HC CDR2 and a HC CDR3 of SEQ ID NO:1 or SEQ ID NO: 9.

120. The method of any of the preceding embodiments, wherein the anti-TCRβV antibody molecule comprises an antigen binding domain comprising:
(i) a VL comprising: a LC CDR1 amino acid sequence of SEQ ID NO: 6 (or an amino acid sequence with not more than 1, 2, 3 or 4 modifications, e.g., substitutions, additions or deletions thereof), a LC CDR2 amino acid sequence of SEQ ID NO:7 (or an amino acid sequence with not more than 1, 2, 3 or 4 modifications, e.g., substitutions, additions or deletions thereof), and/or a LC CDR3 amino acid sequence of SEQ ID NO:8 (or an amino acid sequence with not more than 1, 2, 3 or 4 modifications, e.g., substitutions, additions or deletions thereof); and/or
(ii) a VH comprising: a HC CDR1 amino acid sequence of SEQ ID NO: 3 (or an amino acid sequence with not more than 1, 2, 3 or 4 modifications, e.g., substitutions, additions or deletions thereof), a HC CDR2 amino acid sequence of SEQ ID NO:4 (or an amino acid sequence with not more than 1, 2, 3 or 4 modifications, e.g., substitutions, additions or deletions thereof), and/or a HC CDR3 amino acid sequence of SEQ ID NO:5 (or an amino acid sequence with not more than 1, 2, 3 or 4 modifications, e.g., substitutions, additions or deletions thereof).

121. The method of any of the preceding embodiments, wherein the anti-TCRβV antibody molecule comprises an antigen binding domain comprising: a variable heavy chain (VH) of SEQ ID NO: 9, or a sequence having at least about 85%, 90%, 95%, or 99% sequence identity thereto; and/or a variable light chain (VL) of SEQ ID NO: 10 or SEQ ID NO: 11, or a sequence having at least about 85%, 90%, 95%, or 99% sequence identity thereto.

122. The method of any of the preceding embodiments, wherein the anti-TCRβV antibody molecule comprises an antigen binding domain comprising the VH amino acid sequence of SEQ ID NO: 9 and the VL amino acid sequence of SEQ ID NO: 10.

123. The method of any of the preceding embodiments, wherein the anti-TCRβV antibody molecule comprises an antigen binding domain comprising the VH amino acid sequence of SEQ ID NO: 9 and the VL amino acid sequence of SEQ ID NO: 11.

124. The method of any of the preceding embodiments, wherein the anti-TCRβV antibody molecule comprises an antigen binding domain comprising a single chain Fv (scFv) or a Fab.

125. The method of any of the preceding embodiments, wherein the anti-TCRβV antibody molecule binds to a conformational or a linear epitope on the T cell.

126. The method of any of the preceding embodiments, wherein the anti-TCRβV antibody molecule is a full antibody (e.g., an antibody that includes at least one, and preferably two, complete heavy chains, and at least one, and preferably two, complete light chains), or an antigen-binding fragment (e.g., a Fab, F(ab′)₂, Fv, a single chain Fv fragment, a single domain antibody, a diabody (dAb), a bivalent antibody, or bispecific antibody or fragment thereof, a single domain variant thereof, or a camelid antibody).

127. The method of embodiment 126, wherein the anti-TCRβV antibody molecule comprises a heavy chain constant region chosen from IgG1, IgG2, IgG3, or IgG4, or a fragment thereof.

128. The method of embodiment 126 or 127, wherein the anti-TCRβV antibody molecule comprises a light chain constant region chosen from the light chain constant regions of kappa or lambda, or a fragment thereof.

129. A method of making, e.g., producing or manufacturing, the anti-TCRβV antibody molecule of the method of any of the preceding embodiments, comprising culturing a host cell comprising a nucleic acid encoding the anti-TCRβV antibody molecule, under suitable conditions, e.g., conditions suitable expression of the anti- TCRβV antibody molecule.

130. A pharmaceutical composition comprising the anti-TCRβV antibody molecule of the method of any of the preceding embodiments, and a pharmaceutically acceptable carrier, excipient, or stabilizer.

131. The method of any of embodiments 1-128, wherein the expansion occurs in vivo or ex vivo (e.g., in vitro).

132. The method of any of embodiments 1-128 or 131, wherein the T cell population comprises a T cell, a Natural Killer cell, a B cell, or a myeloid cell.

133. The method of any of embodiments 1-128, 131, or 132, wherein the T cell population comprises a CD4 T cell, a CD8 T cell, e.g., an effector T cell or a memory T cell (e.g., a memory effector T cell (e.g., T_{EM} cell, e.g., T_{EMRA} cell), or a combination thereof.

134. The method of any of embodiments 1-128 or 131-133, wherein the T cell population is obtained from a healthy subject.

135. The method of any of embodiments 1-128 or 131-134, wherein the T cell population is obtained from a subject (e.g., from an apheresis sample from the subject) having a disease, e.g., an infectious disease, e.g., as described herein.

136. The method of any of embodiments 1-128 or 131-135, wherein the method results in an expansion of at least 1.1-10 fold (e.g., at least 1.1, 1.2, 1.3, 1.4, 1.5, 2, 3, 4, 5, 6, 7, 8, 9, or 10 fold expansion).

137. The method of any of embodiments 1-128 or 131-136, further comprising contacting the population of cells with an agent that promotes, e.g., increases, immune cell (e.g., T cell) expansion.

138. The method of any of embodiments 1-128 or 131-137, further comprising contacting the population of cells with an additional therapeutic agent.

139. The method of embodiment 138, wherein the additional therapeutic agent targets the infectious disease.

140. The method of any of embodiments 1-128 or 131-139, further comprising contacting the population of cells with a non-dividing population of cells, e.g., feeder cells, e.g., irradiated allogenic human PBMCs.

141. The method of any of embodiments 1-128 or 131-140, wherein the population of cells is expanded in an appropriate media (e.g., media described herein) that includes one or more cytokines, e.g., IL-2, IL-7, IL-15, or a combination thereof.

142. The method of any of embodiments 1-128 or 131-141, wherein the population of cells is expanded for a period of at least about 4 hours, 6 hours, 10 hours, 12 hours, 15 hours, 18 hours, 20 hours, or 22 hours, or for at least 1, 2, 3, 4, 5, 6, 7, 8, 9, 10, 11, 12, 13, 14, 15, 1,6 17, 18, 19, 20 or 21 days, or for at least about 1 week, 2 weeks, 3 weeks, 4 weeks, 5 weeks, 6 weeks, 7 weeks or 8 weeks.

143. The method of any of embodiments 1-128 or 131-142, wherein expansion of the population of T cells is compared to expansion of a similar population of cells with an antibody that binds to: a CD3 molecule, e.g., CD3 epsilon (CD3e) molecule; or a TCR alpha (TCRα) molecule.

144. The method of any of embodiments 1-128 or 131-143, wherein expansion of the population of T cells is compared to expansion of a similar population of cells not contacted with the anti-TCRβV antibody molecule.

145. The method of any of embodiments 1-128 or 131-144, wherein expansion of the population of T cells, e.g., memory effector T cells, e.g., T_{EM} cells, e.g., T_{EMRA} cells, is compared to expansion of a similar population of cells with an antibody that binds to: a CD3 molecule, e.g., CD3 epsilon (CD3e) molecule; or a TCR alpha (TCRα) molecule.

146. The method of any of embodiments 1-128 or 131-145, wherein the population of expanded T cells, e.g., expanded T effector memory cells, comprises cells which: (i) have a detectable level of CD45RA, e.g., express or re-express CD45RA; (ii) have low or no expression of CCR7; and/or (iii) have a detectable level of CD95, e.g., express CD95, e.g., a population of CD45RA+, CCR7−, CD95+ T cells, optionally wherein the T cells comprise CD3+, CD4+or CD8+T cells.

147. The method of any of embodiments 1-128 or 131-146, wherein the antibody molecule, e.g., humanized antibody molecule, which binds, e.g., specifically binds, to the TCRβV region (the anti-TCRβV antibody molecule) is chosen from:
(A) a humanized antibody molecule which binds, e.g., specifically binds, to a T cell receptor beta variable chain (TCRβV) region chosen from TCRβV V5-6, TCRβV V6-5, TCRβV V7, TCRβV V9, TCRβV V10, TCRβV V12 (e.g., TCRβV V12-4), TCRβV V13, TCRβV V14, TCRβV V19, TCR3V V23-1, or a subfamily member thereof (e.g., as listed in Table 1 or Table 2)
(B) a humanized antibody molecule which:
(i) binds specifically to an epitope on TCRβV, e.g., the same or similar epitope as the epitope recognized by a second anti-TCRβV antibody molecule;
(ii) shows the same or similar binding affinity or specificity, or both, as a second anti-TCRβV antibody molecule;
(iii) inhibits, e.g., competitively inhibits, the binding of a second anti-TCRβV antibody molecule;
(iv) binds the same or an overlapping epitope with an anti-TCRβV antibody molecule as a second anti-TCRβV antibody molecule; or
(v) competes for binding, and/or binds the same epitope, with a second anti-TCRβV antibody molecule,
wherein the second anti-TCRβV antibody molecule comprises an antigen binding domain comprising:
(1) a heavy chain complementarity determining region (HC CDR1), a HC CDR2 and/or a HC CDR3 of SEQ ID NO: 3, SEQ ID NO: 4, and/or SEQ ID NO: 5, and/or
(2) a light chain complementarity determining region 1 (LC CDR1), a LC CDR2, and/or a LC CDR3 of SEQ ID NO: 6, SEQ ID NO: 7, and/or SEQ ID NO: 8;
(1) a heavy chain complementarity determining region (HC CDR1), a HC CDR2 and/or a HC CDR3 of SEQ ID NO: 45, SEQ ID NO: 46, and/or SEQ ID NO: 47, and/or
(2) a light chain complementarity determining region 1 (LC CDR1), a LC CDR2, and/or a LC CDR3 of SEQ ID NO: 51, SEQ ID NO: 52, and/or SEQ ID NO: 53;
(1) a heavy chain complementarity determining region (HC CDR1), a HC CDR2 and/or a HC CDR3 of SEQ ID NO: 48, SEQ ID NO: 49, and/or SEQ ID NO: 50, and/or
(2) a light chain complementarity determining region 1 (LC CDR1), a LC CDR2, and/or a LC CDR3 of SEQ ID NO: 54, SEQ ID NO: 55, and/or SEQ ID NO: 56;
(1) a heavy chain complementarity determining region (HC CDR1), a HC CDR2 and/or a HC CDR3 of SEQ ID NO: 17, SEQ ID NO: 18, and/or SEQ ID NO: 19, and/or
(2) a light chain complementarity determining region 1 (LC CDR1), a LC CDR2, and/or a LC CDR3 of SEQ ID NO: 20, SEQ ID NO: 21, and/or SEQ ID NO: 22;
(1) a heavy chain complementarity determining region (HC CDR1), a HC CDR2 and/or a HC CDR3 of SEQ ID NO: 57, SEQ ID NO: 58, and/or SEQ ID NO: 59, and/or
(2) a light chain complementarity determining region 1 (LC CDR1), a LC CDR2, and/or a LC CDR3 of SEQ ID NO: 63, SEQ ID NO: 64, and/or SEQ ID NO: 65;
(1) a heavy chain complementarity determining region (HC CDR1), a HC CDR2 and/or a HC CDR3 of SEQ ID NO: 60, SEQ ID NO: 61, and/or SEQ ID NO: 62, and/or
(2) a light chain complementarity determining region 1 (LC CDR1), a LC CDR2, and/or a LC CDR3 of SEQ ID NO: 66, SEQ ID NO: 67, and/or SEQ ID NO: 68; or
(1) a heavy chain complementarity determining region (HC CDR1), a HC CDR2 and/or a HC CDR3 of SEQ ID NO: 15, SEQ ID NO: 23, SEQ ID NO: 24, or SEQ ID NO: 25, and/or
(2) a light chain complementarity determining region 1 (LC CDR1), a LC CDR2, and/or a LC CDR3 of SEQ ID NO: 16, SEQ ID NO: 26, SEQ ID NO: 27, SEQ ID NO: 28, SEQ ID NO: 29, or SEQ ID NO: 30; or
(C) a humanized antibody molecule which binds, e.g., specifically binds, to a T cell receptor beta variable chain (TCRβV) region, wherein the anti-TCRβV antibody molecule comprises an antigen binding domain comprising:
(1) a heavy chain complementarity determining region (HC CDR1), a HC CDR2 and/or a HC CDR3 of SEQ ID NO: 3, SEQ ID NO: 4, and/or SEQ ID NO: 5, and/or
(2) a light chain complementarity determining region 1 (LC CDR1), a LC CDR2, and/or a LC CDR3 of SEQ ID NO: 6, SEQ ID NO: 7, and/or SEQ ID NO: 8;
(1) a heavy chain complementarity determining region (HC CDR1), a HC CDR2 and/or a HC CDR3 of SEQ ID NO: 45, SEQ ID NO: 46, and/or SEQ ID NO: 47, and/or
(2) a light chain complementarity determining region 1 (LC CDR1), a LC CDR2, and/or a LC CDR3 of SEQ ID NO: 51, SEQ ID NO: 52, and/or SEQ ID NO: 53;
(1) a heavy chain complementarity determining region (HC CDR1), a HC CDR2 and/or a HC CDR3 of SEQ ID NO: 48, SEQ ID NO: 49, and/or SEQ ID NO: 50, and/or
(2) a light chain complementarity determining region 1 (LC CDR1), a LC CDR2, and/or a LC CDR3 of SEQ ID NO: 54, SEQ ID NO: 55, and/or SEQ ID NO: 56;
(1) a heavy chain complementarity determining region (HC CDR1), a HC CDR2 and/or a HC CDR3 of SEQ ID NO: 17, SEQ ID NO: 18, and/or SEQ ID NO: 19, and/or (2) a light chain complementarity determining region 1 (LC CDR1), a LC CDR2, and/or a LC CDR3 of SEQ ID NO: 20, SEQ ID NO: 21, and/or SEQ ID NO: 22;
(1) a heavy chain complementarity determining region (HC CDR1), a HC CDR2 and/or a HC CDR3 of SEQ ID NO: 57, SEQ ID NO: 58, and/or SEQ ID NO: 59, and/or
(2) a light chain complementarity determining region 1 (LC CDR1), a LC CDR2, and/or a LC CDR3 of SEQ ID NO: 63, SEQ ID NO: 64, and/or SEQ ID NO: 65;
(1) a heavy chain complementarity determining region (HC CDR1), a HC CDR2 and/or a HC CDR3 of SEQ ID NO: 60, SEQ ID NO: 61, and/or SEQ ID NO: 62, and/or
(2) a light chain complementarity determining region 1 (LC CDR1), a LC CDR2, and/or a LC CDR3 of SEQ ID NO: 66, SEQ ID NO: 67, and/or SEQ ID NO: 68; or
(i) a heavy chain complementarity determining region (HC CDR1), a HC CDR2 and/or a HC CDR3 of SEQ ID NO: 15, SEQ ID NO: 23, SEQ ID NO: 24, or SEQ ID NO: 25; and/or
(ii) a light chain complementarity determining region 1 (LC CDR1), a LC CDR2, and/or a LC CDR3 of SEQ ID NO: 16, SEQ ID NO: 26, SEQ ID NO: 27, SEQ ID NO: 28, SEQ ID NO: 29, or SEQ ID NO: 30.

148. The method of any of embodiments 1-128 or 131-147, wherein the anti-TCRβV antibody molecule comprises an antigen binding domain comprising a light chain variable region (VL) comprising one, two or all of a LC CDR1, a LC CDR2 and a LC CDR3 of SEQ ID NO: 16, SEQ ID NO: 26, SEQ ID NO: 27, SEQ ID NO: 28, SEQ ID NO: 29, or SEQ ID NO: 30.

149. The method of any of embodiments 1-128 or 131-148, wherein the anti-TCRβV antibody molecule comprises an antigen binding domain comprising a heavy chain variable region (VH) comprising one, two or all of a HC CDR1, a HC CDR2 and a HC CDR3 of SEQ ID NO: 15, SEQ ID NO: 23, SEQ ID NO: 24, or SEQ ID NO: 25.

150. The method of any of embodiments 1-128 or 131-149, wherein the anti-TCRβV antibody molecule comprises an antigen binding domain comprising:
(i) a VL comprising: a LC CDR1 amino acid sequence of SEQ ID NO: 20 (or an amino acid sequence with not more than 1, 2, 3 or 4 modifications, e.g., substitutions, additions or deletions thereof), a LC CDR2 amino acid sequence of SEQ ID NO:21 (or an amino acid sequence with not more than 1, 2, 3 or 4 modifications, e.g., substitutions, additions or deletions thereof), and/or a LC CDR3 amino acid sequence of SEQ ID NO:22 (or an amino acid sequence with not more than 1, 2, 3 or 4 modifications, e.g., substitutions, additions or deletions thereof); and/or
(ii) a VH comprising: a HC CDR1 amino acid sequence of SEQ ID NO: 17 (or an amino acid sequence with not more than 1, 2, 3 or 4 modifications, e.g., substitutions, additions or deletions thereof), a HC CDR2 amino acid sequence of SEQ ID NO:18 (or an amino acid sequence with not more than 1, 2, 3 or 4 modifications, e.g., substitutions, additions or deletions thereof), and/or a HC CDR3 amino acid sequence of SEQ ID NO:19 (or an amino acid sequence with not more than 1, 2, 3 or 4 modifications, e.g., substitutions, additions or deletions thereof).

151. The method of any of embodiments 1-128 or 131-150, wherein the anti-TCRβV antibody molecule comprises an antigen binding domain comprising:
a variable heavy chain (VH) of SEQ ID NO: 23, SEQ ID NO: 24, or SEQ ID NO: 25, or a sequence having at least about 85%, 90%, 95%, or 99% sequence identity thereto; and/or a variable light chain (VL) of SEQ ID NO: 26, SEQ ID NO: 27, SEQ ID NO: 28, SEQ ID NO: 29, or SEQ ID NO: 30, or a sequence having at least about 85%, 90%, 95%, or 99% sequence identity thereto.

152. The method of any of embodiments 1-128 or 131-151, wherein the anti-TCRβV antibody molecule comprises a light chain comprising a framework region, e.g., framework region 1 (FR1), comprising one, two or all (e.g., three) of:
(i) an Aspartic Acid at position 1, e.g., a substitution at position 1 according to Kabat numbering, e.g., a Alanine to Aspartic Acid substitution; or
(ii) an Asparagine at position 2, e.g., a substitution at position 2 according to Kabat numbering, e.g., a Isoleucine to Asparagine, a Serine to Asparagine, or a Tyrosine to Asparagine substitution; or
(iii) a Leucine at position 4, e.g., a substitution at position 4 according to Kabat numbering, e.g., a Methionine to Leucine substitution,
wherein the substitution is relative to a human germline light chain framework region sequence.

153. The method of any of embodiments 1-128 or 131-153, wherein the anti-TCRβV antibody molecule comprises a light chain comprising a framework region, e.g., framework region 3 (FR3), comprising one, two or all (e.g., three) of:
(i) a Glycine at position 66, e.g., a substitution at position 66 according to Kabat numbering, e.g., a Lysine to Glycine, or a Serine to Glycine substitution; or
(ii) an Asparagine at position 69, e.g., a substitution at position 69 according to Kabat numbering, e.g., a Threonine to Asparagine substitution; or
(iii) a Tyrosine at position 71, e.g., a substitution at position 71 according to Kabat numbering, e.g., a Phenylalanine to Tyrosine, or Alanine to Tyrosine substitution,
wherein the substitution is relative to a human germline light chain framework region sequence.

154. The method of any of embodiments 1-128 or 131-153, wherein the method results in expansion of, e.g., selective or preferential expansion of, T cells expressing a T cell receptor (TCR) comprising a TCR alpha and/or TCR beta molecule, e.g., TCR alpha-beta T cells (αβ T cells).

155. The method of any of embodiments 1-128 or 131-154, wherein the method results in expansion of af3T cells over expansion of T cells expressing a TCR comprising a TCR gamma and/or TCR delta molecule, e.g., TCR gamma-delta T cells (γδ T cells).

In some embodiments, the anti-TCRβV antibody molecules disclosed herein result in lesser or no production of cytokines associated with cytokine release syndrome (CRS), e.g., IL-6, IL-1beta and TNF alpha; and enhanced and/or delayed production of IL-2 and IFNγ. In some embodiments, the anti-TCRβV antibodies disclosed herein result in expansion of an immune cell, e.g., a T cell, or a subset of memory effector T cells known as T_{EMRA}), an NK cell, or other immune cells (e.g., as described herein). Also provided herein are methods of making said anti-TCRβV antibody molecules, and methods of using said anti-TCRβV antibody molecules including, methods of using an anti-TCRβV antibody molecule for expanding an immune cell or an immune cell population. This disclosure further provides multispecific molecules, e.g., bispecific molecules, comprising said anti-TCRβV antibody molecules. In some embodiments, compositions comprising anti-TCRβV antibody molecules of the present disclosure, can be used, e.g., to activate and/or redirect T cells to treat an infectious disease. In some embodiments, compositions comprising anti-TCRβV antibody molecules as disclosed herein limit the unwanted side-effects of CRS, e.g., CRS associated with anti-CD3e targeting.

Accordingly, provided herein are, anti-TCRβV antibody molecules, multispecific or multifunctional molecules (e.g., multispecific or multifunctional antibody molecules) (also referred to herein as a “composition”) that comprise anti-TCRβV antibody molecules, nucleic acids encoding the same, methods of producing the aforesaid molecules, pharmaceutical compositions comprising aforesaid molecules, and methods of treating a disease or disorder, e.g., an infectious disease, e.g., as described herein, using the aforesaid molecules. The antibody molecules and pharmaceutical compositions disclosed herein can be used (alone or in combination with other agents or therapeutic modalities) to treat, prevent and/or diagnose disorders and conditions, e.g., an infectious disease, e.g., as described herein.

In one aspect, the disclosure provides a non-murine, e.g., human or humanized antibody molecule, which binds, e.g., specifically binds, to a T cell receptor beta variable (TCRβV) region. In some embodiments, binding of the anti-TCRβV antibody molecule to a TCRβV region results in one, two, three, four, five, six, seven, eight, nine, ten or more (e.g., all) of the following:
(i) reduced level, e.g., expression level, and/or activity of IL-10;
(ii) reduced level, e.g., expression level, and/or activity of IL-6;
(iii) reduced level, e.g., expression level, and/or activity of TNFα; (iv) increased level, e.g., expression level, and/or activity of IL-2;
(v) a delay, e.g., at least 1, 2, 3, 4, 5, 6, 7, 8, 9, 10 or more hours delay, in increased level, e.g., expression level, and/or activity of IL-2;
(vi) a delay, e.g., at least 1, 2, 3, 4, 5, 6, 7, 8, 9, 10 hours delay, in increased level, e.g., expression level, and/or activity of IFNγ;
(vii) reduced T cell proliferation kinetics;
(viii) reduced cytokine storm, e.g., cytokine release syndrome (CRS), e.g., as measured by an assay of Example 3;
(ix) cell killing, e.g., target cell killing;
(x) increased level, e.g., expression level, and/or activity of IL-15; or
(xi) increased Natural Killer (NK) cell proliferation, e.g., expansion.

In some embodiments, any one or all of (i)-(xi) or any combination thereof resulting from an anti-TCRβV antibody molecule disclosed herein is compared to an antibody that binds to: a CD3 molecule, e.g., CD3 epsilon (CD3e) molecule; or a TCR alpha (TCRα) molecule.

In some embodiments, binding of the anti-TCRβV antibody molecule to a TCRβV region results in secretion, e.g., production of perforin and/or Granzyme B.

In another aspect, the disclosure provides a non-murine, e.g., human or humanized antibody molecule, which binds, e.g., specifically binds, to a T cell receptor beta variable (TCRβV) region. In some embodiments, binding of the anti-TCRβ V antibody molecule results in expansion, e.g., at least about 1.1-50 fold expansion (e.g., at least about 1.5-40 fold, 2-35 fold, 3-30 fold, 5-25 fold, 8-20 fold, or 10-15 fold expansion), of a population of memory T cells, e.g., T effector memory (T_{EM}) cells, e.g., T_{EM} cells expressing CD45RA (T_{EMRA}) cells, e.g., CD4+or CD8+T_{EMRA} cells. In some embodiments, the expansion is at least about 1.1-10 fold expansion (e.g., at least about 1.1, 1.2, 1.3, 1.4, 1.5, 2, 3, 4, 5, 6, 7, 8, 9, or 10 fold expansion).

In some embodiments, expansion of the population of memory effector T cells, e.g., T_{EM} cells, e.g., T_{EMRA} cells, e.g., CD4+or CD8+T_{EMRA} cells, is compared to expansion of a similar population of cells with an antibody that binds to: a CD3 molecule, e.g., CD3 epsilon (CD3e) molecule; or a TCR alpha (TCRα) molecule.

In some embodiments, the population of expanded T effector memory cells comprises cells T cells, e.g., CD3+, CD8+or CD4+T cells. In some embodiments, the population of expanded T effector memory cells comprises CD3+and CD8+T cells. In some embodiments, the population of expanded T effector memory cells comprises CD3+and CD4+T cells.

In some embodiments, the population of expanded T effector memory (T_{EM}) cells comprises cells T cells, e.g., CD3+, CD8+or CD4+T cells, which express or re-express, CD45RA, e.g., CD45RA+. In some embodiments, the population comprises T_{EM} cells expressing CD45RA, e.g., T_{EMRA} cells. In some embodiments, expression of CD45RA on T_{EMRA} cells, e.g., CD4+or CD8+T_{EMRA} cells, can be detected by a method disclosed herein, e.g., flow cytometry.

In some embodiments, T_{EMRA} cells have low or no expression of CCR7, e.g., CCR7- or CCR7 low. In some embodiments, expression of CCR7 on T_{EMRA} cells cannot be detected by a method disclosed herein, e.g., flow cytometry.

In some embodiments, T_{EMRA} cells express CD95, e.g., CD95+. In some embodiments, expression of CD95 on T_{EMRA} cells can be detected by a method disclosed herein, e.g., flow cytometry.

In some embodiments, T_{EMRA} cells express CD45RA, e.g., CD45RA+, have low or no expression of CCR7, e.g., CCR7- or CCR7 low, and express CD95, e.g., CD95+. In some embodiments T_{EMRA} cells can be identified as CD45RA+, CCR7- and CD95+cells. In some embodiments, T_{EMRA} cells comprise CD3+, CD4+or CD8+T cells (e.g., CD3+T cells, CD3+CD8+T cells, or CD3+CD4+T cells).

In some embodiments, binding of the anti-TCRβ V antibody molecule to a TCRβ V region results in one, two, three, four, five, six, seven, eight, nine, ten or more (e.g., all) of the following:
(i) reduced level, e.g., expression level, and/or activity of IL-10;
(ii) reduced level, e.g., expression level, and/or activity of IL-6;
(iii) reduced level, e.g., expression level, and/or activity of TNFα;
(iv) increased level, e.g., expression level, and/or activity of IL-2;
(v) a delay, e.g., at least 1, 2, 3, 4, 5, 6, 7, 8, 9, 10 or more hours delay, in increased level, e.g., expression level, and/or activity of IL-2;
(vi) a delay, e.g., at least 1, 2, 3, 4, 5, 6, 7, 8, 9, 10 hours delay, in increased level, e.g., expression level, and/or activity of IFNγ;
(vii) reduced T cell proliferation kinetics;
(viii) reduced cytokine storm, e.g., cytokine release syndrome (CRS), e.g., as measured by an assay of Example 3;
(ix) cell killing, e.g., target cell killing;
(x) increased level, e.g., expression level, and/or activity of IL-15; or
(xi) increased Natural Killer (NK) cell proliferation, e.g., expansion, compared to an antibody that binds to: a CD3 molecule, e.g., CD3 epsilon (CD3e) molecule; or a TCR alpha (TCRα) molecule.

In some embodiments of any of the compositions disclosed herein, binding of the anti-TCRβV antibody molecule to a TCRβV region results in a reduction of at least 2, 5, 10, 20, 50, 100, or 200 fold, or at least 2-200 fold (e.g., 5-150, 10-100, 20-50 fold) in the expression level and or activity of IL-10 as measured by an assay of Example 3.

In some embodiments of any of the compositions disclosed herein, binding of the anti-TCRβV antibody molecule to a TCRβV region results in a reduction of at least 2, 5, 10, 20, 50, 100, 200, 300, 400, 500, 600, 700, 800, 900, or 1000 fold, or at least 2-1000 fold (e.g., 5-900, 10-800, 20-700, 50-600, 100-500, or 200-400 fold) in the expression level and or activity of IL-6 as measured by an assay of Example 3.

In some embodiments of any of the compositions disclosed herein, binding of the anti-TCRβV antibody molecule to a TCRβV region results in a reduction of at least 2, 5, 10, 20, 50, 100, 200, 300, 400, 500, 600, 700, 800, 900, 1000, or 2000 fold, or at least 2-2000 fold (e.g., 5-1000, 10-900, 20-800, 50-700, 100-600, 200-500, or 300-400 fold) in the expression level and or activity of TNFα as measured by an assay of Example 3.

In some embodiments of any of the compositions disclosed herein, binding of the anti-TCRβV antibody molecule to a TCRβV region results in an increase of at least 2, 5, 10, 20, 50, 100, 200, 300, 400, 500, 600, 700, 800, 900, 1000, or 2000 fold, or at least 2-2000 fold (e.g., 5-1000, 10-900, 20-800, 50-700, 100-600, 200-500, or 300-400 fold) in the expression level and or activity of IL-2 as measured by an assay of Example 3.

In some embodiments of any of the compositions disclosed herein, binding of the anti-TCRβV antibody molecule to a TCRβV region results in an increase of at least 2, 5, 10, 20, 50, 100, 200, 300, 400, 500, 600, 700, 800, 900, 1000, or 2000 fold, or at least 2-2000 fold (e.g., 5-1000, 10-900, 20-800, 50-700, 100-600, 200-500, or 300-400 fold) in the expression level and or activity of IL-15.

In some embodiments of any of the compositions disclosed herein, binding of the anti-TCRβV antibody molecule results in proliferation, e.g., expansion, e.g., at least about 1.1-50 fold expansion (e.g., at least about 1.5-40 fold, 2-35 fold, 3-30 fold, 5-25 fold, 8-20 fold, or 10-15 fold expansion), of a population of Natural Killer (NK) cells. In some embodiments, the expansion of NK cells is at least about 1.1-30 fold expansion (e.g., at least about 1.1, 1.2, 1.3, 1.4, 1.5, 2, 3, 4, 5, 6, 7, 8, 9, 10, 15, 20, 25, or at least about 1.1-5, 5-10, 10-15, 15-20, 20-25, or 25-30 fold expansion). In some embodiments, the expansion of NK cells by, e.g., binding of, the anti-TCRβV antibody molecule is compared to expansion of an otherwise similar population not contacted with the anti-TCRβV antibody molecule.

In some embodiments of any of the compositions disclosed herein, binding of the anti-TCRβV antibody molecule results in cell killing, e.g., target cell killing. In some embodiments, binding of the anti-TCRβV antibody molecule results in cell killing in vitro or in vivo.

In some embodiments of any of the compositions disclosed herein, binding of the anti-TCRβV antibody molecule to a TCRβV region results in an increase or decrease of at least 2, 5, 10, 20, 50, 100, 200, 300, 400, 500, 600, 700, 800, 900, 1000, or 2000 fold, or at least 2-2000 fold (e.g., 5-1000, 10-900, 20-800, 50-700, 100-600, 200-500, or 300-400 fold) of any of the activities described herein compared the activity of Antibody B or murine Antibody C, or a humanized version thereof (e.g., humanized mAb Antibody B-H.1 to B-H.6) as described in U.S. Pat. No. 5,861,155.

In an aspect, provided herein is an antibody molecule which binds, e.g., specifically binds, to a T cell receptor beta variable chain (TCRβV) region (an anti-TCRβV antibody molecule), wherein the anti-TCRβV antibody molecule:
(i) binds specifically to an epitope on TCRβV, e.g., the same or similar epitope as the epitope recognized by an anti-TCRβV antibody molecule as described herein, e.g., a second anti-TCRβV antibody molecule;
(ii) shows the same or similar binding affinity or specificity, or both, as an anti-TCRβV antibody molecule as described herein, e.g., a second anti-TCRβV antibody molecule;
(iii) inhibits, e.g., competitively inhibits, the binding of an anti-TCRβV antibody molecule as described herein, e.g., a second anti-TCRβV antibody molecule;
(iv) binds the same or an overlapping epitope with an anti-TCRβV antibody molecule as described herein, e.g., a second anti-TCRβV antibody molecule; or
(v) competes for binding, and/or binds the same epitope, with an anti-TCRβV antibody molecule as described herein, e.g., a second anti-TCRβV antibody molecule,

In some embodiments, the second anti-TCRβV antibody molecule comprises an antigen binding domain chosen from Table 3 or Table 4, or a sequence substantially identical thereto. In some embodiments, the second anti-TCRβV antibody molecule comprises an antigen binding domain, comprising:
a heavy chain complementarity determining region 1 (HC CDR1), a heavy chain complementarity determining region 2 (HC CDR2) and/or a heavy chain complementarity determining region 3 (HC CDR3) of SEQ ID NO: 1 or SEQ ID NO: 9; and/or a light chain complementarity determining region 1 (LC CDR1), a light chain complementarity determining region 2 (LC CDR2), and/or a light chain complementarity determining region 3 (LC CDR3) of SEQ ID NO: 2, SEQ ID NO: 10 or SEQ ID NO: 11.

In some embodiments of any of the compositions disclosed herein, binding of the anti-TCRβV antibody molecule to a TCRβV region results in a change in any (e.g., one, two, three, four or all) of (i)-(v) that is different, e.g., an increase or decrease, of at least 2, 5, 10, 20, 50, 100-fold, compared the activity of Antibody B or murine Antibody C or a humanized version thereof (e.g., humanized mAb Antibody B-H.1 to B-H.6) as described in U.S. Pat. No. 5,861,155.

In some embodiments of any of the compositions disclosed herein, the anti-TCRβV antibody molecule binds to a TCRBV family (e.g., gene family), e.g., a TCRBV gene family comprising subfamilies, e.g., as described herein. In some embodiments, the TCRBV family, e.g., gene family, comprises: a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 v, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily or a TCRβ V29 subfamily.

In some embodiments, the anti-TCRβV antibody binds to a TCRβ V6 subfamily chosen from: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments the TCRβ V6 subfamily comprises TCRβ V6-5*01.

In some embodiments, the anti-TCRβV antibody binds to a TCRβ V10 subfamily chosen from: TCRβ V10-1*01, TCRβ V10-1*02, TCRβ V10-3*01 or TCRβ V10-2*01.

In some embodiments, the anti-TCRβV antibody binds to a TCRβ V12 subfamily chosen from: TCRβ V12-4*01, TCRβ V12-3*01 or TCRβ V12-5*01.

In some embodiments of any of the compositions disclosed herein, the anti-TCRβV antibody molecule does not bind to TCRβ V12, or binds to TCRβ V12 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of the murine mAb Antibody B or a humanized version thereof (e.g., humanized mAb Antibody B-H.1 to B-H.6) as described in U.S. Pat. No. 5,861,155.

In some embodiments of any of the compositions disclosed herein, the anti-TCRβV antibody molecule binds to TCRβ V12 with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the murine mAb Antibody B or a humanized version thereof (e.g., humanized mAb Antibody B-H.1 to B-H.6) as described in U.S. Pat. No. 5,861,155.

In some embodiments of any of the compositions disclosed herein, the anti-TCRβV antibody molecule binds to a TCRβV region other than TCRβ V12 (e.g., TCRβV region as described herein, e.g., TCRβ V6 subfamily (e.g., TCRβ V6-5*01) with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the murine mAb Antibody B or a humanized version thereof (e.g., humanized mAb Antibody B-H.1 to B-H.6) as described in U.S. Pat. No. 5,861,155.

In some embodiments of any of the compositions disclosed herein, the anti-TCRβV antibody molecule does not comprise the CDRs of the murine mAb Antibody B.

In some embodiments of any of the compositions disclosed herein, the anti-TCRβV antibody binds to a TCRβ V5 subfamily chosen from: TCRβ V5-5*01, TCRβ V5-6*01, TCRβ V5-4*01, TCRβ V5-8*01, TCRβ V5-1*01.

In some embodiments of any of the compositions disclosed herein, the anti-TCRβV antibody binds to a TCRβ V5 subfamily chosen from: TCRβ V5-5*01, TCRβ V5-6*01, TCRβ V5-4*01, TCRβ V5-8*01, TCRβ V5-1*01.

In some embodiments of any of the compositions disclosed herein, the anti-TCRβV antibody molecule does not bind to TCRβ V5-5*01 or TCRβ V5-1*01, or binds to TCRβ V5-5*01 or TCRβ V5-1*01 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of murine Antibody C or a humanized version thereof as described in U.S. Pat. No. 5,861,155.

In some embodiments of any of the compositions disclosed herein, the anti-TCRβV antibody molecule binds to TCRβ V5-5*01 or TCRβ V5-1*0lwith an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of murine Antibody C or a humanized version thereof as described in U.S. Pat. No. 5,861,155.

In some embodiments of any of the compositions disclosed herein, the anti-TCRβV antibody molecule binds to a TCRβV region other than TCRβ V5-5*01 or TCRβ V5-1*01 (e.g., TCRβV region as described herein, e.g., TCRβ V6 subfamily (e.g., TCRβ V6-5*01) with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of murine Antibody C or a humanized version thereof as described in U.S. Pat. No. 5,861,155.

In some embodiments of any of the compositions disclosed herein, the anti-TCRβV antibody molecule does not comprise the CDRs of murine Antibody C.

In some embodiments of any of the compositions disclosed herein, the anti-TCRβV antibody molecule binds to one or more (e.g., all) of the following TCRβV subfamilies:
(i) TCRβ V6, e.g., TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01;
(ii) TCRβ V10, e.g., TCRβ V10-1*01, TCRβ V10-1*02, TCRβ V10-3*01 or TCRβ V10-2*01;
(iii) TCRβ V12, e.g., TCRβ V12-4*01, TCRβ V12-3*01, or TCRβ V12-5*01; or
(iv) TCRβ V5, e.g., TCRβ V5-5*01, TCRβ V5-6*01, TCRβ V5-4*01, TCRβ V5-8*01, TCRβV5-1*01.

In some embodiments, the anti-TCRβV antibody molecule binds to TCRβ V6, e.g., TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments, the anti-TCRβV antibody molecule binds to TCRβ V6-5*01.

In some embodiments, the anti-TCRβV antibody molecule does not bind to TCRβ V12.

In some embodiments, the anti-TCRβV antibody molecule does not bind to TCRβ V5-5*01 or TCRβ V5-1*01.

In another aspect, provided herein is a method of expanding, e.g., increasing the number of, an immune cell population comprising, contacting the immune cell population with an antibody molecule, e.g., humanized antibody molecule, which binds, e.g., specifically binds, to a T cell receptor beta variable chain (TCRβV) region (e.g., anti-TCRβV antibody molecule described herein or a multispecific molecule comprising an anti-TCRβV antibody molecule described herein), thereby expanding the immune cell population.

In some embodiments, the expansion occurs in vivo or ex vivo (e.g., in vitro).

In some embodiments, the immune cell population comprises a T cell, a Natural Killer cell, a B cell, an antigen presenting cell, or a myeloid cell (e.g., a monocyte, a macrophage, a neutrophil or a granulocyte).

In some embodiments, the immune cell population comprises a T cell, e.g., a CD4+T cell, a CD8+T cell, a TCR alpha-beta T cell, or a TCR gamma-delta T cell. In some embodiments, a T cell comprises a memory T cell (e.g., a central memory T cell, or an effector memory T cell (e.g., a T_{EMRA}) or an effector T cell.

In some embodiments, the immune cell population is obtained from a healthy subject.

In some embodiments, the immune cell population is obtained from a subject (e.g., from an apheresis sample from the subject) having a disease, e.g., infectious disease, e.g., as described herein. In some embodiments, the immune cell population obtained from a subject having a disease, e.g., an infectious disease, comprises a T cell, a Natural Killer cell, a B cell, or a myeloid cell.

In some embodiments, the method results in an expansion of at least 1.1-10 fold (e.g., at least 1.1, 1.2, 1.3, 1.4, 1.5, 2, 3, 4, 5, 6, 7, 8, 9, or 10 fold expansion).

In some embodiments, the method further comprises contacting the population of cells with an agent that promotes, e.g., increases, immune cell expansion. In some embodiments, the agent includes an immune checkpoint inhibitor, e.g., as described herein. In some embodiments, the agent includes a 4-1BB (CD127) agonist, e.g., an anti-4-1BB antibody.

In some embodiments, the method further comprises comprising contacting the population of cells with a non-dividing population of cells, e.g., feeder cells, e.g., irradiated allogenic human PBMCs.

In some embodiments, an expansion method described herein comprises expanding the cells for a period of at least about 4 hours, 6 hours, 10 hours, 12 hours, 15 hours, 18 hours, 20 hours, or 22 hours, or for at least 1, 2, 3, 4, 5, 6, 7, 8, 9, 10, 11, 12, 13, 14, 15, 1,6 17, 18, 19, 20 or 21 days, or for at least about 1 week, 2 weeks, 3 weeks, 4 weeks, 5 weeks, 6 weeks, 7 weeks or 8 weeks.

In some embodiments, expansion of the population of immune cells, is compared to expansion of a similar population of cells with an antibody that binds to: a CD3 molecule, e.g., CD3 epsilon (CD3e) molecule; or a TCR alpha (TCRα) molecule.

In some embodiments, expansion of the population of immune cells, is compared to expansion of a similar population of cells not contacted with the anti-TCRβV antibody molecule.

In some embodiments, expansion of the population of memory effector T cells, e.g., TEM cells, e.g., T_{EMRA} cells, is compared to expansion of a similar population of cells with an antibody that binds to: a CD3 molecule, e.g., CD3 epsilon (CD3e) molecule; or a TCR alpha (TCRα) molecule.

In some embodiments, the method results in expansion of, e.g., selective or preferential expansion of, T cells expressing a T cell receptor (TCR) comprising a TCR alpha and/or TCR beta molecule, e.g., TCR alpha-beta T cells (αβ T cells).

In some embodiments, the method results in expansion of af3T cells over expansion of T cells expressing a TCR comprising a TCR gamma and/or TCR delta molecule, e.g., TCR gamma-delta T cells (γδ T cells). In some embodiments, expansion of GOT cells over y6 T cells results in reduced production of cytokines associated with CRS. In some embodiments, expansion of GOT cells over y6 T cells results in immune cells that have reduced capacity to, e.g., are less prone to, induce CRS upon administration into a subject.

In some embodiments, an immune cell population (e.g., T cells (e.g., T_{EMRA} cells or TILs) or NK cells) cultured in the presence of, e.g., expanded with, an anti- TCRβV antibody disclosed herein does not induce CRS when administered into a subject, e.g., a subject having a disease or condition as described herein.

Alternatively or in combination with any of the embodiments disclosed herein, provided herein is an anti-TCRβV antibody molecule which:
(i) binds specifically to an epitope on TCRβV, e.g., the same or similar epitope as the epitope recognized by an anti-TCRβV antibody molecule as described herein, e.g., a second anti-TCRβV antibody molecule;
(ii) shows the same or similar binding affinity or specificity, or both, as an anti-TCRβV antibody molecule as described herein, e.g., a second anti-TCRβV antibody molecule;
(iii) inhibits, e.g., competitively inhibits, the binding of an anti-TCRβV antibody molecule as described herein, e.g., a second anti-TCRβV antibody molecule;
(iv) binds the same or an overlapping epitope with an anti-TCRβV antibody molecule as described herein, e.g., a second anti-TCRβV antibody molecule; or
(v) competes for binding, and/or binds the same epitope, with an anti-TCRβV antibody molecule as described herein, e.g., a second anti-TCRβV antibody molecule,

In some embodiments, the second anti-TCRβV antibody molecule comprises an antigen binding domain chosen from Table 3 or Table 4, or a sequence substantially identical thereto. In some embodiments, the second anti-TCRβV antibody molecule comprises an antigen binding domain, comprising:
a heavy chain complementarity determining region 1 (HC CDR1), a heavy chain complementarity determining region 2 (HC CDR2) and/or a heavy chain complementarity determining region 3 (HC CDR3) of SEQ ID NO: 1 or SEQ ID NO: 9; and/or a light chain complementarity determining region 1 (LC CDR1), a light chain complementarity determining region 2 (LC CDR2), and/or a light chain complementarity determining region 3 (LC CDR3) of SEQ ID NO: 2, SEQ ID NO: 10, or SEQ ID NO: 11.

In another aspect, the disclosure provides a multispecific molecule, e.g., a bispecific molecule, comprising the anti-TCRβV antibody molecule disclosed herein.

In some embodiments, the multispecific molecule further comprises: an infectious disease-targeting moiety, a cytokine molecule, an immune cell engager, e.g., a second immune cell engager, and/or a stromal modifying moiety.

In yet another aspect, disclosed herein is a multispecific molecule, e.g., a bispecific molecule, comprising:
(i) a first moiety comprising a first immune cell engager comprising an anti-TCRβV antibody molecule disclosed herein; and
(ii) a second moiety comprising one or more of: an infectious disease-targeting moiety; a second immune cell engager; a cytokine molecule or a stromal modifying moiety.

In some embodiments of any of the compositions disclosed herein, the anti-TCRβV antibody molecule comprises an antigen binding domain comprising:
(i) a heavy chain complementarity determining region 1 (HC CDR1), a heavy chain complementarity determining region 2 (HC CDR2) and/or a heavy chain complementarity determining region 3 (HC CDR3) of SEQ ID NO: 1 or SEQ ID NO: 9; or
(ii) a light chain complementarity determining region 1 (LC CDR1), a light chain complementarity determining region 2 (LC CDR2), and/or a light chain complementarity determining region 3 (LC CDR3) of SEQ ID NO: 2, SEQ ID NO: 10, or SEQ ID NO: 11.

In some embodiments of any of the compositions disclosed herein, the anti-TCRβV antibody molecule comprises an antigen binding domain comprising a light chain variable region (VL) comprising one, two or all (e.g., three) of a LC CDR1, a LC CDR2 and a LC CDR3 of SEQ ID NO: 2, SEQ ID NO: 10, or SEQ ID NO: 11.

In some embodiments of any of the compositions disclosed herein, the anti-TCRβV antibody molecule comprises an antigen binding domain comprising a heavy chain variable region (VH) comprising one, two or all (e.g., three) of a HC CDR1, a HC CDR2 and a HC CDR3 of SEQ ID NO:1 or SEQ ID NO: 9.

In some embodiments of any of the compositions disclosed herein, the anti-TCRβV antibody molecule comprises an antigen binding domain comprising:
(i) a VL comprising: a LC CDR1 amino acid sequence of SEQ ID NO: 6 (or an amino acid sequence with not more than 1, 2, 3 or 4 modifications, e.g., substitutions, additions or deletions thereof), a LC CDR2 amino acid sequence of SEQ ID NO:7 (or an amino acid sequence with not more than 1, 2, 3 or 4 modifications, e.g., substitutions, additions or deletions thereof), and/or a LC CDR3 amino acid sequence of SEQ ID NO:8 (or an amino acid sequence with not more than 1, 2, 3 or 4 modifications, e.g., substitutions, additions or deletions thereof); and/or
(ii) a VH comprising: a HC CDR1 amino acid sequence of SEQ ID NO: 3 (or an amino acid sequence with not more than 1, 2, 3 or 4 modifications, e.g., substitutions, additions or deletions thereof), a HC CDR2 amino acid sequence of SEQ ID NO: 4 (or an amino acid sequence with not more than 1, 2, 3 or 4 modifications, e.g., substitutions, additions or deletions thereof), and/or a HC CDR3 amino acid sequence of SEQ ID NO: 5 (or an amino acid sequence with not more than 1, 2, 3 or 4 modifications, e.g., substitutions, additions or deletions thereof).

In some embodiments of any of the compositions disclosed herein, the anti-TCRβV antibody molecule comprises an antigen binding domain comprising:
a variable heavy chain (VH) of SEQ ID NO: 9, or a sequence having at least about 75%, 80%, 85%, 90%, 95%, or 99% sequence identity thereto; and/or
a variable light chain (VL) of SEQ ID NO: 10 or SEQ ID NO: 11, or a sequence having at least about 75%, 80%, 85%, 90%, 95%, or 99% sequence identity thereto.

In some embodiments of any of the compositions disclosed herein, the anti-TCRβV antibody molecule comprises an antigen binding domain comprising the VH amino acid sequence of SEQ ID NO: 9 and the VL amino acid sequence of SEQ ID NO: 10.

In some embodiments of any of the compositions disclosed herein, the anti-TCRβV antibody molecule comprises an antigen binding domain comprising the VH amino acid sequence of SEQ ID NO: 9 and the VL amino acid sequence of SEQ ID NO: 11.

In some embodiments of any of the compositions disclosed herein, the anti-TCRβV antibody molecule comprises a heavy chain comprising a framework region, e.g., framework region 3 (FR3), comprising one or both of: (i) a Threonine at position 73, e.g., a substitution at position 73 according to Kabat numbering, e.g., a Glutamic Acid to Threonine substitution; or (ii) a Glycine at position, e.g., a substitution at position 94 according to Kabat numbering, e.g., a Arginine to Glycine substitution. In some embodiments, the substitution is relative to a human germline heavy chain framework region sequence.

In some embodiments of any of the compositions disclosed herein, the anti-TCRβV antibody molecule comprises a light chain comprising a framework region, e.g., framework region 1 (FR1), comprising a Phenylalanine at position 10, e.g., a substitution at position 10 according to Kabat numbering, e.g., a Serine to Phenyalanine substitution. In some embodiments, the substitution is relative to a human germline light chain framework region sequence.

In some embodiments of any of the compositions disclosed herein, the anti-TCRβV antibody molecule comprises a light chain comprising a framework region, e.g., framework region 2 (FR2), comprising one or both of: (i) a Histidine at position 36, e.g., a substitution at position 36 according to Kabat numbering, e.g., a Tyrosine to Histidine substitution; or (ii) an Alanine at position 46, e.g., a substitution at position 46 according to Kabat numbering, e.g., a Arginine to Alanine substitution. In some embodiments, the substitution is relative to a human germline light chain framework region sequence.

In some embodiments of any of the compositions disclosed herein, the anti-TCRβV antibody molecule comprises a light chain comprising a framework region, e.g., framework region 3 (FR3), comprising a Phenylalanine at position 87, e.g., a substitution at position 87 according to Kabat numbering, e.g., a Tyrosine to Phenyalanine substitution. In some embodiments, the substitution is relative to a human germline light chain framework region sequence.

In some embodiments of any of the compositions disclosed herein, the anti-TCRβV antibody molecule binds to TCRβ V6, e.g., TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments the anti-TCRβV antibody molecule binds to TCRβ V6-5*01.

In some embodiments, TCRβ V6, e.g., TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01, is recognized, e.g., bound, by SEQ ID NO: 1 and/or SEQ ID NO: 2. In some embodiments, TCRβ V6, e.g., TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01, is recognized, e.g., bound, by SEQ ID NO: 9 and/or SEQ ID NO: 10. In some embodiments, TCRβ V6, e.g., TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01, is recognized, e.g., bound, by SEQ ID NO: 9 and/or SEQ ID NO: 11. In some embodiments, TCRβ V6-5*01 is recognized, e.g., bound by SEQ ID NO: 9 and/or SEQ ID NO: 10, or a sequence having at least about 75%, 80%, 85%, 90%, 95%, or 99% sequence identity thereto. In some embodiments, TCRβ V6-5*01 is recognized, e.g., bound by SEQ ID NO: 9 and/or SEQ ID NO: 11, or a sequence having at least about 75%, 80%, 85%, 90%, 95%, or 99% sequence identity thereto.

In some embodiments of any of the compositions disclosed herein, the anti-TCRβV antibody molecule comprises an antigen binding domain comprising:
(i) a heavy chain complementarity determining region (HC CDR1), a HC CDR2 and/or a HC CDR3 of SEQ ID NO: 15, SEQ ID NO: 23, SEQ ID NO: 24, or SEQ ID NO: 25; and/or
(ii) a light chain complementarity determining region 1 (LC CDR1), a LC CDR2, and/or a LC CDR3 of SEQ ID NO: 16, SEQ ID NO: 26, SEQ ID NO: 27, SEQ ID NO: 28, SEQ ID NO: 29, or SEQ ID NO: 30.

In some embodiments of any of the compositions disclosed herein, the anti-TCRβV antibody molecule comprises an antigen binding domain comprising a light chain variable region (VL) comprising one, two or all of a LC CDR1, a LC CDR2 and a LC CDR3 of SEQ ID NO: 16, SEQ ID NO: 26, SEQ ID NO: 27, SEQ ID NO: 28, SEQ ID NO: 29, or SEQ ID NO: 30.

In some embodiments of any of the compositions disclosed herein, the anti-TCRβV antibody molecule comprises an antigen binding domain comprising a heavy chain variable region (VH) comprising one, two or all of a HC CDR1, a HC CDR2 and a HC CDR3 of SEQ ID NO: 15, SEQ ID NO: 23, SEQ ID NO: 24, or SEQ ID NO: 25.

In some embodiments of any of the compositions disclosed herein, the anti-TCRβV antibody molecule comprises an antigen binding domain comprising:
(i) a VL comprising: a LC CDR1 amino acid sequence of SEQ ID NO: 20 (or an amino acid sequence with not more than 1, 2, 3 or 4 modifications, e.g., substitutions, additions or deletions thereof), a LC CDR2 amino acid sequence of SEQ ID NO:21 (or an amino acid sequence with not more than 1, 2, 3 or 4 modifications, e.g., substitutions, additions or deletions thereof), and/or a LC CDR3 amino acid sequence of SEQ ID NO:22 (or an amino acid sequence with not more than 1, 2, 3 or 4 modifications, e.g., substitutions, additions or deletions thereof); and/or
(ii) a VH comprising: a HC CDR1 amino acid sequence of SEQ ID NO: 17 (or an amino acid sequence with not more than 1, 2, 3 or 4 modifications, e.g., substitutions, additions or deletions thereof), a HC CDR2 amino acid sequence of SEQ ID NO:18 (or an amino acid sequence with not more than 1, 2, 3 or 4 modifications, e.g., substitutions, additions or deletions thereof), and/or a HC CDR3 amino acid sequence of SEQ ID NO:19 (or an amino acid sequence with not more than 1, 2, 3 or 4 modifications, e.g., substitutions, additions or deletions thereof).

In some embodiments of any of the compositions disclosed herein, the anti-TCRβV antibody molecule comprises an antigen binding domain comprising:
a variable heavy chain (VH) of SEQ ID NO: 23, SEQ ID NO: 24 or SEQ ID NO: 25, or a sequence having at least about 75%, 80%, 85%, 90%, 95%, or 99% sequence identity thereto; and/or
a variable light chain (VL) of SEQ ID NO: 26, SEQ ID NO: 27, SEQ ID NO: 28, SEQ ID NO: 29, or SEQ ID NO: 30, or a sequence having at least about 75%, 80%, 85%, 90%, 95%, or 99% sequence identity thereto.

In some embodiments of any of the compositions disclosed herein, the anti-TCRβV antibody molecule comprises a light chain comprising a framework region, e.g., framework region 1 (FR1), comprising one, two or all (e.g., three) of: (i) an Aspartic Acid at position 1, e.g., a substitution at position 1 according to Kabat numbering, e.g., a Alanine to Aspartic Acid substitution; or (ii) an Asparagine at position 2, e.g., a substitution at position 2 according to Kabat numbering, e.g., a Isoleucine to Asparagine substitution, a Serine to Asparagine substitution, or a Tyrosine to Asparagine substitution; or (iii) a Leucine at position 4, e.g., a substitution at position 4 according to Kabat numbering, e.g., a Methionine to Leucine substitution. In some embodiments, the substitution is relative to a human germline light chain framework region sequence.

In some embodiments of any of the compositions disclosed herein, the anti-TCRβV antibody molecule comprises a light chain comprising a framework region, e.g., framework region 3 (FR3), comprising one, two or all (e.g., three) of: (i) a Glycine as position 66, e.g., a substitution at position 66 according to Kabat numbering, e.g., a Lysine to Glycine substitution, or a Serine to Glycine substitution; or (ii) an Asparagine at position 69, e.g., a substitution at position 69 according to Kabat numbering, e.g., a Threonine to Asparagine substitution; or (iii) a Tyrosine at position 71, e.g., a substitution at position 71 according to Kabat numbering, e.g., a Phenylalanine to Tyrosine substitution, or an Alanine to Tyrosine substitution. In some embodiments, the substitution is relative to a human germline light chain framework region sequence.

In some embodiments of any of the compositions disclosed herein, the anti-TCRβV antibody molecule binds to TCRβ V12, e.g., TCRβ V12-4*01, TCRβ V12-3*01, or TCRβ V12-5*01. In some embodiments the anti-TCRβV antibody molecule binds to TCRβ V12-4*01 or TCRβ V12-3*01.

In some embodiments, TCRβ V12, e.g., TCRβ V12-4*01, TCRβ V12-3*01, or TCRβ V12-5*01 is recognized, e.g., bound, by SEQ ID NO: 15 and/or SEQ ID NO: 16. In some embodiments, TCRβ V12, e.g., TCRβ V12-4*01, TCRβ V12-3*01, or TCRβ V12-5*01, is recognized, e.g., bound, by any one of SEQ ID NOs 23-25, and/or any one of SEQ ID NO: 26-30A, or an amino acid sequence having at least about 75%, 80%, 85%, 90%, 95%, or 99% sequence identity thereto. In some embodiments TCRβ V12-4*01 is recognized, e.g., bound, by any one of SEQ ID NOs 23-25, and/or any one of SEQ ID NO: 26-30, or an amino acid sequence having at least about 75%, 80%, 85%, 90%, 95%, or 99% sequence identity thereto. In some embodiments TCRβ V12-3*01 is recognized, e.g., bound, by any one of SEQ ID NOs 23-25, and/or any one of SEQ ID NO: 26-30, or an amino acid sequence having at least about 75%, 80%, 85%, 90%, 95%, or 99% sequence identity thereto.

In some embodiments of any of the compositions disclosed herein, the anti-TCRβV antibody molecule comprises the anti-TCRβV antibody molecule comprises an antigen binding domain comprising a single chain Fv (scFv) or a Fab.

In some embodiments of any of the compositions disclosed herein, the anti-TCRβV antibody molecule comprises binds to a conformational or a linear epitope on the T cell.

In some embodiments of any of the compositions disclosed herein, the anti-TCRβV antibody molecule is a full antibody (e.g., an antibody that includes at least one, and preferably two, complete heavy chains, and at least one, and preferably two, complete light chains), or an antigen-binding fragment (e.g., a Fab, F(ab′)₂, Fv, a single chain Fv fragment, a single domain antibody, a diabody (dAb), a bivalent antibody, or bispecific antibody or fragment thereof, a single domain variant thereof, or a camelid antibody.

In some embodiments of any of the compositions disclosed herein, the anti-TCRβV antibody molecule comprises the anti-TCRβV antibody molecule comprises a heavy chain constant region chosen from IgG1, IgG2, IgG3, or IgG4, or a fragment thereof.

In some embodiments of any of the compositions disclosed herein, the anti-TCRβV antibody molecule comprises a light chain constant region chosen from the light chain constant regions of kappa or lambda, or a fragment thereof.

In some embodiments, the anti-TCRβV antibody molecule in a multispecific molecule disclosed herein is a first immune cell engager moiety. In some embodiments, the anti-TCRβV antibody molecule does not bind to TCRβ V12, or binds to TCRβ V12 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of the murine mAb Antibody B or a humanized version thereof (e.g., humanized mAb Antibody B-H.1 to B-H.6) as described in U.S. Pat. No. 5,861,155. In some embodiments, the anti-TCRβV antibody molecule binds to TCRβ V12 with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the murine mAb Antibody B or a humanized version thereof (e.g., humanized mAb Antibody B-H.1 to B-H.6) as described in U.S. Pat. No. 5,861,155. In some embodiments, the anti-TCRβV antibody molecule binds to a TCRβV region other than TCRβ V12 (e.g., TCRβV region as described herein, e.g., TCRβ V6 subfamily (e.g., TCRβ V6-5*01) with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the murine mAb Antibody B or a humanized version thereof (e.g., humanized mAb Antibody B-H.1 to B-H.6) as described in U.S. Pat. No. 5,861,155. In some embodiments, the anti-TCRβV antibody molecule does not comprise the CDRs of the murine mAb Antibody B.

In some embodiments, the anti-TCRβV antibody molecule in a multispecific molecule disclosed herein is a first immune cell engager moiety. In some embodiments, the anti-TCRβV antibody molecule does not bind to TCRβ V5-5*01 or TCRβ V5-1*01, or binds to TCRβ V5-5*01 or TCRβ V5-1*01 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of murine Antibody C or a humanized version thereof as described in U.S. Pat. No. 5,861,155. In some embodiments, the anti-TCRβV antibody molecule binds to TCRβ V5-5*01 or TCRβ V5-1*01 with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of murine Antibody C or a humanized version thereof as described in U.S. Pat. No. 5,861,155. In some embodiments, the anti-TCRβV antibody molecule binds to a TCRβV region other than TCRβ V5-5*01 or TCRβ V5-1*01 (e.g., TCRβV region as described herein, e.g., TCRβ V6 subfamily (e.g., TCRβ V6-5*01) with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of murine Antibody C or a humanized version thereof as described in U.S. Pat. No. 5,861,155. In some embodiments, the anti-TCRβV antibody molecule does not comprise the CDRs of murine Antibody C.

In some embodiments, the multispecific molecule further comprises a second immune cell engager moiety. In some embodiments, the first and/or second immune cell engager binds to and activates an immune cell, e.g., an effector cell. In some embodiments, the first and/or second immune cell engager binds to, but does not activate, an immune cell, e.g., an effector cell. In some embodiments, the second immune cell engager is chosen from an NK cell engager, a T cell engager, a B cell engager, a dendritic cell engager, or a macrophage cell engager, or a combination thereof. In some embodiments, the second immune cell engager comprises a T cell engager which binds to CD3, TCRa, TCRy, TCR, ICOS, CD28, CD27, HVEM, LIGHT, CD40, 4-1BB, OX40, DR3, GITR, CD30, TIMI, SLAM, CD2, or CD226.

In some embodiments, a multispecific molecule disclosed herein comprises an infectious disease-targeting moiety. In some embodiment, the infectious disease-targeting moiety comprises an antibody molecule (e.g., Fab or scFv), a receptor molecule (e.g., a receptor, a receptor fragment or functional variant thereof), or a ligand molecule (e.g., a ligand, a ligand fragment or functional variant thereof), or a combination thereof, that binds to an antigen from an infectious agent, e.g., a bacteria (e.g., Mycobacterium tuberculosis), virus (e.g., Epstein-Barr virus (EBV), influenza virus, human immunodeficiency virus (HIV), simian immunodeficiency virus (SIV), human cytomegalovirus (HCMV)), or eukaryotic infectious agent (e.g., a malaria parasite). In some embodiments, the infectious disease-targeting moiety binds to an antigen present on an infectious agent, e.g., a bacteria (e.g., Mycobacterium tuberculosis), virus (e.g., Epstein-Barr virus (EBV), influenza virus, human immunodeficiency virus (HIV), simian immunodeficiency virus (SIV), human cytomegalovirus (HCMV)), or eukaryotic infectious agent (e.g., a malaria parasite).

In some embodiments, the infectious disease-targeting antibody molecule binds to a conformational or a linear epitope on an antigen from an infectious agent, e.g., as described herein.

In some embodiments of any of the compositions or methods disclosed herein, the infectious disease-targeting moiety is an antigen, e.g., an infectious disease antigen, e.g., an antigen from a bacterium (e.g., Mycobacterium tuberculosis), virus (e.g., Epstein-Barr virus (EBV), influenza virus, human immunodeficiency virus (HIV), simian immunodeficiency virus (SIV), human cytomegalovirus (HCMV)), or eukaryotic infectious agent (e.g., a malaria parasite).

In some embodiments of any of the compositions or methods disclosed herein, the infectious disease-targeting moiety binds to an antigen chosen from: EBNA3 (e.g., 339-347), EBNA1 (e.g., 407-417), BZLF1 (e.g., 52-64), matrix protein (e.g., influenza virus matrix protein, e.g., 58-66), HIV Gag (e.g., HIV Gag p17, e.g., 77-85), HIV Env, HIV p24 capsid, SIV Tat (e.g., 28-35), SIV Gag (e.g., 181-189), or HCMV pp65 (e.g., 495-503).

In some embodiments of any of the compositions or methods disclosed herein, the infectious disease includes but not limited to: Epstein-Barr virus (EBV), influenza, human immunodeficiency virus (HIV), simian immunodeficiency virus (SIV), tuberculosis, malaria, or human cytomegalovirus (HCMV).

In some embodiments, a multispecific molecule disclosed herein further comprises a cytokine molecule, e.g., one or two cytokine molecules. In some embodiments, the cytokine molecule is chosen from interleukin-2 (IL-2), interleukin-7 (IL-7), interleukin-12 (IL-12), interleukin-15 (IL-15), interleukin-18 (IL-18), interleukin-21 (IL-21), or interferon gamma, or a fragment, variant or combination thereof. In some embodiments, is a monomer or a dimer. In some embodiments, the cytokine molecule further comprises a receptor dimerizing domain, e.g., an IL15Ralpha dimerizing domain. In some embodiments, the cytokine molecule (e.g., IL-15) and the receptor dimerizing domain (e.g., an IL15Ralpha dimerizing domain) are not covalently linked, e.g., are non-covalently associated.

In some embodiments, a multispecific molecule disclosed herein comprises:
(i) an anti-TCRβV antibody molecule (e.g., an anti-TCRβV antibody molecule as described herein); and
(ii) an infectious disease-targeting antibody molecule (e.g., an antibody molecule that binds to an antigen as described herein, e.g., chosen from one or more of EBNA3 (e.g., 339-347), EBNA1 (e.g., 407-417), BZLF1 (e.g., 52-64), matrix protein (e.g., influenza virus matrix protein, e.g., 58-66), HIV Gag (e.g., HIV Gag p17, e.g., 77-85), HIV Env, HIV p24 capsid, SIV Tat (e.g., 28-35), SIV Gag (e.g., 181-189), or HCMV pp65 (e.g., 495-503)).

In some embodiments, a multispecific molecule disclosed herein further comprises an immunoglobulin constant region (e.g., Fc region) chosen from the heavy chain constant regions of IgG1, IgG2, and IgG4, more particularly, the heavy chain constant region of human IgG1, IgG2 or IgG4. In some embodiments, the immunoglobulin constant region (e.g., an Fc region) is linked, e.g., covalently linked to, one or more of an infectious disease-targeting moiety (e.g., which can bind to one or more of EBNA3 (e.g., 339-347), EBNA1 (e.g., 407-417), BZLF1 (e.g., 52-64), matrix protein (e.g., influenza virus matrix protein, e.g., 58-66), HIV Gag (e.g., HIV Gag p17, e.g., 77-85), HIV Env, HIV p24 capsid, SIV Tat (e.g., 28-35), SIV Gag (e.g., 181-189), or HCMV pp65 (e.g., 495-503)), the immune cell engager, the cytokine molecule, or the stromal modifying moiety. In some embodiments, an interface of a first and second immunoglobulin chain constant regions (e.g., Fc region) is altered, e.g., mutated, to increase or decrease dimerization, e.g., relative to a non-engineered interface. In some embodiments, the dimerization of the immunoglobulin chain constant region (e.g., Fc region) is enhanced by providing an Fc interface of a first and a second Fc region with one or more of: a paired cavity-protuberance (“knob-in-a hole”), an electrostatic interaction, or a strand-exchange, such that a greater ratio of heteromultimer:homomultimer forms, e.g., relative to a non-engineered interface. In some embodiments,

In some embodiments, a multispecific molecule disclosed herein further comprises a linker, e.g., a linker described herein, optionally wherein the linker is selected from: a cleavable linker, a non-cleavable linker, a peptide linker, a flexible linker, a rigid linker, a helical linker, or a non-helical linker.

In some embodiments, the multispecific molecule comprises at least two non-contiguous polypeptide chains.

In some embodiments, the multispecific molecule comprises the following configuration:

A, B-[dimerization module]-C, -D

wherein:
(1) the dimerization module comprises an immunoglobulin constant domain, e.g., a heavy chain constant domain (e.g., a homodimeric or heterodimeric heavy chain constant region, e.g., an Fc region), or a constant domain of an immunoglobulin variable region (e.g., a Fab region); and
(2) A, B, C, and D are independently absent; (i) an antigen binding domain that preferentially binds to a first immune cell engager comprising an anti-TCRβV antibody molecule disclosed herein; (ii) an infectious disease-targeting moiety (e.g., as described herein), (iii) a second immune cell engager chosen from a T cell engager, an NK cell engager, a B cell engager, a dendritic cell engager, or a macrophage cell engager; (iv) a cytokine molecule; or (v) a stromal modifying moiety, provided that:
at least one, two, or three of A, B, C, and D comprises an antigen binding domain that preferentially binds to a TCRβV region disclosed herein, and
any of the remaining A, B, C, and D is absent or comprises one of an infectious disease-targeting moiety, a second immune cell engager, a cytokine molecule, or a stromal modifying moiety.

In some embodiments, the dimerization module comprises one or more immunoglobulin chain constant regions (e.g., Fc regions) comprising one or more of: a paired cavity-protuberance (“knob-in-a hole”), an electrostatic interaction, or a strand-exchange. In some embodiments, the one or more immunoglobulin chain constant regions (e.g., Fc regions) comprise an amino acid substitution at a position chosen from one or more of 347, 349, 350, 351, 366, 368, 370, 392, 394, 395, 397, 398, 399, 405, 407, or 409, e.g., of the Fc region of human IgG1. In some embodiments, the one or more immunoglobulin chain constant regions (e.g., Fc regions) comprise an amino acid substitution chosen from: T366S, L368A, or Y407V (e.g., corresponding to a cavity or hole), or T366W (e.g., corresponding to a protuberance or knob), or a combination thereof.

In some embodiments, the multispecific molecule further comprises a linker, e.g., a linker between one or more of: the antigen binding domain of an anti-TCRβV antibody molecule disclosed herein and the infectious disease-targeting moiety; the antigen binding domain of an anti-TCRβV antibody molecule disclosed herein and the second immune cell engager, the antigen binding domain of an anti-TCRβV antibody molecule disclosed herein and the cytokine molecule, the antigen binding domain of an anti-TCRβV antibody molecule disclosed herein and the stromal modifying moiety, the second immune cell engager and the cytokine molecule, the second immune cell engager and the stromal modifying moiety, the cytokine molecule and the stromal modifying moiety, the antigen binding domain of an anti-TCRβV antibody molecule disclosed herein and the dimerization module, the second immune cell engager and the dimerization module, the cytokine molecule and the dimerization module, the stromal modifying moiety and the dimerization module, the infectious disease-targeting moiety and the dimerization module, the infectious disease-targeting moiety and the cytokine molecule, the infectious disease-targeting moiety and the second immune cell engager, or the infectious disease-targeting moiety and the antigen binding domain of an anti-TCRβV antibody molecule disclosed herein. In some embodiments, the linker is chosen from: a cleavable linker, a non-cleavable linker, a peptide linker, a flexible linker, a rigid linker, a helical linker, or a non-helical linker. In some embodiments, the linker is a peptide linker. In some embodiments, the peptide linker comprises Gly and Ser. In some embodiments, the peptide linker comprises an amino acid sequence chosen from SEQ ID NOs: 3460-3463 or 3467-3470.

In another aspect, the disclosure provides an isolated nucleic acid molecule comprising a nucleotide sequence encoding an anti-TCRβV antibody molecule disclosed herein, or a nucleotide sequence having at least 75%, 80%, 85%, 90%, 95%, or 99% identity thereto.

In another aspect, the disclosure provides an isolated nucleic acid molecule comprising a nucleotide sequence encoding a multispecific molecule disclosed herein, or a nucleotide sequence having at least 75%, 80%, 85%, 90%, 95%, or 99% identity thereto.

In another aspect, the disclosure provides a method of making, e.g., producing, an anti-TCRβV antibody molecule, a multispecific molecule described herein, comprising culturing a host cell described herein, under suitable conditions. In some embodiments of a method of making a multispecific molecule, the conditions comprise, e.g., conditions suitable for gene expression and/or homo- or heterodimerization.

In another aspect, the disclosure provides a pharmaceutical composition comprising an anti-TCRβV antibody molecule, or a multispecific molecule described herein, and a pharmaceutically acceptable carrier, excipient, or stabilizer.

In an aspect, provided herein is a method of treating a disease e.g., an infectious disease, in a subject comprising administering to the subject an effective amount, e.g., a therapeutically effective amount, of an anti-TCRβV antibody molecule or a multispecific molecule comprising an anti-TCRβV antibody molecule disclosed herein, thereby treating the disease.

In a related aspect, provided herein is a composition comprising an anti-TCRβV antibody molecule or a multispecific molecule comprising an anti-TCRβV antibody molecule disclosed herein, for use in the treatment of a disease, e.g., an infectious disease, in a subject.

In some embodiments, the method further comprises administering a second agent, e.g., therapeutic agent, e.g., as described herein. In some embodiments, second agent comprises a therapeutic agent. In some embodiments, therapeutic agent is a biologic agent.

In another aspect, provided herein is a method of targeting, e.g., directing or re-directing, a therapy, e.g., treatment, to a T cell, e.g., in a subject, e.g., having a disease, e.g., an infectious disease, comprising administering an effective amount of: (i) an anti-TCRβV antibody disclosed herein; and (ii) the therapy, e.g., an infectious disease-targeting therapy (e.g., an antibody that binds to an antigen as described herein), e.g., as described herein, thereby targeting the T cell.

In some embodiments, (i) and (ii) are conjugated, e.g., linked.

In some embodiments, (i) and (ii) are administered simultaneously or concurrently.

In some embodiments, the method results in: reduced cytokine release syndrome (CRS) (e.g., lesser duration of CRS or no CRS), or a reduced severity of CRS (e.g., absence of severe CRS, e.g., CRS grade 4 or 5) compared to administration of (ii) alone. In some embodiments, CRS is assessed by an assay of Example 3.

In yet another aspect, the disclosure provides, a method of targeting a T cell, e.g., in a subject having a disease, e.g., an infectious disease, with an anti-TCRβV antibody disclosed herein or a multispecific molecule comprising an anti-TCRβV antibody disclosed herein.

In another aspect, the disclosure provides a method of treating, e.g., preventing or reducing, cytokine release syndrome (CRS) in a subject, e.g., CRS associated with a treatment, e.g., a previously administered treatment, comprising administering to the subject an effective amount of an anti-TCRβV antibody disclosed herein or a multispecific molecule comprising an anti-TCRβV antibody disclosed herein, wherein, the subject has a disease, e.g., an infectious disease, thereby treating, e.g., preventing or reducing, CRS in the subject

In a related aspect, the disclosure provides a composition comprising an anti-TCRβV antibody disclosed herein or a multispecific molecule comprising an anti-TCRβV antibody disclosed herein, for use in the treatment, e.g., prevention or reduction, of cytokine release syndrome (CRS) in a subject, e.g., CRS associated with a treatment, e.g., a previously administered treatment, comprising administering to the subject an effective amount of the anti-TCRβV antibody, wherein the subject has a disease, e.g., an infectious disease.

In some embodiments of a method or composition for use disclosed herein, the anti-TCRβV antibody is administered concurrently with or after the administration of the treatment associated with CRS.

In some embodiments of any of the methods disclosed herein, the anti-TCRβV antibody molecule comprises an antigen binding domain comprising:
(i) a heavy chain complementarity determining region 1 (HC CDR1), a heavy chain complementarity determining region 2 (HC CDR2) and/or a heavy chain complementarity determining region 3 (HC CDR3) of SEQ ID NO: 1 or SEQ ID NO: 9; or
(ii) a light chain complementarity determining region 1 (LC CDR1), a light chain complementarity determining region 2 (LC CDR2), and/or a light chain complementarity determining region 3 (LC CDR3) of SEQ ID NO: 2, SEQ ID NO: 10, or SEQ ID NO: 11.

In some embodiments of any of the methods disclosed herein, the anti-TCRβV antibody molecule comprises an antigen binding domain comprising a light chain variable region (VL) comprising one, two or all (e.g., three) of a LC CDR1, a LC CDR2 and a LC CDR3 of SEQ ID NO: 2, SEQ ID NO: 10, or SEQ ID NO: 11.

In some embodiments of any of the methods disclosed herein, the anti-TCRβV antibody molecule comprises an antigen binding domain comprising a heavy chain variable region (VH) comprising one, two or all (e.g., three) of a HC CDR1, a HC CDR2 and a HC CDR3 of SEQ ID NO:1 or SEQ ID NO: 9.

In some embodiments of any of the methods disclosed herein, the anti-TCRβV antibody molecule comprises an antigen binding domain comprising:
(i) a VL comprising: a LC CDR1 amino acid sequence of SEQ ID NO: 6 (or an amino acid sequence with not more than 1, 2, 3 or 4 modifications, e.g., substitutions, additions or deletions thereof), a LC CDR2 amino acid sequence of SEQ ID NO: 7 (or an amino acid sequence with not more than 1, 2, 3 or 4 modifications, e.g., substitutions, additions or deletions thereof), and/or a LC CDR3 amino acid sequence of SEQ ID NO: 8 (or an amino acid sequence with not more than 1, 2, 3 or 4 modifications, e.g., substitutions, additions or deletions thereof); and/or
(ii) a VH comprising: a HC CDR1 amino acid sequence of SEQ ID NO: 3 (or an amino acid sequence with not more than 1, 2, 3 or 4 modifications, e.g., substitutions, additions or deletions thereof), a HC CDR2 amino acid sequence of SEQ ID NO: 4 (or an amino acid sequence with not more than 1, 2, 3 or 4 modifications, e.g., substitutions, additions or deletions thereof), and/or a HC CDR3 amino acid sequence of SEQ ID NO: 5 (or an amino acid sequence with not more than 1, 2, 3 or 4 modifications, e.g., substitutions, additions or deletions thereof).

In some embodiments of any of the methods disclosed herein, the anti-TCRβV antibody molecule comprises an antigen binding domain comprising: a variable heavy chain (VH) of SEQ ID NO: 9, or a sequence having at least about 75%, 80%, 85%, 90%, 95%, or 99% sequence identity thereto; and/or a variable light chain (VL) of SEQ ID NO: 10 or SEQ ID NO: 11, or a sequence having at least about 75%, 80%, 85%, 90%, 95%, or 99% sequence identity thereto.

In some embodiments of any of the methods disclosed herein, the anti-TCRβV antibody molecule comprises an antigen binding domain comprising the VH amino acid sequence of SEQ ID NO: 9 and the VL amino acid sequence of SEQ ID NO: 10.

In some embodiments of any of the methods disclosed herein, the anti-TCRβV antibody molecule comprises an antigen binding domain comprising the VH amino acid sequence of SEQ ID NO: 9 and the VL amino acid sequence of SEQ ID NO: 11.

In some embodiments of any of the methods disclosed herein, the anti-TCRβV antibody molecule comprises a heavy chain comprising a framework region, e.g., framework region 3 (FR3), comprising one or both of: (i) a Threonine at position 73, e.g., a substitution at position 73 according to Kabat numbering, e.g., a Glutamic Acid to Threonine substitution; or (ii) a Glycine at position, e.g., a substitution at position 94 according to Kabat numbering, e.g., a Arginine to Glycine substitution. In some embodiments, the substitution is relative to a human germline heavy chain framework region sequence.

In some embodiments of any of the methods disclosed herein, the anti-TCRβV antibody molecule comprises a light chain comprising a framework region, e.g., framework region 1 (FR1), comprising a Phenylalanine at position 10, e.g., a substitution at position 10 according to Kabat numbering, e.g., a Serine to Phenyalanine substitution. In some embodiments, the substitution is relative to a human germline light chain framework region sequence.

In some embodiments of any of the methods disclosed herein, the anti-TCRβV antibody molecule comprises a light chain comprising a framework region, e.g., framework region 2 (FR2), comprising one or both of: (i) a Histidine at position 36, e.g., a substitution at position 36 according to Kabat numbering, e.g., a Tyrosine to Histidine substitution; or (ii) an Alanine at position 46, e.g., a substitution at position 46 according to Kabat numbering, e.g., a Arginine to Alanine substitution. In some embodiments, the substitution is relative to a human germline light chain framework region sequence.

In some embodiments of any of the methods disclosed herein, the anti-TCRβV antibody molecule comprises a light chain comprising a framework region, e.g., framework region 3 (FR3), comprising a Phenylalanine at position 87, e.g., a substitution at position 87 according to Kabat numbering, e.g., a Tyrosine to Phenyalanine substitution. In some embodiments, the substitution is relative to a human germline light chain framework region sequence.

In some embodiments of any of the methods disclosed herein, the anti-TCRβV antibody molecule binds to TCRβ V6, e.g., TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments the anti-TCRβV antibody molecule binds to TCRβ V6-5*01.

In some embodiments, TCRβ V6, e.g., TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01, is recognized, e.g., bound, by SEQ ID NO: 1 and/or SEQ ID NO: 2. In some embodiments, TCRβ V6, e.g., TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01, is recognized, e.g., bound, by SEQ ID NO: 9 and/or SEQ ID NO: 10. In some embodiments, TCRβ V6, e.g., TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01, is recognized, e.g., bound, by SEQ ID NO: 9 and/or SEQ ID NO: 11. In some embodiments, TCRβ V6-5*01 is recognized, e.g., bound by SEQ ID NO: 9 and/or SEQ ID NO: 10, or a sequence having at least about 75%, 80%, 85%, 90%, 95%, or 99% sequence identity thereto. In some embodiments, TCRβ V6-5*01 is recognized, e.g., bound by SEQ ID NO: 9 and/or SEQ ID NO: 11, or a sequence having at least about 75%, 80%, 85%, 90%, 95%, or 99% sequence identity thereto.

In some embodiments of any of the methods disclosed herein, the anti-TCRβV antibody molecule comprises an antigen binding domain comprising:
(i) a heavy chain complementarity determining region (HC CDR1), a HC CDR2 and/or a HC CDR3 of SEQ ID NO: 15, SEQ ID NO: 23, SEQ ID NO: 24, or SEQ ID NO: 25; and/or
(ii) a light chain complementarity determining region 1 (LC CDR1), a LC CDR2, and/or a LC CDR3 of SEQ ID NO: 16, SEQ ID NO: 26, SEQ ID NO: 27, SEQ ID NO: 28, SEQ ID NO: 29, or SEQ ID NO: 30.

In some embodiments of any of the methods disclosed herein, the anti-TCRβV antibody molecule comprises an antigen binding domain comprising a light chain variable region (VL) comprising one, two or all of a LC CDR1, a LC CDR2 and a LC CDR3 of SEQ ID NO: 16, SEQ ID NO: 26, SEQ ID NO: 27, SEQ ID NO: 28, SEQ ID NO: 29, or SEQ ID NO: 30.

In some embodiments of any of the methods disclosed herein, the anti-TCRβV antibody molecule comprises an antigen binding domain comprising a heavy chain variable region (VH) comprising one, two or all of a HC CDR1, a HC CDR2 and a HC CDR3 of SEQ ID NO: 15, SEQ ID NO: 23, SEQ ID NO: 24, or SEQ ID NO: 25.

In some embodiments of any of the methods disclosed herein, the anti-TCRβV antibody molecule comprises an antigen binding domain comprising:
(i) a VL comprising: a LC CDR1 amino acid sequence of SEQ ID NO: 20 (or an amino acid sequence with not more than 1, 2, 3 or 4 modifications, e.g., substitutions, additions or deletions thereof), a LC CDR2 amino acid sequence of SEQ ID NO:21 (or an amino acid sequence with not more than 1, 2, 3 or 4 modifications, e.g., substitutions, additions or deletions thereof), and/or a LC CDR3 amino acid sequence of SEQ ID NO: 22 (or an amino acid sequence with not more than 1, 2, 3 or 4 modifications, e.g., substitutions, additions or deletions thereof); and/or (ii) a VH comprising: a HC CDR1 amino acid sequence of SEQ ID NO: 17 (or an amino acid sequence with not more than 1, 2, 3 or 4 modifications, e.g., substitutions, additions or deletions thereof), a HC CDR2 amino acid sequence of SEQ ID NO:18 (or an amino acid sequence with not more than 1, 2, 3 or 4 modifications, e.g., substitutions, additions or deletions thereof), and/or a HC CDR3 amino acid sequence of SEQ ID NO:19 (or an amino acid sequence with not more than 1, 2, 3 or 4 modifications, e.g., substitutions, additions or deletions thereof).

In some embodiments of any of the compositions disclosed herein, the anti-TCRβV antibody molecule comprises an antigen binding domain comprising:
a variable heavy chain (VH) of SEQ ID NO: 23, SEQ ID NO: 24, or SEQ ID NO: 25, or a sequence having at least about 75%, 80%, 85%, 90%, 95%, or 99% sequence identity thereto; and/or
a variable light chain (VL) of SEQ ID NO: 26, SEQ ID NO: 27, SEQ ID NO: 28, SEQ ID NO: 29, or SEQ ID NO:30, or a sequence having at least about 75%, 80%, 85%, 90%, 95%, or 99% sequence identity thereto.

In some embodiments of any of the methods disclosed herein, the anti-TCRβV antibody molecule comprises a light chain comprising a framework region, e.g., framework region 1 (FR1), comprising one, two or all (e.g., three) of: (i) an Aspartic Acid at position 1, e.g., a substitution at position 1 according to Kabat numbering, e.g., a Alanine to Aspartic Acid substitution; or (ii) an Asparagine at position 2, e.g., a substitution at position 2 according to Kabat numbering, e.g., a Isoleucine to Asparagine substitution, a Serine to Asparagine substitution, or a Tyrosine to Asparagine substitution; or (iii) a Leucine at position 4, e.g., a substitution at position 4 according to Kabat numbering, e.g., a Methionine to Leucine substitution. In some embodiments, the substitution is relative to a human germline light chain framework region sequence.

In some embodiments of any of the methods disclosed herein, the anti-TCRβV antibody molecule comprises a light chain comprising a framework region, e.g., framework region 3 (FR3), comprising one, two or all (e.g., three) of: (i) a Glycine as position 66, e.g., a substitution at position 66 according to Kabat numbering, e.g., a Lysine to Glycine substitution, or a Serine to Glycine substitution; or (ii) an Asparagine at position 69, e.g., a substitution at position 69 according to Kabat numbering, e.g., a Threonine to Asparagine substitution; or (iii) a Tyrosine at position 71, e.g., a substitution at position 71 according to Kabat numbering, e.g., a Phenylalanine to Tyrosine substitution, or an Alanine to Tyrosine substitution. In some embodiments, the substitution is relative to a human germline light chain framework region sequence.

In some embodiments of any of the methods disclosed herein, the anti-TCRβV antibody molecule binds to TCRβ V12, e.g., TCRβ V12-4*01, TCRβ V12-3*01, or TCRβ V12-5*01. In some embodiments the anti-TCRβV antibody molecule binds to TCRβ V12-4*01 or TCRβ V12-3*01.

In some embodiments, TCRβ V12, e.g., TCRβ V12-4*01, TCRβ V12-3*01, or TCRβ V12-5*01 is recognized, e.g., bound, by SEQ ID NO: 15 and/or SEQ ID NO: 16. In some embodiments, TCRβ V12, e.g., TCRβ V12-4*01, TCRβ V12-3*01, or TCRβ V12-5*01, is recognized, e.g., bound, by any one of SEQ ID NOs 23-25, and/or any one of SEQ ID NO: 26-30A, or an amino acid sequence having at least about 75%, 80%, 85%, 90%, 95%, or 99% sequence identity thereto. In some embodiments TCRβ V12-4*01 is recognized, e.g., bound, by any one of SEQ ID NOs 23-25, and/or any one of SEQ ID NO: 26-30, or an amino acid sequence having at least about 75%, 80%, 85%, 90%, 95%, or 99% sequence identity thereto. In some embodiments TCRβ V12-3*01 is recognized, e.g., bound, by any one of SEQ ID NOs 23-25, and/or any one of SEQ ID NO: 26-30, or an amino acid sequence having at least about 75%, 80%, 85%, 90%, 95%, or 99% sequence identity thereto.

In some embodiments of any of the methods disclosed herein, the anti-TCRβV antibody molecule comprises the anti-TCRβV antibody molecule comprises an antigen binding domain comprising a single chain Fv (scFv) or a Fab.

In some embodiments of any of the methods disclosed herein, the anti-TCRβV antibody molecule comprises binds to a conformational or a linear epitope on the T cell.

In some embodiments of any of the methods disclosed herein, the anti-TCRβV antibody molecule is a full antibody (e.g., an antibody that includes at least one, and preferably two, complete heavy chains, and at least one, and preferably two, complete light chains), or an antigen-binding fragment (e.g., a Fab, F(ab′)₂, Fv, a single chain Fv fragment, a single domain antibody, a diabody (dAb), a bivalent antibody, or bispecific antibody or fragment thereof, a single domain variant thereof, or a camelid antibody.

In some embodiments of any of the methods disclosed herein, the anti-TCRβV antibody molecule comprises the anti-TCRβV antibody molecule comprises a heavy chain constant region chosen from IgG1, IgG2, IgG3, or IgG4, or a fragment thereof.

In some embodiments of any of the methods disclosed herein, the anti-TCRβV antibody molecule comprises a light chain constant region chosen from the light chain constant regions of kappa or lambda, or a fragment thereof.

In some embodiments of any of the methods disclosed herein, the anti-TCRβV antibody molecule does not bind to TCRβ V12, or binds to TCRβ V12 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of the murine mAb Antibody B or a humanized version thereof (e.g., humanized mAb Antibody B-H.1 to B-H.6) as described in U.S. Pat. No. 5,861,155.

In some embodiments of any of the methods disclosed herein, the anti-TCRβV antibody molecule binds to TCRβ V12 with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the murine mAb Antibody B or a humanized version thereof (e.g., humanized mAb Antibody B-H.1 to B-H.6) as described in U.S. Pat. No. 5,861,155.

In some embodiments of any of the methods disclosed herein, the anti-TCRβV antibody molecule binds to a TCRβV region other than TCRβ V12 (e.g., TCRβV region as described herein, e.g., TCRβ V6 subfamily (e.g., TCRβ V6-5*01) with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the murine mAb Antibody B or a humanized version thereof (e.g., humanized mAb Antibody B-H.1 to B-H.6) as described in U.S. Pat. No. 5,861,155.

In some embodiments of any of the methods disclosed herein, the anti-TCRβV antibody molecule does not comprise the CDRs of the murine mAb Antibody B.

In some embodiments of any of the methods disclosed herein, the anti-TCRβV antibody molecule does not bind to TCRβ V5-5*01 or TCRβ V5-1*01, or binds to TCRβ V5-5*01 or TCRβ V5-1*01 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of murine Antibody C or a humanized version thereof as described in U.S. Pat. No. 5,861,155.

In some embodiments of any of the methods disclosed herein, the anti-TCRβV antibody molecule binds to TCRβ V5-5*01 or TCRβ V5-1*0lwith an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of murine Antibody C or a humanized version thereof as described in U.S. Pat. No. 5,861,155.

In some embodiments of any of the methods disclosed herein, the anti-TCRβV antibody molecule binds to a TCRβV region other than TCRβ V5-5*01 or TCRβ V5-1*01 (e.g., TCRβV region as described herein, e.g., TCRβ V6 subfamily (e.g., TCRβ V6-5*01) with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of murine Antibody C or a humanized version thereof as described in U.S. Pat. No. 5,861,155.

In some embodiments of any of the methods disclosed herein, the anti-TCRβV antibody molecule does not comprise the CDRs of murine Antibody C.

In some embodiments of a method or composition for use disclosed herein, the disease is an infectious disease chosen from: Epstein-Barr virus (EBV), influenza, human immunodeficiency virus (HIV), simian immunodeficiency virus (SIV), tuberculosis, malaria, or human cytomegalovirus (HCMV), or a combination thereof.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIGS. 1A-1B shows the alignment of the Antibody A source mouse VH and VL framework 1, CDR 1, framework 2, CDR 2, framework 3, CDR3, and framework 4 regions with their respective humanized sequences. Kabat CDRs are shown in bold, Chothia CDRs are shown in italics, and combined CDRs are shown in boxes. The framework positions that were back mutated are double underlined. FIG. 1A shows VH sequences for murine Antibody A (SEQ ID NO: 1) and humanized Antibody A-H (SEQ ID NO: 9). FIG. 1B shows VL sequences for murine Antibody A (SEQ ID NO: 2) and humanized Antibody A-H (SEQ ID NO: 10 and SEQ ID NO: 11).

FIGS. 2A-2B shows the alignment of the Antibody B source mouse VH and VL framework 1, CDR 1, framework 2, CDR 2, framework 3, CDR3, and framework 4 regions with their respective humanized sequences. Kabat CDRs are shown in bold, Chothia CDRs are shown in italics, and combined CDRs are shown in boxes. The framework positions that were back mutated are double underlined. FIG. 2A shows the VH sequence for murine Antibody B (SEQ ID NO: 15) and humanized VH sequences B-H.1A to B-H.1C (SEQ ID NOs: 23-25). FIG. 2B shows the VL sequence for murine Antibody B (SEQ ID NO: 16) and humanized VL sequences B-H.1D to B-H.1H (SEQ ID NOs: 26-30).

FIG. 3 depicts the phylogenetic tree of TCRBV gene family and subfamilies with corresponding antibodies mapped. Subfamily identities are as follows: Subfamily A: TCRβ V6; Subfamily B: TCRβ V10; Subfamily C: TCRβ V12; Subfamily D: TCRβ V5; Subfamily E: TCRβ V7; Subfamily F: TCRβ V11; Subfamily G: TCRβ V14; Subfamily H: TCRβ V16; Subfamily I:TCRβ V18; Subfamily J:TCRβ V9; Subfamily K: TCRβ V13; Subfamily L: TCRβ V4; Subfamily M:TCRβ V3; Subfamily N:TCRβ V2; Subfamily 0:TCRβ V15; Subfamily P: TCRβ V30; Subfamily Q: TCRβ V19; Subfamily R:TCRβ V27; Subfamily S:TCRβ V28; Subfamily T: TCRβ V24; Subfamily U: TCRβ V20; Subfamily V: TCRβ V25; and Subfamily W:TCRβ V29 subfamily. Subfamily members are described in detail herein in the Section titled “TCR beta V (TCRβV)”.

FIGS. 4A-4C show human CD3+T cells activated by anti-TCR Vβ13.1 antibody (A-H.1) for 6-days. Human CD3+T cells were isolated using magnetic-bead separation (negative selection) and activated with immobilized (plate-coated) anti-TCR Vβ13.1 (A-H.1) or anti-CD3E (OKT3) antibodies at 100 nM for 6 days. FIG. 4A shows two scatter plots (left: activated with OKT3; and right: activated with A-H.1) of expanded T cells assessed for TCR Vβ13.1 surface expression using anti-TCR Vβ13.1 (A-H.1) followed by a secondary fluorochrome- conjugated antibody for flow cytometry analysis. FIG. 4B shows percentage (%) of TCR Vβ13.1 positive T cells activated by anti-TCR Vβ13.1 (A-H.1) or anti-CD3e (OKT3) plotted against total T cells (CD3+). FIG. 4C shows relative cell count acquired by counting the number of events in each T cell subset gate (CD3 or TCR Vβ13.1) for 20 seconds at a constant rate of 60 μl/min. Data shown as mean value from 3 donors.

FIGS. 5A-5B show cytolytic activity of human CD3+T cells activated by anti-TCR Vβ13.1 antibody (A-H.1) against transformed cell line RPMI 8226. FIG. 5A depicts target cell lysis of human CD3+T cells activated with A-H.lor OKT3. Human CD3+T cells were isolated using magnetic-bead separation (negative selection) and activated with immobilized (plate-coated) A-H.1 or OKT3 at the indicated concentrations for 4 days prior to co-culture with RPMI 8226 cells at a (E:T) ratio of 5:1 for 2 days. Samples were next analyzed for cell lysis of RPMI 8226 cells by FACS staining for CFSE/CD138-labeled, and membrane-impermeable DNA dyes (DRAQ7) using flow cytometry analysis. FIG. 5B shows target cell lysis of human CD3+T cells activated with A-H.1 or OKT3 incubated with RPMI-8226 at a (E:T) ratio of 5:1 for 6 days followed by cell lysis analysis of RPMI 8226 cells as described above. Percentage (%) target cell lysis was determined by normalizing to basal target cell lysis (i.e. without antibody treatment) using the following formula, [(x−basal)/(100%−basal), where xis cell lysis of sample]. Data shown is a representative of n=1 donor.

FIGS. 6A-6B show IFNγ production by human PBMCs activated with the indicated antibodies. Human PBMCs were isolated from whole blood from the indicated number of donors, followed by solid-phase (plate-coated) stimulation with the indicated antibodies at 100Nm. Supernatant was collected on Days 1, 2, 3, 5, or 6. FIG. 6A is a graph comparing the production of IFNγ in human PBMCs activated with the antibodies indicated activated with anti-TCR Vβ13.1 antibodies (A-H.1 or A-H.2) or anti-CD3e antibodies (OKT3 or SP34-2) on Day 1, 2, 3, 5, or 6 post-activation. FIG. 6B shows IFNγ production in human PBMCs activated with the antibodies indicated activated with the indicated anti-TCR Vβ13.1 antibodies or anti-CD3e antibody (OKT3) on Day 1, 2, 3, 5, or 6 post-activation.

FIGS. 7A-7B show IL-2 production by human PBMCs activated with the indicated antibodies. A similar experimental setup as described for FIGS. 6A-6B was used.

FIGS. 8A-8B show IL-6 production by human PBMCs activated with the indicated antibodies. A similar experimental setup as described for FIGS. 6A-6B was used.

FIGS. 9A-9B show TNF-alpha production by human PBMCs activated with the indicated antibodies. A similar experimental setup as described for FIGS. 6A-6B was used.

FIGS. 10A-10B show IL-lbeta production by human PBMCs activated with the indicated antibodies. A similar experimental setup as described for FIGS. 6A-6B was used.

FIGS. 11A-11B are graphs showing delayed kinetics of IFNγ secretion in human PMBCs activated by anti-TCR Vβ13.1 antibody A-H.1 when compared to PBMCs activated by anti-CD3e antibody OKT3. FIG. 11A shows IFNγ secretion data from 4 donors. FIG. 11B shows IFNγ secretion data from 4 additional donors. Data shown is representative of n=8 donors.

FIG. 12 depicts increased CD8+TSCM and Temra T cell subsets in human PBMCs activated by anti-TCR Vβ13.1 antibodies (A-H.1 or A-H.2) compared to PBMCs activated by anti-CD3e antibodies (OKT3 or SP34-2).

FIGS. 13A and 13B are schematics showing the alignments of affinity matured humanized antibody A-H sequences. FIG. 13A shows the alignment of affinity matured humanized Antibody A-H VL sequences (SEQ ID NOs: 3377-3389, respectively, in order of appearance). FIG. 13B shows the alignment of affinity matured humanized Antibody A-H VH sequences (SEQ ID NOs: 3390-3436, respectively, in order of appearance).

### DETAILED DESCRIPTION OF THE INVENTION

Previous studies have shown that even low “activating” doses of anti-CD3e mAb can cause long-term T cell dysfunction and exert immunosuppressive effects. In addition, anti-CD3e mAbs have been associated with side effects that result from massive T cell activation. The large number of activated T cells secrete substantial amounts of cytokines, the most important of which is Interferon gamma (IFNg; IFNγ). This excess amount of IFNγ in turn activates the macrophages which then overproduce proinflammatory cytokines such as IL-1, IL-6 and TNF-alpha, causing a “cytokine storm” known as the cytokine release syndrome (CRS). Thus, the need exists for developing antibodies that are capable of binding and activating only a subset of effector T cells, e.g., to reduce the CRS.

This disclosure provides, inter alfa, antibodies directed to the variable chain of the beta subunit of TCR (TCRβV) which bind and, e.g., activate a subset of T cells. The anti-TCRβV antibody molecules disclosed herein result in lesser or no production of cytokines associated with CRS, e.g., IL-6, IL-lbeta and TNF alpha; and enhanced and/or delayed production of IL-2 and IFNγ. In some embodiments, the anti-TCRβV antibodies disclosed herein result in expansion of a subset of memory effector T cells known as T_{EMRA}. Without wishing to be bound by theory, it is believed that in some embodiments, T_{EMRA} cells can promote cell lysis but not CRS. Accordingly, provided herein are methods of making said anti-TCRβV antibody molecules and uses thereof. Also disclosed herein are multispecific molecules, e.g., bispecific molecules comprising said anti-TCRβV antibody molecules. In some embodiments, compositions comprising anti-TCRβV antibody molecules of the present disclosure, can be used, e.g., to activate and redirect T cells to for treating an infectious disease. In some embodiments, compositions comprising anti-TCRβV antibody molecules as disclosed herein limit the harmful side-effects of CRS, e.g., CRS associated with anti-CD3e targeting.

In some embodiments, the anti-TCRβV antibody molecule does not bind to TCRβ V12, or binds to TCRβ V12 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of the murine mAb Antibody B or a humanized version thereof (e.g., humanized mAb Antibody B-H.1 to B-H.6) as described in U.S. Pat. No. 5,861,155.

In some embodiments, the anti-TCRβV antibody molecule binds to TCRβ V12 with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the murine mAb Antibody B or a humanized version thereof (e.g., humanized mAb Antibody B-H.1 to B-H.6) as described in U.S. Pat. No. 5,861,155.

In some embodiments, the anti-TCRβV antibody molecule binds to a TCRβV region other than TCRβ V12 (e.g., TCRβV region as described herein, e.g., TCRβ V6 subfamily (e.g., TCRβ V6-5*01) with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the murine mAb Antibody B or a humanized version thereof (e.g., humanized mAb Antibody B-H.1 to B-H.6) as described in U.S. Pat. No. 5,861,155.

In some embodiments, the anti-TCRβV antibody molecule does not comprise the CDRs of the murine mAb Antibody B.

In some embodiments, the anti-TCRβV antibody molecule does not bind to TCRβ V5-5*01 or TCRβ V5-1*01, or binds to TCRβ V5-5*01 or TCRβ V5-1*01 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of murine Antibody C or a humanized version thereof as described in U.S. Pat. No. 5,861,155.

In some embodiments, the anti-TCRβV antibody molecule binds to TCRβ V5-5*01 or TCRβ V5-1*01 with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of murine Antibody C or a humanized version thereof as described in U.S. Pat. No. 5,861,155.

In some embodiments, the anti-TCRβV antibody molecule binds to a TCRβV region other than TCRβ V5-5*01 or TCRβ V5-1*01 (e.g., TCRβV region as described herein, e.g., TCRβ V6 subfamily (e.g., TCRβ V6-5*01) with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of murine Antibody C or a humanized version thereof as described in U.S. Pat. No. 5,861,155.

In some embodiments, the anti-TCRβV antibody molecule does not comprise the CDRs of murine Antibody C.

Accordingly, provided herein are, inter alfa, anti-TCRβV antibody molecules, multispecific or multifunctional molecules (e.g., multispecific or multifunctional antibody molecules) that comprise anti-TCRβV antibody molecules, nucleic acids encoding the same, methods of producing the aforesaid molecules, pharmaceutical compositions comprising aforesaid molecules, and methods of treating a disease or disorder, e.g., an infectious disease, using the aforesaid molecules. The antibody molecules and pharmaceutical compositions disclosed herein can be used (alone or in combination with other agents or therapeutic modalities) to treat, prevent and/or diagnose disorders and conditions, e.g., an infectious disease, e.g., as described herein.

### Definitions

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

The term “acquire” or “acquiring” as the terms are used herein, refer to obtaining possession of a physical entity (e.g., a sample, a polypeptide, a nucleic acid, or a sequence), or a value, e.g., a numerical value, by “directly acquiring” or “indirectly acquiring” the physical entity or value. “Directly acquiring” means performing a process (e.g., performing a synthetic or analytical method) to obtain the physical entity or value. “Indirectly acquiring” refers to receiving the physical entity or value from another party or source (e.g., a third party laboratory that directly acquired the physical entity or value). Directly acquiring a physical entity includes performing a process that includes a physical change in a physical substance, e.g., a starting material. Directly acquiring a value includes performing a process that includes a physical change in a sample or another substance, e.g., performing an analytical process which includes a physical change in a substance, e.g., a sample.

As used herein, the term “T cell receptor beta variable chain” or “TCRβV,” refers to an extracellular region of the T cell receptor beta chain which comprises the antigen recognition domain of the T cell receptor. The term TCRβV includes isoforms, mammalian, e.g., human TCRβV, species homologs of human and analogs comprising at least one common epitope with TCRβV. Human TCRβV comprises a gene family comprising subfamilies including, but not limited to: a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, or a TCRβ V29 subfamily. In some embodiments, the TCRβ V6 subfamily comprises: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments, TCRβV comprises TCRβ V6-5*01. TCRβ V6-5*01 is also known as TRBV65; TCRBV6S5; TCRBV13S1, or TCRβ V13.1. The amino acid sequence of TCRβ V6-5*01, e.g., human TCRβ V6-5*01, is known in that art, e.g., as provided by IMGT ID L36092. In some embodiments, TCRβ V6-5*01 is encoded by the nucleic acid sequence of SEQ ID NO: 43, or a sequence having 85%, 90%, 95%, 99% or more identity thereof. In some embodiments, TCRβ V6-5*01 comprises the amino acid sequence of SEQ ID NO: 44, or a sequence having 85%, 90%, 95%, 99% or more identity thereof.

In some embodiments, the multifunctional molecule includes an immune cell engager. “An immune cell engager” refers to one or more binding specificities that bind and/or activate an immune cell, e.g., a cell involved in an immune response. In embodiments, the immune cell is chosen from a T cell, an NK cell, a B cell, a dendritic cell, and/or the macrophage cell. The immune cell engager can be an antibody molecule, a receptor molecule (e.g., a full length receptor, receptor fragment, or fusion thereof (e.g., a receptor-Fc fusion)), or a ligand molecule (e.g., a full length ligand, ligand fragment, or fusion thereof (e.g., a ligand-Fc fusion)) that binds to the immune cell antigen (e.g., the T cell, the NK cell antigen, the B cell antigen, the dendritic cell antigen, and/or the macrophage cell antigen). In embodiments, the immune cell engager specifically binds to the target immune cell, e.g., binds preferentially to the target immune cell. For example, when the immune cell engager is an antibody molecule, it binds to an immune cell antigen (e.g., a T cell antigen, an NK cell antigen, a B cell antigen, a dendritic cell antigen, and/or a macrophage cell antigen) with a dissociation constant of less than about 10 nM.

In some embodiments, the multifunctional molecule includes a cytokine molecule. As used herein, a “cytokine molecule” refers to full length, a fragment or a variant of a cytokine; a cytokine further comprising a receptor domain, e.g., a cytokine receptor dimerizing domain; or an agonist of a cytokine receptor, e.g., an antibody molecule (e.g., an agonistic antibody) to a cytokine receptor, that elicits at least one activity of a naturally-occurring cytokine. In some embodiments the cytokine molecule is chosen from interleukin-2 (IL-2), interleukin-7 (IL-7), interleukin-12 (IL-12), interleukin-15 (IL-15), interleukin-18 (IL-18), interleukin-21 (IL-21), or interferon gamma, or a fragment or variant thereof, or a combination of any of the aforesaid cytokines. The cytokine molecule can be a monomer or a dimer. In embodiments, the cytokine molecule can further include a cytokine receptor dimerizing domain. In other embodiments, the cytokine molecule is an agonist of a cytokine receptor, e.g., an antibody molecule (e.g., an agonistic antibody) to a cytokine receptor chosen from an IL-15Ra or IL-21R.

As used herein, the term “molecule” as used in, e.g., antibody molecule, cytokine molecule, receptor molecule, includes full-length, naturally-occurring molecules, as well as variants, e.g., functional variants (e.g., truncations, fragments, mutated (e.g., substantially similar sequences) or derivatized form thereof), so long as at least one function and/or activity of the unmodified (e.g., naturally-occurring) molecule remains.

In some embodiments, the multifunctional molecule includes a stromal modifying moiety. A “stromal modifying moiety,” as used herein refers to an agent, e.g., a protein (e.g., an enzyme), that is capable of altering, e.g., degrading a component of, the stroma. In embodiments, the component of the stroma is chosen from, e.g., an ECM component, e.g., a glycosaminoglycan, e.g., hyaluronan (also known as hyaluronic acid or HA), chondroitin sulfate, chondroitin, dermatan sulfate, heparin sulfate, heparin, entactin, tenascin, aggrecan and keratin sulfate; or an extracellular protein, e.g., collagen, laminin, elastin, fibrinogen, fibronectin, and vitronectin.

Certain terms are defined below.

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values.

“Antibody molecule” as used herein refers to a protein, e.g., an immunoglobulin chain or fragment thereof, comprising at least one immunoglobulin variable domain sequence. An antibody molecule encompasses antibodies (e.g., full-length antibodies) and antibody fragments. In an embodiment, an antibody molecule comprises an antigen binding or functional fragment of a full-length antibody, or a full-length immunoglobulin chain. For example, a full-length antibody is an immunoglobulin (Ig) molecule (e.g., an IgG antibody) that is naturally occurring or formed by normal immunoglobulin gene fragment recombinatorial processes). In embodiments, an antibody molecule refers to an immunologically active, antigen-binding portion of an immunoglobulin molecule, such as an antibody fragment. An antibody fragment, e.g., functional fragment, is a portion of an antibody, e.g., Fab, Fab', F(ab′)₂, F(ab)₂, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab', and F(ab′)₂ fragments, and single chain variable fragments (scFvs).

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

In embodiments, an antibody molecule is monospecific, e.g., it comprises binding specificity for a single epitope. In some embodiments, an antibody molecule is multispecific, e.g., it comprises a plurality of immunoglobulin variable domain sequences, where a first immunoglobulin variable domain sequence has binding specificity for a first epitope and a second immunoglobulin variable domain sequence has binding specificity for a second epitope. In some embodiments, an antibody molecule is a bispecific antibody molecule. “Bispecific antibody molecule” as used herein refers to an antibody molecule that has specificity for more than one (e.g., two, three, four, or more) epitope and/or antigen.

“Antigen” (Ag) as used herein refers to a molecule that can provoke an immune response, e.g., involving activation of certain immune cells and/or antibody generation. Any macromolecule, including almost all proteins or peptides, can be an antigen. Antigens can also be derived from genomic recombinant or DNA. For example, any DNA comprising a nucleotide sequence or a partial nucleotide sequence that encodes a protein capable of eliciting an immune response encodes an “antigen.” In embodiments, an antigen does not need to be encoded solely by a full length nucleotide sequence of a gene, nor does an antigen need to be encoded by a gene at all. In embodiments, an antigen can be synthesized or can be derived from a biological sample, e.g., a tissue sample, a cell, or a fluid with other biological components. As used, herein an “infectious disease antigen” includes any molecule present on, or associated with, an infectious disease or an agent that causes an infectious disease, e.g., a bacteria, virus, eukaryotic pathogen (e.g., fungus or parasite, e.g., malaria parasite), or portion thereof. Non-limiting examples of infectious disease antigens include proteins, polypeptides, peptides, nucleic acids, sugars, small molecules, lipids, or other molecules associated with, derived from, or comprised in an agent that causes an infectious disease (e.g., EBNA3 (e.g., 339-347), EBNA1 (e.g., 407-417), BZLF1 (e.g., 52-64), matrix protein (e.g., influenza virus matrix protein, e.g., 58-66), HIV Gag (e.g., HIV Gag p17, e.g., 77-85), HIV Env, HIV p24 capsid, SIV Tat (e.g., 28-35), SIV Gag (e.g., 181-189), or HCMV pp65 (e.g., 495-503)). As used, herein an “immune cell antigen” includes any molecule present on, or associated with, an immune cell that can provoke an immune response.

The “antigen-binding site,” or “binding portion” of an antibody molecule refers to the part of an antibody molecule, e.g., an immunoglobulin (Ig) molecule, that participates in antigen binding. In embodiments, the antigen binding site is formed by amino acid residues of the variable (V) regions of the heavy (H) and light (L) chains. Three highly divergent stretches within the variable regions of the heavy and light chains, referred to as hypervariable regions, are disposed between more conserved flanking stretches called “framework regions,” (FRs). FRs are amino acid sequences that are naturally found between, and adjacent to, hypervariable regions in immunoglobulins. In embodiments, in an antibody molecule, the three hypervariable regions of a light chain and the three hypervariable regions of a heavy chain are disposed relative to each other in three dimensional space to form an antigen-binding surface, which is complementary to the three-dimensional surface of a bound antigen. The three hypervariable regions of each of the heavy and light chains are referred to as “complementarity-determining regions,” or “CDRs.” The framework region and CDRs have been defined and described, e.g., in Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917. Each variable chain (e.g., variable heavy chain and variable light chain) is typically made up of three CDRs and four FRs, arranged from amino-terminus to carboxy-terminus in the amino acid order: FR1, CDR1, FR2, CDR2, FR3, CDR3, and FR4.

“Infectious disease,” as used herein can encompass all types of diseases, disorders, or conditions associated with (e.g., caused by) an infectious pathogen. Non-limiting examples of infectious pathogens include bacteria, viruses, eukaryotic pathogens (e.g., fungal pathogens or parasites, e.g., malaria parasite), or portions thereof. Non-limiting examples of infectious diseases include Epstein-Barr virus (EBV), influenza, human immunodeficiency virus (HIV), simian immunodeficiency virus (SIV), tuberculosis, malaria, or human cytomegalovirus (HCMV).

As used herein, an “immune cell” refers to any of various cells that function in the immune system, e.g., to protect against agents of infection and foreign matter. In embodiments, this term includes leukocytes, e.g., neutrophils, eosinophils, basophils, lymphocytes, and monocytes. Innate leukocytes include phagocytes (e.g., macrophages, neutrophils, and dendritic cells), mast cells, eosinophils, basophils, and natural killer cells. Innate leukocytes identify and eliminate pathogens, either by attacking larger pathogens through contact or by engulfing and then killing microorganisms, and are mediators in the activation of an adaptive immune response. The cells of the adaptive immune system are special types of leukocytes, called lymphocytes. B cells and T cells are important types of lymphocytes and are derived from hematopoietic stem cells in the bone marrow. B cells are involved in the humoral immune response, whereas T cells are involved in cell-mediated immune response. The term “immune cell” includes immune effector cells.

“Immune effector cell,” as that term is used herein, refers to a cell that is involved in an immune response, e.g., in the promotion of an immune effector response. Examples of immune effector cells include, but are not limited to, T cells, e.g., alpha/beta T cells and gamma/delta T cells, B cells, natural killer (NK) cells, natural killer T (NK T) cells, and mast cells.

The term “effector function” or “effector response” refers to a specialized function of a cell. Effector function of a T cell, for example, may be cytolytic activity or helper activity including the secretion of cytokines.

The compositions and methods of the present invention encompass polypeptides and nucleic acids having the sequences specified, or sequences substantially identical or similar thereto, e.g., sequences at least 80%, 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 80%, 85%, 90%. 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 80%, 85%, 90%, 91%, 92%, 93%, 94%, 95%, 96%, 97%, 98% or 99% identity to a reference sequence, e.g., a sequence provided herein.

The term “variant” refers to a polypeptide that has a substantially identical amino acid sequence to a reference amino acid sequence, or is encoded by a substantially identical nucleotide sequence. In some embodiments, the variant is a functional variant.

The term “functional variant” refers to a polypeptide that has a substantially identical amino acid sequence to a reference amino acid sequence, or is encoded by a substantially identical nucleotide sequence, and is capable of having one or more activities of the reference amino acid sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and) (BLAST programs (version 2.0) of Altschul, et al. (1990) J. Mol. Biol. 215:403-10. BLAST nucleotide searches can be performed with the NBLAST program, score =100, wordlength =12 to obtain nucleotide sequences homologous to a nucleic acid molecule of the invention. BLAST protein searches can be performed with the)(BLAST program, score =50, wordlength =3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g.,)(BLAST and NBLAST) can be used.

It is understood that the molecules of the present invention may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L- optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

Various aspects of the invention are described in further detail below. Additional definitions are set out throughout the specification.

### Human T Cell Receptor (TCR) Complex

T cell receptors (TCR) can be found on the surface of T cells. TCRs recognize antigens, e.g., peptides, presented on, e.g., bound to, major histocompatibility complex (MHC) molecules on the surface of cells, e.g., antigen-presenting cells. TCRs are heterodimeric molecules and can comprise an alpha chain, a beta chain, a gamma chain or a delta chain. TCRs comprising an alpha chain and a beta chain are also referred to as TCRαβ. The TCR beta chain consists of the following regions (also known as segments): variable (V), diversity (D), joining (J) and constant (C) (see Mayer G. and Nyland J. (2010) Chapter 10: Major Histocompatibility Complex and T-cell Receptors-Role in Immune Responses. In: Microbiology and Immunology on-line, University of South Carolina School of Medicine). The TCR alpha chain consists of V, J and C regions. The rearrangement of the T-cell receptor (TCR) through somatic recombination of V (variable), D (diversity), J (joining), and C (constant) regions is a defining event in the development and maturation of a T cell. TCR gene rearrangement takes place in the thymus.

TCRs can comprise a receptor complex, known as the TCR complex, which comprises a TCR heterodimer comprising of an alpha chain and a beta chain, and dimeric signaling molecules, e.g., CD3 co-receptors, e.g., CD3δ/ε, and/or CD3γ/ε.

TCR beta V (TCRβV)

Diversity in the immune system enables protection against a huge array of pathogens. Since the germline genome is limited in size, diversity is achieved not only by the process of V(D)J recombination but also by junctional (junctions between V-D and D-J segments) deletion of nucleotides and addition of pseudo-random, non-templated nucleotides. The TCR beta gene undergoes gene arrangement to generate diversity.

The TCR V beta repertoire varies between individuals and populations because of, e.g., 7 frequently occurring inactivating polymorphisms in functional gene segments and a large insertion/deletion-related polymorphism encompassing 2 V beta gene segments.

This disclosure provides, inter alia, antibody molecules and fragments thereof, that bind, e.g., specifically bind, to a human TCR beta V chain (TCRβV), e.g., a TCRβV gene family (also referred to as a group), e.g., a TCRβV subfamily (also referred to as a subgroup), e.g., as described herein. TCR beta V families and subfamilies are known in the art, e.g., as described in Yassai et al., (2009) Immunogenetics 61(7)pp:493-502; Wei S. and Concannon P. (1994) Human Immunology 41(3) pp: 201-206. The antibodies described herein can be recombinant antibodies, e.g., recombinant non-murine antibodies, e.g., recombinant human or humanized antibodies.

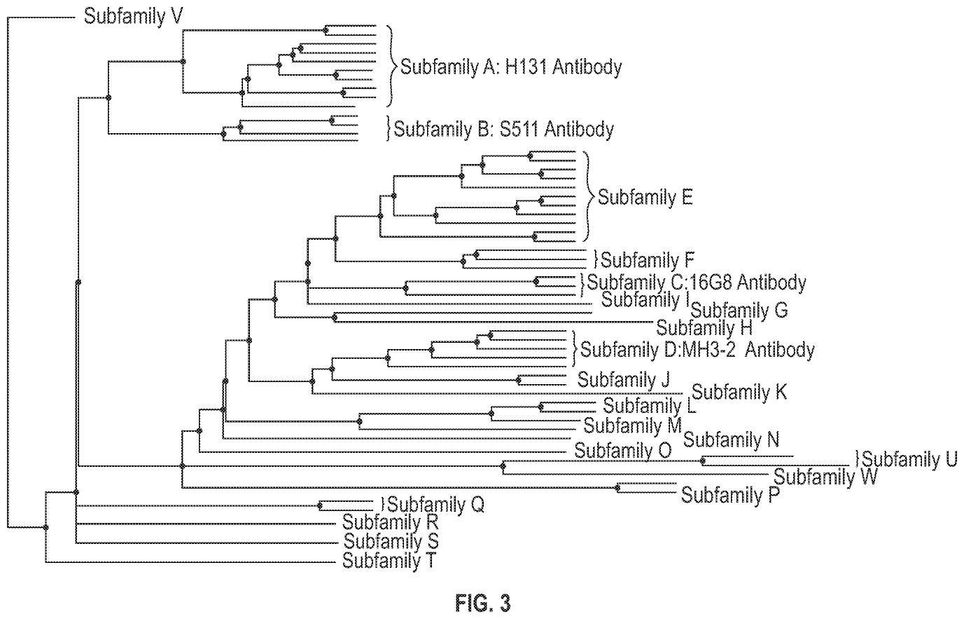

In an aspect, the disclosure provides an anti-TCRβV antibody molecule that binds to human TCRβV, e.g., a TCRβV family, e.g., gene family or a variant thereof. In some embodiments a TCRBV gene family comprises one or more subfamilies, e.g., as described herein, e.g., in FIG. 3, Table 1 or Table 2. In some embodiments, the TCRβV gene family comprises: a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, a TCRβ V29 subfamily, a TCRβ V1 subfamily, a TCRβ V17 subfamily, a TCRβ V21 subfamily, a TCRβ V23 subfamily, or a TCRβ V26 subfamily.

In some embodiments, TCRβ V6 subfamily is also known as TCRβ V13.1. In some embodiments, the TCRβ V6 subfamily comprises: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01, or a variant thereof. In some embodiments, TCRβ V6 comprises TCRβ V6-4*01, or a variant thereof. In some embodiments, TCRβ V6 comprises TCRβ V6-4*02, or a variant thereof. In some embodiments, TCRβ V6 comprises TCRβ V6-9*01, or a variant thereof. In some embodiments, TCRβ V6 comprises TCRβ V6-8*01, or a variant thereof. In some embodiments, TCRβ V6 comprises TCRβ V6-5*01, or a variant thereof. In some embodiments, TCRβ V6 comprises TCRβ V6-6*02, or a variant thereof. In some embodiments, TCRβ V6 comprises TCRβ V6-6*01, or a variant thereof. In some embodiments, TCRβ V6 comprises TCRβ V6-2*01, or a variant thereof. In some embodiments, TCRβ V6 comprises TCRβ V6-3*01, or a variant thereof. In some embodiments, TCRβ V6 comprises TCRβ V6-1*01, or a variant thereof.

In some embodiments, TCRβ V6 comprises TCRβ V6-5*01, or a variant thereof. In some embodiments, TCRβ V6, e.g., TCRβ V6-5*01, is recognized, e.g., bound, by SEQ ID NO: 1 and/or SEQ ID NO: 2. In some embodiments, TCRβ V6, e.g., TCRβ V6-5*01, is recognized, e.g., bound, by SEQ ID NO: 9 and/or SEQ ID NO: 10. In some embodiments, TCRβ V6 is recognized, e.g., bound, by SEQ ID NO: 9 and/or SEQ ID NO: 11.

In some embodiments, TCRβ V10 subfamily is also known as TCRβ V12. In some embodiments, the TCRβ V10 subfamily comprises: TCRβ V10-1*01, TCRβ V10-1*02, TCRβ V10-3*01 or TCRβ V10-2*01, or a variant thereof.

In some embodiments, TCRβ V12 subfamily is also known as TCRβ V8.1. In some embodiments, the TCRβ V12 subfamily comprises: TCRβ V12-4*01, TCRβ V12-3*01, or TCRβ V12-5*01, or a variant thereof. In some embodiments, TCRβ V12 is recognized, e.g., bound, by SEQ ID NO: 15 and/or SEQ ID NO: 16. In some embodiments, TCRβ V12 is recognized, e.g., bound, by any one of SEQ ID NOs 23-25, and/or any one of SEQ ID NO: 26-30:

In some embodiments, the TCRβ V5 subfamily is chosen from: TCRβ V5-5*01, TCRβ V5-6*01, TCRβ V5-4*01, TCRβ V5-8*01, TCRβ V5-1*01, or a variant thereof.

In some embodiments, the TCRβ V7 subfamily comprises TCRβ V7-7*01, TCRβ V7-6*01, TCRβ V7-8*02, TCRβ V7-4*01, TCRβ V7-2*02, TCRβ V7-2*03, TCRβ V7-2*01, TCRβ V7-3 *01, TCRβ V7-9*03, or TCRβ V7-9*01, or a variant thereof.

In some embodiments, the TCRβ V11 subfamily comprises: TCRβ V11-1*01, TCRβ V11-2*01 or TCRβ V11-3*01, or a variant thereof.

In some embodiments, the TCRβ V14 subfamily comprises TCRβ V14*01, or a variant thereof.

In some embodiments, the TCRβ V16 subfamily comprises TCRβ V16*01, or a variant thereof.

In some embodiments, the TCRβ V18 subfamily comprises TCRβ V18*01, or a variant thereof.

In some embodiments, the TCRβ V9 subfamily comprises TCRβ V9*01 or TCRβ V9*02, or a variant thereof.

In some embodiments, the TCRβ V13 subfamily comprises TCRβ V13*01, or a variant thereof.

In some embodiments, the TCRβ V4 subfamily comprises TCRβ V4-2*01, TCRβ V4-3*01, or TCRβ V4-1*01, or a variant thereof.

In some embodiments, the TCRβ V3 subfamily comprises TCRβ V3-1*01, or a variant thereof.

In some embodiments, the TCRβ V2 subfamily comprises TCRβ V2*01, or a variant thereof.

In some embodiments, the TCRβ V15 subfamily comprises TCRβ V15*01, or a variant thereof.

In some embodiments, the TCRβ V30 subfamily comprises TCRβ V30*01, or TCRβ V30*02, or a variant thereof.

In some embodiments, the TCRβ V19 subfamily comprises TCRβ V19*01, or TCRβ V19*02, or a variant thereof.

In some embodiments, the TCRβ V27 subfamily comprises TCRβ V27*01, or a variant thereof.

In some embodiments, the TCRβ V28 subfamily comprises TCRβ V28*01, or a variant thereof.

In some embodiments, the TCRβ V24 subfamily comprises TCRβ V24-1*01, or a variant thereof.

In some embodiments, the TCRβ V20 subfamily comprises TCRβ V20-1*01, or TCRβ V20-1*02, or a variant thereof.

In some embodiments, the TCRβ V25 subfamily comprises TCRβ V25-1*01, or a variant thereof.

In some embodiments, the TCRβ V29 subfamily comprises TCRβ V29-1*01, or a variant thereof,

TABLE 1
List of TCRPBV subfamilies and subfamily members

| Ref- | | |
|---|---|---|
| erence | | |
| in | | |
| FIG. | | |
| 3 | Subfamily | Subfamily members |
| A | TCRβ V6 | TCRβ V6-4*01, TCRβ V6-4*02, TCRβ V6-9*01, |
| | Also referred | TCRβ V6-8*01, TCRβ V6-5*01, TCRβ V6-6*02, |
| | to as: | |
| | TCR VB 13.1 | TCRβ V6-6*01, TCRβ V6-2*01, |
| | | TCRβ V6-3*01 or |
| | | TCRβ V6-1*01. |
| B | TCRβ V10 | TCRβ V10-1*01, TCRβ V10-1*02, |
| | Also referred | TCRβ V10-3*01 or |
| | to as: | TCRβ V10-2*01 |
| | TCRβ V12 | |
| C | TCRβ V12 | TCRβ V12-4*01, TCRβ V12-3*01, or |
| | Also referred | TCRβ V12-5*01 |
| | to as: | |
| | TCRβ V8.1 | |
| D | TCRβ V5 | TCRβ V5-5*01, TCRβ V5-6*01, TCRβ V5-4*01, |
| | | TCRβ V5-8*01, TCRβ V5-1*01 |
| E | TCRβ V7 | TCRβ V7-7*01, TCRβ V7-6*01, TCRβ V7 -8*02, |
| | | TCRβ V7 -4*01, TCRβ V7-2*02, TCRβ V7-2*03, |
| | | TCRβ V7-2*01, TCRβ V7-3*01, TCRβ V7-9*03, |
| | | or |
| | | TCRβ V7-9*01 |
| F | TCRβ V11 | TCRβ V11-1*01, TCRβ V11-2*01 or |
| | | TCRβ V11-3*01 |
| G | TCRβ V14 | TCRβ V14*01 |
| H | TCRβ V16 | TCRβ V16*01 |
| I | TCRβ V18 | TCRβ V18*01 |
| J | TCRβ V9 | TCRβ V9*01 or TCRβ V9*02 |
| K | TCRβ V13 | TCRβ V13*01 |
| L | TCRβ V4 | TCRβ V4-2*01, TCRβ V4-3*01, or |
| | | TCRβ V4-1*01 |
| M | TCRβ V3 | TCRβ V3-1*01 |
| N | TCRβ V2 | TCRβ V2*01 |
| 0 | TCRβ V15 | TCRβ V15*01 |
| P | TCRβ V30 | TCRβ V30*01, or TCRβ V30*02 |
| Q | TCRβ V19 | TCRβ V19*01, or TCRβ V19*02 |
| R | TCRβ V27 | TCRβ V27*01. |
| S | TCRβ V28 | TCRβ V28*01. |
| T | TCRβ V24 | TCRβ V24-1*01 |
| U | TCRβ V20 | TCRβ V20-1*01, or TCRβ V20-1*02 |
| V | TCRβ V25 | TCRβ V25-1*01 |
| W | TCRβ V29 | TCRβ V29-1*01 |

TABLE 2
Additional TCRPV subfamilies

Subfamily
TCRβ V1
TCRβ V17
TCRβ V21
TCRβ V23
TCRβ V26

### Anti-TCRβV Antibodies

Disclosed herein, is the discovery of a novel class of antibodies, i.e. anti-TCRβV antibody molecules disclosed herein, which despite having low sequence similarity (e.g., low sequence identity among the different antibody molecules that recognize different TCRβV subfamilies), recognize a structurally conserved region, e.g., domain, on the TCRβV protein and have a similar function (e.g., a similar cytokine profile). Thus, the anti-TCRβV antibody molecules disclosed herein share a structure-function relationship.

In some embodiments, the anti-TCRβV antibody molecules disclosed herein do not recognize, e.g., bind to, an interface of a TCRβV:TCRalpha complex.

In some embodiments, the anti-TCRβV antibody molecules disclosed herein do not recognize, e.g., bind to, a constant region of a TCRβV protein. An exemplary antibody that binds to a constant region of a TCRBV region is JOVI.1 as described in Viney et al., (Hybridoma. 1992 Dec; 11(6):701-13).

In some embodiments, the anti-TCRβV antibody molecules disclosed herein do not recognize, e.g., bind to, one or more (e.g., all) of a complementarity determining region (e.g., CDR1, CDR2 and/or CDR3) of a TCRβV protein.

In some embodiments, the anti-TCRβV antibody molecules disclosed herein binds (e.g., specifically binds) to a TCRβV region. In some embodiments, binding of anti-TCRβV antibody molecules disclosed herein results in a cytokine profile that differs from a cytokine profile of a T cell engager that binds to a receptor or molecule other than a TCRβV region (“a non-TCRβV-binding T cell engager”). In some embodiments, the non-TCRβV-binding T cell engager comprises an antibody that binds to a CD3 molecule (e.g., CD3 epsilon (CD3e) molecule); or a TCR alpha (TCRα) molecule. In some embodiments, the non-TCRβV-binding T cell engager is an OKT3 antibody or an SP34-2 antibody.

In an aspect, the disclosure provides an anti-TCRβV antibody molecule that binds to human TCRβV, e.g., a TCRβV gene family, e.g., one or more of a TCRβV subfamily, e.g., as described herein, e.g., in FIG. 3, Table 1, or Table 2. In some embodiments, the anti-TCRβV antibody molecule binds to one or more TCRβV subfamilies chosen from: a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, a TCRβ V29 subfamily, a TCRβ V1 subfamily, a TCRβ V17 subfamily, a TCRβ V21 subfamily, a TCRβ V23 subfamily, or a TCRβ V26 subfamily, or a variant thereof.

In some embodiments, the anti-TCRβV antibody molecule binds to a TCRβ V6 subfamily comprising: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01, or a variant thereof. In some embodiments the TCRβ V6 subfamily comprises TCRβ V6-5*01, or a variant thereof. In some embodiments, TCRβ V6 comprises TCRβ V6-4*01, or a variant thereof. In some embodiments, TCRβ V6 comprises TCRβ V6-4*02, or a variant thereof. In some embodiments, TCRβ V6 comprises TCRβ V6-9*01, or a variant thereof. In some embodiments, TCRβ V6 comprises TCRβ V6-8*01, or a variant thereof. In some embodiments, TCRβ V6 comprises TCRβ V6-5*01, or a variant thereof. In some embodiments, TCRβ V6 comprises TCRβ V6-6*02, or a variant thereof. In some embodiments, TCRβ V6 comprises TCRβ V6-6*01, or a variant thereof. In some embodiments, TCRβ V6 comprises TCRβ V6-2*01, or a variant thereof. In some embodiments, TCRβ V6 comprises TCRβ V6-3 *01, or a variant thereof. In some embodiments, TCRβ V6 comprises TCRβ V6-1*01, or a variant thereof.

In some embodiments, the anti-TCRβV antibody molecule binds to a TCRβ V10 subfamily comprising: TCRβ V10-1*01, TCRβ V10-1*02, TCRβ V10-3*01 or TCRβ V10-2*01, or a variant thereof.

In some embodiments, the anti-TCRβV antibody molecule binds to a TCRβ V12 subfamily comprising: TCRβ V12-4*01, TCRβ V12-3*01 or TCRβ V12-5*01, or a variant thereof.

In some embodiments, the anti-TCRβV antibody molecule binds to a TCRβ V5 subfamily comprising: TCRβ V5-5*01, TCRβ V5-6*01, TCRβ V5-4*01, TCRβ V5-8*01, TCRβ V5-1*01, or a variant thereof.

In some embodiments, the anti-TCRβV antibody molecule does not bind to TCRβ V12, or binds to TCRβ V12 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of the 16G8 murine antibody or a humanized version thereof as described in U.S. Pat. No. 5,861,155.

In some embodiments, the anti-TCRβV antibody molecule binds to TCRβ V12 with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the 16G8 murine antibody or a humanized version thereof as described in U.S. Pat. No. 5,861,155.

In some embodiments, the anti-TCRβV antibody molecule binds to a TCRβV region other than TCRβ V12 (e.g., TCRβV region as described herein, e.g., TCRβ V6 subfamily (e.g., TCRβ V6-5*01) with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the 16G8 murine antibody or a humanized version thereof as described in U.S. Pat. No. 5,861,155.

In some embodiments, the anti-TCRβV antibody molecule does not bind to TCRβ V5-5*01 or TCRβ V5-1*01, or binds to TCRβ V5-5*01 or TCRβ V5-1*01 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of murine Antibody C or a humanized version thereof as described in U.S. Pat. No. 5,861,155.

In some embodiments, the anti-TCRβV antibody molecule binds to TCRβ V5-5*01 or TCRβ V5-1*Olwith an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of murine Antibody C or a humanized version thereof as described in U.S. Pat. No. 5,861,155.

In some embodiments, the anti-TCRβV antibody molecule binds to a TCRβV region other than TCRβ V5-5*01 or TCRβ V5-1*01 (e.g., TCRβV region as described herein, e.g., TCRβ V6 subfamily (e.g., TCRβ V6-5*01) with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of murine Antibody C or a humanized version thereof as described in U.S. Pat. No. 5,861,155.

### Anti-TCRI3 V6 Antibodies

Accordingly, in one aspect, the disclosure provides an anti-TCRβV antibody molecule that binds to human TCRβ V6, e.g., a TCRβ V6 subfamily comprising: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments the TCRβ V6 subfamily comprises TCRβ V6-5*01 or a variant thereof. In some embodiments, TCRβ V6 comprises TCRβ V6-4*01, or a variant thereof. In some embodiments, TCRβ V6 comprises TCRβ V6-4*02, or a variant thereof. In some embodiments, TCRβ V6 comprises TCRβ V6-9*01, or a variant thereof. In some embodiments, TCRβ V6 comprises TCRβ V6-8*01, or a variant thereof. In some embodiments, TCRβ V6 comprises TCRβ V6-5*01, or a variant thereof. In some embodiments, TCRβ V6 comprises TCRβ V6-6*02, or a variant thereof. In some embodiments, TCRβ V6 comprises TCRβ V6-6*01, or a variant thereof. In some embodiments, TCRβ V6 comprises TCRβ V6-2*01, or a variant thereof. In some embodiments, TCRβ V6 comprises TCRβ V6-3 *01, or a variant thereof. In some embodiments, TCRβ V6 comprises TCRβ V6-1*01, or a variant thereof.

In some embodiments, TCRβ V6-5*01 is encoded by the nucleic acid sequence of SEQ ID NO: 43, or a sequence having 85%, 90%, 95%, 99% or more identity thereof.

SEQ ID NO: 43
ATGAGCATCGGCCTCCTGTGCTGTGCAGCCTTGTCTCTCCTGTGGGCAG
GTCCAGTGAATGCTGGTGTCACTCAGACCCCAAAATTCCAGGTCCTGAA
GACAGGACAGAGCATGACACTGCAGTGTGCCCAGGATATGAACCATGAA
TACATGTCCTGGTATCGACAAGACCCAGGCATGGGGCTGAGGCTGATTC
ATTACTCAGTTGGTGCTGGTATCACTGACCAAGGAGAAGTCCCCAATGG
CTACAATGTCTCCAGATCAACCACAGAGGATTTCCCGCTCAGGCTGCTG
TCGGCTGCTCCCTCCCAGACATCTGTGTACTTCTGTGCCAGCAGTTACT
C

In some embodiments, TCRβ V6-5*01 comprises the amino acid sequence of SEQ ID NO: 44, or an amino acid sequence having 85%, 90%, 95%, 99% or more identity thereof.

SEQ ID NO: 44
MSIGLLCCAALSLLWAGPVNAGVTQTPKFQVLKTGQSMTLQCAQDMNHE
YMSWYRQDPGMGLRLIHYSVGAGITDQGEVPNGYNVSRSTTEDFPLRLL
SAAPSQTSVYFCASSY

In some embodiments, the anti-TCRβV antibody molecule, e.g., anti-TCRβV6 (e.g., anti-TCRβV6-5*01) antibody molecule, is a non-murine antibody molecule, e.g., a human or humanized antibody molecule. In some embodiments, the anti-TCRβV antibody molecule, e.g., anti-TCRβV6 (e.g., anti-TCRβV6-5*01) antibody molecule is a human antibody molecule. In some embodiments, the anti-TCRβV antibody molecule, e.g., anti-TCRβV6 (e.g., anti-TCRβV6-5*01) antibody molecule is a humanized antibody molecule.

In some embodiments, the anti-TCRβV antibody molecule, e.g., anti-TCRβV6 (e.g., anti-TCRβV6-5*01) antibody molecule, is isolated or recombinant.

In some embodiments, the anti-TCRβV antibody molecule, e.g., anti-TCRβV6 (e.g., anti-TCRβV6-5*01) antibody molecule, comprises at least one antigen-binding region, e.g., a variable region or an antigen-binding fragment thereof, from an antibody described herein, e.g., an antibody chosen from any one of A-H.1 to A-H.68, e.g., A-H.1, A-H.2 or A-H.68, or an antibody described in Table 3, or encoded by a nucleotide sequence in Table 3, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβV6 (e.g., anti-TCRβV6-5*01) antibody molecule, comprises at least one, two, three or four variable regions from an antibody described herein, e.g., an antibody chosen from any one of A-H.1 to A-H.68, e.g., A-H.1, A-H.2 or A-H.68, or an antibody described in Table 3, or encoded by a nucleotide sequence in Table 3, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβV6 (e.g., anti-TCRβV6-5*01) antibody molecule, comprises at least one or two heavy chain variable regions from an antibody described herein, e.g., an antibody chosen from any one of A-H.1 to A-H.68, e.g., A-H.1, A-H.2 or A-H.68, or an antibody molecule described in Table 3, or encoded by a nucleotide sequence in Table 3, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule comprises a heavy chain variable region (VH) having a consensus sequence of SEQ ID NO: 231 or 3290.

In some embodiments, the anti-TCRβV antibody molecule, e.g., anti-TCRβV6 (e.g., anti-TCRβV6-5*01) antibody molecule, comprises at least one or two light chain variable regions from an antibody described herein, e.g., an antibody chosen from any one of A-H.1 to A-H.68, e.g., A-H.1, A-H.2 or A-H.68, or an antibody described in Table 3, or encoded by a nucleotide sequence in Table 3, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule comprises a light chain variable region (VL) having a consensus sequence of SEQ ID NO: 230 or 3289.

In some embodiments, the anti-TCRβV antibody molecule, e.g., anti-TCRβV6 (e.g., anti-TCRβ V6-5*01) antibody molecule, comprises a heavy chain constant region for an IgG4, e.g., a human IgG4. In still another embodiment, the anti-TCRβV antibody molecule, e.g., anti-TCRβ V6 (e.g., anti-TCRβ V6-5*01) antibody molecule includes a heavy chain constant region for an IgG1, e.g., a human IgG1. In one embodiment, the heavy chain constant region comprises an amino sequence set forth in Table 5, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβV6 (e.g., anti-TCRβ V6-5*01) antibody molecule, includes a kappa light chain constant region, e.g., a human kappa light chain constant region. In one embodiment, the light chain constant region comprises an amino sequence set forth in Table 5, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβV6 (e.g., anti-TCRβ V6-5*01) antibody molecule, includes at least one, two, or three complementarity determining regions (CDRs) from a heavy chain variable region (VH) of an antibody described herein, e.g., an antibody chosen from any one of A-H.1 to A-H.68, e.g., A-H.1, A-H.2 or A-H.68, or an antibody described in Table 3, or encoded by a nucleotide sequence in Table 3, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβV6 (e.g., anti-TCRβ V6-5*01) antibody molecule, includes at least one, two, or three CDRs (or collectively all of the CDRs) from a heavy chain variable region comprising an amino acid sequence shown in Table 3, or encoded by a nucleotide sequence shown in Table 3. In one embodiment, one or more of the CDRs (or collectively all of the CDRs) have one, two, three, four, five, six or more changes, e.g., amino acid substitutions or deletions, relative to the amino acid sequence shown in Table 3, or encoded by a nucleotide sequence shown in Table 3.

In some embodiments, the anti-TCRβV antibody molecule, e.g., anti-TCRβV6 (e.g., anti-TCRβ V6-5*01) antibody molecule, includes at least one, two, or three complementarity determining regions (CDRs) from a light chain variable region of an antibody described herein, e.g., an antibody chosen from any one of A-H.1 to A-H.68, e.g., A-H.1, A-H.2 or A-H.68, or an antibody described in Table 3, or encoded by a nucleotide sequence in Table 3, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβV6 (e.g., anti-TCRβ V6-5*01) antibody molecule, includes at least one, two, or three CDRs (or collectively all of the CDRs) from a light chain variable region comprising an amino acid sequence shown in Table 3, or encoded by a nucleotide sequence shown in Table 3. In one embodiment, one or more of the CDRs (or collectively all of the CDRs) have one, two, three, four, five, six or more changes, e.g., amino acid substitutions or deletions, relative to the amino acid sequence shown in Table 3, or encoded by a nucleotide sequence shown in Table 3.

In some embodiments, the anti-TCRβV antibody molecule, e.g., anti-TCRβV6 (e.g., anti-TCRβ V6-5*01) antibody molecule, includes at least one, two, three, four, five or six CDRs (or collectively all of the CDRs) from a heavy and light chain variable region comprising an amino acid sequence shown in Table 3, or encoded by a nucleotide sequence shown in Table 3. In one embodiment, one or more of the CDRs (or collectively all of the CDRs) have one, two, three, four, five, six or more changes, e.g., amino acid substitutions or deletions, relative to the amino acid sequence shown in Table 3, or encoded by a nucleotide sequence shown in Table 3.

In some embodiments, the anti-TCRβV antibody molecule, e.g., anti-TCRβV6 (e.g., anti-TCRβ V6-5*01) antibody molecule, molecule includes all six CDRs from an antibody described herein, e.g., an antibody chosen from any one of A-H.1 to A-H.68, e.g., A-H.1, A-H.2 or A-H.68, or an antibody described in Table 3, or encoded by a nucleotide sequence in Table 3, or closely related CDRs, e.g., CDRs which are identical or which have at least one amino acid alteration, but not more than two, three or four alterations (e.g., substitutions, deletions, or insertions, e.g., conservative substitutions). In some embodiments, the anti-TCRβV antibody molecule, e.g., anti-TCRβV6 (e.g., anti-TCRβ V6-5*01) antibody molecule, may include any CDR described herein.

In some embodiments, the anti-TCRβV antibody molecule, e.g., anti-TCRβV6 (e.g., anti-TCRβ V6-5*01) antibody molecule includes at least one, two, or three CDRs according to Kabat et al. (e.g., at least one, two, or three CDRs according to the Kabat definition as set out in Table 3) from a heavy chain variable region of an antibody described herein, e.g., an antibody chosen from any one of A-H.1 to A-H.68, e.g., A-H.1, A-H.2 or A-H.68, or an antibody described in Table 3, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 3.

In some embodiments, the anti-TCRβV antibody molecule, e.g., anti-TCRβV6 (e.g., anti-TCRβ V6-5*01) antibody molecule includes at least one, two, or three CDRs according to Kabat et al. (e.g., at least one, two, or three CDRs according to the Kabat definition as set out in Table 3) from a light chain variable region of an antibody described herein, e.g., an antibody chosen from any one of A-H.1 to A-H.68, e.g., A-H.1, A-H.2 or A-H.68, or an antibody described in Table 3, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 3.

In some embodiments, the anti-TCRβV antibody molecule, e.g., anti-TCRβV6 (e.g., anti-TCRβ V6-5*01) antibody molecule, includes at least one, two, three, four, five, or six CDRs according to Kabat et al. (e.g., at least one, two, three, four, five, or six CDRs according to the Kabat definition as set out in Table 3) from the heavy and light chain variable regions of an antibody described herein, e.g., an antibody chosen from any one of A-H.1 to A-H.68, e.g., A-H.1, A-H.2 or A-H.68, or an antibody described in Table 3, or encoded by a nucleotide sequence in Table 3; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Kabat et al. shown in Table 3.

In some embodiments, the anti-TCRβV antibody molecule, e.g., anti-TCRβV6 (e.g., anti-TCRβ V6-5*01) antibody molecule, includes all six CDRs according to Kabat et al. (e.g., all six CDRs according to the Kabat definition as set out in Table 3) from the heavy and light chain variable regions of an antibody described herein, e.g., an antibody chosen from any one of A-H.1 to A-H.68, e.g., A-H.1, A-H.2 or A-H.68, or an antibody described in Table 3, or encoded by a nucleotide sequence in Table 3; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Kabat et al. shown in Table 3. In one embodiment, the anti-TCRβV antibody molecule, e.g., anti-TCRβV6 (e.g., anti-TCRβ V6-5*01) antibody molecule, may include any CDR described herein.

In some embodiments, the anti-TCRβV antibody molecule, e.g., anti-TCRβV6 (e.g., anti-TCRβ V6-5*01) antibody molecule, includes at least one, two, or three hypervariable loops that have the same canonical structures as the corresponding hypervariable loop of an antibody described herein, e.g., an antibody chosen from chosen from any one of A-H.1 to A-H.68, e.g., A-H.1, A-H.2 or A-H.68, e.g., the same canonical structures as at least loop 1 and/or loop 2 of the heavy and/or light chain variable domains of an antibody described herein. See, e.g., Chothia et al., (1992) J. Mol. Biol. 227:799-817; Tomlinson et al., (1992) J. Mol. Biol. 227:776-798 for descriptions of hypervariable loop canonical structures. These structures can be determined by inspection of the tables described in these references.

In some embodiments, the anti-TCRβV antibody molecule, e.g., anti-TCRβV6 (e.g., anti-TCRβ V6-5*01) antibody molecule includes at least one, two, or three CDRs according to Chothia et al. (e.g., at least one, two, or three CDRs according to the Chothia definition as set out in Table 3) from a heavy chain variable region of an antibody described herein, e.g., an antibody chosen from any one of A-H.1 to A-H.68, e.g., A-H.1, A-H.2 or A-H.68, or as described in Table 3, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 3.

In some embodiments, the anti-TCRβV antibody molecule, e.g., anti-TCRβV6 (e.g., anti-TCRβ V6-5*01) antibody molecule includes at least one, two, or three CDRs according to Chothia et al. (e.g., at least one, two, or three CDRs according to the Chothia definition as set out in Table 3) from a light chain variable region of an antibody described herein, e.g., an antibody chosen from any one of A-H.1 to A-H.68, e.g., A-H.1, A-H.2 or A-H.68, or an antibody described in Table 3, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 3.

In some embodiments, the anti-TCRβV antibody molecule, e.g., anti-TCRβV6 (e.g., anti-TCRβ V6-5*01) antibody molecule, includes at least one, two, three, four, five, or six CDRs according to Chothia et al. (e.g., at least one, two, three, four, five, or six CDRs according to the Chothia definition as set out in Table 3) from the heavy and light chain variable regions of an antibody described herein, e.g., an antibody chosen from any one of A-H.1 to A-H.68, e.g., A-H.1, A-H.2 or A-H.68, or an antibody described in Table 3, or encoded by the nucleotide sequence in Table 3; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Chothia et al. shown in Table 3.

In some embodiments, the anti-TCRβV antibody molecule, e.g., anti-TCRβV6 (e.g., anti-TCRβ V6-5*01) antibody molecule, includes all six CDRs according to Chothia et al. (e.g., all six CDRs according to the Chothia definition as set out in Table 3) from the heavy and light chain variable regions of an antibody described herein, e.g., an antibody chosen from any one of A-H.1 to A-H.68, e.g., A-H.1, A-H.2 or A-H.68, or an antibody described in Table 3, or encoded by a nucleotide sequence in Table 3; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Chothia et al. shown in Table 3. In one embodiment, the anti-TCRβV antibody molecule, e.g., anti-TCRβV6 (e.g., anti-TCRβ V6-5*01) antibody molecule, may include any CDR described herein.

In some embodiments, the anti-TCRβV antibody molecule, e.g., anti-TCRβV6 (e.g., anti-TCRβ V6-5*01) antibody molecule, molecule includes a combination of CDRs or hypervariable loops defined according to Kabat et al., Chothia et al., or as described in Table 3.

In some embodiments, the anti-TCRβV antibody molecule, e.g., anti-TCRβV6 (e.g., anti-TCRβ V6-5*01) antibody molecule, can contain any combination of CDRs or hypervariable loops according to the Kabat and Chothia definitions.

In some embodiments, a combined CDR as set out in Table 3 is a CDR that comprises a Kabat CDR and a Chothia CDR.

In some embodiments, the anti-TCRβV antibody molecule, e.g., anti-TCRβV6 (e.g., anti-TCRβ V6-5*01) antibody molecule, molecule includes a combination of CDRs or hypervariable loops identified as combined CDRs in Table 3. In some embodiments, the anti-TCRβV antibody molecule, e.g., anti-TCRβV6 (e.g., anti-TCRβ V6-5*01) antibody molecule, can contain any combination of CDRs or hypervariable loops according the “combined” CDRs are described in Table 3.

In an embodiment, e.g., an embodiment comprising a variable region, a CDR (e.g., a combined CDR, Chothia CDR or Kabat CDR), or other sequence referred to herein, e.g., in Table 3, the antibody molecule is a monospecific antibody molecule, a bispecific antibody molecule, a bivalent antibody molecule, a biparatopic antibody molecule, or an antibody molecule that comprises an antigen binding fragment of an antibody, e.g., a half antibody or antigen binding fragment of a half antibody. In certain embodiments the antibody molecule comprises a multispecific molecule, e.g., a bispecific molecule, e.g., as described herein.

In an embodiment, the anti-TCRβV antibody molecule, e.g., anti-TCRβV6 (e.g., anti-TCRβV6-5*01) antibody molecule includes:
(i) one, two or all of a light chain complementarity determining region 1 (LC CDR1), a light chain complementarity determining region 2 (LC CDR2), and a light chain complementarity determining region 3 (LC CDR3) of SEQ ID NO: 2, SEQ ID NO: 10 or SEQ ID NO: 11, and/or
(ii) one, two or all of a heavy chain complementarity determining region 1 (HC CDR1), heavy chain complementarity determining region 2 (HC CDR2), and a heavy chain complementarity determining region 3 (HC CDR3) of SEQ ID NO: 1 or SEQ ID NO: 9.

In some embodiments, the anti-TCRβV antibody molecule, e.g., anti-TCRβV6 (e.g., anti-TCRβ V6-5*01) antibody molecule comprises a LC CDR1, LC CDR2, and LC CDR3 of SEQ ID NO: 2, and a HC CDR1, HC CDR2, and HC CDR3 of SEQ ID NO: 1.

In some embodiments the anti-TCRβV antibody molecule, e.g., anti-TCRβV6 (e.g., anti-TCRβ V6-5*01) antibody molecule comprises a LC CDR1, LC CDR2, and LC CDR3 of SEQ ID NO: 10, and a HC CDR1, HC CDR2, and HC CDR3 of SEQ ID NO: 9.

In some embodiments, the anti-TCRβV antibody molecule, e.g., anti-TCRβV6 (e.g., anti-TCRβ V6-5*01) antibody molecule comprises a LC CDR1, LC CDR2, and LC CDR3 of SEQ ID NO: 11, and a HC CDR1, HC CDR2, and HC CDR3 of SEQ ID NO: 9.

In an embodiment, the anti-TCRβV antibody molecule, e.g., anti-TCRβV6 (e.g., anti-TCRβ V6-5*01) antibody molecule comprises:
(i) a LC CDR1 amino acid sequence of SEQ ID NO: 6, a LC CDR2 amino acid sequence of SEQ ID NO: 7, or a LC CDR3 amino acid sequence of SEQ ID NO: 8; and/or
(ii) a HC CDR1 amino acid sequence of SEQ ID NO: 3, a HC CDR2 amino acid sequence of SEQ ID NO: 4, or a HC CDR3 amino acid sequence of SEQ ID NO: 5.

In some embodiments, the anti-TCRβV antibody molecule, e.g., anti-TCRβV6 (e.g., anti-TCRβ V6-5*01) antibody molecule comprises:
(i) a light chain variable region (VL) comprising a LC CDR1 amino acid sequence of SEQ ID NO: 6, a LC CDR2 amino acid sequence of SEQ ID NO: 7, or a LC CDR3 amino acid sequence of SEQ ID NO: 8; and/or
(ii) a heavy chain variable region (VH) comprising a HC CDR1 amino acid sequence of SEQ ID NO: 3, a HC CDR2 amino acid sequence of SEQ ID NO: 4, or a HC CDR3 amino acid sequence of SEQ ID NO: 5.

In an embodiment, the anti-TCRβV antibody molecule, e.g., anti-TCRβV6 (e.g., anti-TCR(3 V6-5*01) antibody molecule comprises:
(i) a LC CDR1 amino acid sequence of SEQ ID NO: 51, a LC CDR2 amino acid sequence of SEQ ID NO: 52, or a LC CDR3 amino acid sequence of SEQ ID NO: 53; and/or
(ii) a HC CDR1 amino acid sequence of SEQ ID NO: 45, a HC CDR2 amino acid sequence of SEQ ID NO: 46, or a HC CDR3 amino acid sequence of SEQ ID NO: 47.

In some embodiments, the anti-TCRβV antibody molecule, e.g., anti-TCRβV6 (e.g., anti-TCRβ V6-5*01) antibody molecule comprises:
(i) a light chain variable region (VL) comprising a LC CDR1 amino acid sequence of SEQ ID NO: 51, a LC CDR2 amino acid sequence of SEQ ID NO: 52, or a LC CDR3 amino acid sequence of SEQ ID NO: 53; and/or
(ii) a heavy chain variable region (VH) comprising a HC CDR1 amino acid sequence of SEQ ID NO: 45, a HC CDR2 amino acid sequence of SEQ ID NO: 46, or a HC CDR3 amino acid sequence of SEQ ID NO: 47.

In an embodiment, the anti-TCRβV antibody molecule, e.g., anti-TCRβV6 (e.g., anti-TCRβV6-5*01) antibody molecule comprises:
(i) a LC CDR1 amino acid sequence of SEQ ID NO: 54, a LC CDR2 amino acid sequence of SEQ ID NO: 55, or a LC CDR3 amino acid sequence of SEQ ID NO: 56; and/or
(ii) a HC CDR1 amino acid sequence of SEQ ID NO: 48, a HC CDR2 amino acid sequence of SEQ ID NO: 49, or a HC CDR3 amino acid sequence of SEQ ID NO: 50.

In some embodiments, the anti-TCRβV antibody molecule, e.g., anti-TCRβV6 (e.g., anti-TCRβ V6-5*01) antibody molecule comprises:
(i) a light chain variable region (VL) comprising a LC CDR1 amino acid sequence of SEQ ID NO: 54, a LC CDR2 amino acid sequence of SEQ ID NO: 55, or a LC CDR3 amino acid sequence of SEQ ID NO: 56; and/or
(ii) a heavy chain variable region (VH) comprising a HC CDR1 amino acid sequence of SEQ ID NO: 48, a HC CDR2 amino acid sequence of SEQ ID NO: 49, or a HC CDR3 amino acid sequence of SEQ ID NO: 50.

In one embodiment, the light or the heavy chain variable framework (e.g., the region encompassing at least FR1, FR2, FR3, and optionally FR4) of the anti-TCRβV antibody molecule, e.g., anti-TCRβV6 (e.g., anti-TCRβ V6-5*01) antibody molecule can be chosen from: (a) a light or heavy chain variable framework including at least 80%, 85%, 87% 90%, 92%, 93%, 95%, 97%, 98%, or 100% of the amino acid residues from a human light or heavy chain variable framework, e.g., a light or heavy chain variable framework residue from a human mature antibody, a human germline sequence, or a human consensus sequence; (b) a light or heavy chain variable framework including from 20% to 80%, 40% to 60%, 60% to 90%, or 70% to 95% of the amino acid residues from a human light or heavy chain variable framework, e.g., a light or heavy chain variable framework residue from a human mature antibody, a human germline sequence, or a human consensus sequence; (c) a non-human framework (e.g., a rodent framework); or (d) a non-human framework that has been modified, e.g., to remove antigenic or cytotoxic determinants, e.g., deimmunized, or partially humanized. In one embodiment, the light or heavy chain variable framework region (particularly FR1, FR2 and/or FR3) includes a light or heavy chain variable framework sequence at least 70, 75, 80, 85, 87, 88, 90, 92, 94, 95, 96, 97, 98, 99% identical or identical to the frameworks of a VL or VH segment of a human germline gene.

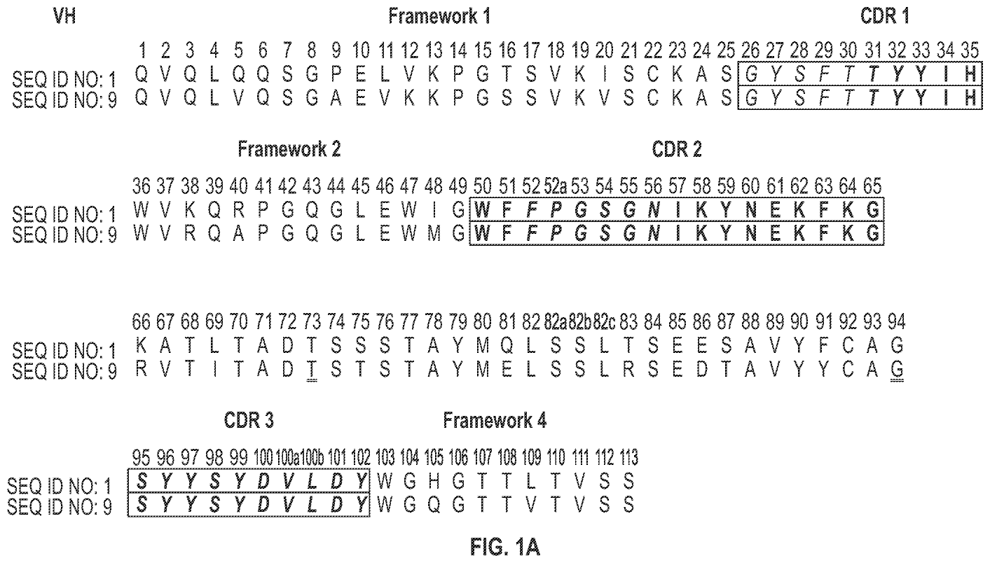

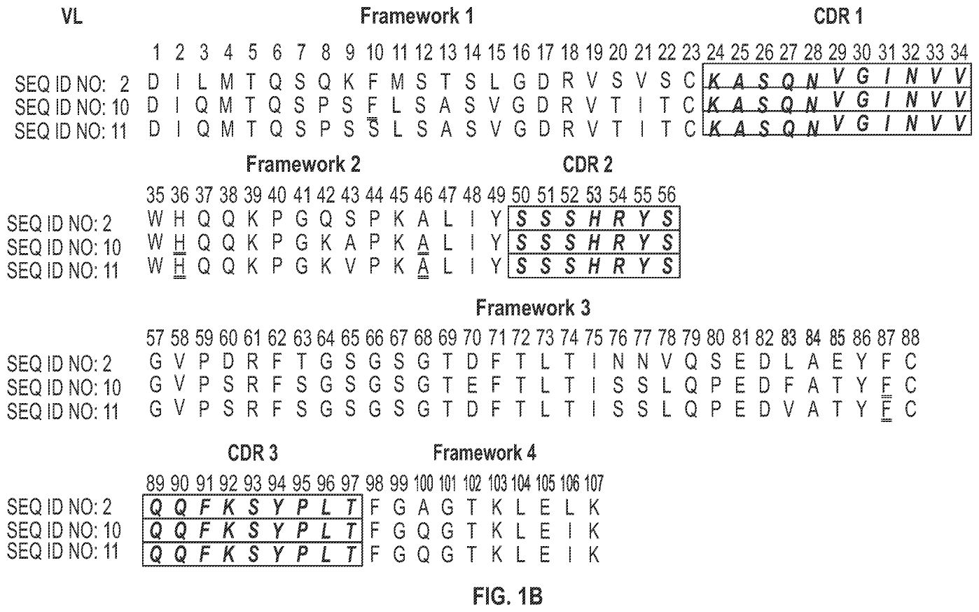

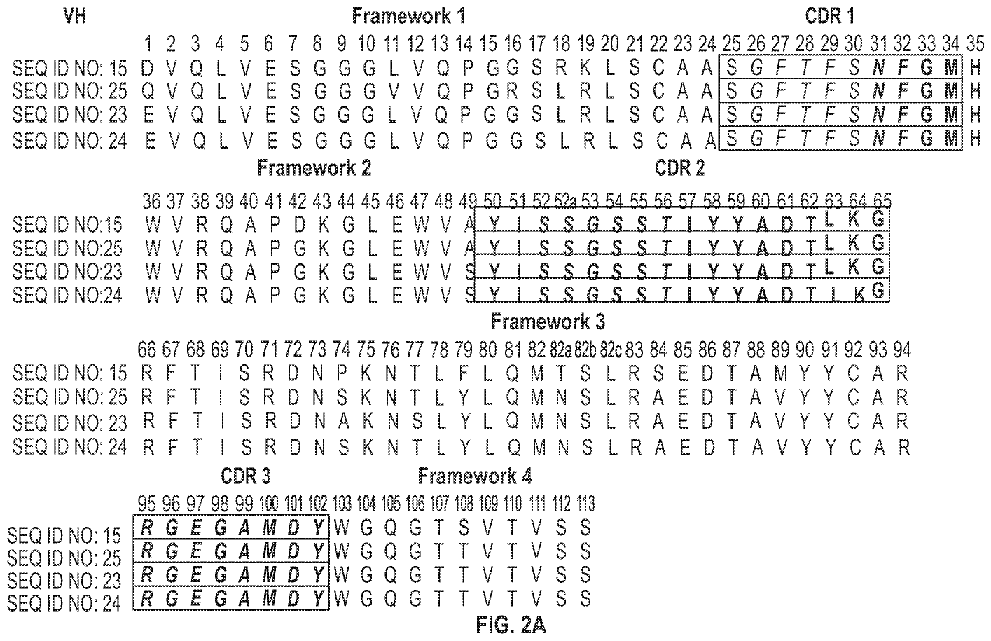

In some embodiments, the anti-TCRβV antibody molecule, e.g., anti-TCRβV6 (e.g., anti-TCRβ V6-5*01) antibody molecule, comprises a heavy chain variable domain having at least one, two, three, four, five, six, seven, ten, fifteen, twenty or more changes, e.g., amino acid substitutions or deletions, from an amino acid sequence of any one of A-H.1 to A-H.68, e.g., A-H.1, A-H.2 or A-H.68, e.g., the amino acid sequence of the FR region in the entire variable region, e.g., shown in FIG. 1A, or in SEQ ID NO: 9.

Alternatively, or in combination with the heavy chain substitutions described herein, the anti-TCRβV antibody molecule, e.g., anti-TCRβV6 (e.g., anti-TCRβ V6-5*01) antibody molecule, comprises a light chain variable domain having at least one, two, three, four, five, six, seven, ten, fifteen, twenty or more amino acid changes, e.g., amino acid substitutions or deletions, from an amino acid sequence of any one of A-H.1 to A-H.68, e.g., A-H.1, A-H.2 or A-H.68, e.g., the amino acid sequence of the FR region in the entire variable region, e.g., shown in FIG. 1B, or in SEQ ID NO: 10 or SEQ ID NO: 11.

In some embodiments, the anti-TCRβV antibody molecule, e.g., anti-TCRβV6 (e.g., anti-TCRβ V6-5*01) antibody molecule, includes one, two, three, or four heavy chain framework regions shown in FIG. 1A, or a sequence substantially identical thereto.

In some embodiments, the anti-TCRβV antibody molecule, e.g., anti-TCRβV6 (e.g., anti-TCRβ V6-5*01) antibody molecule, includes one, two, three, or four light chain framework regions shown in FIG. 1B, or a sequence substantially identical thereto.

In some embodiments, the anti-TCRβV antibody molecule, e.g., anti-TCRβV6 (e.g., anti-TCRβ V6-5*01) antibody molecule, comprises the light chain framework region 1 of A-H.1 or A-H.2, e.g., as shown in FIG. 1B.

In some embodiments, the anti-TCRβV antibody molecule, e.g., anti-TCRβV6 (e.g., anti-TCRβ V6-5*01) antibody molecule, comprises the light chain framework region 2 of A-H.1 or A-H.2, e.g., as shown in FIG. 1B.

In some embodiments, the anti-TCRβV antibody molecule, e.g., anti-TCRβV6 (e.g., anti-TCRβ V6-5*01) antibody molecule, comprises the light chain framework region 3 of A-H.1 or A-H.2, e.g., as shown in FIG. 1B.

In some embodiments, the anti-TCRβV antibody molecule, e.g., anti-TCRβV6 (e.g., anti-TCRβ V6-5*01) antibody molecule, comprises the light chain framework region 4 of A-H.1 or A-H.2, e.g., as shown in FIG. 1B.

In some embodiments, the anti-TCRβV antibody molecule, e.g., anti-TCRβV6 (e.g., anti-TCRβ V6-5*01) antibody molecule, comprises a light chain variable domain comprising a framework region, e.g., framework region 1 (FR1), comprising a change, e.g., a substitution (e.g., a conservative substitution) at position 10 according to Kabat numbering. In some embodiments, the FR1 comprises a Phenylalanine at position 10, e.g., a Serine to Phenyalanine substitution. In some embodiments, the substitution is relative to a human germline light chain framework region sequence.

In some embodiments, the anti-TCRβV antibody molecule, e.g., anti-TCRβV6 (e.g., anti-TCRβ V6-5*01) antibody molecule, comprises a light chain variable domain comprising a framework region, e.g., framework region 2 (FR2), comprising a change, e.g., a substitution (e.g., a conservative substitution) at a position disclosed herein according to Kabat numbering. In some embodiments, FR2 comprises a Histidine at position 36, e.g., a substitution at position 36 according to Kabat numbering, e.g., a Tyrosine to Histidine substitution. In some embodiments, FR2 comprises an Alanine at position 46, e.g., a substitution at position 46 according to Kabat numbering, e.g., an Arginine to Alanine substitution. In some embodiments, the substitution is relative to a human germline light chain framework region sequence.

In some embodiments, the anti-TCRβV antibody molecule, e.g., anti-TCRβV6 (e.g., anti-TCRβ V6-5*01) antibody molecule, comprises a light chain variable domain comprising a framework region, e.g., framework region 3 (FR3), comprising a change, e.g., a substitution (e.g., a conservative substitution) at a position disclosed herein according to Kabat numbering. In some embodiments, FR3 comprises a Phenyalanine at position 87, e.g., a substitution at position 87 according to Kabat numbering, e.g., a Tyrosine to Phenyalanine substitution. In some embodiments, the substitution is relative to a human germline light chain framework region sequence.

In some embodiments, the anti-TCRβV antibody molecule, e.g., anti-TCRβV6 (e.g., anti-TCRβ V6-5*01) antibody molecule, comprises a light chain variable domain comprising: (a) a framework region 1 (FR1) comprising a Phenylalanine at position 10, e.g., a substitution at position 10 according to Kabat numbering, e.g., a Serine to Phenyalanine substitution; (b) a framework region 2 (FR2) comprising a Histidine at position 36, e.g., a substitution at position 36 according to Kabat numbering, e.g., a Tyrosine to Histidine substitution, and a Alanine at position 46, e.g., a substitution at position 46 according to Kabat numbering, e.g., a Arginine to Alanine substitution; and (c) a framework region 3 (FR3) comprising a Phenylalanine at position 87, e.g., a substitution at position 87 according to Kabat numbering, e.g., a Tyrosine to Phenyalanine substitution, e.g., as shown in the amino acid sequence of SEQ ID NO: 10. In some embodiments, the substitution is relative to a human germline light chain framework region sequence.

In some embodiments, the anti-TCRβV antibody molecule, e.g., anti-TCRβV6 (e.g., anti-TCRβ V6-5*01) antibody molecule, comprises a light chain variable domain comprising: (a) a framework region 2 (FR2) comprising a Histidine at position 36, e.g., a substitution at position 36 according to Kabat numbering, e.g., a Tyrosine to Histidine substitution, and a Alanine at position 46, e.g., a substitution at position 46 according to Kabat numbering, e.g., a Arginine to Alanine substitution; and (b) a framework region 3 (FR3) comprising a Phenylalanine at position 87, e.g., a substitution at position 87 according to Kabat numbering, e.g., a Tyrosine to Phenyalanine substitution, e.g., as shown in the amino acid sequence of SEQ ID NO: 11. In some embodiments, the substitution is relative to a human germline light chain framework region sequence.

In some embodiments, the anti-TCRβV antibody molecule, e.g., anti-TCRβV6 (e.g., anti-TCRβ V6-5*01) antibody molecule, comprises a light chain variable domain comprising: (a) a framework region 1 (FR1) comprising a change, e.g., a substitution (e.g., a conservative substitution) at one or more (e.g., all) positions disclosed herein according to Kabat numbering; (b) a framework region 2 (FR2) comprising a change, e.g., a substitution (e.g., a conservative substitution) at one or more (e.g., all) position disclosed herein according to Kabat numbering and (c) a framework region 3 (FR3) comprising a change, e.g., a substitution (e.g., a conservative substitution) at one or more (e.g., all) position disclosed herein according to Kabat numbering. In some embodiments, the substitution is relative to a human germline light chain framework region sequence.

In some embodiments, the anti-TCRβV antibody molecule, e.g., anti-TCRβV6 (e.g., anti-TCRβ V6-5*01) antibody molecule, comprises the heavy chain framework region 1 of A-H.1 or A-H.2, e.g., as shown in FIG. 1A.

In some embodiments, the anti-TCRβV antibody molecule, e.g., anti-TCRβV6 (e.g., anti-TCRβ V6-5*01) antibody molecule, comprises the heavy chain framework region 2 of A-H.1 or A-H.2, e.g., as shown in FIG. 1A

In some embodiments, the anti-TCRβV antibody molecule, e.g., anti-TCRβV6 (e.g., anti-TCRβ V6-5*01) antibody molecule, comprises the heavy chain framework region 3 of A-H.1 or A-H.2, e.g., as shown in FIG. 1A.

In some embodiments, the anti-TCRβV antibody molecule, e.g., anti-TCRβV6 (e.g., anti-TCRβ V6-5*01) antibody molecule, comprises the heavy chain framework region 4 of A-H.1 or A-H.2, e.g., as shown in FIG. 1A.

In some embodiments, the anti-TCRβV antibody molecule, e.g., anti-TCRβV6 (e.g., anti-TCRβ V6-5*01) antibody molecule, comprises a heavy chain variable domain comprising a framework region, e.g., framework region 3 (FR3), comprising a change, e.g., a substitution (e.g., a conservative substitution) at a position disclosed herein according to Kabat numbering. In some embodiments, FR3 comprises a Threonine at position 73, e.g., a substitution at position 73 according to Kabat numbering, e.g., a Glutamic Acid to Threonine substitution. In some embodiments, FR3 comprises a Glycine at position 94, e.g., a substitution at position 94 according to Kabat numbering, e.g., an Arginine to Glycine substitution. In some embodiments, the substitution is relative to a human germline heavy chain framework region sequence.

In some embodiments, the anti-TCRβV antibody molecule, e.g., anti-TCRβV6 (e.g., anti-TCRβ V6-5*01) antibody molecule, comprises a heavy chain variable domain comprising a framework region 3 (FR3) comprising a Threonine at position 73, e.g., a substitution at position 73 according to Kabat numbering, e.g., a Glutamic Acid to Threonine substitution, and a Glycine at position 94, e.g., a substitution at position 94 according to Kabat numbering, e.g., a Arginine to Glycine substitution, e.g., as shown in the amino acid sequence of SEQ ID NO: 10.

In some embodiments, the anti-TCRβV antibody molecule, e.g., anti-TCRβV6 (e.g., anti-TCRβ V6-5*01) antibody molecule, comprises the heavy chain framework regions 1-4 of A-H.1 or A-H.2, e.g., SEQ ID NO: 9, or as shown in FIGS. 1A and 1B.

In some embodiments, the anti-TCRβV antibody molecule, e.g., anti-TCRβV6 (e.g., anti-TCRβ V6-5*01) antibody molecule, comprises the light chain framework regions 1-4 of A-H.1, e.g., SEQ ID NO: 10, or as shown in FIGS. 1A and 1B.

In some embodiments, the anti-TCRβV antibody molecule, e.g., anti-TCRβV6 (e.g., anti-TCRβ V6-5*01) antibody molecule, comprises the light chain framework regions 1-4 of A-H.2, e.g., SEQ ID NO: 11, or as shown in FIGS. 1A and 1B.

In some embodiments, the anti-TCRβV antibody molecule, e.g., anti-TCRβV6 (e.g., anti-TCRβV6-5*01) antibody molecule, comprises the heavy chain framework regions 1-4 of A-H.1, e.g., SEQ ID NO: 9; and the light chain framework regions 1-4 of A-H.1, e.g., SEQ ID NO: 10, or as shown in FIGs. 1A and 1B.

In some embodiments, the anti-TCRβV antibody molecule, e.g., anti-TCRβV6 (e.g., anti-TCRβV6-5*01) antibody molecule, comprises the heavy chain framework regions 1-4 of A-H.2, e.g., SEQ ID NO: 9; and the light chain framework regions 1-4 of A-H.2, e.g., SEQ ID NO: 11, or as shown in FIGs. 1A and IB.

In some embodiments, the heavy or light chain variable domain, or both, of the anti-TCRβV antibody molecule, e.g., anti-TCRβV6 (e.g., anti-TCRβV6-5*01) antibody molecule, includes an amino acid sequence, which is substantially identical to an amino acid disclosed herein, e.g., at least 80%, 85%, 90%, 92%, 95%, 97%, 98%, 99% or higher identical to a variable region of an antibody described herein, e.g., an antibody chosen from any one of A-H.1 to A-H.68, e.g., A-H.1, A-H.2 or A-H.68, or as described in Table 3, or encoded by the nucleotide sequence in Table 3; or which differs at least 1 or 5 residues, but less than 40, 30, 20, or 10 residues, from a variable region of an antibody described herein.

In some embodiments, the anti-TCRβV antibody molecule, e.g., anti-TCRβV6 (e.g., anti-TCRβV6-5*01) antibody molecule, comprises at least one, two, three, or four antigen-binding regions, e.g., variable regions, having an amino acid sequence as set forth in Table 3, or a sequence substantially identical thereto (e.g., a sequence at least about 85%, 90%, 95%, 99% or more identical thereto, or which differs by no more than 1, 2, 5, 10, or 15 amino acid residues from the sequences shown in Table 3. In another embodiment, the anti-TCRβV antibody molecule, e.g., anti-TCRβV6 (e.g., anti-TCRβV6-5*01) antibody molecule includes a VH and/or VL domain encoded by a nucleic acid having a nucleotide sequence as set forth in Table 3, or a sequence substantially identical thereto (e.g., a sequence at least about 85%, 90%, 95%, 99% or more identical thereto, or which differs by no more than 3, 6, 15, 30, or 45 nucleotides from the sequences shown in Table 3.

In some embodiments, the anti-TCRβV antibody molecule, e.g., anti-TCRβV6 (e.g., anti-TCRβV6-5*01) antibody molecule, comprises: a VH domain comprising the amino acid sequence of SEQ ID NO: 9, an amino acid sequence at least about 85%, 90%, 95%, 99% or more identical to the amino acid sequence of SEQ ID NO: 9, or an amino acid sequence which differs by no more than 1, 2, 5, 10, or 15 amino acid residues from the amino acid sequence of SEQ ID NO: 9; and/or a VL domain comprising the amino acid sequence of SEQ ID NO: 10, an amino acid sequence at least about 85%, 90%, 95%, 99% or more identical to the amino acid sequence of SEQ ID NO: 10, or an amino acid sequence which differs by no more than 1, 2, 5, 10, or 15 amino acid residues from the amino acid sequence of SEQ ID NO: 10.

In some embodiments, the anti-TCRβV antibody molecule, e.g., anti-TCRβV6 (e.g., anti-TCRβV6-5*01) antibody molecule, comprises: a VH domain comprising the amino acid sequence of SEQ ID NO: 9, an amino acid sequence at least about 85%, 90%, 95%, 99% or more identical to the amino acid sequence of SEQ ID NO: 9, or an amino acid sequence which differs by no more than 1, 2, 5, 10, or 15 amino acid residues from the amino acid sequence of SEQ ID NO: 9; and/or a VL domain comprising the amino acid sequence of SEQ ID NO: 11, an amino acid sequence at least about 85%, 90%, 95%, 99% or more identical to the amino acid sequence of SEQ ID NO: 11, or an amino acid sequence which differs by no more than 1, 2, 5, 10, or 15 amino acid residues from the amino acid sequence of SEQ ID NO: 11.

In some embodiments, the anti-TCRβV antibody molecule, e.g., anti-TCRβV6 (e.g., anti-TCRβV6-5*01) antibody molecule is a full antibody or fragment thereof (e.g., a Fab, F(ab′)₂, Fv, or a single chain Fv fragment (scFv)). In embodiments, the anti-TCRβV antibody molecule, e.g., anti-TCRβV6 (e.g., anti-TCRβV6-5*01) antibody molecule is a monoclonal antibody or an antibody with single specificity. In some embodiments, the anti-TCRβV antibody molecule, e.g., anti-TCRβV6 (e.g., anti-TCRβV6-5*01) antibody molecule, can also be a humanized, chimeric, camelid, shark, or an in vitro-generated antibody molecule. In some embodiments, the anti-TCRβV antibody molecule, e.g., anti-TCRβV6 (e.g., anti-TCRβV6-5*01) antibody molecule, is a humanized antibody molecule. The heavy and light chains of the anti-TCRβV antibody molecule, e.g., anti-TCRβV6 (e.g., anti-TCRβV6-5*01) antibody molecule, can be full-length (e.g., an antibody can include at least one, and preferably two, complete heavy chains, and at least one, and preferably two, complete light chains) or can include an antigen-binding fragment (e.g., a Fab, F(ab′)₂, Fv, a single chain Fv fragment, a single domain antibody, a diabody (dAb), a bivalent antibody, or bispecific antibody or fragment thereof, a single domain variant thereof, or a camelid antibody).

In some embodiments, the anti-TCRβV antibody molecule, e.g., anti-TCRβV6 (e.g., anti-TCRβ V6-5*01) antibody molecule, is in the form of a multispecific molecule, e.g., a bispecific molecule, e.g., as described herein.

In some embodiments, the anti-TCRβV antibody molecule, e.g., anti-TCRβV6 (e.g., anti-TCRβ V6-5*01) antibody molecule, has a heavy chain constant region (Fc) chosen from, e.g., the heavy chain constant regions of IgG1, IgG2, IgG3, IgG4, IgM, IgAl, IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgG1 or IgG2 (e.g., human IgG1, or IgG2). In some embodiments, the heavy chain constant region is human IgG1. In some embodiments, the Fc region comprises a Fc region variant, e.g., as described herein.

In some embodiments, the anti-TCRβV antibody molecule, e.g., anti-TCRβV6 (e.g., anti-TCRβ V6-5*01) antibody molecule, has a light chain constant region chosen from, e.g., the light chain constant regions of kappa or lambda, preferably kappa (e.g., human kappa). In one embodiment, the constant region is altered, e.g., mutated, to modify the properties of the anti-TCRβV antibody molecule, e.g., anti-TCRβV6 (e.g., anti-TCRβ V6-5*01) antibody molecule (e.g., to increase or decrease one or more of: Fc receptor binding, antibody glycosylation, the number of cysteine residues, effector cell function, or complement function). For example, the constant region is mutated at positions 296 (M to Y), 298 (S to T), 300 (T to E), 477 (H to K) and 478 (N to F) to alter Fc receptor binding (e.g., the mutated positions correspond to positions 132 (M to Y), 134 (S to T), 136 (T to E), 313 (H to K) and 314 (N to F) of SEQ ID NOs: 212 or 214; or positions 135 (M to Y), 137 (S to T), 139 (T to E), 316 (H to K) and 317 (N to F) of SEQ ID NOs: 215, 216, 217, or 218), e.g., relative to human IgG1.

Antibody A-H.1 comprises a heavy chain comprising the amino acid sequence of SEQ ID NO: 3278 and a light chain comprising the amino acid sequence of SEQ ID NO: 72. Antibody A-H.2 comprises a heavy chain comprising the amino acid sequence of SEQ ID NO: 3278 and a light chain comprising the amino acid sequence of SEQ ID NO: 3279. Antibody A-H.68 comprises the amino acid sequence of SEQ ID NO: 1337, or a sequence having at least 85%, 90%, 95%, 96%, 97%, 98%, or 99% identity thereto.

Additional exemplary humanized anti-TCRB V6 antibodies are provided in Table 3. In some embodiments, the anti-TCRβV6 is antibody A, e.g., humanized antibody A (antibody A-H), as provided in Table 3. In some embodiments, the anti-TCRβV antibody comprises one or more (e.g., all three) of a LC CDR1, LC CDR2, and LC CDR3 provided in Table 3; and/or one or more (e.g., all three) of a HC CDR1, HC CDR2, and HC CDR3 provided in Table 3, or a sequence with at least 85%, 90%, 95%, 96%, 97%, 98%, or 99% identity thereto. In some embodiments, antibody A comprises a variable heavy chain (VH) and/or a variable light chain (VL) provided in Table 3, or a sequence with at least 85%, 90%, 95%, 96%, 97%, 98%, or 99% identity thereto.

TABLE 3
Amino acid and nucleotide sequences for murine, chimeric and humanized
antibody molecules which bind to TCRVB 6, e.g., TCRVB 6-5. The antibody
molecules include murine mAb Antibody A, and humanized mAb Antibody A-H
Clones A-H.1 to A-H.68. The amino acid the heavy and light chain CDRs,
and the amino acid and nucleotide sequences of the heavy and light
chain variable regions, and the heavy and light chains are shown.
Antibody A (murine)

| | | |
|---|---|---|
| SEQ ID NO: 3 | HC CDR1 | GYSFTTYYIH |
| | (Combined) | |
| SEQ ID NO: 4 | HC CDR2 | WFFPGSGNIKYNEKFKG |
| | (Combined) | |
| SEQ ID NO: 5 | HC CDR3 | SYYSYDVLDY |
| | (Combined) | |
| SEQ ID NO: 45 | HC CDR1 (Kabat) | TYYIH |
| SEQ ID NO: 46 | HC CDR2 (Kabat) | WFFPGSGNIKYNEKFKG |
| SEQ ID NO: 47 | HC CDR3 (Kabat) | SYYSYDVLDY |
| SEQ ID NO: 48 | HC CDR1 (Chothia) | GYSFTTY |
| SEQ ID NO: 49 | HC CDR2 (Chothia) | FPGSGN |
| SEQ ID NO: 50 | HC CDR3 (Chothia) | SYYSYDVLDY |
| SEQ ID NO: 1 | VH | QVQLQQSGPELVKPGTSVKISCKASGYSFTTYYIHWVKQRPG |
| | | QGLEWIGWFFPGSGNIKYNEKFKGKATLTADTSSSTAYMQLS |
| | | SLTSEESAVYFCAGSYYSYDVLDYWGHGTTLTVSS |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: 51 | LC CDR1 (Kabat) | KASQNVGINVV |
| SEQ ID NO: 52 | LC CDR2 (Kabat) | SSSHRYS |
| SEQ ID NO: 53 | LC CDR3 (Kabat) | QQFKSYPLT |
| SEQ ID NO: 54 | LC CDR1 (Chothia) | KASQNVGINVV |
| SEQ ID NO: 55 | LC CDR2 (chothia) | SSSHRYS |
| SEQ ID NO: 56 | LC CDR3 (chothia) | QQFKSYPLT |
| SEQ ID NO: 2 | VL | DILMTQSQKFMSTSLGDRVSVSCKASQNVGINVVWHQQKPGQ |
| | | SPKALIYSSSHRYSGVPDRFTGSGSGTDFTLTINNVQSEDLA |
| | | EYFCQQFKSYPLTFGAGTKLELK |

Antibody Ahumanized (A-H antibody)
A-H.1 antibody

| | | |
|---|---|---|
| SEQ ID NO: 3 | HC CDR1 | GYSFTTYYIH |
| | (Combined) | |
| SEQ ID NO: 4 | HC CDR2 | WFFPGSGNIKYNEKFKG |
| | (Combined) | |
| SEQ ID NO: 5 | HC CDR3 | SYYSYDVLDY |
| | (Combined) | |
| SEQ ID NO: 9 | VH | QVQLVQSGAEVKKPGSSVKVSCKASGYSFTTYYIHWVRQAPG |
| | | QGLEWMGWFFPGSGNIKYNEKFKGRVTITADTSTSTAYMELS |
| | | SLRSEDTAVYYCAGSYYSYDVLDYWGQGTTVTVSS |
| SEQ ID NO: 12 | DNA VH | CAGGTGCAGCTGGTTCAGTCTGGCGCCGAAGTGAAGAAACCT |
| | | GGCTCCTCCGTGAAGGTGTCCTGCAAGGCTTCCGGCTACTCC |
| | | TTCACCACCTACTACATCCACTGGGTCCGACAGGCCCCTGGA |
| | | CAAGGATTGGAATGGATGGGCTGGTTCTTCCCCGGCTCCGGC |
| | | AACATCAAGTACAACGAGAAGTTCAAGGGCCGCGTGACCATC |
| | | ACCGCCGACACCTCTACCTCTACCGCCTACATGGAACTGTCC |
| | | AGCCTGAGATCTGAGGACACCGCCGTGTACTACTGCGCCGGC |
| | | TCCTACTACTCTTACGACGTGCTGGATTACTGGGGCCAGGGC |
| | | ACCACAGTGACAGTGTCCTCT |
| SEQ ID NO: 69 | VH-IgM constant | METDTLLLWVLLLWVPGSTGQVQLVQSGAEVKKPGSSVKVSC |
| | delta CDC | KASGYSFTTYYIHWVRQAPGQGLEWMGWFFPGSGNIKYNEKF |
| | | KGRVTITADTSTSTAYMELSSLRSEDTAVYYCAGSYYSYDVL |
| | | DYWGQGTTVTVSSGSASAPTLFPLVSCENSPSDTSSVAVGCL |
| | | AQDFLPDSITFSWKYKNNSDISSTRGFPSVLRGGKYAATSQV |
| | | LLPSKDVMQGTDEHVVCKVQHPNGNKEKNVPLPVIAELPPKV |
| | | SVFVPPRDGFFGNPRKSKLICQATGFSPRQIQVSWLREGKQV |
| | | GSGVTTDQVQAEAKESGPTTYKVTSTLTIKESDWLGQSMFTC |
| | | RVDHRGLTFQQNASSMCVPDQDTAIRVFAIPPSFASIFLTKS |
| | | TKLTCLVTDLTTYDSVTISWTRQNGEAVKTHTNISESHPNAT |
| | | FSAVGEASICEDDWNSGERFTCTVTHTDLASSLKQTISRPKG |
| | | VALHRPDVYLLPPAREQLNLRESATITCLVTGFSPADVFVQW |
| | | MQRGQPLSPEKYVTSAPMPEPQAPGRYFAHSILTVSEEEWNT |
| | | GETYTCVVAHEALPNRVTERTVDKSTGKPTLYNVSLVMSDTA |
| | | GTCY |
| SEQ ID NO: 70 | VH-IgGA1 | METDTLLLWVLLLWVPGSTGQVQLVQSGAEVKKPGSSVKVSC |
| | | KASGYSFTTYYIHWVRQAPGQGLEWMGWFFPGSGNIKYNEKF |
| | | KGRVTITADTSTSTAYMELSSLRSEDTAVYYCAGSYYSYDVL |
| | | DYWGQGTTVTVSSASPTSPKVFPLSLCSTQPDGNVVIACLVQ |
| | | GFFPQEPLSVTWSESGQGVTARNFPPSQDASGDLYTTSSQLT |
| | | LPATQCLAGKSVTCHVKHYTNPSQDVTVPCPVPSTPPTPSPS |
| | | TPPTPSPSCCHPRLSLHRPALEDLLLGSEANLTCTLTGLRDA |
| | | SGVTFTWTPSSGKSAVQGPPERDLCGCYSVSSVLPGCAEPWN |
| | | HGKTFTCTAAYPESKTPLTATLSKSGNTFRPEVHLLPPPSEE |
| | | LALNELVTLTCLARGFSPKDVLVRWLQGSQELPREKYLTWAS |
| | | RQEPSQGTTTFAVTSILRVAAEDWKKGDTFSCMVGHEALPLA |
| | | FTQKTIDRLAGKPTHVNVSVVMAEVDGTCY |
| SEQ ID NO: 71 | VH-IgGA2 | METDTLLLWVLLLWVPGSTGQVQLVQSGAEVKKPGSSVKVSC |
| | | KASGYSFTTYYIHWVRQAPGQGLEWMGWFFPGSGNIKYNEKF |
| | | KGRVTITADTSTSTAYMELSSLRSEDTAVYYCAGSYYSYDVL |
| | | DYWGQGTTVTVSSASPTSPKVFPLSLDSTPQDGNVVVACLVQ |
| | | GFFPQEPLSVTWSESGQNVTARNFPPSQDASGDLYTTSSQLT |
| | | LPATQCPDGKSVTCHVKHYTNSSQDVTVPCRVPPPPPCCHPR |
| | | LSLHRPALEDLLLGSEANLTCTLTGLRDASGATFTWTPSSGK |
| | | SAVQGPPERDLCGCYSVSSVLPGCAQPWNHGETFTCTAAHPE |
| | | LKTPLTANITKSGNTFRPEVHLLPPPSEELALNELVTLTCLA |
| | | RGFSPKDVLVRWLQGSQELPREKYLTWASRQEPSQGTTTYAV |
| | | TSILRVAAEDWKKGETFSCMVGHEALPLAFTQKTIDRMAGKP |
| | | THINVSVVMAEADGTCY |
| SEQ ID NO: 3278 | Heavy chain | METDTLLLWVLLLWVPGSTGQVQLVQSGAEVKKPGSSVKVSC |
| | | KASGYSFTTYYIHWVRQAPGQGLEWMGWFFPGSGNIKYNEKF |
| | | KGRVTITADTSTSTAYMELSSLRSEDTAVYYCAGSYYSYDVL |
| | | DYWGQGTTVTVSSASTKGPSVFPLAPSSKSTSGGTAALGCLV |
| | | KDYFPEPVTVSWNSGALTSGVHTFPAVLQSSGLYSLSSVVTV |
| | | PSSSLGTQTYICNVNHKPSNTKVDKRVEPKSCDKTHTCPPCP |
| | | APELLGGPSVFLFPPKPKDTLMISRTPEVTCVVVDVSHEDPE |
| | | VKFNWYVDGVEVHNAKTKPREEQYNSTYRVVSVLTVLHQDWL |
| | | NGKEYKCKVSNKALPAPIEKTISKAKGQPREPQVYTLPPSRE |
| | | EMTKNQVSLTCLVKGFYPSDIAVEWESNGQPENNYKTTPPVL |
| | | DSDGSFFLYSKLTVDKSRWQQGNVFSCSVMHEALHNHYTQKS |
| | | LSLSPGK |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: 10 | VL | DIQMTQSPSFLSASVGDRVTITCKASQNVGINVVWHQQKPGK |
| | | APKALIYSSSHRYSGVPSRFSGSGSGTEFTLTISSLQPEDFA |
| | | TYFCQQFKSYPLTFGQGTKLEIK |
| SEQ ID NO: 13 | DNA VL | GACATCCAGATGACCCAGTCTCCATCCTTCCTGTCCGCCTCT |
| | | GTGGGCGACAGAGTGACCATCACATGCAAGGCCTCTCAGAAC |
| | | GTGGGCATCAACGTCGTGTGGCACCAGCAGAAGCCTGGCAAG |
| | | GCTCCTAAGGCTCTGATCTACTCCTCCAGCCACCGGTACTCT |
| | | GGCGTGCCCTCTAGATTTTCCGGCTCTGGCTCTGGCACCGAG |
| | | TTTACCCTGACAATCTCCAGCCTGCAGCCTGAGGACTTCGCC |
| | | ACCTACTTTTGCCAGCAGTTCAAGAGCTACCCTCTGACCTTT |
| | | GGCCAGGGCACCAAGCTGGAAATCAAG |
| SEQ ID NO: 72 | VL and kappa | METDTLLLWVLLLWVPGSTGDIQMTQSPSFLSASVGDRVTIT |
| | constant region/ | CKASQNVGINVVWHQQKPGKAPKALIYSSSHRYSGVPSRFSG |
| | light chain | SGSGTEFTLTISSLQPEDFATYFCQQFKSYPLTFGQGTKLEI |
| | | KRTVAAPSVFIFPPSDEQLKSGTASVVCLLNNFYPREAKVQW |
| | | KVDNALQSGNSQESVTEQDSKDSTYSLSSTLTLSKADYEKHK |
| | | VYACEVTHQGLSSPVTKSFNRGEC |

A-H.2 antibody

| | | |
|---|---|---|
| SEQ ID NO: 3 | HC CDR1 | GYSFTTYYIH |
| | (Combined) | |
| SEQ ID NO: 4 | HC CDR2 | WFFPGSGNIKYNEKFKG |
| | (Combined) | |
| SEQ ID NO: 5 | HC CDR3 | SYYSYDVLDY |
| | (Combined) | |
| SEQ ID NO: 9 | VH | QVQLVQSGAEVKKPGSSVKVSCKASGYSFTTYYIHWVRQAPG |
| | | QGLEWMGWFFPGSGNIKYNEKFKGRVTITADTSTSTAYMELS |
| | | SLRSEDTAVYYCAGSYYSYDVLDYWGQGTTVTVSS |
| SEQ ID NO: 12 | DNA VH | CAGGTGCAGCTGGTTCAGTCTGGCGCCGAAGTGAAGAAACCT |
| | | GGCTCCTCCGTGAAGGTGTCCTGCAAGGCTTCCGGCTACTCC |
| | | TTCACCACCTACTACATCCACTGGGTCCGACAGGCCCCTGGA |
| | | CAAGGATTGGAATGGATGGGCTGGTTCTTCCCCGGCTCCGGC |
| | | AACATCAAGTACAACGAGAAGTTCAAGGGCCGCGTGACCATC |
| | | ACCGCCGACACCTCTACCTCTACCGCCTACATGGAACTGTCC |
| | | AGCCTGAGATCTGAGGACACCGCCGTGTACTACTGCGCCGGC |
| | | TCCTACTACTCTTACGACGTGCTGGATTACTGGGGCCAGGGC |
| | | ACCACAGTGACAGTGTCCTCT |
| SEQ ID NO: 3278 | Heavy chain | METDTLLLWVLLLWVPGSTGQVQLVQSGAEVKKPGSSVKVSC |
| | | KASGYSFTTYYIHWVRQAPGQGLEWMGWFFPGSGNIKYNEKF |
| | | KGRVTITADTSTSTAYMELSSLRSEDTAVYYCAGSYYSYDVL |
| | | DYWGQGTTVTVSSASTKGPSVFPLAPSSKSTSGGTAALGCLV |
| | | KDYFPEPVTVSWNSGALTSGVHTFPAVLQSSGLYSLSSVVTV |
| | | PSSSLGTQTYICNVNHKPSNTKVDKRVEPKSCDKTHTCPPCP |
| | | APELLGGPSVFLFPPKPKDTLMISRTPEVTCVVVDVSHEDPE |
| | | VKFNWYVDGVEVHNAKTKPREEQYNSTYRVVSVLTVLHQDWL |
| | | NGKEYKCKVSNKALPAPIEKTISKAKGQPREPQVYTLPPSRE |
| | | EMTKNQVSLTCLVKGFYPSDIAVEWESNGQPENNYKTTPPVL |
| | | DSDGSFFLYSKLTVDKSRWQQGNVFSCSVMHEALHNHYTQKS |
| | | LSLSPGK |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: 11 | VL | DIQMTQSPSSLSASVGDRVTITCKASQNVGINVVWHQQKPGK |
| | | VPKALIYSSSHRYSGVPSRFSGSGSGTDFTLTISSLQPEDVA |
| | | TYFCQQFKSYPLTFGQGTKLEIK |
| SEQ ID NO: 14 | DNA VL | GACATCCAGATGACCCAGTCTCCATCCTCTCTGTCCGCCTCT |
| | | GTGGGCGACAGAGTGACCATCACATGCAAGGCCTCTCAGAAC |
| | | GTGGGCATCAACGTCGTGTGGCACCAGCAGAAACCTGGCAAG |
| | | GTGCCCAAGGCTCTGATCTACTCCTCCAGCCACAGATACTCC |
| | | GGCGTGCCCTCTAGATTCTCCGGCTCTGGCTCTGGCACCGAC |
| | | TTTACCCTGACAATCTCCAGCCTGCAGCCTGAGGACGTGGCC |
| | | ACCTACTTTTGCCAGCAGTTCAAGAGCTACCCTCTGACCTTT |
| | | GGCCAGGGCACCAAGCTGGAAATCAAG |
| SEQ ID NO: 3279 | Light chain | METDTLLLWVLLLWVPGSTGDIQMTQSPSSLSASVGDRVTIT |
| | | CKASQNVGINVVWHQQKPGKVPKALIYSSSHRYSGVPSRFSG |
| | | SGSGTDFTLTISSLQPEDVATYFCQQFKSYPLTFGQGTKLEI |
| | | KRTVAAPSVFIFPPSDEQLKSGTASVVCLLNNFYPREAKVQW |
| | | KVDNALQSGNSQESVTEQDSKDSTYSLSSTLTLSKADYEKHK |
| | | VYACEVTHQGLSSPVTKSFNRGEC |

A-H.3 antibody

| | | |
|---|---|---|
| SEQ ID NO: 80 | VH + VL | QVQLVQSGAEVKKPGSSVKVSCKASGTDFKLTYIHWVRQAPG |
| | | QGLEWMGRVSPGSGNTKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | EDRVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 81 | VL | DIQMTQSPSFLSASVGDRVTITCKASQNVEDRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 82 | VH | QVQLVQSGAEVKKPGSSVKVSCKASGTDFKLTYIHWVRQAPG |
| | | QGLEWMGRVSPGSGNTKYNEKFKGRVTITADTSTSTAYMELS |
| | | SLRSEDTAVYYCAGSYYSYDVLDYWGQGTTVTVSS |

A-H.4

| | | |
|---|---|---|
| SEQ ID NO: 83 | VH + VL | QVQLVQSGAEVKKPGSSVKVSCKASGTDFDKIYIHWVRQAPG |
| | | QGLEWMGRISAGSGNVKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | EDRVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 84 | VL | DIQMTQSPSFLSASVGDRVTITCKASQNVEDRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 85 | VH | QVQLVQSGAEVKKPGSSVKVSCKASGTDFDKIYIHWVRQAPG |
| | | QGLEWMGRISAGSGNVKYNEKFKGRVTITADTSTSTAYMELS |
| | | SLRSEDTAVYYCAGSYYSYDVLDYWGQGTTVTVSS |

A-H.5

| | | |
|---|---|---|
| SEQ ID NO: 86 | VH + VL | QVQLVQSGAEVKKPGSSVKVSCKASGHDFRDFYIHWVRQAPG |
| | | QGLEWMGRVYPGSGSYRYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | DDRVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 87 | VL | DIQMTQSPSFLSASVGDRVTITCKASQNVDDRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 88 | VH | QVQLVQSGAEVKKPGSSVKVSCKASGHDFRDFYIHWVRQAPG |
| | | QGLEWMGRVYPGSGSYRYNEKFKGRVTITADTSTSTAYMELS |
| | | SLRSEDTAVYYCAGSYYSYDVLDYWGQGTTVTVSS |

A-H.6

| | | |
|---|---|---|
| SEQ ID NO: 89 | VH + VL | QVQLVQSGAEVKKPGSSVKVSCKASGHDFKLTYIHWVRQAPG |
| | | QGLEWMGRISAGSGNVKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | DNRVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 90 | VL | DIQMTQSPSFLSASVGDRVTITCKASQNVDNRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 91 | VH | QVQLVQSGAEVKKPGSSVKVSCKASGHDFKLTYIHWVRQAPG |
| | | QGLEWMGRISAGSGNVKYNEKFKGRVTITADTSTSTAYMELS |
| | | SLRSEDTAVYYCAGSYYSYDVLDYWGQGTTVTVSS |

A-H.7

| | | |
|---|---|---|
| SEQ ID NO: 92 | VH + VL | QVQLVQSGAEVKKPGSSVKVSCKASGTDFKLTYIHWVRQAPG |
| | | QGLEWMGRIFPGSGNVKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | ENKVAWHQQKPGKAPKALIYSSSHRYKGVPSRFSGSGSGTEF |
| | | TLTISSLQPEDFATYFCQQFKSYPLTFGQGTKLEIK |
| SEQ ID NO: 93 | VL | DIQMTQSPSFLSASVGDRVTITCKASQNVENKVAWHQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 94 | VH | QVQLVQSGAEVKKPGSSVKVSCKASGTDFKLTYIHWVRQAPG |
| | | QGLEWMGRIFPGSGNVKYNEKFKGRVTITADTSTSTAYMELS |
| | | SLRSEDTAVYYCAGSYYSYDVLDYWGQGTTVTVSS |

A-H.8

| | | |
|---|---|---|
| SEQ ID NO: 95 | VH + VL | QVQLVQSGAEVKKPGSSVKVSCKASGTDFDKIYIHWVRQAPG |
| | | QGLEWMGRIFAGSGNTKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | DDRVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 96 | VL | DIQMTQSPSFLSASVGDRVTITCKASQNVDDRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 97 | VH | QVQLVQSGAEVKKPGSSVKVSCKASGTDFDKIYIHWVRQAPG |
| | | QGLEWMGRIFAGSGNTKYNEKFKGRVTITADTSTSTAYMELS |
| | | SLRSEDTAVYYCAGSYYSYDVLDYWGQGTTVTVSS |

A-H.9

| | | |
|---|---|---|
| SEQ ID NO: 98 | VH + VL | QVQLVQSGAEVKKPGSSVKVSCKASGHDFDKFYIHWVRQAPG |
| | | QGLEWMGRVSAGSGNVKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | GNRVAWYQQKPGKAPKALIYSSSHRYSGVPSRFSGSGSGTEF |
| | | TLTISSLQPEDFATYFCQQFKSYPLTFGQGTKLEIK |
| SEQ ID NO: 99 | VL | DIQMTQSPSFLSASVGDRVTITCKASQNVGNRVAWYQQKPGK |
| | | APKALIYSSSHRYSGVPSRFSGSGSGTEFTLTISSLQPEDFA |
| | | TYFCQQFKSYPLTFGQGTKLEIK |
| SEQ ID NO: 100 | VH | QVQLVQSGAEVKKPGSSVKVSCKASGHDFDKFYIHWVRQAPG |
| | | QGLEWMGRVSAGSGNVKYNEKFKGRVTITADTSTSTAYMELS |
| | | SLRSEDTAVYYCAGSYYSYDVLDYWGQGTTVTVSS |

A-H.10

| | | |
|---|---|---|
| SEQ ID NO: 101 | VH + VL | QVQLVQSGAEVKKPGSSVKVSCKASGHDFDKFYIHWVRQAPG |
| | | QGLEWMGRIFAGSGNVKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | GDRVAWYQQKPGKAPKALIYSSSHRYKGVPSRFSGSGSGTEF |
| | | TLTISSLQPEDFATYFCQQFKSYPLTFGQGTKLEIKs |
| SEQ ID NO: 102 | VL | DIQMTQSPSFLSASVGDRVTITCKASQNVGDRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 103 | VH | QVQLVQSGAEVKKPGSSVKVSCKASGHDFDKFYIHWVRQAPG |
| | | QGLEWMGRIFAGSGNVKYNEKFKGRVTITADTSTSTAYMELS |
| | | SLRSEDTAVYYCAGSYYSYDV |
| | | LDYWGQGTTVTVSS |

A-H.11

| | | |
|---|---|---|
| SEQ ID NO: 104 | VH + VL | QVQLVQSGAEVKKPGSSVKVSCKASGTDFKLTYIHWVRQAPG |
| | | QGLEWMGRVSPGSGNVKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | GDRVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 105 | VL | DIQMTQSPSFLSASVGDRVTITCKASQNVGDRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 106 | VH | QVQLVQSGAEVKKPGSSVKVSCKASGTDFKLTYIHWVRQAPG |
| | | QGLEWMGRVSPGSGNVKYNEKFKGRVTITADTSTSTAYMELS |
| | | SLRSEDTAVYYCAGSYYSYDVLDYWGQGTTVTVSS |

A-H.12

| | | |
|---|---|---|
| SEQ ID NO: 107 | VH + VL | QVQLVQSGAEVKKPGSSVKVSCKASGTDFDKIYIHWVRQAPG |
| | | QGLEWMGRVSAGSGNTKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | GNRVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 108 | VL | DIQMTQSPSFLSASVGDRVTITCKASQNVGNRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO 109 | VH | QVQLVQSGAEVKKPGSSVKVSCKASGTDFDKIYIHWVRQAPG |
| | | QGLEWMGRVSAGSGNTKYNEKFKGRVTITADTSTSTAYMELS |
| | | SLRSEDTAVYYCAGSYYSYDVLDYWGQGTTVTVSS |

A-H.13

| | | |
|---|---|---|
| SEQ ID NO: 110 | VH + VL | QVQLVQSGAEVKKPGSSVKVSCKASGTDFKLTYIHWVRQAPG |
| | | QGLEWMGRIFPGSGNVKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | DNRVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 111 | VL | DIQMTQSPSFLSASVGDRVTITCKASQNVDNRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 112 | VH | QVQLVQSGAEVKKPGSSVKVSCKASGTDFKLTYIHWVRQAPG |
| | | QGLEWMGRIFPGSGNVKYNEKFKGRVTITADTSTSTAYMELS |
| | | SLRSEDTAVYYCAGSYYSYDVLDYWGQGTTVTVSS |

A-H.14

| | | |
|---|---|---|
| SEQ ID NO: 113 | VH + VL | QVQLVQSGAEVKKPGSSVKVSCKASGTDFDKIYIHWVRQAPG |
| | | QGLEWMGRISAGSGNTKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | DDRVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 114 | VL | DIQMTQSPSFLSASVGDRVTITCKASQNVDDRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 115 | VH | QVQLVQSGAEVKKPGSSVKVSCKASGTDFDKIYIHWVRQAPG |
| | | QGLEWMGRISAGSGNTKYNEKFKGRVTITADTSTSTAYMELS |
| | | SLRSEDTAVYYCAGSYYSYDVLDYWGQGTTVTVSS |

A-H.15

| | | |
|---|---|---|
| SEQ ID NO: 116 | VH + VL | QVQLVQSGAEVKKPGSSVKVSCKASGTDFRLTYIHWVRQAPG |
| | | QGLEWMGRVSPGSGNTKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | DNKVAWHQQKPGKAPKALIYSSSHRYKGVPSRFSGSGSGTEF |
| | | TLTISSLQPEDFATYFCQQFKSYPLTFGQGTKLEIK |
| SEQ ID NO: 117 | VL | DIQMTQSPSFLSASVGDRVTITCKASQNVDNKVAWHQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 118 | VH | QVQLVQSGAEVKKPGSSVKVSCKASGTDFRLTYIHWVRQAPG |
| | | QGLEWMGRVSPGSGNTKYNEKFKGRVTITADTSTSTAYMELS |
| | | SLRSEDTAVYYCAGSYYSYDVLDYWGQGTTVTVSS |

A-H.16

| | | |
|---|---|---|
| SEQ ID NO: 119 | VH + VL | QVQLVQSGAEVKKPGSSVKVSCKASGGTFRLTYIHWVRQAPG |
| | | QGLEWMGRVYPGSGNTKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | DDRVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 120 | VL | DIQMTQSPSFLSASVGDRVTITCKASQNVDDRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 121 | VH | QVQLVQSGAEVKKPGSSVKVSCKASGGTFRLTYIHWVRQAPG |
| | | QGLEWMGRVYPGSGNTKYNEKFKGRVTITADTSTSTAYMELS |
| | | SLRSEDTAVYYCAGSYYSYDVLDYWGQGTTVTVSS |

A-H.17

| | | |
|---|---|---|
| SEQ ID NO: 122 | VH + VL | QVQLVQSGAEVKKPGSSVKVSCKASGTDFRLTYIHWVRQAPG |
| | | QGLEWMGRIFPGSGNTKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | DDRVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 123 | VL | DIQMTQSPSFLSASVGDRVTITCKASQNVDDRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 124 | VH | QVQLVQSGAEVKKPGSSVKVSCKASGTDFRLTYIHWVRQAPG |
| | | QGLEWMGRIFPGSGNTKYNEKFKGRVTITADTSTSTAYMELS |
| | | SLRSEDTAVYYCAGSYYSYDVLDYWGQGTTVTVSS |

A-H.18

| | | |
|---|---|---|
| SEQ ID NO: 125 | VH + VL | QVQLVQSGAEVKKPGSSVKVSCKASGTDFKLTYIHWVRQAPG |
| | | QGLEWMGRIFPGSGNVKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | EDRVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 126 | VL | DIQMTQSPSFLSASVGDRVTITCKASQNVEDRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 127 | VH | QVQLVQSGAEVKKPGSSVKVSCKASGTDFKLTYIHWVRQAPG |
| | | QGLEWMGRIFPGSGNVKYNEKFKGRVTITADTSTSTAYMELS |
| | | SLRSEDTAVYYCAGSYYSYDVLDYWGQGTTVTVSS |

A-H.19

| | | |
|---|---|---|
| SEQ ID NO: 128 | VH + VL | QVQLVQSGAEVKKPGSSVKVSCKASGGTFRLTYIHWVRQAPG |
| | | QGLEWMGRISAGSGNVKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | GDRVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 129 | VL | DIQMTQSPSFLSASVGDRVTITCKASQNVGDRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 130 | VH | QVQLVQSGAEVKKPGSSVKVSCKASGGTFRLTYIHWVRQAPG |
| | | QGLEWMGRISAGSGNVKYNEKFKGRVTITADTSTSTAYMELS |
| | | SLRSEDTAVYYCAGSYYSYDVLDYWGQGTTVTVSS |

A-H.20

| | | |
|---|---|---|
| SEQ ID NO: 131 | VH + VL | QVQLVQSGAEVKKPGSSVKVSCKASGGTFDKTYIHWVRQAPG |
| | | QGLEWMGRISAGSGNTKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | DDRVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 132 | VL | DIQMTQSPSFLSASVGDRVTITCKASQNVDDRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 133 | VH | QVQLVQSGAEVKKPGSSVKVSCKASGGTFDKTYIHWVRQAPG |
| | | QGLEWMGRISAGSGNTKYNEKFKGRVTITADTSTSTAYMELS |
| | | SLRSEDTAVYYCAGSYYSYDVLDYWGQGTTVTVSS |

A-H.21

| | | |
|---|---|---|
| SEQ ID NO: 134 | VH + VL | QVQLVQSGAEVKKPGSSVKVSCKASGHDFDKFYIHWVRQAPG |
| | | QGLEWMGRISAGSGNTKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | DDRVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 135 | VL | DIQMTQSPSFLSASVGDRVTITCKASQNVDDRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 136 | VH | QVQLVQSGAEVKKPGSSVKVSCKASGHDFDKFYIHWVRQAPG |
| | | QGLEWMGRISAGSGNTKYNEKFKGRVTITADTSTSTAYMELS |
| | | SLRSEDTAVYYCAGSYYSYDVLDYWGQGTTVTVSS |

A-H.22

| | | |
|---|---|---|
| SEQ ID NO: 137 | VH + VL | QVQLVQSGAEVKKPGSSVKVSCKASGTDFKLTYIHWVRQAPG |
| | | QGLEWMGRISAGSGNVKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | DNKVAWHQQKPGKAPKALIYSSSHRYKGVPSRFSGSGSGTEF |
| | | TLTISSLQPEDFATYFCQQFKSYPLTFGQGTKLEIK |
| SEQ ID NO: 138 | VL | DIQMTQSPSFLSASVGDRVTITCKASQNVDNKVAWHQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 139 | VH | QVQLVQSGAEVKKPGSSVKVSCKASGTDFKLTYIHWVRQAPG |
| | | QGLEWMGRISAGSGNVKYNEKFKGRVTITADTSTSTAYMELS |
| | | SLRSEDTAVYYCAGSYYSYDVLDYWGQGTTVTVSS |

A-H.23

| | | |
|---|---|---|
| SEQ ID NO: 140 | VH + VL | QVQLVQSGAEVKKPGSSVKVSCKASGHDFRLTYIHWVRQAPG |
| | | QGLEWMGRISAGSGNVKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | ADRVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 141 | VL | DIQMTQSPSFLSASVGDRVTITCKASQNVADRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 142 | VH | QVQLVQSGAEVKKPGSSVKVSCKASGHDFRLTYIHWVRQAPG |
| | | QGLEWMGRISAGSGNVKYNEKFKGRVTITADTSTSTAYMELS |
| | | SLRSEDTAVYYCAGSYYSYDVLDYWGQGTTVTVSS |

A-H.24

| | | |
|---|---|---|
| SEQ ID NO: 143 | VH + VL | QVQLVQSGAEVKKPGSSVKVSCKASGHDFHLWYIHWVRQAPG |
| | | QGLEWMGRVSAGSGNVKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | DNKVAWHQQKPGKAPKALIYSSSHRYKGVPSRFSGSGSGTEF |
| | | TLTISSLQPEDFATYFCQQFKSYPLTFGQGTKLEIK |
| SEQ ID NO: 144 | VL | DIQMTQSPSFLSASVGDRVTITCKASQNVDNKVAWHQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 145 | VH | QVQLVQSGAEVKKPGSSVKVSCKASGHDFHLWYIHWVRQAPG |
| | | QGLEWMGRVSAGSGNVKYNEKFKGRVTITADTSTSTAYMELS |
| | | SLRSEDTAVYYCAGSYYSYDVLDYWGQGTTVTVSS |

A-H.25

| | | |
|---|---|---|
| SEQ ID NO: 146 | VH + VL | QVQLVQSGAEVKKPGSSVKVSCKASGHDFHLWYIHWVRQAPG |
| | | QGLEWMGRVFAGSGNTKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | EDKVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 147 | VL | DIQMTQSPSFLSASVGDRVTITCKASQNVEDKVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 148 | VH | QVQLVQSGAEVKKPGSSVKVSCKASGHDFHLWYIHWVRQAPG |
| | | QGLEWMGRVFAGSGNTKYNEKFKGRVTITADTSTSTAYMELS |
| | | SLRSEDTAVYYCAGSYYSYDVLDYWGQGTTVTVSS |

A-H.26

| | | |
|---|---|---|
| SEQ ID NO: 149 | VH + VL | QVQLVQSGAEVKKPGSSVKVSCKASGTDFKLTYIHWVRQAPG |
| | | QGLEWMGRIFPGSGNTKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | DDRVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 150 | VL | DIQMTQSPSFLSASVGDRVTITCKASQNVDDRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 151 | VH | QVQLVQSGAEVKKPGSSVKVSCKASGTDFKLTYIHWVRQAPG |
| | | QGLEWMGRIFPGSGNTKYNEKFKGRVTITADTSTSTAYMELS |
| | | SLRSEDTAVYYCAGSYYSYDVLDYWGQGTTVTVSS |

A-H.27

| | | |
|---|---|---|
| SEQ ID NO: 153 | VH + VL | QVQLVQSGAEVKKPGSSVKVSCKASGTDFKLTYIHWVRQAPG |
| | | QGLEWMGRVSAGSGNVKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | GNRVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 154 | VL | DIQMTQSPSFLSASVGDRVTITCKASQNVGNRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 155 | VH | QVQLVQSGAEVKKPGSSVKVSCKASGTDFKLTYIHWVRQAPG |
| | | QGLEWMGRVSAGSGNVKYNEKFKGRVTITADTSTSTAYMELS |
| | | SLRSEDTAVYYCAGSYYSYDVLDYWGQGTTVTVSS |

A-H.28

| | | |
|---|---|---|
| SEQ ID NO: 156 | VH + VL | QVQLVQSGAEVKKPGSSVKVSCKASGTDFKLTYIHWVRQAPG |
| | | QGLEWMGRISPGSGNTKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | GDRVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 157 | VL | DIQMTQSPSFLSASVGDRVTITCKASQNVGDRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 158 | VH | QVQLVQSGAEVKKPGSSVKVSCKASGTDFKLTYIHWVRQAPG |
| | | QGLEWMGRISPGSGNTKYNEKFKGRVTITADTSTSTAYMELS |
| | | SLRSEDTAVYYCAGSYYSYDVLDYWGQGTTVTVSS |

A-H.29

| | | |
|---|---|---|
| SEQ ID NO: 159 | VH + VL | QVQLVQSGAEVKKPGSSVKVSCKASGHDFHLWYIHWVRQAPG |
| | | QGLEWMGRISPGSGNVKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | GDRVAWHQQKPGKAPKALIYSSSHRYKGVPSRFSGSGSGTEF |
| | | TLTISSLQPEDFATYFCQQFKSYPLTFGQGTKLEIK |
| SEQ ID NO: 160 | VL | DIQMTQSPSFLSASVGDRVTITCKASQNVGDRVAWHQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 161 | VH | QVQLVQSGAEVKKPGSSVKVSCKASGHDFHLWYIHWVRQAPG |
| | | QGLEWMGRISPGSGNVKYNEKFKGRVTITADTSTSTAYMELS |
| | | SLRSEDTAVYYCAGSYYSYDVLDYWGQGTTVTVSS |

A-H.31

| | | |
|---|---|---|
| SEQ ID NO: 162 | VH + VL | QVQLVQSGAEVKKPGSSVKVSCKASGHDFKLTYIHWVRQAPG |
| | | QGLEWMGRISAGSGNVKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | DDRVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 163 | VL | DIQMTQSPSFLSASVGDRVTITCKASQNVDDRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 164 | VH | QVQLVQSGAEVKKPGSSVKVSCKASGHDFKLTYIHWVRQAPG |
| | | QGLEWMGRISAGSGNVKYNEKFKGRVTITADTSTSTAYMELS |
| | | SLRSEDTAVYYCAGSYYSYDVLDYWGQGTTVTVSS |

A-H.31

| | | |
|---|---|---|
| SEQ ID NO: 165 | VH + VL | QVQLVQSGAEVKKPGSSVKVSCKASGTDFHLWYIHWVRQAPG |
| | | QGLEWMGRVFAGSGSYRYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | DDRVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 166 | VL | DIQMTQSPSFLSASVGDRVTITCKASQNVDDRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 167 | VH | QVQLVQSGAEVKKPGSSVKVSCKASGTDFHLWYIHWVRQAPG |
| | | QGLEWMGRVFAGSGSYRYNEKFKGRVTITADTSTSTAYMELS |
| | | SLRSEDTAVYYCAGSYYSYDVLDYWGQGTTVTVSS |

A-H.32

| | | |
|---|---|---|
| SEQ ID NO: 168 | VH + VL | QVQLVQSGAEVKKPGSSVKVSCKASGTDFDKIYIHWVRQAPG |
| | | QGLEWMGRISAGSGNTKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | ADRVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 169 | VL | DIQMTQSPSFLSASVGDRVTITCKASQNVADRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 170 | VH | QVQLVQSGAEVKKPGSSVKVSCKASGTDFDKIYIHWVRQAPG |
| | | QGLEWMGRISAGSGNTKYNEKFKGRVTITADTSTSTAYMELS |
| | | SLRSEDTAVYYCAGSYYSYDVLDYWGQGTTVTVSS |

A-H.33

| | | |
|---|---|---|
| SEQ ID NO: 171 | VH + VL | QVQLVQSGAEVKKPGSSVKVSCKASGTDFKLTYIHWVRQAPG |
| | | QGLEWMGRISAGSGNTKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | EDRVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 172 | VL | DIQMTQSPSFLSASVGDRVTITCKASQNVEDRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 173 | VH | QVQLVQSGAEVKKPGSSVKVSCKASGTDFKLTYIHWVRQAPG |
| | | QGLEWMGRISAGSGNTKYNEKFKGRVTITADTSTSTAYMELS |
| | | SLRSEDTAVYYCAGSYYSYDV |
| | | LDYWGQGTTVTVSS |

A-H.34

| | | |
|---|---|---|
| SEQ ID NO: 174 | VH + VL | QVQLVQSGAEVKKPGSSVKVSCKASGTDFRLTYIHWVRQAPG |
| | | QGLEWMGRISPGSGNTKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | GNRVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 175 | VL | DIQMTQSPSFLSASVGDRVTITCKASQNVGNRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 176 | VH | QVQLVQSGAEVKKPGSSVKVSCKASGTDFRLTYIHWVRQAPG |
| | | QGLEWMGRISPGSGNTKYNEKFKGRVTITADTSTSTAYMELS |
| | | SLRSEDTAVYYCAGSYYSYDVLDYWGQGTTVTVSS |

A-H.35

| | | |
|---|---|---|
| SEQ ID NO: 177 | VH + VL | QVQLVQSGAEVKKPGSSVKVSCKASGHDFDKTYIHWVRQAPG |
| | | QGLEWMGRVSAGSGNVKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | EDRVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 178 | VL | DIQMTQSPSFLSASVGDRVTITCKASQNVEDRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 179 | VH | QVQLVQSGAEVKKPGSSVKVSCKASGHDFDKTYIHWVRQAPG |
| | | QGLEWMGRVSAGSGNVKYNEKFKGRVTITADTSTSTAYMELS |
| | | SLRSEDTAVYYCAGSYYSYDVLDYWGQGTTVTVSS |

A-H.36

| | | |
|---|---|---|
| SEQ ID NO: 180 | VH + VL | QVQLVQSGAEVKKPGSSVKVSCKASGHDFKLTYIHWVRQAPG |
| | | QGLEWMGRVSPGSGNTKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | EDRVAWHQQKPGKAPKALIYSSSHRYKGVPSRFSGSGSGTEF |
| | | TLTISSLQPEDFATYFCQQFKSYPLTFGQGTKLEIK |
| SEQ ID NO: 181 | VL | DIQMTQSPSFLSASVGDRVTITCKASQNVEDRVAWHQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 182 | VH | QVQLVQSGAEVKKPGSSVKVSCKASGHDFKLTYIHWVRQAPG |
| | | QGLEWMGRVSPGSGNTKYNEKFKGRVTITADTSTSTAYMELS |
| | | SLRSEDTAVYYCAGSYYSYDVLDYWGQGTTVTVSS |

A-H.37

| | | |
|---|---|---|
| SEQ ID NO: 183 | VH + VL | QVQLVQSGAEVKKPGSSVKVSCKASGHDFDKTYIHWVRQAPG |
| | | QGLEWMGRIYPGSGNVKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | ADRVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 184 | VL | DIQMTQSPSFLSASVGDRVTITCKASQNVADRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 185 | VH | QVQLVQSGAEVKKPGSSVKVSCKASGHDFDKTYIHWVRQAPG |
| | | QGLEWMGRIYPGSGNVKYNEKFKGRVTITADTSTSTAYMELS |
| | | SLRSEDTAVYYCAGSYYSYDV |
| | | LDYWGQGTTVTVSS |

A-H.38

| | | |
|---|---|---|
| SEQ ID NO: 186 | VH + VL | QVQLVQSGAEVKKPGSSVKVSCKASGTDFDKTYIHWVRQAPG |
| | | QGLEWMGRISAGSGNVKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | DDRVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 187 | VL | DIQMTQSPSFLSASVGDRVTITCKASQNVDDRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 188 | VH | QVQLVQSGAEVKKPGSSVKVSCKASGTDFDKTYIHWVRQAPG |
| | | QGLEWMGRISAGSGNVKYNEKFKGRVTITADTSTSTAYMELS |
| | | SLRSEDTAVYYCAGSYYSYDVLDYWGQGTTVTVSS |

A-H.39

| | | |
|---|---|---|
| SEQ ID NO: 189 | VH + VL | QVQLVQSGAEVKKPGSSVKVSCKASGTDFDKIYIHWVRQAPG |
| | | QGLEWMGRISAGSGNIKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | DDRVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 190 | VL | DIQMTQSPSFLSASVGDRVTITCKASQNVDDRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 191 | VH | QVQLVQSGAEVKKPGSSVKVSCKASGTDFDKIYIHWVRQAPG |
| | | QGLEWMGRISAGSGNIKYNEKFKGRVTITADTSTSTAYMELS |
| | | SLRSEDTAVYYCAGSYYSYDVLDYWGQGTTVTVSS |

A-H.40

| | | |
|---|---|---|
| SEQ ID NO: 192 | VH + VL | QVQLVQSGAEVKKPGSSVKVSCKASGTDFDKIYIHWVRQAPG |
| | | QGLEWMGRISAGSGNVKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | GDRVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 193 | VL | DIQMTQSPSFLSASVGDRVTITCKASQNVGDRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 194 | VH | QVQLVQSGAEVKKPGSSVKVSCKASGTDFDKIYIHWVRQAPG |
| | | QGLEWMGRISAGSGNVKYNEKFKGRVTITADTSTSTAYMELS |
| | | SLRSEDTAVYYCAGSYYSYDVLDYWGQGTTVTVSS |

A-H.41

| | | |
|---|---|---|
| SEQ ID NO: 195 | VH + VL | QVQLVQSGAEVKKPGSSVKVSCKASGGTFKLTYIHWVRQAPG |
| | | QGLEWMGRVSAGSGNVKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | DDRVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 196 | VL | DIQMTQSPSFLSASVGDRVTITCKASQNVDDRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 197 | VH | QVQLVQSGAEVKKPGSSVKVSCKASGGTFKLTYIHWVRQAPG |
| | | QGLEWMGRVSAGSGNVKYNEKFKGRVTITADTSTSTAYMELS |
| | | SLRSEDTAVYYCAGSYYSYDVLDYWGQGTTVTVSS |

A-H.42

| | | |
|---|---|---|
| SEQ ID NO: 198 | VH + VL | QVQLVQSGAEVKKPGSSVKVSCKASGTDFKLTYIHWVRQAPG |
| | | QGLEWMGRISPGSGNVKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | DNRVAWHQQKPGKAPKALIYSSSHRYKGVPSRFSGSGSGTEF |
| | | TLTISSLQPEDFATYFCQQFKSYPLTFGQGTKLEIK |
| SEQ ID NO: 199 | VL | DIQMTQSPSFLSASVGDRVTITCKASQNVDNRVAWHQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 200 | VH | QVQLVQSGAEVKKPGSSVKVSCKASGTDFKLTYIHWVRQAPG |
| | | QGLEWMGRISPGSGNVKYNEKFKGRVTITADTSTSTAYMELS |
| | | SLRSEDTAVYYCAGSYYSYDVLDYWGQGTTVTVSS |

A-H.43

| | | |
|---|---|---|
| SEQ ID NO: 201 | VH + VL | QVQLVQSGAEVKKPGSSVKVSCKASGHDFDKFYIHWVRQAPG |
| | | QGLEWMGRVSAGSGNTKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | DNRVAWYQQKPGKAPKALIYSSSHRYKGVPSRFSGSGSGTEF |
| | | TLTISSLQPEDFATYFCQQFKSYPLTFGQGTKLEIK |
| SEQ ID NO: 202 | VL | DIQMTQSPSFLSASVGDRVTITCKASQNVDNRVAWYQQKPGK |
| | | APKALIYSSSHRYKGVPSRFSGSGSGTEFTLTISSLQPEDFA |
| | | TYFCQQFKSYPLTFGQGTKLEIK |
| SEQ ID NO: 203 | VH | QVQLVQSGAEVKKPGSSVKVSCKASGHDFDKFYIHWVRQAPG |
| | | QGLEWMGRVSAGSGNTKYNEKFKGRVTITADTSTSTAYMELS |
| | | SLRSEDTAVYYCAGSYYSYDVLDYWGQGTTVTVSS |

A-H.44

| | | |
|---|---|---|
| SEQ ID NO: 204 | VH + VL | QVQLVQSGAEVKKPGSSVKVSCKASGTDFDKFYIHWVRQAPG |
| | | QGLEWMGRVSAGSGNVKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | GDRVVWYQQKPGKAPKALIYSSSHRYKGVPSRFSGSGSGTEF |
| | | TLTISSLQPEDFATYFCQQFKSYPLTFGQGTKLEIK |
| SEQ ID NO: 205 | VH | QVQLVQSGAEVKKPGSSVKVSCKASGTDFDKFYIHWVRQAPG |
| | | QGLEWMGRVSAGSGNVKYNEKFKGRVTITADTSTSTAYMELS |
| | | SLRSEDTAVYYCAGSYYSYDVLDYWGQGTTVTVSS |

A-H.45

| | | |
|---|---|---|
| SEQ ID NO: 206 | VH + VL | QVQLVQSGAEVKKPGSSVKVSCKASGYSFTTYYIHWVRQAPG |
| | | QGLEWMGWFSAGSGNTKYNEKFKGRVTITADTSTSTAYMELS |
| | | SLRSEDTAVYYCAVSYYSYDVLDYWGQGTTVTVSSGGGGSGG |
| | | GGSGGGGSGGGGSDIQMTQSPSFLSASVGDRVTITCKASQNV |
| | | GINVVWHQQKPGKAPKALIYSSSHRYSGVPSRFSGSGSGTEF |
| | | TLTISSLQPEDFATYFCQQFKSYPLTFGQGTKLEIK |
| SEQ ID NO: 207 | VH | QVQLVQSGAEVKKPGSSVKVSCKASGYSFTTYYIHWVRQAPG |
| | | QGLEWMGWFSAGSGNTKYNEKFKGRVTITADTSTSTAYMELS |
| | | SLRSEDTAVYYCAVSYYSYDVLDYWGQGTTVTVSS |

A-H.46

| | | |
|---|---|---|
| SEQ ID NO: 208 | VH + VL | QVQLVQSGAEVKKPGSSVKVSCKASGYSFTTYYIHWVRQAPG |
| | | QGLEWMGWFSAGSGNTKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | GINVVWHQQKPGKAPKALIYSSSHRYSGVPSRFSGSGSGTEF |
| | | TLTISSLQPEDFATYFCQQFKSYPLTFGQGTKLEIK |
| SEQ ID NO: 209 | VH | QVQLVQSGAEVKKPGSSVKVSCKASGYSFTTYYIHWVRQAPG |
| | | QGLEWMGWFSAGSGNTKYNEKFKGRVTITADTSTSTAYMELS |
| | | SLRSEDTAVYYCAGSYYSYDVLDYWGQGTTVTVSS |

A-H.47

| | | |
|---|---|---|
| SEQ ID NO: 210 | VH + VL | QVQLVQSGAEVKKPGSSVKVSCKASGYSFTTYYIHWVRQAPG |
| | | QGLEWMGWFFPGSGNTKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | GINVVWHQQKPGKAPKALIYSSSHRYSGVPSRFSGSGSGTEF |
| | | TLTISSLQPEDFATYFCQQFHSYPLTFGQGTKLEIK |
| SEQ ID NO: 211 | VH | QVQLVQSGAEVKKPGSSVKVSCKASGYSFTTYYIHWVRQAPG |
| | | QGLEWMGWFFPGSGNTKYNEKFKGRVTITADTSTSTAYMELS |
| | | SLRSEDTAVYYCAGSYYSYDVLDYWGQGTTVTVSS |

A-H.48

| | | |
|---|---|---|
| SEQ ID NO: 212 | VH + VL | QVQLVQSGAEVKKPGSSVKVSCKASGYSFTTYYIHWVRQAPG |
| | | QGLEWMGWFSPGSGNTKYNEKFKGRVTITADTSTSTAYMELS |
| | | SLRSEDTAVYYCAVSYYSYDVLDYWGQGTTVTVSSGGGGSGG |
| | | GGSGGGGSGGGGSDIQMTQSPSFLSASVGDRVTITCKASQNV |
| | | GINVVWHQQKPGKAPKALIYSSSHRYSGVPSRFSGSGSGTEF |
| | | TLTISSLQPEDFATYFCQQFKSYPLTFGQGTKLEIK |
| SEQ ID NO: 213 | VH | QVQLVQSGAEVKKPGSSVKVSCKASGYSFTTYYIHWVRQAPG |
| | | QGLEWMGWFSPGSGNTKYNEKFKGRVTITADTSTSTAYMELS |
| | | SLRSEDTAVYYCAVSYYSYDVLDYWGQGTTVTVSS |

A-H.49

| | | |
|---|---|---|
| SEQ ID NO: 214 | VH + VL | QVQLVQSGAEVKKPGSSVKVSCKASGYSFTTYYIHWVRQAPG |
| | | QGLEWMGWFSPGSGNTKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | GINVVWHQQKPGKAPKALIYSSSHRYSGVPSRFSGSGSGTEF |
| | | TLTISSLQPEDFATYFCQQFKSYPLTFGQGTKLEIK |
| SEQ ID NO: 215 | VH | QVQLVQSGAEVKKPGSSVKVSCKASGYSFTTYYIHWVRQAPG |
| | | QGLEWMGWFSPGSGNTKYNEKFKGRVTITADTSTSTAYMELS |
| | | SLRSEDTAVYYCAGSYYSYDVLDYWGQGTTVTVSS |

A-H.50

| | | |
|---|---|---|
| SEQ ID NO: 216 | VH + VL | QVQLVQSGAEVKKPGSSVKVSCKASGYSFTTYYIHWVRQAPG |
| | | QGLEWMGRIFPGSGNIKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | GINVVWHQQKPGKAPKALIYSSSHRYSGVPSRFSGSGSGTEF |
| | | TLTISSLQPEDFATYFCQQFKSYPLTFGQGTKLEIK |
| SEQ ID NO: 217 | VH | QVQLVQSGAEVKKPGSSVKVSCKASGYSFTTYYIHWVRQAPG |
| | | QGLEWMGRIFPGSGNIKYNEKFKGRVTITADTSTSTAYMELS |
| | | SLRSEDTAVYYCAGSYYSYDVLDYWGQGTTVTVSS |

A-H.51

| | | |
|---|---|---|
| SEQ ID NO: 218 | VH + VL | QVQLVQSGAEVKKPGSSVKVSCKASGYSFTTYYIHWVRQAPG |
| | | QGLEWMGWFFPGSGNIKYNEKFKGRVTITADTSTSTAYMELS |
| | | SLRSEDTAVYYCAGSIYSAGVLDYWGQGTTVTVSSGGGGSGG |
| | | GGSGGGGSGGGGSDIQMTQSPSFLSASVGDRVTITCKASQNV |
| | | GINVVWHQQKPGKAPKALIYSSSHRYSGVPSRFSGSGSGTEF |
| | | TLTISSLQPEDFATYFCQQFKSYPLTFGQGTKLEIK |
| SEQ ID NO: 219 | VH | QVQLVQSGAEVKKPGSSVKVSCKASGYSFTTYYIHWVRQAPG |
| | | QGLEWMGWFFPGSGNIKYNEKFKGRVTITADTSTSTAYMELS |
| | | SLRSEDTAVYYCAGSIYSAGVLDYWGQGTTVTVSS |

A-H.52

| | | |
|---|---|---|
| SEQ ID NO: 220 | VH + VL | QVQLVQSGAEVKKPGSSVKVSCKASGYSFTLGYIHWVRQAPG |
| | | QGLEWMGWFFPGSGNIKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | GINVVWHQQKPGKAPKALIYSSSHRYSGVPSRFSGSGSGTEF |
| | | TLTISSLQPEDFATYFCQQFKSYPLTFGQGTKLEIK |
| SEQ ID NO: 221 | VH | QVQLVQSGAEVKKPGSSVKVSCKASGYSFTLGYIHWVRQAPG |
| | | QGLEWMGWFFPGSGNIKYNEKFKGRVTITADTSTSTAYMELS |
| | | SLRSEDTAVYYCAGSYYSYDVLDYWGQGTTVTVSS |

A.H.53

| | | |
|---|---|---|
| SEQ ID NO: 222 | VH + VL | QVQLVQSGAEVKKPGSSVKVSCKASGYSFRLTYIHWVRQAPG |
| | | QGLEWMGWFFPGSGNIKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | GINVVWHQQKPGKAPKALIYSSSHRYSGVPSRFSGSGSGTEF |
| | | TLTISSLQPEDFATYFCQQFKSYPLTFGQGTKLEIK |
| SEQ ID NO: 223 | VH | QVQLVQSGAEVKKPGSSVKVSCKASGYSFRLTYIHWVRQAPG |
| | | QGLEWMGWFFPGSGNIKYNEKFKGRVTITADTSTSTAYMELS |
| | | SLRSEDTAVYYCAGSYYSYDVLDYWGQGTTVTVSS |

A-H.54

| | | |
|---|---|---|
| SEQ ID NO: 224 | VH + VL | QVQLVQSGAEVKKPGSSVKVSCKASGYSFHNWYIHWVRQAPG |
| | | QGLEWMGWFFPGSGNIKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | GINVVWHQQKPGKAPKALIYSSSHRYSGVPSRFSGSGSGTEF |
| | | TLTISSLQPEDFATYFCQQFKSYPLTFGQGTKLEIK |
| SEQ ID NO: 225 | VH | QVQLVQSGAEVKKPGSSVKVSCKASGYSFHNWYIHWVRQAPG |
| | | QGLEWMGWFFPGSGNIKYNEKFKGRVTITADTSTSTAYMELS |
| | | SLRSEDTAVYYCAGSYYSYDVLDYWGQGTTVTVSS |

A-H.55 antibody

| | | |
|---|---|---|
| SEQ ID NO: 3 | HC CDR1 | GYSFTTYYIH |
| | (Combined) | |
| SEQ ID NO: 4 | HC CDR2 | WFFPGSGNIKYNEKFKG |
| | (Combined) | |
| SEQ ID NO: 5 | HC CDR3 | SYYSYDVLDY |
| | (Combined) | |
| SEQ ID NO: 45 | HC CDR1 (Kabat) | TYYIH |
| SEQ ID NO: 46 | HC CDR2 (Kabat) | WFFPGSGNIKYNEKFKG |
| SEQ ID NO: 47 | HC CDR3 (Kabat) | SYYSYDVLDY |
| SEQ ID NO: 48 | HC CDR1 (Chothia) | GYSFTTY |
| SEQ ID NO: 49 | HC CDR2 (Chothia) | FPGSGN |
| SEQ ID NO: 50 | HC CDR3 (Chothia) | SYYSYDVLDY |
| SEQ ID NO: 1100 | VH | QVQLVQSGAEVKKPGSSVKVSCKASGYSFTTYYIHWVRQAPG |
| | | QGLEWMGWFFPGSGNIKYNEKFKGRVTITADTSTSTAYMELS |
| | | SLRSEDTAVYYCAGSYYSYDVLDYWGQGTTVTVSS |
| SEQ ID NO: 6 | LC CDR1 (Combined) | KASQNVGINVV |
| SEQ ID NO: 7 | LC CDR2 (Combined) | SSSHRYS |
| SEQ ID NO: 8 | LC CDR3 (Combined) | QQFKSYPLT |
| SEQ ID NO: 51 | LC CDR1 (Kabat) | KASQNVGINVV |
| SEQ ID NO: 52 | LC CDR2 (Kabat) | SSSHRYS |
| SEQ ID NO: 53 | LC CDR3 (Kabat) | QQFKSYPLT |
| SEQ ID NO: 54 | LC CDR1 (Chothia) | KASQNVGINVV |
| SEQ ID NO: 55 | LC CDR2 (chothia) | SSSHRYS |
| SEQ ID NO: 56 | LC CDR3 (chothia) | QQFKSYPLT |
| SEQ ID NO: 1101 | VL | QSVLTQPPSVSEAPRQRVTISCKASQNVGINVVWHQQLPGKA |
| | | PKALIYSSSHRYSGVSDRFSGSGSGTSFSLAISGLQSEDEAD |
| | | YFCQQFKSYPLTFGTGTKVTVL |

A-H.56

| | | |
|---|---|---|
| SEQ ID NO: 1309 | VH + VL (ScFv) | QVQLVQSGAEVKKPGSSVKVSCKASGHDFDKFYIHWVRQAPG |
| | | QGLEWMGRVSAGSGNVKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | GNRVAWYQQKPGKAPKALIYSSSHRYKGVPSRFSGSGSGTEF |
| | | TLTISSLQPEDFATYFCQQFKSYPLTFGQGTKLEIK |

A-H.57

| | | |
|---|---|---|
| SEQ ID NO: 1326 | VH + VL (ScFv) | QVQLVQSGAEVKKPGSSVKVSCKASGHDFRLTYIHWVRQAPG |
| | | QGLEWMGRVSAGSGNTKYNEKFKGRVTITADTSTSTAYMELS |
| | | SLRSEDTAVYYCAVSYYSYDVLDYWGQGTTVTVSSGGGGSGG |
| | | GGSGGGGSGGGGSDIQMTQSPSFLSASVGDRVTITCKASQNV |
| | | GDRVVWHQQKPGKAPKALIYSSSHRYKGVPSRFSGSGSGTEF |
| | | TLTISSLQPEDFATYFCQQFKSYPLTFGQGTKLEIK |

A-H.58

| | | |
|---|---|---|
| SEQ ID NO: 1327 | VH + VL (ScFv) | QVQLVQSGAEVKKPGSSVKVSCKASGHDFRLTYIHWVRQAPG |
| | | QGLEWMGRVSAGSGNVKYNEKFKGRVTITADTSTSTAYMELS |
| | | SLRSEDTAVYYCAVSYYSYDVLDYWGQGTTVTVSSGGGGSGG |
| | | GGSGGGGSGGGGSDIQMTQSPSFLSASVGDRVTITCKASQNV |
| | | GNRVVWHQQKPGKAPKALIYSSSHRYKGVPSRFSGSGSGTEF |
| | | TLTISSLQPEDFATYFCQQFKSYPLTFGQGTKLEIK |

A-H.59

| | | |
|---|---|---|
| SEQ ID NO: 1328 | VH + VL (ScFv) | QVQLVQSGAEVKKPGSSVKVSCKASGHDFRLTYIHWVRQAPG |
| | | QGLEWMGRIYAGSGNVKYNEKFKGRVTITADTSTSTAYMELS |
| | | SLRSEDTAVYYCAVSYYSYDVLDYWGQGTTVTVSSGGGGSGG |
| | | GGSGGGGSGGGGSDIQMTQSPSFLSASVGDRVTITCKASQNV |
| | | ADRVVWHQQKPGKAPKALIYSSSHRYKGVPSRFSGSGSGTEF |
| | | TLTISSLQPEDFATYFCQQFKSYPLTFGQGTKLEIK |

A-H.60

| | | |
|---|---|---|
| SEQ ID NO: 1329 | VH + VL (ScFv) | QVQLVQSGAEVKKPGSSVKVSCKASGHDFKLTYIHWVRQAPG |
| | | QGLEWMGRVSAGSGNTKYNEKFKGRVTITADTSTSTAYMELS |
| | | SLRSEDTAVYYCAVSYYSYDVLDYWGQGTTVTVSSGGGGSGG |
| | | GGSGGGGSGGGGSDIQMTQSPSFLSASVGDRVTITCKASQNV |
| | | GDRVAWHQQKPGKAPKALIYSSSHRYKGVPSRFSGSGSGTEF |
| | | TLTISSLQPEDFATYFCQQFKSYPLTFGQGTKLEIK |

A-H.61

| | | |
|---|---|---|
| SEQ ID NO: 1330 | VH + VL (ScFv) | QVQLVQSGAEVKKPGSSVKVSCKASGHDFKLTYIHWVRQAPG |
| | | QGLEWMGRVSAGSGNTKYNEKFKGRVTITADTSTSTAYMELS |
| | | SLRSEDTAVYYCAVSYYSYDVLDYWGQGTTVTVSSGGGGSGG |
| | | GGSGGGGSGGGGSDIQMTQSPSFLSASVGDRVTITCKASQNV |
| | | DNRVAWHQQKPGKAPKALIYSSSHRYKGVPSRFSGSGSGTEF |
| | | TLTISSLQPEDFATYFCQQFKSYPLTFGQGTKLEIK |

A-H.62

| | | |
|---|---|---|
| SEQ ID NO: 1331 | VH + VL (ScFv) | QVQLVQSGAEVKKPGSSVKVSCKASGHDFRLTYIHWVRQAPG |
| | | QGLEWMGRVSAGSGNVKYNEKFKGRVTITADTSTSTAYMELS |
| | | SLRSEDTAVYYCAVSYYSYDVLDYWGQGTTVTVSSGGGGSGG |
| | | GGSGGGGSGGGGSDIQMTQSPSFLSASVGDRVTITCKASQNV |
| | | ADRVVWHQQKPGKAPKALIYSSSHRYKGVPSRFSGSGSGTEF |
| | | TLTISSLQPEDFATYFCQQFKSYPLTFGQGTKLEIK |

A-H.63

| | | |
|---|---|---|
| SEQ ID NO: 1332 | VH + VL (ScFv) | QVQLVQSGAEVKKPGSSVKVSCKASGHDFRLTYIHWVRQAPG |
| | | QGLEWMGRVYAGSGNTKYNEKFKGRVTITADTSTSTAYMELS |
| | | SLRSEDTAVYYCAVSYYSYDVLDYWGQGTTVTVSSGGGGSGG |
| | | GGSGGGGSGGGGSDIQMTQSPSFLSASVGDRVTITCKASQNV |
| | | EDRVVWHQQKPGKAPKALIYSSSHRYKGVPSRFSGSGSGTEF |
| | | TLTISSLQPEDFATYFCQQFKSYPLTFGQGTKLEIK |

A-H.64

| | | |
|---|---|---|
| SEQ ID NO: 1333 | VH + VL (ScFv) | QVQLVQSGAEVKKPGSSVKVSCKASGHDFKLTYIHWVRQAPG |
| | | QGLEWMGRVSAGSGNTKYNEKFKGRVTITADTSTSTAYMELS |
| | | SLRSEDTAVYYCAVSYYSYDVLDYWGQGTTVTVSSGGGGSGG |
| | | GGSGGGGSGGGGSDIQMTQSPSFLSASVGDRVTITCKASQNV |
| | | ADRVVWHQQKPGKAPKALIYSSSHRYKGVPSRFSGSGSGTEF |
| | | TLTISSLQPEDFATYFCQQFKSYPLTFGQGTKLEIK |

A-H.65

| | | |
|---|---|---|
| SEQ ID NO: 1334 | VH + VL (ScFv) | QVQLVQSGAEVKKPGSSVKVSCKASGHDFKLTYIHWVRQAPG |
| | | QGLEWMGRISAGSGNTKYNEKFKGRVTITADTSTSTAYMELS |
| | | SLRSEDTAVYYCAVSYYSYDVLDYWGQGTTVTVSSGGGGSGG |
| | | GGSGGGGSGGGGSDIQMTQSPSFLSASVGDRVTITCKASQNV |
| | | GDRVVWHQQKPGKAPKALIYSSSHRYKGVPSRFSGSGSGTEF |
| | | TLTISSLQPEDFATYFCQQFKSYPLTFGQGTKLEIK |
| A-H.66 | | |
| SEQ ID NO: 1335 | VH + VL (ScFv) | QVQLVQSGAEVKKPGSSVKVSCKASGHDFKLTYIHWVRQAPG |
| | | QGLEWMGRIYAGSGNTKYNEKFKGRVTITADTSTSTAYMELS |
| | | SLRSEDTAVYYCAVSYYSYDVLDYWGQGTTVTVSSGGGGSGG |
| | | GGSGGGGSGGGGSDIQMTQSPSFLSASVGDRVTITCKASQNV |
| | | GDRVVWHQQKPGKAPKALIYSSSHRYKGVPSRFSGSGSGTEF |
| | | TLTISSLQPEDFATYFCQQFKSYPLTFGQGTKLEIK |

A-H.67

| | | |
|---|---|---|
| SEQ ID NO: 1336 | VH + VL (ScFv) | QVQLVQSGAEVKKPGSSVKVSCKASGTDFKLTYIHWVRQAPG |
| | | QGLEWMGRIFPGSGNVKYNEKFKGRVTITADTSTSTAYMELS |
| | | SLRSEDTAVYYCAVSYYSYDVLDYWGQGTTVTVSSGGGGSGG |
| | | GGSGGGGSGGGGSDIQMTQSPSFLSASVGDRVTITCKASQNV |
| | | DNRVAWYQQKPGKAPKALIYSSSHRYKGVPSRFSGSGSGTEF |
| | | TLTISSLQPEDFATYFCQQFKSYPLTFGQGTKLEIK |

A-H.68

| | | |
|---|---|---|
| SEQ ID NO: 1337 | VH + VL (ScFv) | QVQLVQSGAEVKKPGSSVKVSCKASGHDFRLTYIHWVRQAPG |
| | | QGLEWMGRISAGSGNVKYNEKFKGRVTITADTSTSTAYMELS |
| | | SLRSEDTAVYYCAVSYYSYDVLDYWGQGTTVTVSSGGGGSGG |
| | | GGSGGGGSGGGGSDIQMTQSPSFLSASVGDRVTITCKASQNV |
| | | ADRVAWYQQKPGKAPKALIYSSSHRYKGVPSRFSGSGSGTEF |
| | | TLTISSLQPEDFATYFCQQFKSYPLTFGQGTKLEIK |

A-H.69

| | | |
|---|---|---|
| SEQ ID NO: 1344 | VH + VL (ScFv) | QVQLVQSGAEVKKPGSSVKVSCKASGTDFKLTYIHWVRQAPG |
| | | QGLEWMGRIFPGSGNVKYNEKFKGRVTITADTSTSTAYMELS |
| | | SLRSEDTAVYYCAGSYYSYDVLDYWGQGTTVTVSSGGGGSGG |
| | | GGSGGGGSGGGGSDIQMTQSPSFLSASVGDRVTITCKASQNV |
| | | DNRVAWYQQKPGKAPKALIYSSSHRYKGVPSRFSGSGSGTEF |
| | | TLTISSLQPEDFATYFCQQFKSYPLTFGQGTKLEIK |

A-H humanized-matured VH

| | | |
|---|---|---|
| SEQ ID NO: 1310 | VH-humanized | QVQLVQSGAEVKKPGSSVKVSCKASGTDFKLTYIHWVRQAPG |
| | matured 1 | QGLEWMGRIFPGSGNVKYNEKFKGRVTITADTSTSTAYMELS |
| | | SLRSEDTAVYYCAGSYYSYDVLDYWGQGTTVTVSS |
| SEQ ID NO: 1311 | VH-humanized | QVQLVQSGAEVKKPGSSVKVSCKASGTDFKLTYIHWVRQAPG |
| | matured 2 | QGLEWMGRIFPGSGNVKYNEKFKGRVTITADTSTSTAYMELS |
| | | SLRSEDTAVYYCAVSYYSYDVLDYWGQGTTVTVSS |
| SEQ ID NO: 1312 | VH-humanized | QVQLVQSGAEVKKPGSSVKVSCKASGHDFRLTYIHWVRQAPG |
| | matured 3 | QGLEWMGRISAGSGNVKYNEKFKGRVTITADTSTSTAYMELS |
| | | SLRSEDTAVYYCAVSYYSYDVLDYWGQGTTVTVSS |

A-H humanized-matured VL

| | | |
|---|---|---|
| SEQ ID NO: 1313 | VL-humanized | DIQMTQSPSFLSASVGDRVTITCKASQNVDNRVAWYQQKPGK |
| | matured 1 | APKALIYSSSHRYKGVPSRFSGSGSGTEFTLTISSLQPEDFA |
| | | TYFCQQFKSYPLTFGQGTKLEIK |
| SEQ ID NO: 1314 | VL-humanized | DIQMTQSPSFLSASVGDRVTITCKASQNVADRVAWYQQKPGK |
| | matured 2 | APKALIYSSSHRYKGVPSRFSGSGSGTEFTLTISSLQPEDFA |
| | | TYFCQQFKSYPLTFGQGTKLEIK |

In some embodiments, the anti-TCRβV antibody molecule, e.g., anti-TCRβV6 (e.g., anti-TCRβ V6-5*01) antibody molecule comprises a VH and/or a VL of an antibody described in Table 3, or a sequence with at least 80%, 85%, 90%, 95%, 96%, 97%, 98%, 99% or more identity thereto.

In some embodiments, the anti-TCRβV antibody molecule, e.g., anti-TCRβV6 (e.g., anti-TCRβ V6-5*01) antibody molecule comprises a VH and a VL of an antibody described in Table 3, or a sequence with at least 80%, 85%, 90%, 95%, 96%, 97%, 98%, 99% or more identity thereto.

In some embodiments, an anti-TCRβV antibody disclosed herein has an antigen binding domain having a VL having a consensus sequence of SEQ ID NO: 230, wherein position 30 is G, E, A or D; position 31 is N or D; position 32 is R or K; position 36 is Y or H; and/or position 56 is K or S.

In some embodiments, an anti-TCRVb antibody disclosed herein has an antigen binding domain having a VH having a consensus sequence of SEQ ID NO: 231, wherein: position 27 is H or T or G or Y; position 28 is D or T or S; position 30 is H or R or D or K or T; position 31 is L or D or K or T or N; position 32 is W or F or T or I or Y or G; position 49 is R or W; position 50 is V or I or F; position 51 is F or S or Y; position 52 is A or P; position 56 is N or S; position 57 is T or V or Y or I; position 58 is K or R; position 97 is G or V; position 99 is Y or I; position 102 is Y or A; and/or position 103 is D or G.

### Anti-TCRβV12 Antibodies

Accordingly, in one aspect, the disclosure provides an anti-TCRβV antibody molecule that binds to human TCRβ V12, e.g., a TCRβ V12 subfamily comprising: TCRβ V12-4*01, TCRβ V12-3*01 or TCRβ V12-5*01. In some embodiments the TCRβ V12 subfamily comprises TCRβ V12-4*01. In some embodiments the TCRβ V12 subfamily comprises TCRβ V12-3*01.

In some embodiments, the anti-TCRβV antibody molecule, e.g., anti-TCRβV12 antibody molecule, is a non-murine antibody molecule, e.g., a human or humanized antibody molecule. In some embodiments, the anti-TCRβV antibody molecule, e.g., anti-TCRβV12 antibody molecule is a human antibody molecule. In some embodiments, the anti-TCRβV antibody molecule, e.g., anti-TCRβV12 antibody molecule is a humanized antibody molecule.

In some embodiments, the anti-TCRβV antibody molecule, e.g., anti-TCRβV12 antibody molecule, is isolated or recombinant.

In some embodiments, the anti-TCRβV antibody molecule, e.g., anti-TCRβV12 antibody molecule, comprises at least one antigen-binding region, e.g., a variable region or an antigen-binding fragment thereof, from an antibody described herein, e.g., an antibody described in Table 4, or encoded by a nucleotide sequence in Table 4, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβV12 antibody molecule, comprises at least one, two, three or four variable regions from an antibody described herein, e.g., an antibody as described in Table 4, or encoded by a nucleotide sequence in Table 4, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβV12 antibody molecule, comprises at least one or two heavy chain variable regions from an antibody described herein, e.g., an antibody as described in Table 4, or encoded by a nucleotide sequence in Table 4, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβV12 antibody molecule, comprises at least one or two light chain variable regions from an antibody described herein, e.g., an antibody as described in Table 4, or encoded by a nucleotide sequence in Table 4, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβV12 antibody molecule, comprises a heavy chain constant region for an IgG4, e.g., a human IgG4. In still another embodiment, the anti-TCRβV antibody molecule, e.g., anti-TCRβV12 antibody molecule, includes a heavy chain constant region for an IgG1, e.g., a human IgG1. In one embodiment, the heavy chain constant region comprises an amino sequence set forth in Table 5, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβV12 antibody molecule, includes a kappa light chain constant region, e.g., a human kappa light chain constant region. In one embodiment, the light chain constant region comprises an amino sequence set forth in Table 5, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβV12 antibody molecule, includes at least one, two, or three complementarity determining regions (CDRs) from a heavy chain variable region of an antibody described herein, e.g., an antibody as described in Table 4, or encoded by the nucleotide sequence in Table 4, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβV12 antibody molecule, includes at least one, two, or three CDRs (or collectively all of the CDRs) from a heavy chain variable region comprising an amino acid sequence shown in Table 4, or encoded by a nucleotide sequence shown in Table 4. In one embodiment, one or more of the CDRs (or collectively all of the CDRs) have one, two, three, four, five, six or more changes, e.g., amino acid substitutions or deletions, relative to the amino acid sequence shown in Table 4, or encoded by a nucleotide sequence shown in Table 4.

In some embodiments, the anti-TCRβV antibody molecule, e.g., anti-TCRβV12 antibody molecule, includes at least one, two, or three complementarity determining regions (CDRs) from a light chain variable region of an antibody described herein, e.g., an antibody as described in Table 4, or encoded by the nucleotide sequence in Table 4, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβV12 antibody molecule, includes at least one, two, or three CDRs (or collectively all of the CDRs) from a light chain variable region comprising an amino acid sequence shown in Table 4, or encoded by a nucleotide sequence shown in Table 4. In one embodiment, one or more of the CDRs (or collectively all of the CDRs) have one, two, three, four, five, six or more changes, e.g., amino acid substitutions or deletions, relative to the amino acid sequence shown in Table 4, or encoded by a nucleotide sequence shown in Table 4.

In some embodiments, the anti-TCRβV antibody molecule, e.g., anti-TCRβV12 antibody molecule, includes at least one, two, three, four, five or six CDRs (or collectively all of the CDRs) from a heavy and light chain variable region comprising an amino acid sequence shown in Table 4, or encoded by a nucleotide sequence shown in Table 4. In one embodiment, one or more of the CDRs (or collectively all of the CDRs) have one, two, three, four, five, six or more changes, e.g., amino acid substitutions or deletions, relative to the amino acid sequence shown in Table 4, or encoded by a nucleotide sequence shown in Table 4.

In some embodiments, the anti-TCRβV antibody molecule, e.g., anti-TCRβV12 antibody molecule, molecule includes all six CDRs from an antibody described herein, e.g., an antibody as described in Table 4, or encoded by the nucleotide sequence in Table 4, or closely related CDRs, e.g., CDRs which are identical or which have at least one amino acid alteration, but not more than two, three or four alterations (e.g., substitutions, deletions, or insertions, e.g., conservative substitutions). In some embodiments, the anti-TCRβV antibody molecule, e.g., anti-TCRβV12 antibody molecule, may include any CDR described herein.

In some embodiments, the anti-TCRβV antibody molecule, e.g., anti-TCRβV12 antibody molecule includes at least one, two, or three CDRs according to Kabat et al. (e.g., at least one, two, or three CDRs according to the Kabat definition as set out in Table 4) from a heavy chain variable region of an antibody described herein, e.g., an antibody chosen as described in Table 4, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 4.

In some embodiments, the anti-TCRβV antibody molecule, e.g., anti-TCRβV12 antibody molecule includes at least one, two, or three CDRs according to Kabat et al. (e.g., at least one, two, or three CDRs according to the Kabat definition as set out in Table 4) from a light chain variable region of an antibody described herein, e.g., an antibody as described in Table 4, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 4.

In some embodiments, the anti-TCRβV antibody molecule, e.g., anti-TCRβV12 antibody molecule includes at least one, two, three, four, five, or six CDRs according to Kabat et al. (e.g., at least one, two, three, four, five, or six CDRs according to the Kabat definition as set out in Table 4) from the heavy and light chain variable regions of an antibody described herein, e.g., an antibody as described in Table 4, or encoded by the nucleotide sequence in Table 4; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Kabat et al. shown in Table 4.

In some embodiments, the anti-TCRβV antibody molecule, e.g., anti-TCRβV12 antibody molecule includes all six CDRs according to Kabat et al. (e.g., all six CDRs according to the Kabat definition as set out in Table 4) from the heavy and light chain variable regions of an antibody described herein, e.g., an antibody as described in Table 4, or encoded by the nucleotide sequence in Table 4; or encoded by the nucleotide sequence in Table 4; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Kabat et al. shown in Table 4. In some embodiments, the anti-TCRβV antibody molecule, e.g., anti-TCRβV12 antibody molecule may include any CDR described herein.

In some embodiments, the anti-TCRβV antibody molecule, e.g., anti-TCRβV12 antibody molecule includes at least one, two, or three hypervariable loops that have the same canonical structures as the corresponding hypervariable loop of an antibody described herein, e.g., an antibody described in Table 4, e.g., the same canonical structures as at least loop 1 and/or loop 2 of the heavy and/or light chain variable domains of an antibody described herein. See, e.g., Chothia et al., (1992) J. Mol. Biol. 227:799-817; Tomlinson et al., (1992) J. Mol. Biol. 227:776-798 for descriptions of hypervariable loop canonical structures. These structures can be determined by inspection of the tables described in these references.

In some embodiments, the anti-TCRβV antibody molecule, e.g., anti-TCRβV12 antibody molecule includes at least one, two, or three CDRs according to Chothia et al. (e.g., at least one, two, or three CDRs according to the Chothia definition as set out in Table 4) from a heavy chain variable region of an antibody described herein, e.g., an antibody chosen as described in Table 4, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 4.

In some embodiments, the anti-TCRβV antibody molecule, e.g., anti-TCRβV12 antibody molecule includes at least one, two, or three CDRs according to Chothia et al. (e.g., at least one, two, or three CDRs according to the Chothia definition as set out in Table 4) from a light chain variable region of an antibody described herein, e.g., an antibody as described in Table 4, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 4.

In some embodiments, the anti-TCRβV antibody molecule, e.g., anti-TCRβV12 antibody molecule includes at least one, two, three, four, five, or six CDRs according to Chothia et al. (e.g., at least one, two, three, four, five, or six CDRs according to the Chothia definition as set out in Table 4) from the heavy and light chain variable regions of an antibody described herein, e.g., an antibody as described in Table 4, or encoded by the nucleotide sequence in Table 4; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Chothia et al. shown in Table 4.

In some embodiments, the anti-TCRβV antibody molecule, e.g., anti-TCRβV12 antibody molecule includes all six CDRs according to Chothia et al. (e.g., all six CDRs according to the Chothia definition as set out in Table 4) from the heavy and light chain variable regions of an antibody described herein, e.g., an antibody as described in Table 4, or encoded by the nucleotide sequence in Table 4; or encoded by the nucleotide sequence in Table 4; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Chothia et al. shown in Table 4. In some embodiments, the anti-TCRβV antibody molecule, e.g., anti-TCRβV12 antibody molecule may include any CDR described herein.

In some embodiments, the anti-TCRβV antibody molecule, e.g., anti-TCRβV12 antibody molecule includes at least one, two, or three CDRs according to a combined CDR (e.g., at least one, two, or three CDRs according to the combined CDR definition as set out in Table 4) from a heavy chain variable region of an antibody described herein, e.g., an antibody chosen as described in Table 4, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to combined CDR shown in Table 4.

In some embodiments, the anti-TCRβV antibody molecule, e.g., anti-TCRβV12 antibody molecule includes at least one, two, or three CDRs according to a combined CDR (e.g., at least one, two, or three CDRs according to the combined CDR definition as set out in Table 4) from a light chain variable region of an antibody described herein, e.g., an antibody as described in Table 4, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to a combined CDR shown in Table 4.

In some embodiments, the anti-TCRβV antibody molecule, e.g., anti-TCRβV12 antibody molecule includes at least one, two, three, four, five, or six CDRs according to a combined CDR. (e.g., at least one, two, three, four, five, or six CDRs according to the combined CDR definition as set out in Table 4) from the heavy and light chain variable regions of an antibody described herein, e.g., an antibody as described in Table 4, or encoded by the nucleotide sequence in Table 4; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to a combined CDR shown in Table 4.

In some embodiments, the anti-TCRβV antibody molecule, e.g., anti-TCRβV12 antibody molecule includes all six CDRs according to a combined CDR (e.g., all six CDRs according to the combined CDR definition as set out in Table 4) from the heavy and light chain variable regions of an antibody described herein, e.g., an antibody as described in Table 4, or encoded by the nucleotide sequence in Table 4; or encoded by the nucleotide sequence in Table 4; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to a combined CDR shown in Table 4. In some embodiments, the anti-TCRβV antibody molecule, e.g., anti-TCRβV12 antibody molecule may include any CDR described herein.

In some embodiments, a combined CDR as set out in Table 3 is a CDR that comprises a Kabat CDR and a Chothia CDR.

In some embodiments, the anti-TCRβV antibody molecule, e e.g., anti-TCRβV12 antibody molecule, molecule includes a combination of CDRs or hypervariable loops identified as combined CDRs in Table 3. In some embodiments, the anti-TCRβV antibody molecule, e.g., anti-TCRβV12 antibody molecule, can contain any combination of CDRs or hypervariable loops according the “combined” CDRs are described in Table 3.

In some embodiments, the anti-TCRβV antibody molecule, e.g., anti-TCRβV12 antibody molecule includes a combination of CDRs or hypervariable loops defined according to the Kabat et al. and Chothia et al., or as described in Table 3

In some embodiments, the anti-TCRβV antibody molecule, e.g., anti-TCRβV12 antibody molecule can contain any combination of CDRs or hypervariable loops according to the Kabat and Chothia definitions.

In an embodiment, e.g., an embodiment comprising a variable region, a CDR (e.g., a combined CDR, Chothia CDR or Kabat CDR), or other sequence referred to herein, e.g., in Table 4, the antibody molecule is a monospecific antibody molecule, a bispecific antibody molecule, a bivalent antibody molecule, a biparatopic antibody molecule, or an antibody molecule that comprises an antigen binding fragment of an antibody, e.g., a half antibody or antigen binding fragment of a half antibody. In certain embodiments the antibody molecule comprises a multispecific molecule, e.g., a bispecific molecule, e.g., as described herein.

In some embodiments, the anti-TCRβV antibody molecule, e.g., anti-TCRβV12 antibody molecule includes:
(i) one, two or all of a light chain complementarity determining region 1 (LC CDR1), a light chain complementarity determining region 2 (LC CDR2), and a light chain complementarity determining region 3 (LC CDR3) of SEQ ID NO: 16, SEQ ID NO: 26, SEQ ID NO: 27, SEQ ID NO: 28, SEQ ID NO: 29 or SEQ ID NO: 30, and/or
(ii) one, two or all of a heavy chain complementarity determining region 1 (HC CDR1), heavy chain complementarity determining region 2 (HC CDR2), and a heavy chain complementarity determining region 3 (HC CDR3) of SEQ ID NO: 15, SEQ ID NO: 23, SEQ ID NO: 24, or SEQ ID NO: 25.

In some embodiments, the anti-TCRβV antibody molecule, e.g., anti-TCRβV12 antibody molecule comprises:
(i) a LC CDR1 amino acid sequence of SEQ ID NO: 20, a LC CDR2 amino acid sequence of SEQ ID NO: 21, or a LC CDR3 amino acid sequence of SEQ ID NO: 22; and/or
(ii) a HC CDR1 amino acid sequence of SEQ ID NO: 17, a HC CDR2 amino acid sequence of SEQ ID NO: 18, or a HC CDR3 amino acid sequence of SEQ ID NO: 19.

In some embodiments, the anti-TCRβV antibody molecule, e.g., anti-TCRβV12 antibody molecule comprises:
(i) a light chain variable region (VL) comprising a LC CDR1 amino acid sequence of SEQ ID NO: 20, a LC CDR2 amino acid sequence of SEQ ID NO: 21, and a LC CDR3 amino acid sequence of SEQ ID NO: 2; and/or
(ii) a heavy chain variable region (VH) comprising a HC CDR1 amino acid sequence of SEQ ID NO: 17, a HC CDR2 amino acid sequence of SEQ ID NO: 18, and a HC CDR3 amino acid sequence of SEQ ID NO: 19.

In some embodiments, the anti-TCRβV antibody molecule, e.g., anti-TCRβV12 antibody molecule comprises:
(i) a LC CDR1 amino acid sequence of SEQ ID NO: 63, a LC CDR2 amino acid sequence of SEQ ID NO: 64, or a LC CDR3 amino acid sequence of SEQ ID NO: 65; and/or
(ii) a HC CDR1 amino acid sequence of SEQ ID NO: 57, a HC CDR2 amino acid sequence of SEQ ID NO: 58, or a HC CDR3 amino acid sequence of SEQ ID NO: 59.

In some embodiments, the anti-TCRβV antibody molecule, e.g., anti-TCRβV12 antibody molecule comprises:
(i) a light chain variable region (VL) comprising a LC CDR1 amino acid sequence of SEQ ID NO: 63, a LC CDR2 amino acid sequence of SEQ ID NO: 64, or a LC CDR3 amino acid sequence of SEQ ID NO: 65; and/or
(ii) a heavy chain variable region (VH) comprising a HC CDR1 amino acid sequence of SEQ ID NO: 57, a HC CDR2 amino acid sequence of SEQ ID NO: 58, or a HC CDR3 amino acid sequence of SEQ ID NO: 59.

In some embodiments, the anti-TCRβV antibody molecule, e.g., anti-TCRβV12 antibody molecule comprises:
(i) a LC CDR1 amino acid sequence of SEQ ID NO: 66, a LC CDR2 amino acid sequence of SEQ ID NO: 67, or a LC CDR3 amino acid sequence of SEQ ID NO: 68; and/or
(ii) a HC CDR1 amino acid sequence of SEQ ID NO: 60, a HC CDR2 amino acid sequence of SEQ ID NO: 61, or a HC CDR3 amino acid sequence of SEQ ID NO: 62.

In some embodiments, the anti-TCRβV antibody molecule, e.g., anti-TCRβV12 antibody molecule comprises:
(i) a light chain variable region (VL) comprising a LC CDR1 amino acid sequence of SEQ ID NO: 63, a LC CDR2 amino acid sequence of SEQ ID NO: 64, or a LC CDR3 amino acid sequence of SEQ ID NO: 65; and/or
(ii) a heavy chain variable region (VH) comprising a HC CDR1 amino acid sequence of SEQ ID NO: 57, a HC CDR2 amino acid sequence of SEQ ID NO: 58, or a HC CDR3 amino acid sequence of SEQ ID NO: 59.

In one embodiment, the light or the heavy chain variable framework (e.g., the region encompassing at least FR1, FR2, FR3, and optionally FR4) of the anti-TCRβV antibody molecule, e.g., anti-TCRβV12 antibody molecule can be chosen from: (a) a light or heavy chain variable framework including at least 80%, 85%, 87% 90%, 92%, 93%, 95%, 97%, 98%, or 100% of the amino acid residues from a human light or heavy chain variable framework, e.g., a light or heavy chain variable framework residue from a human mature antibody, a human germline sequence, or a human consensus sequence; (b) a light or heavy chain variable framework including from 20% to 80%, 40% to 60%, 60% to 90%, or 70% to 95% of the amino acid residues from a human light or heavy chain variable framework, e.g., a light or heavy chain variable framework residue from a human mature antibody, a human germline sequence, or a human consensus sequence; (c) a non-human framework (e.g., a rodent framework); or (d) a non-human framework that has been modified, e.g., to remove antigenic or cytotoxic determinants, e.g., deimmunized, or partially humanized. In one embodiment, the light or heavy chain variable framework region (particularly FR1, FR2 and/or FR3) includes a light or heavy chain variable framework sequence at least 70, 75, 80, 85, 87, 88, 90, 92, 94, 95, 96, 97, 98, 99% identical or identical to the frameworks of a VL or VH segment of a human germline gene.

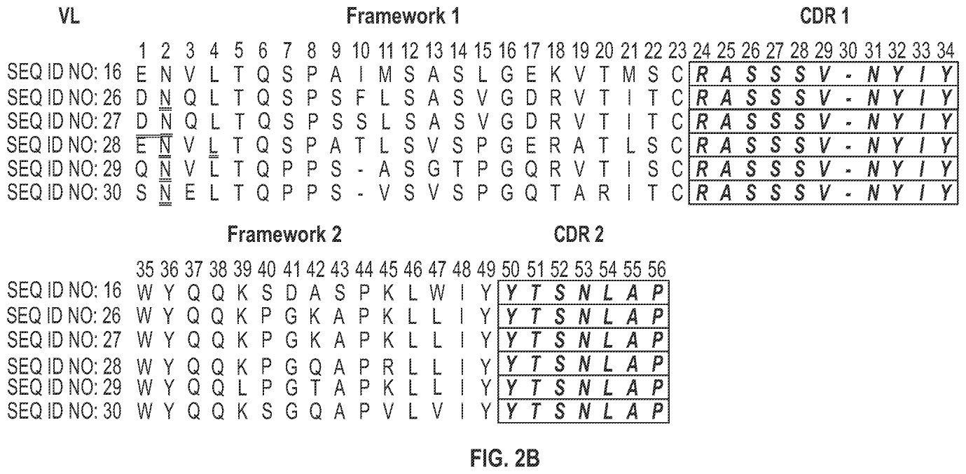

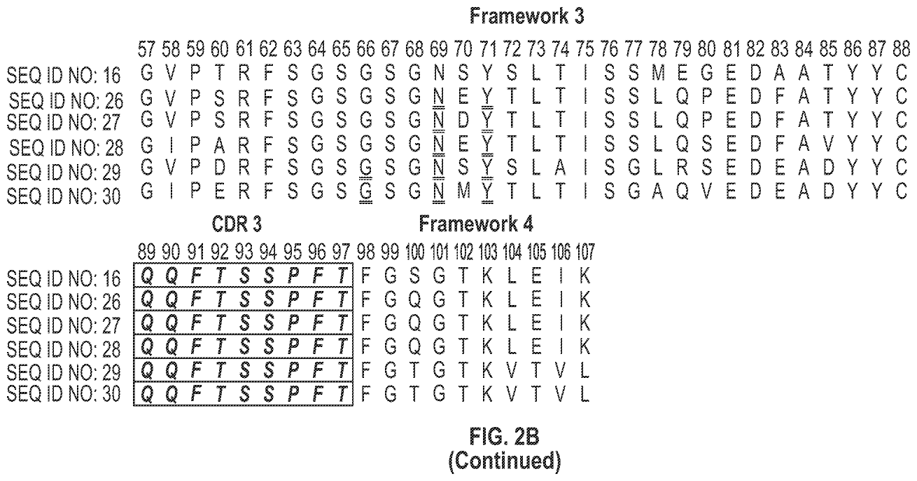

In some embodiments, the anti-TCRβV antibody molecule, e.g., anti-TCRβV12 antibody molecule, comprises a heavy chain variable domain having at least one, two, three, four, five, six, seven, ten, fifteen, twenty or more changes, e.g., amino acid substitutions or deletions, from an amino acid sequence described in Table 4 .e.g., the amino acid sequence of the FR region in the entire variable region, e.g., shown in FIGS. 2A and 2B, or in SEQ ID NOs: 23-25.

Alternatively, or in combination with the heavy chain substitutions described herein the anti-TCRβV antibody molecule, e.g., anti-TCRβV12 antibody molecule comprises a light chain variable domain having at least one, two, three, four, five, six, seven, ten, fifteen, twenty or more amino acid changes, e.g., amino acid substitutions or deletions, from an amino acid sequence of an antibody described herein .e.g., the amino acid sequence of the FR region in the entire variable region, e.g., shown in FIGS. 2A and 2B, or in SEQ ID NOs: 26-30.

In some embodiments, the anti-TCRβV antibody molecule, e.g., anti-TCRβV12 antibody molecule includes one, two, three, or four heavy chain framework regions shown in FIG. 2A, or a sequence substantially identical thereto.

In some embodiments, the anti-TCRβV antibody molecule, e.g., anti-TCRβV12 antibody molecule includes one, two, three, or four light chain framework regions shown in FIG. 2B, or a sequence substantially identical thereto.

In some embodiments, the anti-TCRβV antibody molecule, e.g., anti-TCRβV12 antibody molecule comprises the light chain framework region 1 e.g., as shown in FIG. 2B.

In some embodiments, the anti-TCRβV antibody molecule, e.g., anti-TCRβV12 antibody molecule comprises the light chain framework region 2 e.g., as shown in FIG. 2B.

In some embodiments, the anti-TCRβV antibody molecule, e.g., anti-TCRβV12 antibody molecule comprises the light chain framework region 3, e.g., as shown in FIG. 2B.

In some embodiments, the anti-TCRβV antibody molecule, e.g., anti-TCRβV12 antibody molecule comprises the light chain framework region 4, e.g., as shown in FIG. 2B.

In some embodiments, the anti-TCRβV antibody molecule, e.g., anti-TCRβV12 antibody molecule comprises a light chain comprising a framework region, e.g., framework region 1 (FR1), comprising a change, e.g., a substitution (e.g., a conservative substitution) at one or more, e.g., all, position disclosed herein according to Kabat numbering. In some embodiments, FR1 comprises an Aspartic Acid at position 1, e.g., a substitution at position 1 according to Kabat numbering, e.g., an Alanine to Aspartic Acid substitution. In some embodiments, FR1 comprises an Asparagine at position 2, e.g., a substitution at position 2 according to Kabat numbering, e.g., an Isoleucine to Asparagine substitution, Serine to Asparagine substitution or Tyrosine to Asparagine substitution. In some embodiments, FR1 comprises a Leucine at position 4, e.g., a substitution at position 4 according to Kabat numbering, e.g., a Methionine to Leucine substitution.

In some embodiments, the anti-TCRβV antibody molecule, e.g., anti-TCRβV12 antibody molecule comprises a light chain comprising a framework region, e.g., framework region 1 (FR1), comprising a substitution at position 1 according to Kabat numbering, e.g., an Alanine to Aspartic Acid substitution, a substitution at position 2 according to Kabat numbering, e.g., an Isoleucine to Asparagine substitution, Serine to Asparagine substitution or Tyrosine to Asparagine substitution, and a substitution at position 4 according to Kabat numbering, e.g., a Methionine to Leucine substitution. In some embodiments, the anti-TCRβV antibody molecule, e.g., anti-TCRβV12 antibody molecule comprises a light chain comprising a framework region, e.g., framework region 1 (FR1), comprising a substitution at position 1 according to Kabat numbering, e.g., an Alanine to Aspartic Acid substitution, and a substitution at position 2 according to Kabat numbering, e.g., an Isoleucine to Asparagine substitution, Serine to Asparagine substitution or Tyrosine to Asparagine substitution. In some embodiments, the anti-TCRβV antibody molecule, e.g., anti-TCRβV12 antibody molecule comprises a light chain comprising a framework region, e.g., framework region 1 (FR1), comprising a substitution at position 1 according to Kabat numbering, e.g., an Alanine to Aspartic Acid substitution, and a substitution at position 4 according to Kabat numbering, e.g., a Methionine to Leucine substitution. In some embodiments, the anti-TCRβV antibody molecule, e.g., anti-TCRβV12 antibody molecule comprises a light chain comprising a framework region, e.g., framework region 1 (FR1), comprising a substitution at position 2 according to Kabat numbering, e.g., an Isoleucine to Asparagine substitution, Serine to Asparagine substitution or Tyrosine to Asparagine substitution, and a substitution at position 4 according to Kabat numbering, e.g., a Methionine to Leucine substitution. In some embodiments, the substitution is relative to a human germline light chain framework region sequence.

In some embodiments, the anti-TCRβV antibody molecule, e.g., anti-TCRβV12 antibody molecule comprises a light chain comprising a framework region, e.g., framework region 3 (FR3), comprising a change, e.g., a substitution (e.g., a conservative substitution) at one or more, e.g., all, position disclosed herein according to Kabat numbering. In some embodiments, FR3 comprises a Glycine at position 66, e.g., a substitution at position 66 according to Kabat numbering, e.g., a Lysine to Glycine substitution, or a Serine to Glycine substitution. In some embodiments, FR3 comprises an Asparagine at position 69, e.g., a substitution at position 69 according to Kabat numbering, e.g., a Tyrosine to Asparagine substitution. In some embodiments, FR3 comprises a Tyrosine at position 71, e.g., a substitution at position 71 according to Kabat numbering, e.g., a Phenylalanine to Tyrosine substitution, or an Alanine to Tyrosine substitution.

In some embodiments, the anti-TCRβV antibody molecule, e.g., anti-TCRβV12 antibody molecule comprises a light chain comprising a framework region, e.g., framework region 3 (FR3), comprising a substitution at position 66 according to Kabat numbering, e.g., a Lysine to Glycine substitution, or a Serine to Glycine substitution, and a substitution at position 69 according to Kabat numbering, e.g., a Tyrosine to Asparagine substitution. . In some embodiments, the anti-TCRβV antibody molecule, e.g., anti-TCRβV12 antibody molecule comprises a light chain comprising a framework region, e.g., framework region 3 (FR3), comprising a substitution at position 66 according to Kabat numbering, e.g., Lysine to Glycine substitution, or a Serine to Glycine substitution, and a substitution at position 71 according to Kabat numbering, e.g., a Phenylalanine to Tyrosine substitution, or an Alanine to Tyrosine substitution. In some embodiments, the anti-TCRβV antibody molecule, e.g., anti-TCRβV12 antibody molecule comprises a light chain comprising a framework region, e.g., framework region 3 (FR3), comprising a substitution at position 69 according to Kabat numbering, e.g., a Tyrosine to Asparagine substitution and a substitution at position 71 according to Kabat numbering, e.g., a Phenylalanine to Tyrosine substitution, or an Alanine to Tyrosine substitution. In some embodiments, the anti-TCRβV antibody molecule, e.g., anti-TCRβV12 antibody molecule comprises a light chain comprising a framework region, e.g., framework region 3 (FR3), comprising a substitution at position 66 according to Kabat numbering, e.g., a Lysine to Glycine substitution, or a Serine to Glycine substitution, a substitution at position 69 according to Kabat numbering, e.g., a Tyrosine to Asparagine substitution and a substitution at position 71 according to Kabat numbering, e.g., a Phenylalanine to Tyrosine substitution, or an Alanine to Tyrosine substitution. In some embodiments, the substitution is relative to a human germline light chain framework region sequence.

In some embodiments, the anti-TCRβV antibody molecule, e.g., anti-TCRβV12 antibody molecule comprises a light chain comprising: a framework region 1 (FR1) comprising a substitution at position 2 according to Kabat numbering, e.g., a Isoleucine to Asparagine substitution; and a framework region 3 (FR3), comprising a substitution at position 69 according to Kabat numbering, e.g., a Threonine to Asparagine substitution and a substitution at position 71 according to Kabat numbering, e.g., a Phenylalanine to Tyrosine substitution, e.g., as shown in the amino acid sequence of SEQ ID NO: 26. In some embodiments, the substitution is relative to a human germline light chain framework region sequence.

In some embodiments, the anti-TCRβV antibody molecule, e.g., anti-TCRβV12 antibody molecule comprises a light chain comprising: (a) a framework region 1 (FR1) comprising a substitution at position 1 according to Kabat numbering, e.g., a Alanine to Aspartic Acid substitution, and a substitution at position 2 according to Kabat numbering, e.g., a Isoleucine to Asparagine substitution; and (b) a framework region 3 (FR3), comprising a substitution at position 69 according to Kabat numbering, e.g., a Threonine to Asparagine substitution and a substitution at position 71 according to Kabat numbering, e.g., a Phenylalanine to Tyrosine substitution, e.g., as shown in the amino acid sequence of SEQ ID NO: 27. In some embodiments, the substitution is relative to a human germline light chain framework region sequence.

In some embodiments, the anti-TCRβV antibody molecule, e.g., anti-TCRβV12 antibody molecule comprises a light chain comprising: (a) a framework region 1 (FR1) comprising a substitution at position 2 according to Kabat numbering, e.g., a Serine to Asparagine substitution; and a substitution at position 4 according to Kabat numbering, e.g., a Methionine to Leucine substitution; and (b) a framework region 3 (FR3), comprising a substitution at position 69 according to Kabat numbering, e.g., a Threonine to Asparagine substitution and a substitution at position 71 according to Kabat numbering, e.g., a Phenylalanine to Tyrosine substitution, e.g., as shown in the amino acid sequence of SEQ ID NO: 28. In some embodiments, the substitution is relative to a human germline light chain framework region sequence.

In some embodiments, the anti-TCRβV antibody molecule, e.g., anti-TCRβV12 antibody molecule comprises a light chain comprising: (a) a framework region 1 (FR1) comprising a substitution at position 2 according to Kabat numbering, e.g., a Serine to Asparagine substitution; and (b) a framework region 3 (FR3) comprising a substitution at position 66 according to Kabat numbering, e.g., a Lysine to Glycine substitution; a substitution at position 69 according to Kabat numbering, e.g., a Threonine to Asparagine substitution; and a substitution at position 71 according to Kabat numbering, e.g., a Alanine to Tyrosine substitution, e.g., as shown in the amino acid sequence of SEQ ID NO: 29. In some embodiments, the substitution is relative to a human germline light chain framework region sequence.

In some embodiments, the anti-TCRβV antibody molecule, e.g., anti-TCRβV12 antibody molecule comprises a light chain comprising: (a) a framework region 1 (FR1) comprising a substitution at position 2 according to Kabat numbering, e.g., a Tyrosine to Asparagine substitution; and (b) a framework region 3 (FR3) comprising a substitution at position 66 according to Kabat numbering, e.g., a Serine to Glycine substitution; a substitution at position 69 according to Kabat numbering, e.g., a Threonine to Asparagine substitution; and a substitution at position 71 according to Kabat numbering, e.g., a Alanine to Tyrosine substitution, e.g., as shown in the amino acid sequence of SEQ ID NO: 29. In some embodiments, the substitution is relative to a human germline light chain framework region sequence.

In some embodiments, the anti-TCRβV antibody molecule, e.g., anti-TCRβV12 antibody molecule comprises a light chain variable domain comprising: (a) a framework region 1 (FR1) comprising a change, e.g., a substitution (e.g., a conservative substitution) at one or more (e.g., all) positions disclosed herein according to Kabat numbering, and (b) a framework region 3 (FR3) comprising a change, e.g., a substitution (e.g., a conservative substitution) at one or more (e.g., all) position disclosed herein according to Kabat numbering. In some embodiments, the substitution is relative to a human germline light chain framework region sequence.

In some embodiments, the anti-TCRβV antibody molecule, e.g., anti-TCRβV12 antibody molecule comprises the heavy chain framework region 1, e.g., as shown in FIG. 2A.

In some embodiments, the anti-TCRβV antibody molecule, e.g., anti-TCRβV12 antibody molecule comprises the heavy chain framework region 2, e.g., as shown in FIG. 2A.

In some embodiments, the anti-TCRβV antibody molecule, e.g., anti-TCRβV12 antibody molecule comprises the heavy chain framework region 3, e.g., as shown in FIG. 2A.

In some embodiments, the anti-TCRβV antibody molecule, e.g., anti-TCRβV12 antibody molecule comprises the heavy chain framework region 4, e.g., as shown in FIG. 2A.

In some embodiments, the anti-TCRβV antibody molecule, e.g., anti-TCRβV12 antibody molecule comprises the heavy chain framework regions 1-4, e.g., SEQ ID NOS: 20-23, or as shown in FIG. 2A.

In some embodiments, the anti-TCRβV antibody molecule, e.g., anti-TCRβV12 antibody molecule comprises the light chain framework regions 1-4, e.g., SEQ ID NOs: 26-30, or as shown in FIG. 2B.

In some embodiments, the anti-TCRβV antibody molecule, e.g., anti-TCRβV12 antibody molecule comprises the heavy chain framework regions 1-4, e.g., SEQ ID NOs: 23-25; and the light chain framework regions 1-4, e.g., SEQ ID NOs: 26-30, or as shown in FIGS. 2A and 2B.

In some embodiments, the heavy or light chain variable domain, or both, of , the anti-TCRβV antibody molecule, e.g., anti-TCRβV12 antibody molecule includes an amino acid sequence, which is substantially identical to an amino acid disclosed herein, e.g., at least 80%, 85%, 90%, 92%, 95%, 97%, 98%, 99% or higher identical to a variable region of an antibody described herein, e.g., an antibody as described in Table 4, or encoded by the nucleotide sequence in Table 4; or which differs at least 1 or 5 residues, but less than 40, 30, 20, or 10 residues, from a variable region of an antibody described herein.

In some embodiments, the anti-TCRβV antibody molecule, e.g., anti-TCRβV12 antibody molecule comprises at least one, two, three, or four antigen-binding regions, e.g., variable regions, having an amino acid sequence as set forth in Table 4, or a sequence substantially identical thereto (e.g., a sequence at least about 85%, 90%, 95%, 99% or more identical thereto, or which differs by no more than 1, 2, 5, 10, or 15 amino acid residues from the sequences shown in Table 4. In another embodiment, the anti-TCRβV antibody molecule, e.g., anti-TCRβV12 antibody molecule includes a VH and/or VL domain encoded by a nucleic acid having a nucleotide sequence as set forth in Table 4, or a sequence substantially identical thereto (e.g., a sequence at least about 85%, 90%, 95%, 99% or more identical thereto, or which differs by no more than 3, 6, 15, 30, or 45 nucleotides from the sequences shown in Table 4.

In some embodiments, the anti-TCRβV antibody molecule, e.g., anti-TCRβV12 antibody molecule comprises:
a VH domain comprising an amino acid sequence chosen from the amino acid sequence of SEQ ID NO: 23, SEQ ID NO: 24 or SEQ ID NO: 25, an amino acid sequence at least about 85%, 90%, 95%, 99% or more identical to the amino acid sequence SEQ ID NO: 23, SEQ ID NO: 24 or SEQ ID NO: 25, or an amino acid sequence which differs by no more than 1, 2, 5, 10, or 15 amino acid residues from the amino acid sequence of SEQ ID NO: 23, SEQ ID NO: 24, or SEQ ID NO: 25; and/or
a VL domain comprising an amino acid sequence chosen from the amino acid sequence of SEQ ID NO: 26, SEQ ID NO: 27, SEQ ID NO: 28, SEQ ID NO: 29, or SEQ ID NO: 30, an amino acid sequence at least about 85%, 90%, 95%, 99% or more identical to the amino acid sequence of SEQ ID NO: 26, SEQ ID NO: 27, SEQ ID NO: 28, SEQ ID NO: 29, or SEQ ID NO: 30, or an amino acid sequence which differs by no more than 1, 2, 5, 10, or 15 amino acid residues from the amino acid sequence of SEQ ID NO: 26, SEQ ID NO: 27, SEQ ID NO: 28, SEQ ID NO: 29, or SEQ ID NO: 30.

In some embodiments, the anti-TCRβV antibody molecule, e.g., anti-TCRβV12 antibody molecule comprises:
a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and
a VL domain comprising the amino acid sequence of SEQ ID NO: 26, an amino acid sequence at least about 85%, 90%, 95%, 99% or more identical to the amino acid sequence SEQ ID NO: 26, or an amino acid sequence which differs by no more than 1, 2, 5, 10, or 15 amino acid residues from the amino acid sequence of SEQ ID NO: 26.

In some embodiments, the anti-TCRβV antibody molecule, e.g., anti-TCRβV12 antibody molecule comprises:
a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and
a VL domain comprising the amino acid sequence of SEQ ID NO: 27, an amino acid sequence at least about 85%, 90%, 95%, 99% or more identical to the amino acid sequence SEQ ID NO: 27, or an amino acid sequence which differs by no more than 1, 2, 5, 10, or 15 amino acid residues from the amino acid sequence of SEQ ID NO: 27.

In some embodiments, the anti-TCRβV antibody molecule, e.g., anti-TCRβV12 antibody molecule comprises:
a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and
a VL domain comprising the amino acid sequence of SEQ ID NO: 28, an amino acid sequence at least about 85%, 90%, 95%, 99% or more identical to the amino acid sequence SEQ ID NO: 28, or an amino acid sequence which differs by no more than 1, 2, 5, 10, or 15 amino acid residues from the amino acid sequence of SEQ ID NO: 28.

In some embodiments, the anti-TCRβV antibody molecule, e.g., anti-TCRβV12 antibody molecule comprises:
a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and a VL domain comprising the amino acid sequence of SEQ ID NO: 29, an amino acid sequence at least about 85%, 90%, 95%, 99% or more identical to the amino acid sequence SEQ ID NO: 29, or an amino acid sequence which differs by no more than 1, 2, 5, 10, or 15 amino acid residues from the amino acid sequence of SEQ ID NO: 29.

In some embodiments, the anti-TCRβV antibody molecule, e.g., anti-TCRβV12 antibody molecule comprises: a VH domain comprising the amino acid sequence of SEQ ID NO: 23, an amino acid sequence at least about 85%, 90%, 95%, 99% or more identical to the amino acid sequence SEQ ID NO: 23, or an amino acid sequence which differs by no more than 1, 2, 5, 10, or 15 amino acid residues from the amino acid sequence of SEQ ID NO: 23; and a VL domain comprising the amino acid sequence of SEQ ID NO: 30, an amino acid sequence at least about 85%, 90%, 95%, 99% or more identical to the amino acid sequence SEQ ID NO: 30, or an amino acid sequence which differs by no more than 1, 2, 5, 10, or 15 amino acid residues from the amino acid sequence of SEQ ID NO: 30.

In some embodiments, the anti-TCRβV antibody molecule, e.g., anti-TCRβV12 antibody molecule comprises:
a VH domain comprising the amino acid sequence of SEQ ID NO: 24, an amino acid sequence at least about 85%, 90%, 95%, 99% or more identical to the amino acid sequence SEQ ID NO: 24, or an amino acid sequence which differs by no more than 1, 2, 5, 10, or 15 amino acid residues from the amino acid sequence of SEQ ID NO: 24; and
a VL domain comprising the amino acid sequence of SEQ ID NO: 26, an amino acid sequence at least about 85%, 90%, 95%, 99% or more identical to the amino acid sequence SEQ ID NO: 26, or an amino acid sequence which differs by no more than 1, 2, 5, 10, or 15 amino acid residues from the amino acid sequence of SEQ ID NO: 26.

In some embodiments, the anti-TCRβV antibody molecule, e.g., anti-TCRβV12 antibody molecule comprises:
a VH domain comprising the amino acid sequence of SEQ ID NO: 24, an amino acid sequence at least about 85%, 90%, 95%, 99% or more identical to the amino acid sequence SEQ ID NO: 24, or an amino acid sequence which differs by no more than 1, 2, 5, 10, or 15 amino acid residues from the amino acid sequence of SEQ ID NO: 24; and
a VL domain comprising the amino acid sequence of SEQ ID NO: 27, an amino acid sequence at least about 85%, 90%, 95%, 99% or more identical to the amino acid sequence SEQ ID NO: 27, or an amino acid sequence which differs by no more than 1, 2, 5, 10, or 15 amino acid residues from the amino acid sequence of SEQ ID NO: 27.

In some embodiments, the anti-TCRβV antibody molecule, e.g., anti-TCRβV12 antibody molecule comprises:
a VH domain comprising the amino acid sequence of SEQ ID NO: 24, an amino acid sequence at least about 85%, 90%, 95%, 99% or more identical to the amino acid sequence SEQ ID NO: 24, or an amino acid sequence which differs by no more than 1, 2, 5, 10, or 15 amino acid residues from the amino acid sequence of SEQ ID NO: 24; and
a VL domain comprising the amino acid sequence of SEQ ID NO: 28, an amino acid sequence at least about 85%, 90%, 95%, 99% or more identical to the amino acid sequence SEQ ID NO: 28, or an amino acid sequence which differs by no more than 1, 2, 5, 10, or 15 amino acid residues from the amino acid sequence of SEQ ID NO: 28.

In some embodiments, the anti-TCRβV antibody molecule, e.g., anti-TCRβV12 antibody molecule comprises:
a VH domain comprising the amino acid sequence of SEQ ID NO: 24, an amino acid sequence at least about 85%, 90%, 95%, 99% or more identical to the amino acid sequence SEQ ID NO: 24, or an amino acid sequence which differs by no more than 1, 2, 5, 10, or 15 amino acid residues from the amino acid sequence of SEQ ID NO: 24; and
a VL domain comprising the amino acid sequence of SEQ ID NO: 29, an amino acid sequence at least about 85%, 90%, 95%, 99% or more identical to the amino acid sequence SEQ ID NO: 29, or an amino acid sequence which differs by no more than 1, 2, 5, 10, or 15 amino acid residues from the amino acid sequence of SEQ ID NO: 29.

In some embodiments, the anti-TCRβV antibody molecule, e.g., anti-TCRβV12 antibody molecule comprises:
a VH domain comprising the amino acid sequence of SEQ ID NO: 24, an amino acid sequence at least about 85%, 90%, 95%, 99% or more identical to the amino acid sequence SEQ ID NO: 24, or an amino acid sequence which differs by no more than 1, 2, 5, 10, or 15 amino acid residues from the amino acid sequence of SEQ ID NO: 24; and
a VL domain comprising the amino acid sequence of SEQ ID NO: 30, an amino acid sequence at least about 85%, 90%, 95%, 99% or more identical to the amino acid sequence SEQ ID NO: 30, or an amino acid sequence which differs by no more than 1, 2, 5, 10, or 15 amino acid residues from the amino acid sequence of SEQ ID NO: 30.

In some embodiments, the anti-TCRβV antibody molecule, e.g., anti-TCRβV12 antibody molecule comprises:
a VH domain comprising the amino acid sequence of SEQ ID NO: 25, an amino acid sequence at least about 85%, 90%, 95%, 99% or more identical to the amino acid sequence SEQ ID NO: 25, or an amino acid sequence which differs by no more than 1, 2, 5, 10, or 15 amino acid residues from the amino acid sequence of SEQ ID NO: 25; and
a VL domain comprising the amino acid sequence of SEQ ID NO: 26, an amino acid sequence at least about 85%, 90%, 95%, 99% or more identical to the amino acid sequence SEQ ID NO: 26, or an amino acid sequence which differs by no more than 1, 2, 5, 10, or 15 amino acid residues from the amino acid sequence of SEQ ID NO: 26.

In some embodiments, the anti-TCRβV antibody molecule, e.g., anti-TCRβV12 antibody molecule comprises:
a VH domain comprising the amino acid sequence of SEQ ID NO: 25, an amino acid sequence at least about 85%, 90%, 95%, 99% or more identical to the amino acid sequence SEQ ID NO: 25, or an amino acid sequence which differs by no more than 1, 2, 5, 10, or 15 amino acid residues from the amino acid sequence of SEQ ID NO: 25; and
a VL domain comprising the amino acid sequence of SEQ ID NO: 27, an amino acid sequence at least about 85%, 90%, 95%, 99% or more identical to the amino acid sequence SEQ ID NO: 27, or an amino acid sequence which differs by no more than 1, 2, 5, 10, or 15 amino acid residues from the amino acid sequence of SEQ ID NO: 27.

In some embodiments, the anti-TCRβV antibody molecule, e.g., anti-TCRβV12 antibody molecule comprises:
a VH domain comprising the amino acid sequence of SEQ ID NO: 25, an amino acid sequence at least about 85%, 90%, 95%, 99% or more identical to the amino acid sequence SEQ ID NO: 25, or an amino acid sequence which differs by no more than 1, 2, 5, 10, or 15 amino acid residues from the amino acid sequence of SEQ ID NO: 25; and
a VL domain comprising the amino acid sequence of SEQ ID NO: 28, an amino acid sequence at least about 85%, 90%, 95%, 99% or more identical to the amino acid sequence SEQ ID NO: 28, or an amino acid sequence which differs by no more than 1, 2, 5, 10, or 15 amino acid residues from the amino acid sequence of SEQ ID NO: 28.

In some embodiments, the anti-TCRβV antibody molecule, e.g., anti-TCRβV12 antibody molecule comprises:
a VH domain comprising the amino acid sequence of SEQ ID NO: 25, an amino acid sequence at least about 85%, 90%, 95%, 99% or more identical to the amino acid sequence SEQ ID NO: 25, or an amino acid sequence which differs by no more than 1, 2, 5, 10, or 15 amino acid residues from the amino acid sequence of SEQ ID NO: 25; and
a VL domain comprising the amino acid sequence of SEQ ID NO: 29, an amino acid sequence at least about 85%, 90%, 95%, 99% or more identical to the amino acid sequence SEQ ID NO: 29, or an amino acid sequence which differs by no more than 1, 2, 5, 10, or 15 amino acid residues from the amino acid sequence of SEQ ID NO: 29.

In some embodiments, the anti-TCRβV antibody molecule, e.g., anti-TCRβV12 antibody molecule comprises:
a VH domain comprising the amino acid sequence of SEQ ID NO: 25, an amino acid sequence at least about 85%, 90%, 95%, 99% or more identical to the amino acid sequence SEQ ID NO: 25, or an amino acid sequence which differs by no more than 1, 2, 5, 10, or 15 amino acid residues from the amino acid sequence of SEQ ID NO: 25; and
a VL domain comprising the amino acid sequence of SEQ ID NO: 30, an amino acid sequence at least about 85%, 90%, 95%, 99% or more identical to the amino acid sequence SEQ ID NO: 30, or an amino acid sequence which differs by no more than 1, 2, 5, 10, or 15 amino acid residues from the amino acid sequence of SEQ ID NO: 30.

In some embodiments, the anti-TCRβV antibody molecule, e.g., anti-TCRβV12 antibody molecule is a full antibody or fragment thereof (e.g., a Fab, F(ab′)₂, Fv, or a single chain Fv fragment (scFv)). In embodiments, the anti-TCRβV antibody molecule, e.g., anti-TCRβV6 (e.g., anti-TCRβV6-5*01) antibody molecule is a monoclonal antibody or an antibody with single specificity. In some embodiments, the anti-TCRβV antibody molecule, e.g., anti-TCRβV12 antibody molecule, can also be a humanized, chimeric, camelid, shark, or an in vitro-generated antibody molecule. In some embodiments, the anti-TCRβV antibody molecule, e.g., anti-TCRβV12 antibody molecule is a humanized antibody molecule. The heavy and light chains of the anti-TCRβV antibody molecule, e.g., anti-TCRβV12 antibody molecule can be full-length (e.g., an antibody can include at least one, and preferably two, complete heavy chains, and at least one, and preferably two, complete light chains) or can include an antigen-binding fragment (e.g., a Fab, F(ab′)₂, Fv, a single chain Fv fragment, a single domain antibody, a diabody (dAb), a bivalent antibody, or bispecific antibody or fragment thereof, a single domain variant thereof, or a camelid antibody).

In some embodiments, the anti-TCRβV antibody molecule, e.g., anti-TCRβV12 antibody molecule is in the form of a multispecific molecule, e.g., a bispecific molecule, e.g., as described herein.

In some embodiments, the anti-TCRβV antibody molecule, e.g., anti-TCRβV12 antibody molecule has a heavy chain constant region (Fc) chosen from, e.g., the heavy chain constant regions of IgG1, IgG2, IgG3, IgG4, IgM, IgA1, IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgG1 or IgG2 (e.g., human IgG1, or IgG2). In some embodiments, the heavy chain constant region is human IgG1.

In some embodiments, the anti-TCRβV antibody molecule, e.g., anti-TCRβV12 antibody molecule has a light chain constant region chosen from, e.g., the light chain constant regions of kappa or lambda, preferably kappa (e.g., human kappa). In one embodiment, the constant region is altered, e.g., mutated, to modify the properties of the anti-TCRβV antibody molecule, e.g., anti-TCRβV12 antibody molecule (e.g., to increase or decrease one or more of: Fc receptor binding, antibody glycosylation, the number of cysteine residues, effector cell function, or complement function). For example, the constant region is mutated at positions 296 (M to Y), 298 (S to T), 300 (T to E), 477 (H to K) and 478 (N to F) to alter Fc receptor binding (e.g., the mutated positions correspond to positions 132 (M to Y), 134 (S to T), 136 (T to E), 313 (H to K) and 314 (N to F) of SEQ ID NOs: 212 or 214; or positions 135 (M to Y), 137 (S to T), 139 (T to E), 316 (H to K) and 317 (N to F) of SEQ ID NOs: 215, 216, 217, or 218).

Antibody B-H.1 comprises a first chain comprising the amino acid sequence of SEQ ID NO: 3280 and a second chain comprising the amino acid sequence of SEQ ID NO: 3281.

Additional exemplary anti-TCRβV12 antibodies of the disclosure are provided in Table 4. In some embodiments, the anti-TCRβV12 is antibody B, e.g., humanized antibody B (antibody B-H), as provided in Table 4. In some embodiments, the anti-TCRβV antibody comprises one or more (e.g., all three) of a LC CDR1, LC CDR2, and LC CDR3 provided in Table 4; and/or one or more (e.g., all three) of a HC CDR1, HC CDR2, and HC CDR3 provided in Table 4, or a sequence with at least 95% identity thereto. In some embodiments, antibody B comprises a variable heavy chain (VH) and/or a variable light chain (VL) provided in Table 4, or a sequence with at least 95% identity thereto.

TABLE 4
Amino acid and nucleotide sequences for murine and humanized antibody
molecules which bind to TCRVB 12, e.g., TCRVB 12-3 or TCRVB 12-4. The
antibody molecules include murine mAb Antibody B and humanized mAb
Antibody B-H.1 to B-H.6. The amino acid the heavy and light chain CDRs,
and the amino acid and nucleotide sequences of the heavy and light
chain variable regions, and the heavy and light chains are shown.
Antibody B (murine)

| | | |
|---|---|---|
| SEQ ID NO: 17 | HC CDR1 (Combined) | GFTFSNFGMH |
| SEQ ID NO: 18 | HC CDR2 (Combined) | YISSGSSTIYYADTLKG |
| SEQ ID NO: 19 | HC CDR3 (Combined) | RGEGAMDY |
| SEQ ID NO: 57 | HC CDR1 (Kabat) | NFGMH |
| SEQ ID NO: 58 | HC CDR2 (Kabat) | YISSGSSTIYYADTLKG |
| SEQ ID NO: 59 | HC CDR3 (Kabat) | RGEGAMDY |
| SEQ ID NO: 60 | HC CDR1 (Chothia) | GFTFSNF |
| SEQ ID NO: 61 | HC CDR2 (Chothia) | SSGSST |
| SEQ ID NO: 62 | HC CDR3 (Chothia) | RGEGAMDY |
| SEQ ID NO: 15 | VH | DVQLVESGGGLVQPGGSRKLSCAASGFTFSNFGMHWVRQAPD |
| | | KGLEWVAYISSGSSTIYYADTLKGRFTISRDNPKNTLFLQMT |
| | | SLRSEDTAMYYCARRGEGAMDYWGQGTSVTVSS |
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3(Combined) | QQFTSSPFT |
| SEQ ID NO: 63 | LC CDR1 (Kabat) | RASSSVNYIY |
| SEQ ID NO: 64 | LC CDR2 (Kabat) | YTSNLAP |
| SEQ ID NO: 65 | LC CDR3 (Kabat) | QQFTSSPFT |
| SEQ ID NO: 66 | LC CDR1 (Chothia) | RASSSVNYIY |
| SEQ ID NO: 67 | LC CDR2 (Chothia) | YTSNLAP |
| SEQ ID NO: 68 | LC CDR3 (Chothia) | QQFTSSPFT |
| SEQ ID NO: 16 | VL | ENVLTQSPAIMSASLGEKVTMSCRASSSVNYIYWYQQKSDAS |
| | | PKLWIYYTSNLAPGVPTRFSGSGSGNSYSLTISSMEGEDAAT |
| | | YYCQQFTSSPFTFGSGTKLEIK |

Antibody B humanized (B-H)
Antibody B-H.1A HC-1

| | | |
|---|---|---|
| SEQ ID NO: 17 | HC CDR1 (Combined) | GFTFSNFGMH |
| SEQ ID NO: 18 | HC CDR2 (Combined) | YISSGSSTIYYADTLKG |
| SEQ ID NO: 19 | HC CDR3 (Combined) | RGEGAMDY |
| SEQ ID NO: 23 | VH | EVQLVESGGGLVQPGGSLRLSCAASGFTFSNFGMHWVRQAPG |
| | | KGLEWVSYISSGSSTIYYADTLKGRFTISRDNAKNSLYLQMN |
| | | SLRAEDTAVYYCARRGEGAMDYWGQGTTVTVSS |
| SEQ ID NO: 31 | DNA VH | GAGGTGCAGCTGGTTGAATCTGGCGGAGGATTGGTTCAGCCT |
| | | GGCGGCTCTCTGAGACTGTCTTGTGCCGCTTCTGGCTTCACC |
| | | TTCTCCAACTTCGGCATGCACTGGGTCCGACAGGCCCCTGGA |
| | | AAAGGACTGGAATGGGTGTCCTACATCTCCTCCGGCTCCTCC |
| | | ACCATCTACTACGCTGACACCCTGAAGGGCAGATTCACCATC |
| | | TCTCGGGACAACGCCAAGAACTCCCTGTACCTGCAGATGAAC |
| | | AGCCTGAGAGCCGAGGACACCGCCGTGTACTACTGTGCTAGA |
| | | AGAGGCGAGGGCGCCATGGATTATTGGGGCCAGGGAACCACA |
| | | GTGACCGTGTCTAGC |

Antibody B-H.1B HC-2

| | | |
|---|---|---|
| SEQ ID NO: 17 | HC CDR1 (Combined) | GFTFSNFGMH |
| SEQ ID NO: 18 | HC CDR2 (Combined) | YISSGSSTIYYADTLKG |
| SEQ ID NO: 19 | HC CDR3 (Combined) | RGEGAMDY |
| SEQ ID NO: 24 | VH | EVQLVESGGGLVQPGGSLRLSCAASGFTFSNFGMHWVRQAPG |
| | | KGLEWVSYISSGSSTIYYADTLKGRFTISRDNSKNTLYLQMN |
| | | SLRAEDTAVYYCARRGEGAMDYWGQGTTVTVSS |
| SEQ ID NO: 32 | DNA VH | GAGGTGCAGCTGGTTGAATCTGGCGGAGGATTGGTTCAGCCT |
| | | GGCGGCTCTCTGAGACTGTCTTGTGCCGCTTCTGGCTTCACC |
| | | TTCTCCAACTTCGGCATGCACTGGGTCCGACAGGCCCCTGGA |
| | | AAAGGACTGGAATGGGTGTCCTACATCTCCTCCGGCTCCTCC |
| | | ACCATCTACTACGCTGACACCCTGAAGGGCAGATTCACCATC |
| | | AGCCGGGACAACTCCAAGAACACCCTGTACCTGCAGATGAAC |
| | | TCCCTGAGAGCCGAGGACACCGCCGTGTACTACTGTGCTAGA |
| | | AGAGGCGAGGGCGCCATGGATTATTGGGGCCAGGGAACCACA |
| | | GTGACCGTGTCTAGC |

Antibody B-H.1C HC-3

| | | |
|---|---|---|
| SEQ ID NO: 17 | HC CDR1 (Combined) | GFTFSNFGMH |
| SEQ ID NO: 18 | HC CDR2 (Combined) | YISSGSSTIYYADTLKG |
| SEQ ID NO: 19 | HC CDR3 (Combined) | RGEGAMDY |
| SEQ ID NO: 25 | VH | QVQLVESGGGVVQPGRSLRLSCAASGFTFSNFGMHWVRQAPG |
| | | KGLEWVAYISSGSSTIYYADTLKGRFTISRDNSKNTLYLQMN |
| | | SLRAEDTAVYYCARRGEGAMDYWGQGTTVTVSS |
| SEQ ID NO: 33 | DNA VH | CAGGTGCAGCTGGTGGAATCTGGTGGCGGAGTTGTGCAGCCT |
| | | GGCAGATCCCTGAGACTGTCTTGTGCCGCCTCTGGCTTCACC |
| | | TTCTCCAACTTCGGCATGCACTGGGTCCGACAGGCCCCTGGA |
| | | AAAGGATTGGAGTGGGTCGCCTACATCTCCTCCGGCTCCTCC |
| | | ACCATCTACTACGCTGACACCCTGAAGGGCAGATTCACCATC |
| | | AGCCGGGACAACTCCAAGAACACCCTGTACCTGCAGATGAAC |
| | | TCCCTGAGAGCCGAGGACACCGCCGTGTACTACTGTGCTAGA |
| | | AGAGGCGAGGGCGCCATGGATTATTGGGGCCAGGGAACCACA |
| | | GTGACCGTGTCTAGC |

Antibody B-H.1D LC-1

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3(Combined) | QQFTSSPFT |
| SEQ ID NO: 26 | VL | DNQLTQSPSFLSASVGDRVTITCRASSSVNYIYWYQQKPGKA |
| | | PKLLIYYTSNLAPGVPSRFSGSGSGNEYTLTISSLQPEDFAT |
| | | YYCQQFTSSPFTFGQGTKLEIK |
| SEQ ID NO: 34 | DNA VL | GATAACCAGCTGACCCAGTCTCCTAGCTTCCTGTCTGCCTCT |
| | | GTGGGCGACAGAGTGACAATTACCTGCCGGGCCTCCTCCTCC |
| | | GTGAACTACATCTACTGGTATCAGCAGAAGCCCGGCAAGGCC |
| | | CCTAAGCTGCTGATCTACTACACCTCCAATCTGGCCCCTGGC |
| | | GTGCCCTCTAGATTTTCCGGATCTGGCTCCGGCAACGAGTAT |
| | | ACCCTGACAATCTCCAGCCTGCAGCCTGAGGACTTCGCCACC |
| | | TACTACTGCCAGCAGTTCACCTCCTCTCCATTCACCTTTGGC |
| | | CAGGGCACCAAGCTGGAAATCAAA |

Antibody B-H.1E LC-2

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3(Combined) | QQFTSSPFT |
| SEQ ID NO: 27 | VL | DNQLTQSPSSLSASVGDRVTITCRASSSVNYIYWYQQKPGKA |
| | | PKLLIYYTSNLAPGVPSRFSGSGSGNDYTLTISSLQPEDFAT |
| | | YYCQQFTSSPFTFGQGTKLEIK |
| SEQ ID NO: 35 | DNA VL | ATAACCAGCTGACCCAGTCTCCTTCCAGCCTGTCTGCTTCTG |
| | | TGGGCGACAGAGTGACAATTACCTGCCGGGCCTCCTCCTCCG |
| | | TGAACTACATCTACTGGTATCAGCAGAAGCCCGGCAAGGCCC |
| | | CTAAGCTGCTGATCTACTACACCTCCAATCTGGCCCCTGGCG |
| | | TGCCCTCTAGATTTTCCGGATCTGGCTCCGGCAACGACTATA |
| | | CCCTGACAATCTCCAGCCTGCAGCCTGAGGACTTCGCCACCT |
| | | ACTACTGCCAGCAGTTCACCTCCTCTCCATTCACCTTTGGCC |
| | | AGGGCACCAAGCTGGAAATCAAA |

Antibody B-H.1F LC-3

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3(Combined) | QQFTSSPFT |
| SEQ ID NO: 28 | VL | ENVLTQSPATLSVSPGERATLSCRASSSVNYIYWYQQKPGQA |
| | | PRLLIYYTSNLAPGIPARFSGSGSGNEYTLTISSLQSEDFAV |
| | | YYCQQFTSSPFTFGQGTKLEIK |
| SEQ ID NO: 36 | DNA VL | GAGAATGTGCTGACCCAGTCTCCTGCCACACTGTCTGTTAGC |
| | | CCTGGCGAGAGAGCTACCCTGAGCTGCAGAGCCTCTTCCTCC |
| | | GTGAACTACATCTACTGGTATCAGCAGAAGCCCGGCCAGGCT |
| | | CCTAGACTGCTGATCTACTACACCTCCAATCTGGCCCCTGGC |
| | | ATCCCTGCCAGATTTTCCGGATCTGGCTCCGGCAACGAGTAT |
| | | ACCCTGACCATCTCCAGCCTGCAGTCCGAGGACTTTGCTGTG |
| | | TACTATTGCCAGCAGTTCACAAGCAGCCCTTTCACCTTTGGC |
| | | CAGGGCACCAAGCTGGAAATCAAA |

Antibody B-H.1G LC-4

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3(Combined) | QQFTSSPFT |
| SEQ ID NO: 29 | VL | QNVLTQPPSASGTPGQRVTISCRASSSVNYIYWYQQLPGTAP |
| | | KLLIYYTSNLAPGVPDRFSGSGSGNSYSLAISGLRSEDEADY |
| | | YCQQFTSSPFTFGTGTKVTVL |
| SEQ ID NO: 37 | DNA VL | CAGAATGTGCTGACCCAACCTCCTTCCGCCTCTGGCACACCT |
| | | GGACAGAGAGTGACAATCTCCTGCCGGGCCTCCTCCTCCGTG |
| | | AACTACATCTACTGGTATCAGCAGCTGCCCGGCACCGCTCCT |
| | | AAACTGCTGATCTACTACACCTCCAATCTGGCCCCTGGCGTG |
| | | CCCGATAGATTTTCCGGATCTGGCTCCGGCAACTCCTACAGC |
| | | CTGGCTATCTCTGGCCTGAGATCTGAGGACGAGGCCGACTAC |
| | | TACTGCCAGCAGTTCACCTCCTCTCCATTCACCTTTGGCACC |
| | | GGCACCAAAGTGACAGTTCTT |

Antibody B-H.1H LC-5

| | | |
|---|---|---|
| SEQ ID NO: 20 | LC CDR1 (Combined) | RASSSVNYIY |
| SEQ ID NO: 21 | LC CDR2 (Combined) | YTSNLAP |
| SEQ ID NO: 22 | LC CDR3(Combined) | QQFTSSPFT |
| SEQ ID NO: 30 | VL | SNELTQPPSVSVSPGQTARITCRASSSVNYIYWYQQKSGQAP |
| | | VLVIYYTSNLAPGIPERFSGSGSGNMYTLTISGAQVEDEADY |
| | | YCQQFTSSPFTFGTGTKVTVL |
| SEQ ID NO: 38 | DNA VL | TCTAATGAGCTGACCCAGCCTCCTTCCGTGTCCGTGTCTCCT |
| | | GGACAGACCGCCAGAATTACCTGCCGGGCCTCCTCCTCCGTG |
| | | AACTACATCTACTGGTATCAGCAGAAGTCCGGCCAGGCTCCT |
| | | GTGCTCGTGATCTACTACACCTCCAATCTGGCCCCTGGCATC |
| | | CCTGAGAGATTCTCCGGATCTGGCTCCGGCAACATGTACACC |
| | | CTGACCATCTCTGGCGCCCAGGTGGAAGATGAGGCCGACTAC |
| | | TACTGCCAGCAGTTCACCTCCTCTCCATTCACCTTTGGCACC |
| | | GGCACCAAAGTGACAGTTCTT |

Antibody B-H.1

| | | |
|---|---|---|
| SEQ ID NO: 3280 | Chain1: Fc only | METDTLLLWVLLLWVPGSTGDKTHTCPPCPAPELLGGPSVFL |
| | | FPPKPKDTLMISRTPEVTCVVVDVSHEDPEVKFNWYVDGVEV |
| | | HNAKTKPREEQYNSTYRVVSVLTVLHQDWLNGKEYKCKVSNK |
| | | ALPAPIEKTISKAKGQPREPQVYTLPPCREEMTKNQVSLWCL |
| | | VKGFYPSDIAVEWESNGQPENNYKTTPPVLDSDGSFFLYSKL |
| | | TVDKSRWQQGNVFSCSVMHEALHNRFTQKSLSLSPGK |
| SEQ ID NO: 3281 | Chain2. humanized | METDTLLLWVLLLWVPGSTGEVQLVESGGGLVQPGGSLRLSC |
| | B-H scFv | AASGFTFSNFGMHWVRQAPGKGLEWVSYISSGSSTIYYADTL |
| | | KGRFTISRDNSKNTLYLQMNSLRAEDTAVYYCARRGEGAMDY |
| | | WGQGTTVTVSSGGGGSGGGGSGGGGSGGGGSDNQLTQSPSFL |
| | | SASVGDRVTITCRASSSVNYIYWYQQKPGKAPKLLIYYTSNL |
| | | APGVPSRFSGSGSGNEYTLTISSLQPEDFATYYCQQFTSSPF |
| | | TFGQGTKLEIKGGGGSDKTHTCPPCPAPELLGGPSVFLFPPK |
| | | PKDTLMISRTPEVTCVVVDVSHEDPEVKFNWYVDGVEVHNAK |
| | | TKPREEQYNSTYRVVSVLTVLHQDWLNGKEYKCKVSNKALPA |
| | | PIEKTISKAKGQPREPQVCTLPPSREEMTKNQVSLSCAVKGF |
| | | YPSDIAVEWESNGQPENNYKTTPPVLDSDGSFFLVSKLTVDK |
| | | SRWQQGNVFSCSVMHEALHNHYTQKSLSLSPGKGGGGSGGGG |
| | | SGLNDIFEAQKIEWHE |
| SEQ ID NO: 1343 | scFv | EVQLVESGGGLVQPGGSLRLSCAASGFTFSNFGMHWVRQAPG |
| | | KGLEWVSYISSGSSTIYYADTLKGRFTISRDNSKNTLYLQMN |
| | | SLRAEDTAVYYCARRGEGAMDYWGQGTTVTVSSGGGGSGGGG |
| | | SGGGGSGGGGSDNQLTQSPSFLSASVGDRVTITCRASSSVNY |
| | | IYWYQQKPGKAPKLLIYYTSNLAPGVPSRFSGSGSGNEYTLT |
| | | ISSLQPEDFATYYCQQFTSSPFTFGQGTKLEIK |

Antibody B-H.2

| | | |
|---|---|---|
| SEQ ID NO: 1338 | scFv | EVQLVESGGGLVQPGGSLRLSCAASGFTFSNFGMHWVRQAPG |
| | | KGLEWVSYISSGSSTIYYADTLKGRFTISRDNSKNTLYLQMN |
| | | SLRAEDTAVYYCARRGEGAMDYWGQGTTVTVSSGGGGSGGGG |
| | | SGGGGSGGGGSDNQLTQSPSSLSASVGDRVTITCRASSSVNY |
| | | IYWYQQKPGKAPKLLIYYTSNLAPGVPSRFSGSGSGNDYTLT |
| | | ISSLQPEDFATYYCQQFTSSPFTFGQGTKLEIK |

Antibody B-H.3

| | | |
|---|---|---|
| SEQ ID NO: 1339 | scFv | EVQLVESGGGLVQPGGSLRLSCAASGFTFSNFGMHWVRQAPG |
| | | KGLEWVSYISSGSSTIYYADTLKGRFTISRDNSKNTLYLQMN |
| | | SLRAEDTAVYYCARRGEGAMDYWGQGTTVTVSSGGGGSGGGG |
| | | SGGGGSGGGGSSNELTQPPSVSVSPGQTARITCRASSSVNYI |
| | | YWYQQKSGQAPVLVIYYTSNLAPGIPERFSGSGSGNMYTLTI |
| | | SGAQVEDEADYYCQQFTSSPFTFGTGTKVTVL |

Antibody B-H.4

| | | |
|---|---|---|
| SEQ ID NO: 1340 | scFv | QVQLVESGGGVVQPGRSLRLSCAASGFTFSNFGMHWVRQAPG |
| | | KGLEWVAYISSGSSTIYYADTLKGRFTISRDNSKNTLYLQMN |
| | | SLRAEDTAVYYCARRGEGAMDYWGQGTTVTVSSGGGGSGGGG |
| | | SGGGGSGGGGSDNQLTQSPSFLSASVGDRVTITCRASSSVNY |
| | | IYWYQQKPGKAPKLLIYYTSNLAPGVPSRFSGSGSGNEYTLT |
| | | ISSLQPEDFATYYCQQFTSSPFTFGQGTKLEIK |

Antibody B-H.5

| | | |
|---|---|---|
| SEQ ID NO: 1341 | scFv | QVQLVESGGGVVQPGRSLRLSCAASGFTFSNFGMHWVRQAPG |
| | | KGLEWVAYISSGSSTIYYADTLKGRFTISRDNSKNTLYLQMN |
| | | SLRAEDTAVYYCARRGEGAMDYWGQGTTVTVSSGGGGSGGGG |
| | | SGGGGSGGGGSDNQLTQSPSSLSASVGDRVTITCRASSSVNY |
| | | IYWYQQKPGKAPKLLIYYTSNLAPGVPSRFSGSGSGNDYTLT |
| | | ISSLQPEDFATYYCQQFTSSPFTFGQGTKLEIK |

Antibody B-H.6

| | | |
|---|---|---|
| SEQ ID NO: 1342 | scFv | QVQLVESGGGVVQPGRSLRLSCAASGFTFSNFGMHWVRQAPG |
| | | KGLEWVAYISSGSSTIYYADTLKGRFTISRDNSKNTLYLQMN |
| | | SLRAEDTAVYYCARRGEGAMDYWGQGTTVTVSSGGGGSGGGG |
| | | SGGGGSGGGGSSNELTQPPSVSVSPGQTARITCRASSSVNYI |
| | | YWYQQKSGQAPVLVIYYTSNLAPGIPERFSGSGSGNMYTLTI |
| | | SGAQVEDEADYYCQQFTSSPFTFGTGTKVTVL |

TABLE 5
Constant region amino acid sequences of
human IgG heavy chains and human kappa
light chain

| | | |
|---|---|---|
| Human | LC | RTVAAPSVFI FPPSDEQLKS |
| kappa | | GTASVVCLLN NFYPREAKVQ |
| constant | | WKVDNALQSG NSQESVTEQD |
| region | | SKDSTYSLSS TLTLSKADYE |
| SEQ ID | | KHKVYACEVT HQGLSSPVTK |
| NO: 39 | | SFNRGEC |
| IgG4 | HC | ASTKGPSVFPLAPCSRSTSES |
| (S228P) | | TAALGCLVKDYFPEPVTVSWN |
| mutant | | SGALTSIGVHTFPAVLQSSGL |
| constant | | YSLSSVVTVPSSSLGTKTYTC |
| region | | NVDHKPSNTKVDIKRVESKYG |
| (EU | | PPCPPCPAPEFLGGPSVFLFP |
| Numbering) | | PKPKDTLMISRTPEVTCVVIV |
| SEQ ID | | DVSQEDPEVQFNWYVDGVEVH |
| NO: 40 | | NAKTKPREEQFNSTYRVVSVL |
| | | TVILHQDWLNGKEYKCKVSNK |
| | | GLPSSIEKTISKAKGQPREPQ |
| | | VYTLPPSQIEEMTKNQVSLTC |
| | | LVKGFYPSDIAVEWESNGQPE |
| | | NNYKTTPPVLDSDIGSFFLYS |
| | | RLTVDKSRWQEGNVFSCSVMH |
| | | EALHNHYTQKSLSLSLG |
| IgG1 | HC | ASTKGPSVFPLAPSSKSTSGG |
| wild | | TAALGCLVKDYFPEPVTVSWN |
| type | | SGALTISGVHTFPAVLQSSGL |
| SEQ ID | | YSLSSVVTVPSSSLGTQTYIC |
| NO: 41 | | NVNHKPSNTKVDIKRVEPKSC |
| | | DKTHTCPPCPAPELLGGPSVF |
| | | LFPPKPKDTLMISRTPEVTIC |
| | | VVVDVSHEDPEVKFNWYVDGV |
| | | EVHNAKTKPREEQYNSTYRVV |
| | | SVILTVLHQDWLNGKEYKCKV |
| | | SNKALPAPIEKTISKAKGQPR |
| | | EPQVYTLPIPSREEMTKNQVS |
| | | LTCLVKGFYPSDIAVEWESNG |
| | | QPENNYKTTPPVLDISDGSFF |
| | | LYSKLTVDKSRWQQGNVFSCS |
| | | VMHEALHNHYTQKSLSLSPIG |
| | | K |
| IgG1 | HC | ASTKGPSVFPLAPSSKSTSGG |
| (N297A) | | TAALGCLVKDYFPEPVTVSWN |
| mutant | | SGALTISGVHTFPAVLQSSGL |
| constant | | YSLSSVVTVPSSSLGTQTYIC |
| region | | NVNHKPSNTKVDIKRVEPKSC |
| (EU | | DKTHTCPPCPAPELLGGPSVF |
| Numbering) | | LFPPKPKDTLMISRTPEVTIC |
| SEQ ID | | VVVDVSHEDPEVKFNWYVDGV |
| NO: 42 | | EVHNAKTKPREEQYASTYRVV |
| | | SVILTVLHQDWLNGKEYKCKV |
| | | SNKALPAPIEKTISKAKGQPR |
| | | EPQVYTLPIPSREEMTKNQVS |
| | | LTCLVKGFYPSDIAVEWESNG |
| | | QPENNYKTTPPVLDISDGSFF |
| | | LYSKLTVDKSRWQQGNVFSCS |
| | | VMHEALHNHYTQKSLSLSPIG |
| | | K |
| IgM | HC | GSASAPTLFPLVSCENSPSDT |
| constant | | SSVAVGCLAQDFLPDSITFSW |
| delta | | KYKNNSIDISSTRGFPSVLRG |
| CDC | | GKYAATSQVLLPSKDVMQGTD |
| (P311A, | | EHVVCKVQHPNIGNKEKNVPL |
| P313S) | | PVIAELPPKVSVFVPPRDGFF |
| SEQ ID | | GNPRKSKLICQATGFSPRIQI |
| NO: 73 | | QVSWLREGKQVGSGVTTDQVQ |
| | | AEAKESGPTTYKVTSTLTIKE |
| | | SDIWLGQSMFTCRVDHRGLTF |
| | | QQNASSMCVPDQDTAIRVFAI |
| | | PPSFASIFILTKSTKLTCLVT |
| | | DLTTYDSVTISWTRQNGEAVK |
| | | THTNISESHPNATFSIAVGEA |
| | | SICEDDWNSGERFTCTVTHTD |
| | | LASSLKQTISRPKGVALHRPD |
| | | IVYLLPPAREQLNLRESATIT |
| | | CLVTGFSPADVFVQWMQRGQP |
| | | LSPEKYIVTSAPMPEPQAPGR |
| | | YFAHSILTVSEEEWNTGETYT |
| | | CVVAHEALPNRVITERTVDKS |
| | | TGKPTLYNVSLVMSDTAGTCY |
| IgGA1 | HC | ASPTSPKVFPLSLCSTQPDGN |
| SEQ ID | | VVIACLVQGFFPQEPLSVTWS |
| NO: 74 | | ESGQGVITARNFPPSQDASGD |
| | | LYTTSSQLTLPATQCLAGKSV |
| | | TCHVKHYTNPSQIDVTVPCPV |
| | | PSTPPTPSPSTPPTPSPSCCH |
| | | PRLSLHRPALEDLLLGSEANI |
| | | LTCTLTGLRDASGVTFTWTPS |
| | | SGKSAVQGPPERDLCGCYSVS |
| | | SVLPGICAEPWNHGKTFTCTA |
| | | AYPESKTPLTATLSKSGNTFR |
| | | PEVHLLPPPSEEILALNELVT |
| | | LTCLARGFSPKDVLVRWLQGS |
| | | QELPREKYLTWASRQEPISQG |
| | | TTTFAVTSILRVAAEDWKKGD |
| | | TFSCMVGHEALPLAFTQKTID |
| | | RLIAGKPTHVNVSVVMAEVDG |
| | | TCY |
| IgGA2 | HC | ASPTSPKVFPLSLDSTPQDG |
| SEQ ID | | NVVVACLVQGFFPQEPLSVT |
| NO: 75 | | WSESGQNIVTARNFPPSQDA |
| | | SGDLYTTSSQLTLPATQCPD |
| | | GKSVTCHVKHYTNSSIQDVT |
| | | VPCRVPPPPPCCHPRLSLHR |
| | | PALEDLLLGSEANLTCTLTG |
| | | LRDAISGATFTWTPSSGKSA |
| | | VQGPPERDLCGCYSVSSVLP |
| | | GCAQPWNHGETIFTCTAAHP |
| | | ELKTPLTANITKSGNTFRPE |
| | | VHLLPPPSEELALNELVTLT |
| | | CILARGFSPKDVLVRWLQGS |
| | | QELPREKYLTWASRQEPSQG |
| | | TTTYAVTSIILRVAAEDWKK |
| | | GETFSCMVGHEALPLAFTQK |
| | | TIDRMAGKPTHINVSVIVMA |
| | | EADGTCY |
| Human | HC | MKNHLLFWGVLAVFIKAVHV |
| Ig_J | | KAQEDERIVLVDNKCKCARI |
| chain | | TSRIIRSISEDPNEDIVERN |
| SEQ ID | | IRIIVPLNNRENISDPTSPL |
| NO: 76 | | RTRFVYHLSDLCKKCDPTIE |
| | | VELDNQIVTATQSNICDEDS |
| | | ATETCYTYDRNKCYTAVVPL |
| | | VYGGETIKMVETALTPDACY |
| | | PD |

### Anti-TCRβV5 Antibodies

Accordingly, in one aspect, the disclosure provides an anti-TCRβV antibody molecule that binds to human TCRβ V5. In some embodiments, the TCRβ V5 subfamily comprises TCRβ V5-5*01, TCRβ V5-6*01, TCRβ V5-4*01, TCRβ V5-8*01, TCRβ V5-1*01, or a variant thereof.

Exemplary anti-TCRβV5 antibodies of the disclosure are provided in Table 6. In some embodiments, the anti-TCRβV5 is antibody C, e.g., humanized antibody C (antibody C-H), as provided in Table 6. In some embodiments, the anti-TCRβV antibody comprises one or more (e.g., all three) of a LC CDR1, LC CDR2, and LC CDR3 provided in Table 6; and/or one or more (e.g., all three) of a HC CDR1, HC CDR2, and HC CDR3 provided in Table 6, or a sequence with at least 95% identity thereto. In some embodiments, antibody C comprises a variable heavy chain (VH) and/or a variable light chain (VL) provided in Table 6, or a sequence with at least 95% identity thereto.

TABLE 6
Amino acid sequences for anti TCRβ V5 antibodies
Amino acid and nucleotide sequences for murine and humanized
antibody molecules which bind to TCRVB 5 (e.g., TCRVB 5-5 or
TCRVB 5-6). The amino acid the heavy and light chain CDRs,
and the amino acid and nucleotide sequences of the heavy and
light chain variable regions, and the heavy and light chains
are shown.
Murine antibody C

| | | |
|---|---|---|
| SEQ ID | HC CDR1 | AYGVN |
| NO: 1315 | (Kabat) | |
| SEQ ID | HC CDR2 | M1WGDGNTDYNSALRS |
| NO: 1316 | (Kabat) | |
| SEQ ID | HC CDR3 | DRVTATLYAMDY |
| NO: 1317 | (Kabat) | |
| SEQ ID | HC CDR1 | GFSLTAY |
| NO: 1318 | (Chothia) | |
| SEQ ID | HC CDR2 | WGDGN |
| NO: 1319 | (Chothia) | |
| SEQ ID | HC CDR3 | DRVTATLYAMDY |
| NO: 1317 | (Chothia) | |
| SEQ ID | HC CDR1 | GFSLTAYGVN |
| NO: 1320 | (Combined) | |
| SEQ ID | HC CDR2 | MIWGDGNTDYNSALRS |
| NO: 1316 | (Combined) | |
| SEQ ID | HC CDR3 | DRVTATLYAMDY |
| NO: 1317 | (Combined) | |
| SEQ ID | LC CDR1 | SASQGISNYLN |
| NO: 1321 | (Kabat) | |
| SEQ ID | LC CDR2 | YTSSLHS |
| NO: 1322 | (Kabat) | |
| SEQ ID | LC CDR3 | QQYSRLPRT |
| NO: 1323 | (Kabat) | |
| SEQ ID | LC CDR1 | SASQGISNYLN |
| NO: 1321 | (Chothia) | |
| SEQ ID | LC CDR2 | YTSSLHS |
| NO: 1322 | (Chothia) | |
| SEQ ID | LC CDR3 | QQYSRLPRT |
| NO: 1323 | (Chothia) | |
| SEQ ID | LC CDR1 | SASQGISNYLN |
| NO: 1321 | (Combined) | |
| SEQ ID | LC CDR2 | YTSSLHS |
| NO: 1322 | (Combined) | |
| SEQ ID | LC CDR3 | QQYSRLPRT |
| NO: 1323 | (Combined) | |
| SEQ ID | VH | DIQMTQTTSSLSASLGDRVTISCSASQGISNYLNW |
| NO: 232 | | YQQKPDGTVKLLIYYTSSLHSGVPSRFSGSGSGT |
| | | DYSLTISNLEPEDIATYYCQQYSKLPKTFGGGTK |
| | | VEIK |
| SEQ ID | VL | QVQLKESGPGLVAPSQSLSITCTVSGFSLTAYGV |
| NO: 233 | | NWVRQPPGKGLEWLGMIWGDGNTDYNSALKSR |
| | | LSISKDNSKSQVFLKMNSLQTDDTARYYCARDR |
| | | VTATLYAMDYWGQGTSVTVSS |

Humanized antibody C C-H-1 antibody

| | | |
|---|---|---|
| SEQ ID | HC CDR1 | AYGVN |
| NO: 1315 | (Kabat) | |
| SEQ ID | HC CDR2 | MIWGDGNTDYNSALKS |
| NO: 1316 | (Kabat) | |
| SEQ ID | HC CDR3 | DRVTATLYAMDY |
| NO: 1317 | (Kabat) | |
| SEQ ID | HC CDR1 | GFSLTAY |
| NO: 1318 | (Chothia) | |
| SEQ ID | HC CDR2 | WGDGN |
| NO: 1319 | (Chothia) | |
| SEQ ID | HC CDR3 | DRVTATLYAMDY |
| NO: 1317 | (Chothia) | |
| SEQ ID | HC CDR1 | GFSLTAYGVN |
| NO: 1320 | (Combined) | |
| SEQ ID | HC CDR2 | MIWGDGNTDYNSALKS |
| NO: 1316 | (Combined) | |
| SEQ ID | HC CDR3 | DRVTATLYAMDY |
| NO: 1317 | (Combined) | |
| SEQ ID | LC CDR1 | SASQGISNYLN |
| NO: 1321 | (Kabat) | |
| SEQ ID | LC CDR2 | YTSSLHS |
| NO: 1322 | (Kabat) | |
| SEQ ID | LC CDR3 | QQYSKLPRT |
| NO: 1323 | (Kabat) | |
| SEQ ID | LC CDR1 | SASQGISNYLN |
| NO: 1321 | (Chothia) | |
| SEQ ID | LC CDR2 | YTSSLHS |
| NO: 1322 | (Chothia) | |
| SEQ ID | LC CDR3 | QQYSKLPRT |
| NO: 1323 | (Chothia) | |
| SEQ ID | LC CDR1 | SASQGISNYLN |
| NO: 1321 | (Combined) | |
| SEQ ID | LC CDR2 | YTSSLHS |
| NO: 1322 | (Combined) | |
| SEQ ID | LC CDR3 | QQYSKLPRT |
| NO: 1323 | (Contained) | |
| SEQ ID | VL | DIQMTQSPSSLSASVGDRVTITCSASQGISNYLNW |
| NO: 1324 | | YQQTPGKAPKLLIYYTSSLHSGVPSRFSGSGSGTD |
| | | YTFTISSLQPEDIATYYCQQYSKLPRTFGQGTKLQI |
| | | T |
| SEQ ID | VH | QVQLQESGPGLVRPSQTLSLTCTVSGFSLTAYGV |
| NO: 1325 | | |
| | | NWVRQPPGRGLEWLGMIWGDGNTDYNSALKSR |
| | | VTMLKDTSKNQFSLRLSSVTAADTAVYYCARDR |
| | | VTATLYAMDYW GQGSLVTVSS |

Humanized antibody C Variable light chain (VL)

| | | | |
|---|---|---|---|
| SEQ ID | VL | C-H-VL.1 | DIQMTQSPSFLSASVGDRVTITCSASQGISNYLNWYQQ |
| NO: 3000 | | | KPGKAVKLLIYYTSSLHSGVPSRFSGSGSGTEYTLTISS |
| | | | LQPEDFATYYCQQYSKLPRTFGGGTKVEIK |
| SEQ ID | VL | C-H-VL.2 | DIQMTQSPSSLSASVGDRVTITCSASQGISNYLNWYQQ |
| NO: 3001 | | | KPGKAVKLLIYYTSSLHSGVPSRFSGSGSGTDYTLTISS |
| | | | LQPEDFATYYCQQYSKLPRTFGGGTKVEIK |
| SEQ ID | VL | C-H-VL.3 | DIQMTQSPSSLSASVGDRVTITCSASQGISNYLNWYQQ |
| NO: 3002 | | | KPGKVVKLLIYYTSSLHSGVPSRFSGSGSGTDYTLTISS |
| | | | LQPEDVATYYCQQYSKLPRTFGGGTKVEIK |
| SEQ ID | VL | C-H-VL.4 | DIQMTQSPSSLSASVGDRVTITCSASQGISNYLNWYQQ |
| NO: 3003 | | | KPGQAVKLLIYYTSSLHSGVPSRFSGSGSGTDYTLTISS |
| | | | LQPEDVATYYCQQYSKLPRTFGGGTKVEIK |
| SEQ ID | VL | C-H-VL.5 | DIQMTQSPSSLSASVGDRVTITCSASQGISNYLNWYQQ |
| NO: 3004 | | | KPGKAVKLLIYYTSSLHSGVPSRFSGSGSGTDYTFTISS |
| | | | LQPEDIATYYCQQYSKLPRTFGGGTKVEIK |
| SEQ ID | VL | C-H-VL.6 | DIQMTQSPSSLSASVGDRVTITCSASQGISNYLNWYQQ |
| NO: 3005 | | | KPGKTVKLLIYYTSSLHSGIPSRFSGSGSGTDYTLTIRSL |
| | | | QPEDFATYYCQQYSKLPRTFGGGTKVEIK |
| SEQ ID | VL | C-H-VL.7 | AIQMTQSPSSLSASVGDRVTITCSASQGISNYLNWYQQ |
| NO: 3006 | | | KPGKAVKLLIYYTSSLHSGVPSRFSGSGSGTDYTLTISS |
| | | | LQPEDFATYYCQQYSKLPRTFGGGTKVEIK |
| SEQ ID | VL | C-H-VL.8 | DIQMTQSPSSVSASVGDRVTITCSASQGISNYLNWYQQ |
| NO: 3007 | | | KPGKAVKLLIYYTSSLHSGVPSRFSGSGSGTDYTLTISS |
| | | | LQPEDFATYYCQQYSKLPRTFGGGTKVEIK |
| SEQ ID | VL | C-H-VL.9 | DIQMTQSPSSLSASVGDRVTITCSASQGISNYLNWYQQ |
| NO: 3008 | | | KPGKAVKRLIYYTSSLHSGVPSRFSGSGSGTEYTLTISN |
| | | | LQPEDFATYYCQQYSKLPRTFGGGTKVEIK |
| SEQ ID | VL | C-H-VL.10 | AIRMTQSPFSLSASVGDRVTITCSASQGISNYLNWYQQ |
| NO: 3009 | | | KPAKAVKLFIYYTSSLHSGVPSRFSGSGSGTDYTLTISS |
| | | | LQPEDFATYYCQQYSKLPRTFGGGTKVEIK |
| SEQ ID | VL | C-H-VL.11 | DIQMTQSPSSLSASVGDRVTITCSASQGISNYLNWYQQ |
| NO: 3010 | | | KPGKAVKRLIYYTSSLHSGVPSRFSGSGSGTEYTLTISS |
| | | | LQPEDFATYYCQQYSKLPRTFGGGTKVEIK |
| SEQ ID | VL | C-H-VL.12 | DIQMTQSPSTLSASVGDRVTITCSASQGISNYLNWYQQ |
| NO: 3011 | | | KPGKAVKLLIYYTSSLHSGVPSRFSGSGSGTEYTLTISS |
| | | | LQPDDFATYYCQQYSKLPRTFGGGTKVEIK |
| SEQ ID | VL | C-H-VL.13 | DIQMTQSPSSLSASVGDRVTITCSASQGISNYLNWYQQ |
| NO: 3012 | | | KPGKAVKSLIYYTSSLHSGVPSRFSGSGSGTDYTLTISS |
| | | | LQPEDFATYYCQQYSKLPRTFGGGTKVEIK |
| SEQ ID | VL | C-H-VL.14 | DIQMTQSPSSLSASVGDRVTITCSASQGISNYLNWYQQ |
| NO: 3013 | | | KPGKAVKSLIYYTSSLHSGVPSKFSGSGSGTDYTLTISS |
| | | | LQPEDFATYYCQQYSKLPRTFGGGTKVEIK |
| SEQ ID | VL | C-H-VL.15 | DIQMTQSPSSLSASVGDRVTITCSASQGISNYLNWYQQ |
| NO: 3014 | | | KPEKAVKSLIYYTSSLHSGVPSRFSGSGSGTDYTLTISS |
| | | | LQPEDFATYYCQQYSKLPRTFGGGTKVEIK |
| SEQ ID | VL | C-H-VL.16 | DIQMTQSPSAMSASVGDRVTITCSASQGISNYLNWYQ |
| NO: 3015 | | | QKPGKVVKRLIYYTSSLHSGVPSRFSGSGSGTEYTLTIS |
| | | | SLQPEDFATYYCQQYSKLPRTFGGGTKVEIK |
| SEQ ID | VL | C-H-VL.17 | DIVMTQSPDSLAVSLGERATENCSASQGISNYLNWYQQ |
| NO: 3016 | | | KPGQPVKLLIYYTSSLHSGVPDRFSGSGSGTDYTLTISS |
| | | | LQAEDVAVYYCQQYSKLPRTFGGGTKVEIK |
| SEQ ID | VL | C-H-VL.18 | EIVMTQSPGTLSLSPGERATLSCSASQGISNYLNWYQQ |
| NO: 3017 | | | KPGQAVKLLIYYTSSLHSGIPDRFSGSGSGTDYTLTISR |
| | | | LEPEDFAVYYCQQYSKLPRTFGGGTKVEIK |
| SEQ ID | VL | C-H-VL.19 | EIVMTQSPPTLSLSPGERVTLSCSASQGISNYLNWYQQ |
| NO: 3018 | | | KPGQAVKLLIYYTSSLHSGIPARFSGSGSGTDYTLTISS |
| | | | LQPEDFAVYYCQQYSKLPRTFGGGTKVEIK |
| SEQ ID | VL | C-H-VL.20 | EIVMTQSPPTLSLSPGERVTLSCSASQGISNYLNWYQQ |
| NO: 3019 | | | KPGQAVKLLIYYTSSLHSSIPARFSGSGSGTDYTLTISSL |
| | | | QPEDFAVYYCQQYSKLPRTFGGGTKVEIK |
| SEQ ID | VL | C-H-VL.21 | EIVMTQSPATLSLSPGERATLSCSASQGISNYLNWYQQ |
| NO: 3020 | | | KPGQAVKLLIYYTSSLHSGIPARFSGSGSGTDYTLTISS |
| | | | LEPEDFAVYYCQQYSKLPRTFGGGTKVEIK |
| SEQ ID | VL | C-H-VL.22 | EIVMTQSPATLSLSPGERATLSCSASQGISNYLNWYQQ |
| NO: 3021 | | | KPGQAVKLLIYYTSSLHSGIPARFSGSGSGTDYTLTISR |
| | | | LEPEDFAVYYCQQYSKLPRTFGGGTKVEIK |
| SEQ ID | VL | C-H-VL.23 | EIVMTQSPATLSLSPGERATLSCSASQGISNYLNWYQQ |
| NO: 3022 | | | KPGQAVKLLIYYTSSLHSGIPDRFSGSGSGTDYTLTISR |
| | | | LEPEDFAVYYCQQYSKLPRTFGGGTKVEIK |
| SEQ ID | VL | C-H-VL.24 | EIVMTQSPATLSLSPGERATLSCSASQGISNYLNWYQQ |
| NO: 3023 | | | KPGLAVKLLIYYTSSLHSGIPDRFSGSGSGTDYTLTISR |
| | | | LEPEDFAVYYCQQYSKLPRTFGGGTKVEIK |
| SEQ ID | VL | C-H-VL.25 | DIQMIQSPSFLSASVGDRVSIICSASQGISNYLNWYLQK |
| NO: 3024 | | | PGKSVKLFIYYTSSLHSGVSSRFSGRGSGTDYTLTIISLK |
| | | | PEDFAAYYCQQYSKLPRTFGGGTKVEIK |
| SEQ ID | VL | C-H-VL.26 | EIVMTQSPATLSLSPGERATLSCSASQGISNYLNWYQQ |
| NO: 3025 | | | KPGQAVKLLIYYTSSLHSGIPARFSGSGSGTDYTLTISS |
| | | | LQPEDFAVYYCQQYSKLPRTFGGGTKVEIK |
| SEQ ID | VL | C-H-VL.27 | EIVMTQSPATLSLSPGERATLSCSASQGISNYLNWYQQ |
| NO: 3026 | | | KPGQAVKLLIYYTSSLHSGIPARFSGSGPGTDYTLTISS |
| | | | LEPEDFAVYYCQQYSKLPRTFGGGTKVEIK |
| SEQ ID | VL | C-H-VL.28 | DIVMTQTPLSLSVTPGQPASISCSASQGISNYLNWYLQ |
| NO: 3027 | | | KPGQSVKLLIYYTSSLHSGVPDRFSGSGSGTDYTLKISR |
| | | | VEAEDVGVYYCQQYSKLPRTFGGGTKVEIK |
| SEQ ID | VL | C-H-VL.29 | DIVMTQTPLSLSVTPGQPASISCSASQGISNYLNWYLQ |
| NO: 3028 | | | KPGQPVKLLIYYTSSLHSGVPDRFSGSGSGTDYTLKISR |
| | | | VEAEDVGVYYCQQYSKLPRTFGGGTKVEIK |
| SEQ ID | VL | C-H-VL.30 | DIVMTQSPAFLSVTPGEKVTITCSASQGISNYLNWYQQ |
| NO: 3029 | | | KPDQAVKLLIYYTSSLHSGVPSRFSGSGSGTDYTFTISS |
| | | | LEAEDAATYYCQQYSKLPRTFGGGTKVEIK |
| SEQ ID | VL | C-H-VL.31 | DIVMTQSPLSLPVTPGEPASISCSASQGISNYLNWYLQK |
| NO: 3030 | | | PGQSVKLLIYYTSSLHSGVPDRFSGSGSGTDYTLKISRV |
| | | | EAEDVGVYYCQQYSKLPRTFGGGTKVEIK |
| SEQ ID | VL | C-H-VL.32 | DIVMTQTPLSLPVTPGEPASISCSASQGISNYLNWYLQK |
| NO: 3031 | | | PGQSVKLLIYYTSSLHSGVPDRFSGSGSGTDYTLKISRV |
| | | | EAEDVGVYYCQQYSKLPRTFGGGTKVEIK |
| SEQ ID | VL | C-H-VL.33 | EIVMTQSPATLSVSPGERATLSCSASQGISNYLNWYQQ |
| NO: 3032 | | | KPGQAVKLLIYYTSSLHSGIPARFSGSGSGTEYTLTISIL |
| | | | QSEDFAVYYCQQYSKLPRTFGGGTKVEIK |
| SEQ ID | VL | C-H-VL.34 | EIVMTQSPATLSVSPGERATLSCSASQGISNYLNWYQQ |
| NO: 3033 | | | KPGQAVKLLIYYTSSLHSGIPARFSGSGSGTEYTLTISSL |
| | | | QSEDFAVYYCQQYSKLPRTFGGGTKVEIK |
| SEQ ID | VL | C-H-VL.35 | DIVMTQSPLSLPVTLGQPASISCSASQGISNYLNWYQQ |
| NO: 3034 | | | RPGQSVKRLIYYTSSLHSGVPDRFSGSGSGTDYTLKISR |
| | | | VEAEDVGVYYCQQYSKLPRTFGGGTKVEIK |
| SEQ ID | VL | C-H-VL.36 | EITMTQSPAFMSATPGDKVNISCSASQGISNYLNWYQQ |
| NO: 3035 | | | KPGEAVKFIIYYTSSLHSGIPPRFSGSGYGTDYTLTINNI |
| | | | ESEDAAYYYCQQYSKLPRTFGGGTKVEIK |
| SEQ ID | VL | C-H-VL.37 | DIVMTQTPLSSPVTLGQPASISCSASQGISNYLNWYQQ |
| NO: 3036 | | | RPGQPVKLLIYYTSSLHSGVPDRFSGSGAGTDYTLKISR |
| | | | VEAEDVGVYYCQQYSKLPRTFGGGTKVEIK |
| SEQ ID | VL | C-H-VL.38 | EIVMTQSPDFQSVTPKEKVTITCSASQGISNYLNWYQQ |
| NO: 3037 | | | KPDQSVKLLIYYTSSLHSGVPSRFSGSGSGTDYTLTINS |
| | | | LEAEDAATYYCQQYSKLPRTFGGGTKVEIK |
| SEQ ID | VL | C-H-VL.39 | EIVMTQTPLSLSITPGEQASISCSASQGISNYLNWYLQK |
| NO: 3038 | | | ARPVVKLLIYYTSSLHSGVPDRFSGSGSGTDYTLKISRV |
| | | | EAEDFGVYYCQQYSKLPRTFGGGTKVEIK |
| SEQ ID | VL | C-H-VL.40 | EIVMTQTPLSLSITPGEQASMSCSASQGISNYLNWYLQ |
| NO: 3039 | | | KARPWKLLIYYTSSLHSGVPDRFSGSGSGTDYTLKISR |
| | | | VEAEDFGVYYCQQYSKLPRTFGGGTKVEIK |

Humanized antibody C Variable HEAVY chain (VH)

| | | | |
|---|---|---|---|
| SEQ ID | VH | C-H-VH.l | QVTLKESGPVLVKPTETLTLTCTVSGFSLTAYGVNWV |
| NO: 3040 | | | RQPPGKALEWLGMIWGDGNTDYNSALKSRLTISKDNS |
| | | | KSQVVLTMTNMDPVDTATYYCARDRVTATLYAMDY |
| | | | WGQGTLVTVSS |
| SEQ ID | VH | C-H-VH.2 | QVTLKESGPALVKPTETLTLTCTVSGFSLTAYGVNWV |
| NO: 3041 | | | RQPPGKALEWLGMIWGDGNTDYNSALKSRLIISKDNS |
| | | | KSQVVLTMTNMDPVDTATYYCARDRVTATLYAMDY |
| | | | WGQGTLVTVSS |
| SEQ ID | VH | C-H-VH.3 | QVTLKESGPALVKPTQTLTLTCTVSGFSLTAYGVNWV |
| NO: 3042 | | | RQPPGKALEWLGMIWGDGNTDYNSALKSRLTISKDNS |
| | | | KSQVVLTMTNMDPVDTATYYCARDRVTATLYAMDY |
| | | | WGQGTLVTVSS |
| SEQ ID | VH | C-H-VH.4 | QVQLQESGPGLVKPSGTLSLTCAVSGFSLTAYGVNWV |
| NO: 3043 | | | RQPPGKGLEWLGMIWGDGNTDYNSALKSRLTISKDNS |
| | | | KSQVSLKLSSVTAADTAVYYCARDRVTATLYAMDYW |
| | | | GQGTLVTVSS |
| SEQ ID | VH | C-H-VH.5 | QVTLKESGPTLVKPTQTLTLTCTVSGFSLTAYGVNWV |
| NO: 3044 | | | RQPPGKALEWLGMIWGDGNTDYNSALKSRLTITKDNS |
| | | | KSQWLTMTNMDPVDTATYYCARDRVTATLYAMDY |
| | | | WGQGTLVTVSS |
| SEQ ID | VH | C-H-VH.6 | QVTLKESGPALVKPTQTLTLTCTVSGFSLTAYGVNWV |
| NO: 3045 | | | RQPPGKALEWLGMIWGDGNTDYNSALKSRLTITKDNS |
| | | | KSQVVLTMTNMDPVDTATYYCARDRVTATLYAMDY |
| | | | WGQGTLVTVSS |
| SEQ ID | VH | C-H-VH.7 | QVQLQESGPGLVKPSQTLSLTCTVSGFSLTAYGVNWV |
| NO: 3046 | | | RQPPGKGLEWLGMIWGDGNTDYNSALKSRLTISKDNS |
| | | | KSQVSLKLSSVTAADTAVYYCARDRVTATLYAMDYW |
| | | | GQGTLVTVSS |
| SEQ ID | VH | C-H-VH.8 | QVQLQESGPGLVKPSETLSLTCTVSGFSLTAYGVNWV |
| NO: 3047 | | | RQPPGKGLEWLGMIWGDGNTDYNSALKSRLTISKDNS |
| | | | KSQVSLKLSSVTAADTAVYYCARDRVTATLYAMDYW |
| | | | GQGTLVTVSS |
| SEQ ID | VH | C-H-VH.9 | QVQLQESGPGLVKPSQTLSLTCAVSGFSLTAYGVNWV |
| NO: 3048 | | | RQPPGKGLEWLGMIWGDGNTDYNSALKSRLTISKDNS |
| | | | KSQVSLKLSSVTAADTAVYYCARDRVTATLYAMDYW |
| | | | GQGTLVTVSS |
| SEQ ID | VH | C-H- | QVQLQESGPGLVKPSDTLSLTCTVSGFSLTAYGVNWV |
| NO: 3049 | | VH.10 | RQPPGKGLEWLGMIWGDGNTDYNSALKSRLTISKDNS |
| | | | KSQVSLKLSSVTAADTAVYYCARDRVTATLYAMDYW |
| | | | GQGTLVTVSS |
| | | C-H- | QVQLQESGPGLVKPSQTLSLTCTVSGFSLTAYGVNWV |
| SEQ ID | VH | VH.11 | RQHPGKGLEWLGMIWGDGNTDYNSALKSRLTISKDN |
| NO: 3050 | | | SKSQVSLKLSSVTAADTAVYYCARDRVTATLYAMDY |
| | | | WGQGTLVTVSS |
| SEQ ID | VH | C-H- | QVQLQESGPGLVKPSQTLSLTCTVSGFSLTAYGVNWV |
| NO: 3051 | | VH.12 | RQPAGKGLEWLGMIWGDGNTDYNSALKSRLTISKDN |
| | | | SKSQVSLKLSSVTAADTAVYYCARDRVTATLYAMDY |
| | | | WGQGTLVTVSS |
| SEQ ID | VH | C-H- | QVQLQESGPGLVKPSQTLSLTCAVSGFSLTAYGVNWV |
| NO: 3052 | | VH.13 | RQPPGKGLEWLGMIWGDGNTDYNSALKSRLTISKDNS |
| | | | KSQVSLKLSSVTAVDTAVYYCARDRVTATLYAMDYW |
| | | | GQGTLVTVSS |
| SEQ ID | VH | C-H- | QVQLQESGPGLVKPSETLSLTCTVSGFSLTAYGVNWV |
| NO: 3053 | | VH.14 | RQPPGKGLEWLGMIWGDGNTDYNSALKSRLTISKDNS |
| | | | KSHVSLKLSSVTAADTAVYYCARDRVTATLYAMDYW |
| | | | GQGTLVTVSS |
| SEQ ID | VH | C-H- | QVQLQESGPGLVKPSETLSLTCAVSGFSLTAYGVNWV |
| NO: 3054 | | VH.15 | RQPPGKGLEWLGMIWGDGNTDYNSALKSRLTISKDNS |
| | | | KSQVSLKLSSVTAADTAVYYCARDRVTATLYAMDYW |
| | | | GQGTLVTVSS |
| SEQ ID | VH | C-H- | QVQLQESGPGLVKPSQTLSLTCAVYGFSLTAYGVNWV |
| NO: 3055 | | VH.16 | RQPPGKGLEWLGMIWGDGNTDYNSALKSRLTISKDNS |
| | | | KSQVSLKLSSVTAADTAVYYCARDRVTATLYAMDYW |
| | | | GQGTLVTVSS |
| SEQ ID | VH | C-H- | RVQLQESGPGLVKPSETLSLTCTVSGFSLTAYGVNWV |
| NO: 3056 | | VH.17 | RQPPGKGLEWLGMIWGDGNTDYNSALKSRLTISKDNS |
| | | | KSQVPLKLSSVTAADTAVYYCARDRVTATLYAMDYW |
| | | | GQGTLVTVSS |
| SEQ ID | VH | C-H- | QVQLQESGPGLVKPSQTLSLTCTVSGFSLTAYGVNWV |
| NO: 3057 | | VH.18 | RQHPGKGLEWLGMIWGDGNTDYNSALKSLLTISKDNS |
| | | | KSQVSLKLSSVTAADTAVYYCARDRVTATLYAMDYW |
| | | | GQGTLVTVSS |
| SEQ ID | VH | C-H- | QVQLQESGPGLVKPSDTLSLTCAVSGFSLTAYGVNWV |
| NO: 3058 | | VH.19 | RQPPGKGLEWLGMIWGDGNTDYNSALKSRLTISKDNS |
| | | | KSQVSLKLSSVTALDTAVYYCARDRVTATLYAMDYW |
| | | | GQGTLVTVSS |
| SEQ ID | VH | C-H- | QVQLQESGPGLVKPSDTLSLTCAVSGFSLTAYGVNWV |
| NO: 3059 | | VH.20 | RQPPGKGLEWLGMIWGDGNTDYNSALKSRLTISKDNS |
| | | | KSQVSLKLSSVTAVDTAVYYCARDRVTATLYAMDYW |
| | | | GQGTLVTVSS |
| SEQ ID | VH | C-H- | QVQLQESGSGLVKPSQTLSLTCAVSGFSLTAYGVNWV |
| NO: 3060 | | VH.21 | RQPPGKGLEWLGMIWGDGNTDYNSALKSRLTISKDNS |
| | | | KSQVSLKLSSVTAADTAVYYCARDRVTATLYAMDYW |
| | | | GQGTLVTVSS |
| SEQ ID | VH | C-H- | EVQLVESGGGLVQPGRSLRLSCTVSGFSLTAYGVNWV |
| NO: 3061 | | VH.22 | RQAPGKGLEWLGMIWGDGNTDYNSALKSRLTISKDN |
| | | | SKSIVYLQMNSLKTEDTAVYYCARDRVTATLYAMDY |
| | | | WGQGTLVTVSS |
| SEQ ID | VH | C-H- | EVQLVESGGGLVQPGPSLRLSCTVSGFSLTAYGVNWV |
| NO: 3062 | | VH.23 | RQAPGKGLEWLGMIWGDGNTDYNSALKSRLTISKDN |
| | | | SKSIVYLQMNSLKTEDTAVYYCARDRVTATLYAMDY |
| | | | WGQGTLVTVSS |
| SEQ ID | VH | C-H- | QVQLQESGSGLVKPSQTLSLTCAVSGFSLTAYGVNWV |
| NO: 3063 | | VH.24 | RQSPGKGLEWLGMIWGDGNTDYNSALKSRLTISKDNS |
| | | | KSQVSLKLSSVTAADTAVYYCARDRVTATLYAMDYW |
| | | | GQGTLVTVSS |
| SEQ ID | VH | C-H- | QVQLQESGPGLVKPSETLSLTCTVSGFSLTAYGVNWV |
| NO: 3064 | | VH.25 | RQPAGKGLEWLGMIWGDGNTDYNSALKSRLTISKDN |
| | | | SKSQVSLKLSSVTAADTAVYYCARDRVTATLYAMDY |
| | | | WGQGTLVTVSS |
| SEQ ID | VH | C-H- | EVQLVESGGGLVKPGRSLRLSCTVSGFSLTAYGVNWV |
| NO: 3065 | | VH.26 | RQAPGKGLEWLGMIWGDGNTDYNSALKSRLTISKDN |
| | | | SKSIVYLQMNSLKTEDTAVYYCARDRVTATLYAMDY |
| | | | WGQGTLVTVSS |
| SEQ ID | VH | C-H- | QVQLQESGPGLVKPSETLSLTCAVYGFSLTAYGVNWV |
| NO: 3066 | | VH.27 | RQPPGKGLEWLGMIWGDGNTDYNSALKSRLTISKDNS |
| | | | KSQVYLKLSSVTAADTAVYYCARDRVTATLYAMDY |
| | | | WGQGTLVTVSS |
| SEQ ID | VH | C-H- | QVQLQESGPGLVKPSDTLSLTCAVSGFSLTAYGVNWV |
| NO: 3067 | | VH.28 | RQPPGKGLEWLGMIWGDGNTDYNSALKSRLTISKDNS |
| | | | KSQVSLKLSSVTAVDTGVYYCARDRVTATLYAMDYW |
| | | | GQGTLVTVSS |
| SEQ ID | VH | C-H- | EVQLVESGGGLVQPGGSLRLSCAVSGFSLTAYGVNWV |
| NO: 3068 | | VH.29 | RQAPGKGLEWLGMIWGDGNTDYNSALKSRLTISKDN |
| | | | SKSSVYLQMNSLKTEDTAVYYCARDRVTATLYAMDY |
| | | | WGQGTLVTVSS |
| SEQ ID | VH | C-H- | EVQLVESGGGLVKPGGSLRLSCAVSGFSLTAYGVNWV |
| NO: 3069 | | VH.30 | RQAPGKGLEWLGMIWGDGNTDYNSALKSRLTISKDN |
| | | | SKSTVYLQMNSLKTEDTAVYTCARDRVTATLYAMDY |
| | | | WGQGTLVTVSS |
| SEQ ID | VH | C-H- | QVQLQQSGPGLVKPSQTLSLTCAVSGFSLTAYGVNWV |
| NO: 3070 | | VH.31 | RQSPSRGLEWLGMIWGDGNTDYNSALKSRLTINKDNS |
| | | | KSQVSLQLNSVTPEDTAVYYCARDRVTATLYAMDYW |
| | | | GQGTLVTVSS |
| SEQ ID | VH | C-H- | QVQLVESGGGLVQPGGSLRLSCSVSGFSLTAYGVNWV |
| NO: 3071 | | VH.32 | RQAPGKGLEWLGMIWGDGNTDYNSALKSRLTISKDN |
| | | | SKSTVYLQMNSLRAEDTAVYYCARDRVTATLYAMDY |
| | | | WGQGTLVTVSS |
| SEQ ID | VH | C-H- | QVQLQQWGAGLLKPSETLSLTCAVYGFSLTAYGVNW |
| NO: 3072 | | VH.33 | VRQPPGKGLEWLGMIWGDGNTDYNSALKSRLTISKD |
| | | | NSKSQVSLKLSSVTAADTAVYYCARDRVTATLYAMD |
| | | | YWGQGTLVTVSS |
| SEQ ID | VH | C-H- | QVQLVESGGGWQPGRSLRLSCAVSGFSLTAYGVNW |
| NO: 3073 | | VH.34 | VRQAPGKGLEWLGMIWGDGNTDYNSALKSRLTISKD |
| | | | NSTSTVFLQMNSLRAEDTAVYYCARDRVTATLYAMD |
| | | | YWGQGTLVTVSS |
| SEQ ID | VH | C-H- | EVQLVESGGGLVQPGGSLRLSCAVSGFSLTAYGVNWV |
| NO: 3074 | | VH.35 | RQAPGKGLEWLGMIWGDGNTDYNSALKSRLTISKDN |
| | | | SKSTVYLQMNSLRAEDTAVYYCARDRVTATLYAMDY |
| | | | WGQGTLVTVSS |
| SEQ ID | VH | C-H- | EVQLVESGGGLVQPGGSLRLSCAVSGFSLTAYGVNWV |
| NO: 3075 | | VH.36 | RQAPGKGLEWLGMIWGDGNTDYNSALKSRLTISKDN |
| | | | AKSSVYLQMNSLRDEDTAVYYCARDRVTATLYAMD |
| | | | YWGQGTLVTVSS |
| SEQ ID | VH | C-H- | EVQLLESGGGLVQPGGSLRLSCAVSGFSLTAYGVNWV |
| NO: 3076 | | VH.37 | RQAPGKGLEWLGMIWGDGNTDYNSALKSRLTISKDN |
| | | | SKSTVYLQMNSLRAEDTAVYYCARDRVTATLYAMDY |
| | | | WGQGTLVTVSS |
| SEQ ID | VH | C-H- | QVQLVESGGGLVKPGGSLRLSCAVSGFSLTAYGVNW |
| NO: 3077 | | VH.38 | VRQAPGKGLEWLGMIWGDGNTDYNSALKSRLTISKD |
| | | | NAKSSVYLQMNSLRAEDTAVYYCARDRVTATLYAM |
| | | | DYWGQGTLVTVSS |
| SEQ ID | VH | C-H- | EVQLVESGGGLVQPGGSLKLSCAVSGFSLTAYGVNW |
| NO: 3078 | | VH.39 | VRQASGKGLEWLGMIWGDGNTDYNSALKSRLTISKD |
| | | | NSKSTVYLQMNSLKTEDTAVYYCARDRVTATLYAMD |
| | | | YWGQGTLVTVSS |
| SEQ ID | VH | C-H- | QVQLLESGGGLVKPGGSLRLSCAVSGFSLTAYGVNWV |
| NO: 3079 | | VH.40 | RQAPGKGLEWLGMIWGDGNTDYNSALKSRLTISKDN |
| | | | AKSSVYLQMNSLRAEDTAVYYCARDRVTATLYAMD |
| | | | YWGQGTLVTVSS |
| SEQ ID | VH | C-H- | QVQLVESGGGVVQPGRSLRLSCAVSGFSLTAYGVNW |
| NO: 3080 | | VH.41 | VRQAPGKGLEWLGMIWGDGNTDYNSALKSRLTISKD |
| | | | NSKSTVYLQMNSLRAEDTAVYYCARDRVTATLYAMD |
| | | | YWGQGTLVTVSS |
| SEQ ID | VH | C-H- | QVQLVESGGGVVQPGRSLRLSCAVSGFSLTAYGVNW |
| NO: 3081 | | VH.42 | VRQAPGKGLEWLGMIWGDGNTDYNSALKSRLTISKD |
| | | | NSKSRVYLQMNSLRAEDTAVYCARDRVTATLYAMD |
| | | | YWGQGTLVTVSS |
| SEQ ID | VH | C-H- | QVQLVESGGGWQPGRSLRLSCAVSGFSLTAYGVNW |
| NO: 3082 | | VH.43 | VRQAPGKGLEWLGMIWGDGNTDYNSALKSRLAISKD |
| | | | NSKSTVYLQMNSLRAEDTAVYYCARDRVTATLYAMD |
| | | | YWGQGTLVTVSS |
| SEQ ID | VH | C-H- | QVQLVESGGGWQPGGSLRLSCAVSGFSLTAYGVNW |
| NO: 3083 | | VH.44 | VRQAPGKGLEWLGMIWGDGNTDYNSALKSRLTISKD |
| | | | NSKSTVYLQMNSLRAEDTA\YYCARDRVTATLYAMD |
| | | | YWGQGTLVTVSS |
| SEQ ID | VH | C-H- | EVQLVESGGGLVQPGGSLRLSCAVSGFSLTAYGVNWV |
| NO: 3084 | | VH.45 | RQAPGKGLEWLGMIWGDGNTDYNSALKSRLTISKDN |
| | | | AKSTVYLQMNSLRAEDTAVYYCARDRVTATLYAMD |
| | | | YWGQGTLVTVSS |
| SEQ ID | VH | C-H- | EVQLVESGGGLVQPGGSLRLSCAVSGFSLTAYGVNWV |
| NO: 3085 | | VH.46 | RQAPGKGLEWLGMIWGDGNTDYNSALKSRLTISKDN |
| | | | AKSSVYLQMNSLRAEDTAVYYCARDRVTATLYAMD |
| | | | YWGQGTLVTVSS |
| SEQ ID | VH | C-H- | EVQLVESGGVVVQPGGSLRLSCAVSGFSLTAYGVNW |
| NO: 3086 | | VH.47 | VRQAPGKGLEWLGMIWGDGNTDYNSALKSRLTISKD |
| | | | NSKSSVYLQMNSLRTEDTALYYCARDRVTATLYAMD |
| | | | YWGQGTLVTVSS |
| SEQ ID | VH | C-H- | EVQLVESGGGLVQPGGSLRLSCAVSGFSLTAYGVNWV |
| NO: 3087 | | VH.48 | RQAPGKGLEWLGMIWGDGNTDYNSALKSRLTISKHN |
| | | | SKSTVYLQMNSLRAEDTAVYYCARDRVTATLYAMDY |
| | | | WGQGTLVTVSS |
| SEQ ID | VH | C-H- | EVQLVESGGGLVKPGGSLRLSCAVSGFSLTAYGVNWV |
| NO: 3088 | | VH.49 | RQAPGKGLEWLGMIWGDGNTDYNSALKSRLTISKDN |
| | | | AKSSVYLQMNSLRAEDTAVYYCARDRVTATLYAMD |
| | | | YWGQGTLVTVSS |
| SEQ ID | VH | C-H- | EVQLVESGGGLIQPGGSLRLSCAVSGFSLTAYGVNWV |
| NO: 3089 | | VH.50 | RQPPGKGLEWLGMIWGDGNTDYNSALKSRLTISKDNS |
| | | | KSTVYLQMNSLRAEDTAVYYCARDRVTATLYAMDY |
| | | | WGQGTLVTVSS |

Exemplary anti-TCRβV5 antibodies of the disclosure are provided in Table 7. In some embodiments, the anti-TCRβV5 is antibody E, e.g., humanized antibody E (antibody E-H), as provided in Table 7. In some embodiments, the anti-TCRβV antibody comprises one or more (e.g., all three) of a LC CDR1, LC CDR2, and LC CDR3 provided in Table 7; and/or one or more (e.g., all three) of a HC CDR1, HC CDR2, and HC CDR3 provided in Table 7, or a sequence with at least 95% identity thereto. In some embodiments, antibody E comprises a variable heavy chain (VH) and/or a variable light chain (VL) provided in Table 7, or a sequence with at least 95% identity thereto.

In some embodiments, antibody E comprises a heavy chain comprising the amino acid sequence of SEQ ID NO: 3284 and/or a light chain comprising the amino acid sequence of SEQ ID NO: 3285, or sequence with at least 95% identity thereto.

TABLE 7
Amino acid sequences for anti
TCRβ V5 antibodies
Amino acid and nucleotide sequences for
murine and humanized antibody molecules
which bind to TCRVB 5 (e.g., TCRVB 5-5
or TCRVB 5-6). The amino acid the heavy
and light chain CDRs, and the amino acid
and nucleotide sequences of the heavy
and light chain variable regions, and
the heavy and light chains are shown.
Murine antibody E

SSWMN

| | | |
|---|---|---|
| SEQ ID | HC CDR1 | |
| NO: 1298 | (Rabat) | |
| SEQ ID | HC CDR2 | RIYPGDGDT |
| NO: 1299 | (Rabat) | RYNGRFRG |
| SEQ ID | HC CDR3 | RGTGGWYFDV |
| NO: 1300 | (Rabat) | |
| SEQ ID | HC CDR1 | GYAFSSS |
| NO: 1302 | (Chothia) | |
| SEQ ID | HC CDR2 | YPGDGD |
| NO: 1303 | (Chothia) | |
| SEQ ID | HC CDR3 | RGTGGWYFDV |
| NO: 1301 | (Chothia) | |
| SEQ ID | HC CDR1 | GYAFSSSWMN |
| NO: 1304 | (Combined) | |
| SEQ ID | HC CDR2 | RIYPGDGDTR |
| NO: 1299 | (Combined) | YNGRFRG |
| SEQ ID | HC CDR3 | RGTGGWYFDV |
| NO: 1301 | (Combined) | |
| SEO ID | LC CDR1 | RASESVDSS |
| NO: 1305 | (Rabat) | GNSFMH |
| SEQ ID | LC CDR2 | RASNLES |
| NO: 1306 | (Rabat) | |
| SEQ ID | LC CDR3 | QQSFDDPFT |
| NO: 1307 | (Rabat) | |
| SEQ ID | LC CDR1 | SESVDSSGNSF |
| NO: 1308 | (Chothia) | |
| SEQ ID | LC CDR2 | RASNLES |
| NO: 1306 | (Chothia) | |
| SEQ ID | LC CDR3 | QQSFDDPFT |
| NO: 1307 | (Chothia) | |
| SEQ ID | LC CDR1 | RASESVDSSGNSFMH |
| NO: 1305 | (Combined) | |
| SEQ ID | LC CDR2 | RASNLES |
| NO: 1306 | (Combined) | |
| SEQ ID | LC CDR3 | QQSFDDPFT |
| NO: 1307 | (Combined) | |
| SEQ ID | VH | QVQLQQSGPELVKPGASVK |
| NO: 3091 | | ISCKASGYAFSSSWMNWVK |
| | | QRPGQGLEWIGRIYPGDGD |
| | | TKYNGKFKGKATLTADKSS |
| | | STAYMHLSSLTSVDSAVYF |
| | | CARRGTGGWYFDVWGAGTT |
| | | VTVSS |
| SEQ ID | Heavy | METDTLLLWVLLLWVPGST |
| NO: 3284 | chain | GQVQLQQSGPELVKPGASV |
| | | KISCKASGYAFSSSWMNWV |
| | | KQRPGQGLEWIGRIYPGDG |
| | | DTKYNGKFKGKATLTADKS |
| | | SSTAYMHLSSLTSVDSAVY |
| | | FCARRGTGGWYFDVWGAGT |
| | | TVTVSSAKTTAPSVYPLAP |
| | | VCGDTTGSSVTLGCLVKGY |
| | | FPEPVTLTWNSGSLSSGVH |
| | | TFPAVLQSDLYTLSSSVTV |
| | | TSSTWPSQS1TCNVAHPAS |
| | | STKVDKKIEPRGPTIKPCP |
| | | PCKCPAPNLLGGPSVFIFP |
| | | PKIKDVLMISLSPIVTCVV |
| | | VDVSEDDPDVQISWFVNNV |
| | | EVHTAQTQTHREDYNSTLR |
| | | VVSALPIQHQDWMSGKEFK |
| | | CKVNNKDLPAPIERTISKP |
| | | KGSVRAPQVYVLPPPEEEM |
| | | TKKQVTLTCMVTDFMPEDI |
| | | YVEWTNNGKTELNYKNTEP |
| | | VLDSDGSYFMYSKLRVEKK |
| | | NWVERNSYSCSVVHEGLHN |
| | | HHTTKSFSRTPGK |
| SEQ ID | VL | DIVLTQSPASLAVSLGQRA |
| NO: 3092 | | TISCRASESVDSSGNSFMH |
| | | WYQQKPGQPPQLLIYRASN |
| | | LESGIPARFSGSGSRTDFT |
| | | LTINPVEADDVATFYCQQS |
| | | FDDPFTFGSGTKLEIK |
| SEQ ID | Light | METDTLLLWVLLLWVPGST |
| NO: 3285 | chain | GDIVLTQSPASLAVSLGQR |
| | | ATISCRASESVDSSGNSFM |
| | | HWYQQKPGQPPQLLIYRAS |
| | | NLESGIPARFSGSGSRTDF |
| | | TLTINPVEADDVATFYCQQ |
| | | SFDDPFTFGSGTKLEIKRA |
| | | DAAPTVSIFPPSSEQLTSG |
| | | GASVVCFLNNFYPKDINVK |
| | | WKIDGSERQNGVLNSWTDQ |
| | | DSKDSTYSMSSTLTLTKDE |
| | | YERHNSYTCEATHKTSTSP |
| | | IVKSFNRNEC |

Humanized antibody E (E-H antibody)
Variable light chain (VL)

| | | | |
|---|---|---|---|
| SEQ ID | VL | E-H.1 | DIVLTQSPDSLAVSLGE |
| NO: 3093 | | | RATINCRASESVDSSGN |
| | | | SFMHWYQQKPGQPPQLL |
| | | | IYRASNLESGVPDRFSG |
| | | | SGSRTDFTLTISSLQAE |
| | | | DVAVYYCQQSFDDPFTF |
| | | | GQGTKLEIK |
| SEQ ID | VL | E-H.2 | EIVLTQSPATLSLSPGE |
| NO: 3094 | | | RATLSCRASESVDSSGN |
| | | | SFMHWYQQKPGQAPQLL |
| | | | IYRASNLESGIPARFSG |
| | | | SGSRTDFTLTISSLEPE |
| | | | DFAVYYCQQSFDDPFTF |
| | | | GQGTKLEIK |
| SEQ ID | VL | E-H.3 | EIVLTQSPATLSLSPG |
| NO: 3095 | | | ERATLSCRASESVDSS |
| | | | GNSFMHWYQQKPGQAP |
| | | | QLLIYRASNLESGIPA |
| | | | RFSGS |
| | | | GSRTDFTLTISRLE |
| | | | PEDFAVYYCQQSFD |
| | | | DPFTFGQGTKLEIK |
| SEQ ID | VL | E-H.4 | EIVLTQSPATLSLS |
| NO: 3096 | | | PGERATLSCRASES |
| | | | VDSSGNSFMHWYQQ |
| | | | KPGQAPQLLIYRAS |
| | | | NLESGIPARFSGSG |
| | | | SRTDFTLTISSLQP |
| | | | EDFAVYYCQQSFDD |
| | | | PFTFGQGTKLEIK |
| SEQ ID | VL | E-H.5 | DIQLTQSPSSLSAS |
| NO: 3097 | | | VGDRVTITCRASES |
| | | | VDSSGNSFMHWYQQ |
| | | | KPGQAPQLLIYRAS |
| | | | NLESGVPSRFSGSG |
| | | | SRTDFTLTISSLQP |
| | | | EDVATYYCQQSFDD |
| | | | PFTFGQGTKLEIK |
| SEQ ID | VL | E-H.6 | EIVLTQSPATLSLS |
| NO: 3098 | | | PGERATLSCRASES |
| | | | VDSSGNSFMHWYQQ |
| | | | KPGQAPQLLIYRAS |
| | | | NLESGIPARFSGSG |
| | | | PRTDFTLTISSLEP |
| | | | EDFAVYYCQQSFDD |
| | | | PFTFGQGTKLEIK |
| SEQ ID | VL | E-H.7 | EIVLTQSPATLSLS |
| NO: 3099 | | | PGERATLSCRASES |
| | | | VDSSGNSFMHWYQQ |
| | | | KPGQAPQLLIYRAS |
| | | | NLESGIPDRFSGSG |
| | | | SRTDFTLTISRLEP |
| | | | EDFAVYYCQQSFDD |
| | | | PFTFGQGTKLEIK |
| SEQ ID | VL | E-H.8 | DIQLTQSPSSLSAS |
| NO: 3100 | | | VGDRVTITCRASES |
| | | | VDSSGNSFMHWYQQ |
| | | | KPGKVPQLLIYRAS |
| | | | NLESGVPSRFSGSG |
| | | | SRTDFTLTISSLQP |
| | | | EDVATYYCQOSFDD |
| | | | PFTFGQGTKLEIK |
| SEQ ID | VL | E-H.9 | DIQLTQSPSSLSAS |
| NO: 3101 | | | VGDRVTITCRASES |
| | | | VDSSGNSFMHWYQQ |
| | | | KPGKTPQLLIYRAS |
| | | | NLESGIPSRFSGSG |
| | | | SRTDFTLTIRSLQP |
| | | | EDFATYYCQQSFDD |
| | | | PFTFGQGTKLEIK |
| SEQ ID | VL | E-H.10 | EIVLTQSPGTLSLS |
| NO: 3102 | | | PGERATLSCRASES |
| | | | VDSSGNSFMHWYQQ |
| | | | KPGQAPQLLIYRAS |
| | | | NLESGIPDRFSGSG |
| | | | SRTDFTLTISRLEP |
| | | | EDFAVYYCQQSFDD |
| | | | PFTFGQGTKLEIK |
| SEQ ID | VL | E-H.11 | EIVLTQSPATLSLS |
| NO: 3103 | | | PGERATLSCRASES |
| | | | VDSSGNSFMHWYQQ |
| | | | KPGLAPQLLIYRAS |
| | | | NLESGIPDRFSGSG |
| | | | SRTDFTLTISRLEP |
| | | | EDFAVYYCQQSFDD |
| | | | PFTFGQGTKLEIK |
| SEQ ID | VL | E-H.12 | DIQLTQSPSSLSAS |
| NO: 3104 | | | VGDRVTITCRASES |
| | | | VDSSGNSFMHWYQQ |
| | | | KPGKAPQLLIYRAS |
| | | | NLESGVPSRFSGSG |
| | | | SRTDFTLTISSLQP |
| | | | EDFATYYCQQSFDD |
| | | | PFTFGQGTKLEIK |
| SEQ ID | VL | E-H.13 | DIQLTQSPSSVSA |
| NO: 3105 | | | SVGDRVTITCRAS |
| | | | ESVDSSGNSFMHW |
| | | | YQQKPGKAPQLLI |
| | | | YRASNLESGVPSR |
| | | | FSGSGSRTDFTLT |
| | | | ISSLQPEDFATYY |
| | | | CQQSFDDPFTFGQ |
| | | | GTKLEIK |
| SEQ ID | VL | E-H.14 | AIQLTQSPSSLSA |
| NO: 3106 | | | SVGDRVTITCRAS |
| | | | ESVDSSGNSFMHW |
| | | | YQQKPGKAPQLLI |
| | | | YRASNLESGVPSR |
| | | | FSGSGSRTDFTLT |
| | | | ISSLQPEDFATYY |
| | | | CQQSFDDPFTFGQ |
| | | | GTKLEIK |
| SEQ ID | VL | E-H.15 | DIQLTQSPSFLSA |
| NO: 3107 | | | SVGDRVTITCRAS |
| | | | ESVDSSGNSFMHW |
| | | | YQQKPGKAPQLLI |
| | | | YRASNLESGVPSR |
| | | | FSGSGSRTEFTLT |
| | | | ISSLQPEDFATYY |
| | | | CQQSFDDPFTFGQ |
| | | | GTKLEIK |
| SEQ ID | VL | E-H.16 | DIQLTQSPSSLSASV |
| NO: 3108 | | | GDRVTITCRASESVD |
| | | | SSGNSFMHWYQQKPG |
| | | | KAPQLLIYRASNLES |
| | | | GVPSRFSGSGSRTDF |
| | | | TFTISSLQPEDIATY |
| | | | YCQQSFDDPFTFGQG |
| | | | TKLEIK |
| SEQ ID | VL | E-H.17 | EIVLTQSPATLSVSP |
| NO: 3109 | | | GERATLSCRASESVD |
| | | | SSGNSFMHWYQQKPG |
| | | | QAPQLLIYRASNLES |
| | | | GIPARFSGSGSRTEF |
| | | | TLTISILQSEDFAVY |
| | | | YCQQSFDDPFTFGQG |
| | | | TKLEIK |
| SEQ ID | VL | E-H.18 | EIVLTQSPATLSVSP |
| NO: 3110 | | | GERATLSCRASESVD |
| | | | SSGNSFMHWYQQKPG |
| | | | QAPQLLIYRASNLES |
| | | | GIPARFSGSGSRTEF |
| | | | TLTISSLOSEDFAVY |
| | | | YCQQSFDDPFTFGQG |
| | | | TKLEIK |
| SEQ ID | VL | E-H.19 | AIRLTQSPFSLSAS |
| NO: 3111 | | | VGDRVTITCRASES |
| | | | VDSSGNSFMHWYQQ |
| | | | KPAKAPQLFIYRAS |
| | | | NLESGVPSRFSGSG |
| | | | SRTDFTLTISSLQP |
| | | | EDFATYYCQQSFDD |
| | | | PFTFGQGTKLEIK |
| SEQ ID | VL | E-H.20 | DIQLTQSPSSLSAS |
| NO: 3112 | | | VGDRVTITCRASES |
| | | | VDSSGNSFMHWYQQ |
| | | | KPGKAPQSLIYRAS |
| | | | NLESGVPSRFSGSG |
| | | | SRTDFTLTISSLQP |
| | | | EDFATYYCQQSFDD |
| | | | PFTFGQGTKLEIK |
| SEQ ID | VL | E-H.21 | DIQLTQSPSSLSAS |
| NO: 3113 | | | VGDRVTITCRASES |
| | | | VDSSGNSFMHWYQQ |
| | | | KPGKAPQRLIYRAS |
| | | | NLESGVPSRFSGSG |
| | | | SRTEFTLTISNLQP |
| | | | EDFATYYCQQSFDD |
| | | | PFTFGQGTKLEIK |
| SEQ ID | VL | E-H.22 | DIQLTQSPSTLSAS |
| NO: 3114 | | | VGDRVTITCRASES |
| | | | VDSSGNSFMHWYQQ |
| | | | KPGKAPQLLIYRAS |
| | | | NLESGVPSRFSGSG |
| | | | SRTEFTLTISSLQP |
| | | | DDFATYYCQQSFDD |
| | | | PFTFGQGTKLEIK |
| SEQ ID | VL | E-H.23 | EIVLTQSPDFQSVT |
| NO: 3115 | | | PKEKVTITCRASES |
| | | | VDSSGNSFMHWYQQ |
| | | | KPDQSPQLLIYRAS |
| | | | NLESGVPSRFSGSG |
| | | | SRTDFTLTINSLEA |
| | | | EDAATYYCQQSFDD |
| | | | PFTFGQGTKLEIK |
| SEQ ID | VL | E-H.24 | DIQLTQSPSSLSAS |
| NO: 3116 | | | VGDRVTITCRASES |
| | | | VDSSGNSFMHWYQQ |
| | | | KPGKAPQSLIYRAS |
| | | | NLESGVPSKFSGSG |
| | | | SRTDFTLTISSLQP |
| | | | EDFATYYCQQSFDD |
| | | | PFTFGQGTKLEIK |
| SEQ ID | VL | E-H.25 | DIQLTQSPSSLSAS |
| NO: 3117 | | | VGDRVTITCRASES |
| | | | VDSSGNSFMHWYQQ |
| | | | KPGKAPQRLIYRAS |
| | | | NLESGVPSRFSGSG |
| | | | SRTEFTLTISSLQP |
| | | | EDFATYYCQQSFDD |
| | | | PFTFGQGTKLEIK |
| SEQ ID | VL | E-H.26 | DIVLTQTPLSLSVT |
| NO: 3118 | | | PGQPASISCRASES |
| | | | VDSSGNSFMHWYLQ |
| | | | KPGQPPQLLIYRAS |
| | | | NLESGVPDRFSGSG |
| | | | SRTDFTLKISRVEA |
| | | | EDVGVYYCQQSFDD |
| | | | PFTFGQGTKLEIK |
| SEQ ID | VL | E-H.27 | DIQLTQSPSSLSAS |
| NO: 3119 | | | VGDRVTITCRASFS |
| | | | VDSSGNSFMHWYQQ |
| | | | KPEKAPQSLIYRAS |
| | | | NLESGVPSRFSGSG |
| | | | SRTDFTLTISSLQP |
| | | | EDFATYYCQQSFDD |
| | | | PFTFGQGTKLEIK |
| SEQ ID | VL | E-H.28 | EIVLTQSPPTLSLS |
| NO: 3120 | | | PGERVTLSCRASES |
| | | | VDSSGNSFMHWYQQ |
| | | | KPGQAPQLLIYRAS |
| | | | NLESGIPARFSGSG |
| | | | SRTDFTLTISSLQP |
| | | | EDFAVYYCQQSFDD |
| | | | PFTFGQGTKLEIK |
| SEQ ID | VL | E-H.29 | DIQLTQSPSAMSA |
| NO: 3121 | | | SVGDRVTITCRAS |
| | | | ESVDSSGNSFMHW |
| | | | YQQKPGKVPQRLI |
| | | | YRASNLESGVPSR |
| | | | FSGSGSRTEFTLT |
| | | | ISSLQPEDFATYY |
| | | | CQQSFDDPFTFGQ |
| | | | GTKLEIK |
| SEQ ID | VL | E-H.30 | DIVLTQSPLSLPV |
| NO: 3122 | | | TPGEPASISCRAS |
| | | | ESVDSSGNSFMHW |
| | | | YLQKPGQSPQLLI |
| | | | YRASNLESGVPDR |
| | | | FSGSGSRTDFTLK |
| | | | ISRVEAEDVGVYY |
| | | | CQQSFDDPFTFGQ |
| | | | GTKLEIK |
| SEQ ID | VL | E-H.31 | DIVLTQTPLSLPV |
| NO: 3123 | | | TPGEPASISCRAS |
| | | | ESVDSSGNSFMHW |
| | | | YLQKPGQSPQLLI |
| | | | YRASNLESGVPDR |
| | | | FSGSGSRTDFTLK |
| | | | ISRVEAEDVGVYY |
| | | | CQQSFDDPFTFGQ |
| | | | GTKLEIK |
| SEQ ID | VL | E-H.32 | DIVLTQTPLSLSV |
| NO: 3124 | | | TPGQPASISCRAS |
| | | | ESVDSSGNSFMHW |
| | | | YLQKPGQSPQLLI |
| | | | YRASNLESGVPDR |
| | | | FSGSGSRTDFTLK |
| | | | ISRVEAEDVGVYY |
| | | | CQQSFDDPFTFGQ |
| | | | GTKLEIK |
| SEQ ID | VL | E-H.33 | EIVLTQSPPTLSL |
| NO: 3125 | | | SPGERVTLSCRAS |
| | | | ESVDSSGNSFMHW |
| | | | YQQKPGQAPQLLI |
| | | | YRASNLESSIPAR |
| | | | FSGSGSRTDFTLT |
| | | | ISSLQPEDFAVYY |
| | | | CQQSFDDPFTFGQ |
| | | | GTKLEIK |
| SEQ ID | VL | E-H.34 | DIVLTQSPLSLPV |
| NO: 3126 | | | TLGQPASISCRAS |
| | | | ESVDSSGNSFMHW |
| | | | YQQKPGQSPQRLI |
| | | | YRASNLESGVPDR |
| | | | FSGSGSRTDFTLK |
| | | | ISRVEAEDVGVYY |
| | | | CQQSFDDPFTFGQ |
| | | | GTKLEIK |
| SEQ ID | VL | E-H.35 | DIVLTQTPLSSPV |
| NO: 3127 | | | TLGQPASISCRAS |
| | | | ESVDSSGNSFMHW |
| | | | YQQKPGQPPQLLI |
| | | | YRASNLESGVPDR |
| | | | FSGSGARTDFTLK |
| | | | ISRVEAEDVGVYY |
| | | | CQQSFDDPFTFGQ |
| | | | GTKLEIK |
| SEQ ID | VL | E-H.36 | DIVLTQSPAFLSV |
| NO: 3128 | | | TPGEKVTITCRAS |
| | | | ESVDSSGNSFMHW |
| | | | YQQKPDQAPQLLI |
| | | | YRASNLESGVPSR |
| | | | FSGSGSRTDFTFT |
| | | | ISSLEAEDAATYY |
| | | | CQQSFDDPFTFGQ |
| | | | GTKLEIK |
| SEQ ID | VL | E-H.37 | DIQLIQSPSFLSA |
| NO: 3129 | | | SVGDRVSIICRAS |
| | | | ESVDSSGNSFMHW |
| | | | YLQKPGKSPQLFI |
| | | | YRASNLESGVSSR |
| | | | FSGRGSRTDFTLT |
| | | | IISLKPEDFAAYY |
| | | | CQQSFDDPFTFGQ |
| | | | GTKLEIK |
| SEQ ID | VL | E-H.38 | EIVLTQTPLSLSI |
| NO: 3130 | | | TPGEQASISCRAS |
| | | | ESVDSSGNSFMHW |
| | | | YLQKARPVPQLLI |
| | | | YRASNLESGVPDR |
| | | | FSGSGSRTDFTLK |
| | | | ISRVEAEDFGVYY |
| | | | CQQSFDDPFTFGQ |
| | | | GTKLEIK |
| SEQ ID | VL | E-H.39 | EIVLTQTPLSLSI |
| NO: 3131 | | | TPGEQASMSCRAS |
| | | | ESVDSSGNSFMHW |
| | | | YLQKARPVPQLLI |
| | | | YRASNLESGVPDR |
| | | | FSGSGSRTDFTLK |
| | | | ISRVEAEDFGVYY |
| | | | CQQSFDDPFTFGQ |
| | | | GTKLEIK |
| SEQ ID | VL | E-H.40 | EITLTQSPAFMSA |
| NO: 3132 | | | TPGDKVNISCRAS |
| | | | ESVDSSGNSFMHW |
| | | | YQQKPGEAPQFII |
| | | | YRASNLESGIPPR |
| | | | FSGSGYRTDFTLT |
| | | | INNIESEDAAYYY |
| | | | CQQSFDDPFTFGQ |
| | | | GTKLEIK |

Variable HEAVY chain (VH)

| | | | |
|---|---|---|---|
| SEQ ID | VH | E-H.1 | QVQLVQSGAEVKKP |
| NO: 3133 | | | GASVKVSCKASGYA |
| | | | FSSSWMNWVRQAPG |
| | | | QGLEWIGRIYPGDG |
| | | | DTKYNGKFKGRATL |
| | | | TADKSTSTAYMELS |
| | | | SLRSEDTAVYYCAR |
| | | | RGTGGWYFDVWGQG |
| | | | TTVTVSS |
| SEQ ID | VH | E-H.2 | QVQLVQSGAEVKKP |
| NO: 3134 | | | GSSVKVSCKASGYA |
| | | | FSSSWMNWVRQAPG |
| | | | QGLEWIGRIYPGDG |
| | | | DTKYNGKFKGRATL |
| | | | TADKSTSTAYMELS |
| | | | SLRSEDTAVYYCAR |
| | | | RGTGGWYFDVWGQG |
| | | | TTVTVSS |
| SEQ ID | VH | E-H.3 | QVQLVQSGAEVKKP |
| NO: 3135 | | | GASVKVSCKASGYA |
| | | | FSSSWMNWVRQAPG |
| | | | KGLEWIGRIYPGDG |
| | | | DTKYNGKFKGRATL |
| | | | TADKSTSTAYMELS |
| | | | SLRSEDTAVYYCAR |
| | | | RGTGGWYFDVWGQG |
| | | | TTVTVSS |
| SEQ ID | VH | E-H.4 | QVQLVQSGAEVKKP |
| NO: 3136 | | | GASVKVSCKASGYA |
| | | | FSSSWMNWVRQAPG |
| | | | QELEWIGRIYPGDG |
| | | | DTKYNGKFKGRATL |
| | | | TADKSISTAYMELS |
| | | | SLRSEDTATYYCAR |
| | | | RGTGGWYFDVWGQG |
| | | | TTVTVSS |
| SEQ ID | VH | E-H.5 | EVQLVQSGAEVKKP |
| NO: 3137 | | | GATVKISCKASGYA |
| | | | FSSSWMNWVQQAPG |
| | | | KGLEWIGRIYPGDG |
| | | | DTKYNGKFKGRATL |
| | | | TADKSTSTAYMELS |
| | | | SLRSEDTAVYYCAR |
| | | | RGTGGWYFDVWGQG |
| | | | TTVTVSS |
| SEQ ID | VH | E-H.6 | QVQLVQSGAEVKKT |
| NO: 3138 | | | GSSVKVSCKASGYA |
| | | | FSSSWMNWVRQAPG |
| | | | QALEWIGRIYPGDG |
| | | | DTKYNGKFKGRATL |
| | | | TADKSMSTAYMELS |
| | | | SLRSEDTAMYYCAR |
| | | | RGTGGWYFDVWGQG |
| | | | TTVTVSS |
| SEQ ID | VH | E-H.7 | QVQLVQSGAEVKKP |
| NO: 3139 | | | GASVKVSCKASGYA |
| | | | FSSSWMNWVRQAPG |
| | | | QRLEW1GRIYPGDG |
| | | | DTKYNGKFKGRATL |
| | | | TADKSASTAYMELS |
| | | | SLRSEDMAVYYCAR |
| | | | RGTGGWYFDVWGQG |
| | | | TTVTVSS |
| SEQ ID | VH | E-H.8 | QVQLVQSGAEVKKP |
| NO: 3140 | | | GASVKVSCKASGYA |
| | | | FSSSWMNWVRQAPG |
| | | | QGLEWIGRIYPGDG |
| | | | DTKYNGKFKGRATL |
| | | | TADKSTSTAYMELR |
| | | | SLRSDDMAVYYCAR |
| | | | RGTGGWYFDVWGQG |
| | | | TFVTVSS |
| SEQ ID | VH | E-H.9 | QVQLVQSGAEVKKP |
| NO: 3141 | | | GASVKVSCKASGYA |
| | | | FSSSWMNWVRQAPG |
| | | | QRLEWIGRIYPGDG |
| | | | DTKYNGKFKGRATL |
| | | | TADKSASTAYMELS |
| | | | SLRSEDTAVYYCAR |
| | | | RGTGGWYFDVWGQG |
| | | | TTVTVSS |
| SEQ ID | VH | E-H.10 | QVQLVQSGAEVKKP |
| NO: 3142 | | | GASVKVSCKASGYA |
| | | | FSSSWMNWVRQAPG |
| | | | QGLEWIGRIYPGDG |
| | | | DTKYNGKFKGRATL |
| | | | TADKSTSTAYMELR |
| | | | SLRSDDTAVYYCAR |
| | | | RGTGGWYFDVWGQG |
| | | | TTVTVSS |
| SEQ ID | VH | E-H.11 | QVQLVQSGAEVKKP |
| NO: 3143 | | | GASVKVSCKASGYA |
| | | | FSSSWMNWVRQAPG |
| | | | QGLEWIGRIYPGDG |
| | | | DTKYNGKFKGRATL |
| | | | TADKSISTAYMELS |
| | | | RLRSDDTAVYYCAR |
| | | | RGTGGWYFDVWGQG |
| | | | TTVTVSS |
| SEQ ID | VH | E-H.12 | QVQLVQSGAEVKKP |
| NO: 3144 | | | GASVKVSCKASGYA |
| | | | FSSSWMNWVRQAPG |
| | | | QGLEWIGRIYPGDG |
| | | | DTKYNGKFKGRATL |
| | | | TADKSISTAYMELS |
| | | | RLRSDDTVVYYCAR |
| | | | RGTGGWYFDVWGQG |
| | | | TTVTVSS |
| SEQ ID | VH | E-H.13 | QVQLVQSGAEVKKP |
| NO: 3145 | | | GASVKVSCKASGYA |
| | | | FSSSWMNWVRQAPG |
| | | | QGLEWIGRIYPGDG |
| | | | DTKYNGKFKGWATL |
| | | | TADKSISTAYMELS |
| | | | RLRSDDTAVYYCAR |
| | | | RGTGGWYFDVWGQG |
| | | | TTVTVSS |
| SEQ ID | VH | E-H.14 | QVQLVQSGAEVKKP |
| NO: 3146 | | | GASVKVSCKASGYA |
| | | | FSSSWMNWVRQATG |
| | | | QGLEWIGRIYPGDG |
| | | | DTKYNGKFKGRATL |
| | | | TANKSISTAYMELS |
| | | | SLRSEDTAVYYCAR |
| | | | RGTGGWYFDVWGQG |
| | | | TTVTVSS |
| SEQ ID | VH | E-H.15 | QVQLVQSGSELKKP |
| NO: 3147 | | | GASVKVSCKASGYA |
| | | | FSSSWMNWVRQAPG |
| | | | QGLEWIGRIYPGDG |
| | | | DTKYNGKFKGRAVL |
| | | | SADKSVSTAYLQIS |
| | | | SLKAEDTAVYYCAR |
| | | | RGTGGWYFDVWGQG |
| | | | TTVTVSS |
| SEQ ID | VH | E-H.16 | QVQLVQSGPEVKKP |
| NO: 3148 | | | GTSVKVSCKASGYA |
| | | | FSSSWMNWVRQARG |
| | | | QRLEWIGRIYPGDG |
| | | | DTKYNGKFKGRATL |
| | | | TADKSTSTAYMELS |
| | | | SLRSEDTAVYYCAR |
| | | | RGTGGWYFDVWGQG |
| | | | TTVTVSS |
| SEQ ID | VH | E-H.17 | EVQLVQSGAEVKKP |
| NO: 3149 | | | GESLKISCKASGYA |
| | | | FSSSWMNWVRQMPG |
| | | | KGLEWIGRIYPGDG |
| | | | DTKYNGKFKGQATL |
| | | | SADKSISTAYLQWS |
| | | | SLKASDTAMYYCAR |
| | | | RGTGGWYFDVWGQG |
| | | | TTVTVSS |
| SEQ ID | VH | E-H.18 | QVQLVQSGSELKKP |
| NO: 3150 | | | GASVKVSCKASGYA |
| | | | FSSSWMNWVRQAPG |
| | | | QGLEWIGRIYPGDG |
| | | | DTKYNGKFKGRAVL |
| | | | SADKSVSMAYLQIS |
| | | | SLKAEDTAVYYCAR |
| | | | RGTGGWYFDVWGQG |
| | | | TTVTVSS |
| SEQ ID | VH | E-H.19 | QVQLVQSGHEVKQP |
| NO: 3151 | | | GASVKVSCKASGYA |
| | | | FSSSWMNWVPQAPG |
| | | | QGLEWIGRIYPGDG |
| | | | DTKYNGKFKGRAVL |
| | | | SADKSASTAYLQIS |
| | | | SLKAEDMAMYYCAR |
| | | | RGTGGWYFDVWGQG |
| | | | TTVTVSS |
| SEQ ID | VH | E-H.20 | EVQLVQSGAEVKKP |
| NO: 3152 | | | GESLKISCKASGYA |
| | | | FSSSWMNWVRQMPG |
| | | | KGLEWIGRIYPGDG |
| | | | DTKYNGKFKGQATL |
| | | | SADKPISTAYLQWS |
| | | | SLKASDTAMYYCAR |
| | | | RGTGGWYFDVWGQG |
| | | | TTVTVSS |
| SEQ ID | VH | E-H.21 | EVQLVQSGAEVKKP |
| NO: 3153 | | | GESLRISCKASGYA |
| | | | FSSSWMNWVRQMPG |
| | | | KGLEWIGRIYPGDG |
| | | | DTKYNGKFKGQATL |
| | | | SADKSISTAYLQWS |
| | | | SLKASDTAMYYCAR |
| | | | RGTGGWYFDVWGQG |
| | | | TTVTVSS |
| SEQ ID | VH | E-H.22 | EVQLVQSGAEVKKP |
| NO: 3154 | | | GESLRISCKASGYA |
| | | | FSSSWMNWVRQMPG |
| | | | KGLEWIGRIYPGDG |
| | | | DTKYNGKFKGHATL |
| | | | SADKSISTAYLQWS |
| | | | SLKASDTAMYYCAR |
| | | | RGTGGWYFDVWGQG |
| | | | TTVTVSS |
| SEQ ID | VH | E-H.23 | QVQLVQSGAEVKKT |
| NO: 3155 | | | GSSVKVSCKASGYA |
| | | | FSSSWMNWVRQAPR |
| | | | QALEWIGRIYPGDG |
| | | | DTKYNGKFKGRATL |
| | | | TADKSMSTAYMELS |
| | | | SLRSEDTAMYYCAR |
| | | | RGTGGWYFDVWGQG |
| | | | TTVTVSS |
| SEQ ID | VH | E-H.24 | EVQLVESGGGLVQP |
| NO: 3156 | | | GRSLRLSCTASGYA |
| | | | FSSSWMNWVRQAPG |
| | | | KGLEWIGRIYPGDG |
| | | | DTKYNGKFKGRATL |
| | | | SADKSKSIAYLQMN |
| | | | SLKTEDTAVYYCAR |
| | | | RGTGGWYFDVWGQG |
| | | | TTVTVSS |
| SEQ ID | VH | E-H.25 | EVQLVESGGGLVQP |
| NO: 3157 | | | GPSLRLSCTASGYA |
| | | | FSSSWMNWVRQAPG |
| | | | KGLEWIGRIYPGDG |
| | | | DTKYNGKFK |
| | | | GRATLSADKSKSIA |
| | | | YLQMNSLKTEDTAV |
| | | | YYCARRGTGGWYFD |
| | | | VWGQGTTVTVSS |
| SEQ ID | VH | E-H.26 | QVQLQESGPGLVKP |
| NO: 3158 | | | SQTLSLTCTASGYA |
| | | | FSSSWMNWVRQPPG |
| | | | KGLEWIGRIYPGDG |
| | | | DTKYNGKFKGRATL |
| | | | SADKSKSQASLKLS |
| | | | SVTAADTAVYYCAR |
| | | | RGTGGWYFDVWGQG |
| | | | TTVTVSS |
| SEQ ID | VH | E-H.27 | QVQLQESGPGLVKP |
| NO: 3159 | | | SGTLSLTCAASGYA |
| | | | FSSSWMNWVRQPPG |
| | | | KGLEWIGRIYPGDG |
| | | | DTKYNGKFKGRATL |
| | | | SADKSKSQASLKLS |
| | | | SVTAADTAVYYCAR |
| | | | RGTGGWYFDVWGQG |
| | | | TTVTVSS |
| SEQ ID | VH | E-H.28 | EVQLVESGGGLVKP |
| NO: 3160 | | | GRSLRLSCTASGYA |
| | | | FSSSWMNWVRQAPG |
| | | | KGLEWIGRIYPGDG |
| | | | DTKYNGKFKGRATL |
| | | | SADKSKSIAYLQMN |
| | | | SLKTEDTAVYYCAR |
| | | | RGTGGWYFDVWGQG |
| | | | TTVTVSS |
| SEQ ID | VH | E-H.29 | EVQLVESGGGLVQP |
| NO: 3161 | | | GGSLKLSCAASGYA |
| | | | FSSSWMNWVRQASG |
| | | | KGLEWIGRIYPGDG |
| | | | DTKYNGKFKGRATL |
| | | | SADKSKSTAYLQMN |
| | | | SLKTEDTAVYYCAR |
| | | | RGTGGWYFDVWGQG |
| | | | TTVTVSS |
| SEQ ID | VH | E-H.30 | QVQLQESGPGLVKP |
| NO: 3162 | | | SQTLSLTCAASGYA |
| | | | FSSSWMNWVRQPPG |
| | | | KGLEWIGRIYPGDG |
| | | | DTKYNGKFKGRATL |
| | | | SADKSKSQASLKLS |
| | | | SVTAADTAVYYCAR |
| | | | RGTGGWYFDVWGQG |
| | | | TTVTVSS |
| SEQ ID | VH | E-H.31 | EVQLVESGGGLVKP |
| NO: 3163 | | | GGSLRLSCAASGYA |
| | | | FSSSWMNWVRQAPG |
| | | | KGLEWIGRIYPGDG |
| | | | DTKYNGKFKGRATL |
| | | | SADKSKSTAYLQMN |
| | | | SLKTEDTAVYYCAR |
| | | | RGTGGWYFDVWGQG |
| | | | TTVTVSS |
| SEQ ID | VH | E-H.32 | EVQLVESGGALVKP |
| NO: 3164 | | | GGSLRLSCAASGYA |
| | | | FSSSWMNWVRQAPG |
| | | | KGLEWIGRIYPGDG |
| | | | DTKYNGKFKGRATL |
| | | | SADKSKSTAYLQMN |
| | | | SLKTEDTAVYYCAR |
| | | | RGTGGWYFDVWGQG |
| | | | TTVTVSS |
| SEQ ID | VH | E-H.33 | QVQLQESGPGLVKP |
| NO: 3165 | | | SQTLSLTCAAYGYA |
| | | | FSSSWMNWVRQPPG |
| | | | KGLEWIGRIYPGDG |
| | | | DTKYNGKFKGRATL |
| | | | SADKSKSQASLKLS |
| | | | SVTAADTAVYYCAR |
| | | | RGTGGWYFDVWGQG |
| | | | TTVTVSS |
| SEQ ID | VH | E-H.34 | QVQLQESGSGLVKP |
| NO: 3166 | | | SQTLSLTCAASGYA |
| | | | FSSSWMNWVRQPPG |
| | | | KGLEWIGRIYPGDG |
| | | | DTKYNGKFKGRATL |
| | | | SADKSKSQASLKLS |
| | | | SVTAADTAVYYCAR |
| | | | RGTGGWYFDVWGQG |
| | | | TTVTVSS |
| SEQ ID | VH | E-H.35 | EVQLVESGGGLVQPG |
| NO: 3167 | | | GSLRLSCAASGYAFS |
| | | | SSWMNWVRQAPGKGL |
| | | | EWIGRIYPGDGDTKY |
| | | | NGKFKGRATLSADKS |
| | | | KSSAYLQMNSLKTED |
| | | | TAVYYCARRGTGGWY |
| | | | FDVWGQGTTVTVSS |
| SEQ ID | VH | E-H.36 | QVQLQESGPGLVKPS |
| NO: 3168 | | | DTLSLTCTASGYAFS |
| | | | SSWMNWVRQPPGKGL |
| | | | EWIGRIYPGDGDTKY |
| | | | NGKFKGRATLSADK |
| | | | SKSQASLKLSSVTA |
| | | | ADTAVYYCARRGTG |
| | | | GWYFDVWGQGTTVT |
| | | | VSS |
| SEQ ID | VH | E-H.37 | QVQLQESGPGLVKPS |
| NO: 3169 | | | QTLSLTCTASGYAFS |
| | | | SSWMNWVRQHPGKGL |
| | | | EWIGRIYPGDGDTKY |
| | | | NGKFKGRATLSADKS |
| | | | KSQASLKLSSVTAAD |
| | | | TAVYYCARRGTGGWY |
| | | | FDVWGQGTTVTVSS |
| SEQ ID | VH | E-H.38 | QVQLQESGPGLVK |
| NO: 3170 | | | PSQTLSLTCTASG |
| | | | YAFSSSWMNWVRQ |
| | | | HPGKGLEWIGRIY |
| | | | PGDGDTKYNGKFK |
| | | | GLATLSADKSKSQ |
| | | | ASLKLSSVTAADT |
| | | | AVYYCARRGTGGW |
| | | | YFDVWGQGTTVTV |
| | | | SS |
| SEQ ID | VH | E-H.39 | QVQLVESGGGVVQ |
| NO: 3171 | | | PGRSLRLSCAASG |
| | | | YAFSSSWMNWVRQ |
| | | | APGKGLEWIGRIY |
| | | | PGDGDTKYNGKFK |
| | | | GRATLSADKSKST |
| | | | AYLQMSSLRAEDT |
| | | | AVYYCARRGTGGW |
| | | | YFDVWGQGTTVTV |
| | | | SS |
| SEQ ID | VH | E-H.40 | QVQLVESGGGLVK |
| NO: 3172 | | | PGGSLRLSCAASG |
| | | | YAFSSSWMNWVRQ |
| | | | APGKGLEWIGRIY |
| | | | PGDGDTKYNGKFK |
| | | | GRATLSADKAKSS |
| | | | AYLQMNSLRAEDT |
| | | | AVYYCARRGTGGW |
| | | | YFDVWGQGTTVTV |
| | | | SS |
| SEQ ID | VH | E-H.41 | QVQLVESGGGLVQ |
| NO: 3173 | | | PGGSLRLSCSASG |
| | | | YAFSSSWMNWVRQ |
| | | | APGKGLEWIGRIY |
| | | | PGDGDTKYNGKFK |
| | | | GRATLSADKSKST |
| | | | AYLQMNSLRAEDT |
| | | | AVYYCARRGTGGW |
| | | | YFDVWGQGTTVTV |
| | | | SS |
| SEQ ID | VH | E-H.42 | QVQLLESGGGLVK |
| NO: 3174 | | | PGGSLRLSCAASG |
| | | | YAFSSSWMNWVRQ |
| | | | APGKGLEWIGRIY |
| | | | PGDGDTKYNGKFK |
| | | | GRATLSADKAKSS |
| | | | AYLQMNSLRAEDT |
| | | | AVYYCARRGTGGW |
| | | | YFDVWGQGTTVTV |
| | | | SS |
| SEQ ID | VH | E-H.43 | EVQLVESGGGLVQ |
| NO: 3175 | | | PGGSLRLSCSASG |
| | | | YAFSSSWMNWVRQ |
| | | | APGKGLEWIGRIY |
| | | | PGDGDTKYNGKFK |
| | | | GRATLSADKSKST |
| | | | AYLQMSSLRAEDT |
| | | | AVYYCARRGTGGW |
| | | | YFDVWGQGTTVTV |
| | | | SS |
| SEQ ID | VH | E-H.44 | QVQLQESGPGLVK |
| NO: 3176 | | | PSDTLSLTCAASG |
| | | | YAFSSSWMNWVRQ |
| | | | PPGKGLEWIGRIY |
| | | | PGDGDTKYNGKFK |
| | | | GRATLSADKSKSQ |
| | | | ASLKLSSVTAVDT |
| | | | AVYYCARRGTGGW |
| | | | YFDVWGQGTTVTV |
| | | | SS |
| SEQ ID | VH | E-H.45 | QVQLQESGPGLVK |
| NO: 3177 | | | PSQTLSLTCAASG |
| | | | YAFSSSWMNWVRQ |
| | | | PPGKGLEWIGRIY |
| | | | PGDGDTKYNGKFK |
| | | | GRATLSADKSKSQ |
| | | | ASLKLSSVTAVDT |
| | | | AVYYCARRGTGGW |
| | | | YFDVWGQGTTVTV |
| | | | SS |
| SEQ ID | VH | E-H.46 | EVQLVESGGGLVQ |
| NO: 3178 | | | PGGSLRLSCSASG |
| | | | YAFSSSWMNWVRQ |
| | | | APGKGLEWIGRIY |
| | | | PGDGDTKYNGKFK |
| | | | GRATLSADKSKST |
| | | | AYVQMSSLRAEDT |
| | | | AVYYCARRGTGGW |
| | | | YFDVWGQGTTVTV |
| | | | SS |
| SEQ ID | VH | E-H.47 | QVQLVDSGGGVVQ |
| NO: 3179 | | | PGRSLRLSCAASG |
| | | | YAFSSSWMNWVRQ |
| | | | APGKGLEWIGRIY |
| | | | PGDGDTKYNGKFK |
| | | | GRATLSADKSKST |
| | | | AYLQMNSLRAEDT |
| | | | AVYYCARRGTGGW |
| | | | YFDVWGQGTTVTV |
| | | | SS |
| SEQ ID | VH | E-H.48 | QVQLVESGGGVVQ |
| NO: 3180 | | | PGRSLRLSCAASG |
| | | | YAFSSSWMNWVRQ |
| | | | APGKGLEWIGRIY |
| | | | PGDGDTKYNGKFK |
| | | | GRATLSADKSKST |
| | | | AYLQMNSLRAEGT |
| | | | AVYYCARRGTGGW |
| | | | YFDVWGQGTTVTV |
| | | | SS |
| SEQ ID | VH | E-H.49 | QVQLVESGGGVVQ |
| NO: 3181 | | | PGRSLRLSCAASG |
| | | | YAFSSSWMNWVRQ |
| | | | APGKGLEWIGRIY |
| | | | PGDGDTKYNGKFK |
| | | | GRATLSADKSKST |
| | | | AYLQMNSLRAEDT |
| | | | AVYYCARRGTGGW |
| | | | YFDVWGQGTTVTV |
| | | | SS |
| SEQ ID | VH | E-H.50 | EVQLVESGGGLVQ |
| NO: 3182 | | | PGGSLRLSCAASG |
| | | | YAFSSSWMNWVRQ |
| | | | APGKGLEWIGRIY |
| | | | PGDGDTKYNGKFK |
| | | | GRATLSADKSKST |
| | | | AYLQMNSLRAEDT |
| | | | AVYYCARRGTGGW |
| | | | YFDVWGQGTTVTV |
| | | | SS |

In some embodiments, the anti-TCRβV5 antibody molecule comprises a VH and/or a VL of an antibody described in Table 6, or a sequence with at least 80%, 85%, 90%, 95%, 96%, 97%, 98%, 99% or more identity thereto.

In some embodiments, the anti-TCRβV5 antibody molecule comprises a VH and a VL of an antibody described in Table 6, or a sequence with at least 80%, 85%, 90%, 95%, 96%, 97%, 98%, 99% or more identity thereto.

In some embodiments, the anti-TCRβV5 antibody molecule comprises a VH and/or a VL of an antibody described in Table 7, or a sequence with at least 80%, 85%, 90%, 95%, 96%, 97%, 98%, 99% or more identity thereto.

In some embodiments, the anti-TCRβV5 antibody molecule comprises a VH and a VL of an antibody described in Table 7, or a sequence with at least 80%, 85%, 90%, 95%, 96%, 97%, 98%, 99% or more identity thereto.

### Anti-TCRβV10 Antibodies

Accordingly, in one aspect, the disclosure provides an anti-TCRβV antibody molecule that binds to a human TCRβ V10 subfamily member. In some embodiments, TCRβ V10 subfamily is also known as TCRβ V12. In some embodiments, the TCRβ V10 subfamily comprises: TCRβ V10-1*01, TCRβ V10-1*02, TCRβ V10-3*01 or TCRβ V10-2*01, or a variant thereof.

Exemplary anti-TCRβV10 antibodies of the disclosure are provided in Table 8. In some embodiments, the anti-TCRβV10 is antibody D, e.g., humanized antibody D (antibody D-H), as provided in Table 8. In some embodiments, antibody D comprises one or more (e.g., three) light chain CDRs and/or one or more (e.g., three) heavy chain CDRs provided in Table 8, or a sequence with at least 95% identity thereto. In some embodiments, antibody D comprises a variable heavy chain (VH) and/or a variable light chain (VL) provided in Table 8, or a sequence with at least 95% identity thereto.

TABLE 8
Amino acid sequences for anti
TCRβ V10 antibodies
Amino acid and nucleotide sequences
for murine and humanized antibody
molecules which bindto TCRBV 10
(e.g., TCRBV 10-1,TCRBV 10-2
Or TCRBV 10-3). The amino acid
the heavy and light chain CDRs,
and the amino acid and nucleotide
sequences of the heavy and light
chain variable regions, and the
heavy and light chains are shown.
Murine antibody D

| | | |
|---|---|---|
| SEQ ID NO: 1288 | HC CDR1 | SYGMS |
| | (Rabat) | |
| SEQ ID NO: 1289 | HC CDR2 | LISSGGSYTYYTDSVRG |
| | (Rabat) | |
| SEQ ID NO: 1290 | HC CDR3 | HGGNFFDY |
| | (Rabat) | |
| SEQ ID NO: 1291 | HC CDR1 | GFTFRSY |
| | (Chothia) | |
| SEQ ID NO: 1292 | HC CDR2 | SSGGSY |
| | (Chothia) | |
| SEQ ID NO: 1290 | HC CDR3 | HGGNFFDY |
| | (Chothia) | |
| SEQ ID NO: 1293 | HC CDR1 | GFTFRSYGMS |
| | (Combined) | |
| SEQ ID NO: 1289 | HC CDR2 | LISSGGSYTYYTDSVRG |
| | (Combined) | |
| SEQ ID NO: 1290 | HC CDR3 | HGGNFFDY |
| | (Combined) | |
| SEQ ID NO: 1294 | LC CDR1 | SVSSSVSYMH |
| | (Rabat) | |
| SEQ ID NO: 1295 | LC CDR2 | DTSRLAS |
| | (Rabat) | |
| SEQ ID NO: 1296 | LC CDR3 | QQWSSNPQYT |
| | (Rabat) | |
| SEQ ID NO: 1297 | LC CDR1 | SSSVSY |
| | (Chothia) | |
| SEQ ID NO: 1295 | LC CDR2 | DTSRLAS |
| | (Chothia) | |
| SEQ ID NO: 1296 | LC CDR3 | QQWSSNPQYT |
| | (Chothia) | |
| SEQ ID NO: 1294 | LC CDR1 | SVSSSVSYMH |
| | (Combined) | |
| SEQ ID NO: 1295 | LC CDR2 | DTSRLAS |
| | (Combined) | |
| SEQ ID NO: 1296 | LC CDR3 | QQWSSNPQYT |
| | (Combined) | |
| SEQ ID NO: 3183 | VH | EVQLVESGGDLVKPGGSLKLSCAVSGFTFRSYG |
| | | MSWVRQTPDKRLEWVALISSGGSYTYYTDSVKG |
| | | RFTISRDNAKNTLYLQMSSLKSEDTAIYYCSRHG |
| | | GNFFDYWGQGTTLTVSS |
| SEQ ID NO: 3184 | VL | QIVLTQSPSIMSASPGEKVTMTCSVSSSVSYMHW |
| | | YQQKSGTSPKRWIYDTSKLASGVPARFSGSGSGT |
| | | SYSLTISSMEAEDAATYYCQQWSSNPQYTFGGG |
| | | TKLEIK |

Humanized antibody D (D-H antibody)
Variable light chain (VL)

| | | | |
|---|---|---|---|
| SEQ ID NO: 3185 | VL | D-VL-H.1 | DIVLTQSPAFLSVTPGEKVTITCSVSSSVSYMHW |
| | | | YQQKPDQAPKLLIYDTSKLASGVPSRFSGSGSGT |
| | | | DYTFTISSLEAEDAATYYCQQWSSNPQYTFGQGT |
| | | | KLEIK |
| SEQ ID NO: 3186 | VL | D-VL-H.2 | AIQLTQSPSSLSASVGDRVTITCSVSSSVSYMHW |
| | | | YQQKPGKAPKLLIYDTSKLASGVPSRFSGSGSGT |
| | | | DYTLTISSLQPEDFATYYCQQWSSNPQYTFGQGT |
| | | | KLEIK |
| SEQ ID NO: 3187 | VL | D-VL-H.3 | DIQLTQSPSFLSASVGDRVTITCSVSSSVSYMHW |
| | | | YQQKPGKAPKLLIYDTSKLASGVPSRFSGSGSGT |
| | | | EYTLTISSLQPEDFATYYCQQWSSNPQYTFGQGT |
| | | | KLEIK |
| SEQ ID NO: 3188 | VL | D-VL-H.4 | DIQLTQSPSSLSASVGDRVTITCSVSSSVSYMHW |
| | | | YQQKPGKAPKLLIYDTSKLASGVPSRFSGSGSGT |
| | | | DYTLTISSLQPEDFATYYCQQWSSNPQYTFGQGT |
| | | | KLEIK |
| SEQ ID NO: 3189 | VL | D-VL-H.5 | DIQLTQSPSSVSASVGDRVTITCSVSSSVSYMHW |
| | | | YQQKPGKAPKLLIYDTSKLASGVPSRFSGSGSGT |
| | | | DYTLTISSLQPEDFATYYCQQWSSNPQYTFGQGT |
| | | | KLEIK |
| SEQ ID NO: 3190 | VL | D-VL-H.6 | DIQLTQSPSSLSASVGDRVTITCSVSSSVSYMHW |
| | | | YQQKPGKVPKLLIYDTSKLASGVPSRFSGSGSGT |
| | | | DYTLTISSLQPEDVATYYCQQWSSNPQYTFGQGT |
| | | | KLEIK |
| SEQ ID NO: 3191 | VL | D-VL-H.7 | DIQLTQSPSSLSASVGDRVTITCSVSSSVSYMHW |
| | | | YQQKPGQAPKLLIYDTSKLASGVPSRFSGSGSGT |
| | | | DYTLTISSLQPEDVATYYCQQWSSNPQYTFGQGT |
| | | | KLEIK |
| SEQ ID NO: 3192 | VL | D-VL-H.8 | EIVLTQSPDFQSVTPKEKVTITCSVSSSVSYMHW |
| | | | YQQKPDQSPKLLIYDTSKLASGVPSRFSGSGSGT |
| | | | DYTLTINSLEAEDAATYYCQQWSSNPQYTFGQG |
| | | | TKLEIK |
| SEQ ID NO: 3193 | VL | D-VL-H.9 | AIRLTQSPFSLSASVGDRVTITCSVSSSVSYMHW |
| | | | YQQKPAKAPKLFIYDTSKLASGVPSRFSGSGSGT |
| | | | DYTLTISSLQPEDFATYYCQQWSSNPQYTFGQGT |
| | | | KLEIK |
| SEQ ID NO: 3194 | VL | D-VL-H.10 | DIQLTQSPSSLSASVGDRVTITCSVSSSVSYMHW |
| | | | YQQKPGKAPKLLIYDTSKLASGVPSRFSGSGSGT |
| | | | DYTFTISSLQPEDIATYYCQQWSSNPQYTFGQGT |
| | | | KLEIK |
| SEQ ID NO: 3195 | VL | D-VL-H.11 | EIVLTQSPATLSLSPGERATLSCSVSSSVSYMHW |
| | | | YQQKPGQAPKLLIYDTSKLASGIPARFSGSGSGT |
| | | | DYTLTISSLEPEDFAVYYCQQWSSNPQYTFGQGT |
| | | | KLEIK |
| SEQ ID NO: 3196 | VL | D-VL-H.12 | DIQLTQSPSTLSASVGDRVTITCSVSSSVSYMHW |
| | | | YQQKPGKAPKLLIYDTSKLASGVPSRFSGSGSGT |
| | | | EYTLTISSLQPDDFATYYCQQWSSNPQYTFGQGT |
| | | | KLEIK |
| SEQ ID NO: 3197 | VL | D-VL-H.13 | DIQLTQSPSSLSASVGDRVTITCSVSSSVSYMHW |
| | | | YQQKPGKTPKLLIYDTSKLASGIPSRFSGSGSGTD |
| | | | YTLTIRSLQPEDFATYYCQQWSSNPQYTFGQGTK |
| | | | LEIK |
| SEQ ID NO: 3198 | VL | D-VL-H.14 | EIVLTQSPPTLSLSPGERVTLSCSVSSSVSYMHWY |
| | | | QQKPGQAPKLLIYDTSKLASGIPARFSGSGSGTD |
| | | | YTLTISSLQPEDFAVYYCQQWSSNPQYTFGQGTK |
| | | | LEIK |
| SEQ ID NO: 3199 | VL | D-VL-H.15 | DIQLTQSPSSLSASVGDRVTITCSVSSSVSYMHW |
| | | | YQQKPGKAPKRLIYDTSKLASGVPSRFSGSGSGT |
| | | | EYTLTISSLQPEDFATYYCQQWSSNPQYTFGQGT |
| | | | KLEIK |
| SEQ ID NO: 3200 | VL | D-VL-H.16 | EIVLTQSPATLSLSPGERATLSCSVSSSVSYMHW |
| | | | YQQKPGQAPKLLIYDTSKLASGIPARFSGSGPGT |
| | | | DYTLTISSLEPEDFAVYYCQQWSSNPQYTFGQGT |
| | | | KLEIK |
| SEQ ID NO: 3201 | VL | D-VL-H.17 | EIVLTQSPATLSLSPGERATLSCSVSSSVSYMHW |
| | | | YQQKPGQAPKLLIYDTSKLASGIPARFSGSGSGT |
| | | | DYTLTISRLEPEDFAVYYCQQWSSNPQYTFGQGT |
| | | | KLEIK |
| SEQ ID NO: 3202 | VL | D-VL-H.18 | EIVLTQSPATLSLSPGERATLSCSVSSSVSYMHW |
| | | | YQQKPGQAPKLLIYDTSKLASGIPARFSGSGSGT |
| | | | DYTLTISSLQPEDFAVYYCQQWSSNPQYTFGQGT |
| | | | KLEIK |
| SEQ ID NO: 3203 | VL | D-VL-H.19 | EIVLTQSPATLSVSPGERATLSCSVSSSVSYMHW |
| | | | YQQKPGQAPKLLIYDTSKLASGIPARFSGSGSGTE |
| | | | YTLTISSLQSEDFAVYYCQQWSSNPQYTFGQGTK |
| | | | LEIK |
| SEQ ID NO: 3204 | VL | D-VL-H.20 | EIVLTQSPATLSVSPGERATLSCSVSSSVSYMHW |
| | | | YQQKPGQAPKLLIYDTSKLASGIPARFSGSGSGTE |
| | | | YTLTISILQSEDFAVYYCQQWSSNPQYTFGQGTK |
| | | | LEIK |
| SEQ ID NO: 3205 | VL | D-VL-H.21 | EIVLTQSPPTLSLSPGERVTLSCSVSSSVSYMHWY |
| | | | QQKPGQAPKLLIYDTSKLASSIPARFSGSGSGTDY |
| | | | TLTISSLQPEDFAVYYCQQWSSNPQYTFGQGTKL |
| | | | EIK |
| SEQ ID NO: 3206 | VL | D-VL-H.22 | DIQLTQSPSSLSASVGDRVTITCSVSSSVSYMHW |
| | | | YQQKPGKAPKSLIYDTSKLASGVPSRFSGSGSGT |
| | | | DYTLTISSLQPEDFATYYCQQWSSNPQYTFGQGT |
| | | | KLEIK |
| SEQ ID NO: 3207 | VL | D-VL-H.23 | DIQLTQSPSSLSASVGDRVTITCSVSSSVSYMHW |
| | | | YQQKPGKAPKRLIYDTSKLASGVPSRFSGSGSGT |
| | | | EYTLTISNLQPEDFATYYCQQWSSNPQYTFGQGT |
| | | | KLEIK |
| SEQ ID NO: 3208 | VL | D-VL-H.24 | DIQLTQSPSAMSASVGDRVTITCSVSSSVSYMHW |
| | | | YQQKPGKVPKRLIYDTSKLASGVPSRFSGSGSGT |
| | | | EYTLTISSLQPEDFATYYCQQWSSNPQYTFGQGT |
| | | | KLEIK |
| SEQ ID NO: 3209 | VL | D-VL-H.25 | EIVLTQSPATLSLSPGERATLSCSVSSSVSYMHW |
| | | | YQQKPGQAPKLLIYDTSKLASGIPDRFSGSGSGT |
| | | | DYTLTISRLEPEDFAVYYCQQWSSNPQYTFGQGT |
| | | | KLEIK |
| SEQ ID NO: 3210 | VL | D-VL-H.26 | EIVLTQSPATLSLSPGERATLSCSVSSSVSYMHW |
| | | | YQQKPGLAPKLLIYDTSKLASGIPDRFSGSGSGTD |
| | | | YTLTISRLEPEDFAVYYCQQWSSNPQYTFGQGTK |
| | | | LEIK |
| SEQ ID NO: 3211 | VL | D-VL-H.27 | EIVLTQSPGTLSLSPGERATLSCSVSSSVSYMHW |
| | | | YQQKPGQAPKLLIYDTSKLASGIPDRFSGSGSGT |
| | | | DYTLTISRLEPEDFAVYYCQQWSSNPQYTFGQGT |
| | | | KLEIK |
| SEQ ID NO: 3212 | VL | D-VL-H.28 | DIQLTQSPSSLSASVGDRVTITCSVSSSVSYMHW |
| | | | YQQKPGKAPKSLIYDTSKLASGVPSKFSGSGSGT |
| | | | DYTLTISSLQPEDFATYYCQQWSSNPQYTFGQGT |
| | | | KLEIK |
| SEQ ID NO: 3213 | VL | D-VL-H.29 | DIQLTQSPSSLSASVGDRVTITCSVSSSVSYMHW |
| | | | YQQKPEKAPKSLIYDTSKLASGVPSRFSGSGSGT |
| | | | DYTLTISSLQPEDFATYYCQQWSSNPQYTFGQGT |
| | | | KLEIK |
| SEQ ID NO: 3214 | VL | D-VL-H.30 | DIVLTQSPDSLAVSLGERATINCSVSSSVSYMHW |
| | | | YQQKPGQPPKLLIYDTSKLASGVPDRFSGSGSGT |
| | | | DYTLTISSLQAEDVAVYYCQQWSSNPQYTFGQG |
| | | | TKLEIK |
| SEQ ID NO: 3215 | VL | D-VL-H.31 | EIVLTQTPLSLSITPGEQASMSCSVSSSVSYMHWY |
| | | | LQKARPVPKLLIYDTSKLASGVPDRFSGSGSGTD |
| | | | YTLKISRVEAEDFGVYYCQQWSSNPQYTFGQGT |
| | | | KLEIK |
| SEQ ID NO: 3216 | VL | D-VL-H.32 | EIVLTQTPLSLSITPGEQASISCSVSSSVSYMHWY |
| | | | LQKARPVPKLLIYDTSKLASGVPDRFSGSGSGTD |
| | | | YTLKISRVEAEDFGVYYCQQWSSNPQYTFGQGT |
| | | | KLEIK |
| SEQ ID NO: 3217 | VL | D-VL-H.33 | DIVLTQSPLSLPVTPGEPASISCSVSSSVSYMHWY |
| | | | LQKPGQSPKLLIYDTSKLASGVPDRFSGSGSGTD |
| | | | YTLKISRVEAEDVGVYYCQQWSSNPQYTFGQGT |
| | | | KLEIK |
| SEQ ID NO: 3218 | VL | D-VL-H.34 | DIVLTQSPLSLPVTLGQPASISCSVSSSVSYMHWY |
| | | | QQRPGQSPKRLIYDTSKLASGVPDRFSGSGSGTD |
| | | | YTLKISRVEAEDVGVYYCQQWSSNPQYTFGQGT |
| | | | KLEIK |
| SEQ ID NO: 3219 | VL | D-VL-H.35 | DIVLTQTPLSLPVTPGEPASISCSVSSSVSYMHWY |
| | | | LQKPGQSPKLLIYDTSKLASGVPDRFSGSGSGTD |
| | | | YTLKISRVEAEDVGVYYCQQWSSNPQYTFGQGT |
| | | | KLEIK |
| SEQ ID NO: 3220 | VL | D-VL-H.36 | DIVLTQTPLSLSVTPGQPASISCSVSSSVSYMHWY |
| | | | LQKPGQSPKLLIYDTSKLASGVPDRFSGSGSGTD |
| | | | YTLKISRVEAEDVGVYYCQQWSSNPQYTFGQGT |
| | | | KLEIK |
| SEQ ID NO: 3221 | VL | D-VL-H.37 | DIVLTQTPLSLSVTPGQPASISCSVSSSVSYMHWY |
| | | | LQKPGQPPKLLIYDTSKLASGVPDRFSGSGSGTD |
| | | | YTLKISRVEAEDVGVYYCQQWSSNPQYTFGQGT |
| | | | KLEIK |
| SEQ ID NO: 3222 | VL | D-VL-H.38 | DIQLIQSPSFLSASVGDRVSIICSVSSSVSYMHWY |
| | | | LQKPGKSPKLFIYDTSKLASGVSSRFSGRGSGTD |
| | | | YTLTIISLKPEDFAAYYCQQWSSNPQYTFGQGTK |
| | | | LEIK |
| SEQ ID NO: 3223 | VL | D-VL-H.39 | DIVLTQTPLSSPVTLGQPASISCSVSSSVSYMHWY |
| | | | QQRPGQPPKLLIYDTSKLASGVPDRFSGSGAGTD |
| | | | YTLKISRVEAEDVGVYYCQQWSSNPQYTFGQGT |
| | | | KLEIK |
| SEQ ID NO: 3224 | VL | D-VL-H.40 | EITLTQSPAFMSATPGDKVNISCSVSSSVSYMHW |
| | | | YQQKPGEAPKFIIYDTSKLASGIPPRFSGSGYGTD |
| | | | YTLTINNIESEDAAYYYCQQWSSNPQYTFGQGT |
| | | | KLEIK |

Variable HEAVY chain (VH)

| | | | |
|---|---|---|---|
| SEQ ID NO: 3225 | VH | D-VH-H.1 | EVQLVESGGGLVKPGGSLRLSCAVSGFTFRSYG |
| | | | MSWVRQAPGKGLEWVALISSGGSYTYYTDSVK |
| | | | GRFTISRDNSKNTLYLQMNSLKTEDTAVYYCSR |
| | | | HGGNFFDYWGQGTTVTVSS |
| SEQ ID NO: 3226 | VH | D-VH-H.2 | EVQLVESGGALVKPGGSLRLSCAVSGFTFRSYG |
| | | | MSWVRQAPGKGLEWVALISSGGSYTYYTDSVK |
| | | | GRFTISRDNSKNTLYLQMNSLKTEDTAVYYCSR |
| | | | HGGNFFDYWGQGTTVTVSS |
| SEQ ID NO: 3227 | VH | D-VH-H.3 | EVQLVESGGGLVQPGGSLRLSCAVSGFTFRSYG |
| | | | MSWVRQAPGKGLEWVALISSGGSYTYYTDSVK |
| | | | GRFTISRDNAKNTLYLQMNSLRAEDTAVYYCSR |
| | | | HGGNFFDYWGQGTTVTVSS |
| SEQ ID NO: 3228 | VH | D-VH-H.4 | EVQLVESGGGLVQPGGSLRLSCAVSGFTFRSYG |
| | | | MSWVRQAPGKGLEWVALISSGGSYTYYTDSVK |
| | | | GRFTISRDNAKNSLYLQMNSLRAEDTAVYYCSR |
| | | | HGGNFFDYWGQGTTVTVSS |
| SEQ ID NO: 3229 | VH | D-VH-H.5 | EVQLVESGGGLVQPGGSLRLSCAVSGFTFRSYG |
| | | | MSWVRQAPGKGLEWVALISSGGSYTYYTDSVK |
| | | | GRFTISRDNSKNSLYLQMNSLKTEDTAVYYCSR |
| | | | HGGNFFDYWGQGTTVTVSS |
| SEQ ID NO: 3230 | VH | D-VH-H.6 | EVQLVESGGGLVQPGGSLRLSCAVSGFTFRSYG |
| | | | MSWVRQAPGKGLEWVALISSGGSYTYYTDSVK |
| | | | GRFTISRDNAKNSLYLQMNSLRAEDMAVYYCSR |
| | | | HGGNFFDYWGQGTTVTVSS |
| SEQ ID NO: 3231 | VH | D-VH-H.7 | EVQLVESGGGLVQPGGSLRLSCAVSGFTFRSYG |
| | | | MSWVRQAPGKGLEWVALISSGGSYTYYTDSVK |
| | | | GQFTISRDNAKNTLYLQMNSLRAEDMAVYYCSR |
| | | | HGGNFFDYWGQGTTVTVSS |
| SEQ ID NO: 3232 | VH | D-VH-H.8 | EVQLVESGGGLVKPGRSLRLSCTVSGFTFRSYGM |
| | | | SWVRQAPGKGLEWVALISSGGSYTYYTDSVKGR |
| | | | FTISRDNSKNILYLQMNSLKTEDTAVYYCSRHGG |
| | | | NFFDYWGQGTTVTVSS |
| SEQ ID NO: 3233 | VH | D-VH-H.9 | EVQLVESGGGLVKPGGSLRLSCAVSGFTFRSYG |
| | | | MSWVRQAPGKGLEWVALISSGGSYTYYTDSVK |
| | | | GRFTISRDNAKNSLYLQiVlNSLRAEDTAVYYCSR |
| | | | HGGNFFDYWGQGTTVTVSS |
| SEQ ID NO: 3234 | VH | D-VH-H.10 | EVQLVESGGGLVQPGGSLKLSCAVSGFTFRSYG |
| | | | MSWVRQASGKGLEWVALISSGGSYTYYTDSVK |
| | | | GRFTISRDNSKNTLYLQMNSLKTEDTAVYYCSR |
| | | | HGGNFFDYWGQGTTVTVSS |
| SEQ ID NO: 3235 | VH | D-VH-H.11 | QVQLVESGGGVVQPGGSLRLSCAVSGFTFRSYG |
| | | | MSWVRQAPGKGLEWVALISSGGSYTYYTDSVK |
| | | | GRFTISRDNSKNTLYLQMNSLRAEDTAVYYCSR |
| | | | HGGNFFDYWGQGTTVTVSS |
| SEQ ID NO: 3236 | VH | D-VH-H.12 | QVQLVESGGGVVQPGRSLRLSCAVSGFTFRSYG |
| | | | MSWVRQAPGKGLEWVALISSGGSYTYYTDSVK |
| | | | GRFTISRDNSKNTLYLQMSSLRAEDTAVYYCSRH |
| | | | GGNFFDYWGQGTTVTVSS |
| SEQ ID NO: 3237 | VH | D-VH-H.13 | EVQLVESGGGLVQPGGSLRLSCPVSGFTFRSYGM |
| | | | SWVRQAPGKGLEWVALISSGGSYTYYTDSVKGR |
| | | | FTISRDNANNSLYLQMNSLRAEDTAVYYCSRHG |
| | | | GNFFDYWGQGTTVTVSS |
| SEQ ID NO: 3238 | VH | D-VH-H.14 | EVQLVESGGGLVQPGRSLRLSCTVSGFTFRSYGM |
| | | | SWVRQAPGKGLEWVALISSGGSYTYYTDSVKGR |
| | | | FTISRDNSKNILYLQMNSLKTEDTAVYYCSRHGG |
| | | | NFFDYWGQGTTVTVSS |
| SEQ ID NO: 3239 | VH | D-VH-H.15 | EVQLVESGGGLVQPGPSLRLSCTVSGFTFRSYGM |
| | | | SWVRQAPGKGLEWVALISSGGSYTYYTDSVKGR |
| | | | FTISRDNSKNILYLQMNSLKTEDTAVYYCSRHGG |
| | | | NFFDYWGQGTTVTVSS |
| SEQ ID NO: 3240 | VH | D-VH-H.16 | EVQLVESGGGLVQPGGSLRLSCAVSGFTFRSYG |
| | | | MSWVRQAPGKGLEWVALISSGGSYTYYTDSVK |
| | | | GRFTISRDNSKNTLYLQMNSLRAEDTAVYYCSR |
| | | | HGGNFFDYWGQGTTVTVSS |
| SEQ ID NO: 3241 | VH | D-VH-H.17 | EVQLVESGGGLVQPGGSLRLSCAVSGFTFRSYG |
| | | | MSWVRQAPGKGLEWVALISSGGSYTYYTDSVK |
| | | | GRFTISRDNAKNSLYLQMNSLRDEDTAVYYCSR |
| | | | HGGNFFDYWGQGTTVTVSS |
| SEQ ID NO: 3242 | VH | D-VH-H.18 | QVQLVESGGGLVKPGGSLRLSCAVSGFTFRSYG |
| | | | MSWVRQAPGKGLEWVALISSGGSYTYYTDSVK |
| | | | GRFTISRDNAKNSLYLQMNSLRAEDTAVYYCSR |
| | | | HGGNFFDYWGQGTTVTVSS |
| SEQ ID NO: 3243 | VH | D-VH-H.19 | QVQLVESGGGVVQPGRSLRLSCAVSGFTFRSYG |
| | | | MSWVRQAPGKGLEWVALISSGGSYTYYTDSVK |
| | | | GRFTISRDNSKNTLYLQMNSLRAEDTAVYYCSR |
| | | | HGGNFFDYWGQGTTVTVSS |
| SEQ ID NO: 3244 | VH | D-VH-H.20 | EVQLLESGGGLVQPGGSLRLSCAVSGFTFRSYG |
| | | | MSWVRQAPGKGLEWVALISSGGSYTYYTDSVK |
| | | | GRFTISRDNSKNTLYLQMNSLRAEDTAVYYCSR |
| | | | HGGNFFDYWGQGTTVTVSS |
| SEQ ID NO: 3245 | VH | D-VH-H.21 | EVQLVESGGGLVQPGGSLRLSCAVSGFTFRSYG |
| | | | MSWVRQAPGKGLEWVALISSGGSYTYYTDSVK |
| | | | GRFTISRHNSKNTLYLQMNSLRAEDTAVYYCSR |
| | | | HGGNFFDYWGQGTTVTVSS |
| SEQ ID NO: 3246 | VH | D-VH-H.22 | EVQLVESGGGLIQPGGSLRLSCAVSGFTFRSYGM |
| | | | SWVRQPPGKGLEWVALISSGGSYTYYTDSVKGR |
| | | | FTISRDNSKNTLYLQMNSLRAEDTAVYYCSRHG |
| | | | GNFFDYWGQGTTVTVSS |
| SEQ ID NO: 3247 | VH | D-VH-H.23 | EVQLVESGGGLIQPGGSLRLSCAVSGFTFRSYGM |
| | | | SWVRQAPGKGLEWVALISSGGSYTYYTDSVKGR |
| | | | FTISRDNSKNTLYLQMNSLRAEDTAVYYCSRHG |
| | | | GNFFDYWGQGTTVTVSS |
| SEQ ID NO: 3248 | VH | D-VH-H.24 | EVQLVESGGGLVQPGRSLRLSCAVSGFTFRSYG |
| | | | MSWVRQAPGKGLEWVALISSGGSYTYYTDSVK |
| | | | GRFTISRDNAKNSLYLQMNSLRAEDTALYYCSR |
| | | | HGGNFFDYWGQGTTVTVSS |
| SEQ ID NO: 3249 | VH | D-VH-H.25 | QVQLVESGGGVVQPGRSLRLSCAVSGFTFRSYG |
| | | | MSWVRQAPGKGLEWVALISSGGSYTYYTDSVK |
| | | | GRFTISRDNSKNRLYLQMNSLRAEDTAVYYCSR |
| | | | HGGNFFDYWGQGTTVTVSS |
| SEQ ID NO: 3250 | VH | D-VH-H.26 | QVQLVESGGGVVQPGRSLRLSCAVSGFTFRSYG |
| | | | MSWVRQAPGKGLEWVALISSGGSYTYYTDSVK |
| | | | GRFTISRDNSKNTLYLQMNSLRAEGTAVYYCSR |
| | | | HGGNFFDYWGQGTTVTVSS |
| SEQ ID NO: 3251 | VH | D-VH-H.27 | QVQLVESGGGVVQPGRSLRLSCAVSGFTFRSYG |
| | | | MSWVRQAPGKGLEWVALISSGGSYTYYTDSVK |
| | | | GRFAISRDNSKNTLYLQMNSLRAEDTAVYYCSR |
| | | | HGGNFFDYWGQGTTVTVSS |
| SEQ ID NO: 3252 | VH | D-VH-H.28 | QVQLVDSGGGVVQPGRSLRLSCAVSGFTFRSYG |
| | | | MSWVRQAPGKGLEWVALISSGGSYTYYTDSVK |
| | | | GRFTISRDNSKNTLYLQMNSLRAEDTAVYYCSR |
| | | | HGGNFFDYWGQGTTVTVSS |
| SEQ ID NO: 3253 | VH | D-VH-H.29 | EVQLVESGGGVVRPGGSLRLSCAVSGFTFRSYG |
| | | | MSWVRQAPGKGLEWVALISSGGSYTYYTDSVK |
| | | | GRFTISRDNAKNSLYLQMNSLRAEDTALYHCSR |
| | | | HGGNFFDYWGQGTTVTVSS |
| SEQ ID NO: 3254 | VH | D-VH-H.30 | EVQLVESGGVVVQPGGSLRLSCAVSGFTFRSYG |
| | | | MSWVRQAPGKGLEWVALISSGGSYTYYTDSVK |
| | | | GRFTISRDNSKNSLYLQMNSLRAEDTALYYCSRH |
| | | | GGNFFDYWGQGTTVTVSS |
| SEQ ID NO: 3255 | VH | D-VH-H.31 | EVQLVESGGGVVQPGGSLRLSCAVSGFTFRSYG |
| | | | MSWVRQAPGKGLEWVALISSGGSYTYYTDSVK |
| | | | GRFTISRDNSKNSLYLQMNSLRTEDTALYYCSRH |
| | | | GGNFFDYWGQGTTVTVSS |
| SEQ ID NO: 3256 | VH | D-VH-H.32 | EVQLVESGGVVVQPGGSLRLSCAVSGFTFRSYG |
| | | | MSWVRQAPGKGLEWVALISSGGSYTYYTDSVK |
| | | | GRFTISRDNSKNSLYLQMNSLRTEDTALYYCSRH |
| | | | GGNFFDYWGQGTTVTVSS |
| SEQ ID NO: 3257 | VH | D-VH-H.33 | EVQLVETGGGLIQPGGSLRLSCAVSGFTFRSYGM |
| | | | SWVRQAPGKGLEWVALISSGGSYTYYTDSVKGR |
| | | | FTISRDNSKNTLYLQMNSLRAEDTAVYYCSRHG |
| | | | GNFFDYWGQGTTVTVSS |
| SEQ ID NO: 3258 | VH | D-VH-H.34 | EVQLVESGGGLVQPGGSLRLSCAVSGFTFRSYG |
| | | | MSWVRQATGKGLEWVALISSGGSYTYYTDSVK |
| | | | GRFTISRENAKNSLYLQMNSLRAGDTAVYYCSR |
| | | | GGNFFDYWGQGTTVTVSS |
| SEQ ID NO: 3259 | VH | D-VH-H.35 | EVQLVESRGVLVQPGGSLRLSCAVSGFTFRSYG |
| | | | MSWVRQAPGKGLEWVALISSGGSYTYYTDSVK |
| | | | GRFTISRDNSKNTLHLQMNSLRAEDTAVYYCSR |
| | | | HGGNFFDYWGQGTTVTVSS |
| SEQ ID NO: 3260 | VH | D-VH-H.36 | EVQLVESGGGLVQPGRSLRLSCAVSGFTFRSYG |
| | | | MSWVRQAPGKGLEWVALISSGGSYTYYTDSVK |
| | | | GRFTISRDNAKNSLYLQMNSLRAEDMALYYCSR |
| | | | HGGNFFDYWGQGTTVTVSS |
| SEQ ID NO: 3261 | VH | D-VH-H.37 | QVQLVESGGGLVQPGGSLRLSCSVSGFTFRSYG |
| | | | MSWVRQAPGKGLEWVALISSGGSYTYYTDSVK |
| | | | GRFTISRDNSKNTLYLQMNSLRAEDTAVYYCSR |
| | | | HGGNFFDYWGQGTTVTVSS |
| SEQ ID NO: 3262 | VH | D-VH-H.38 | EVQLVESGGGLVQPGGSLRLSCSVSGFTFRSYGM |
| | | | SWVRQAPGKGLEWVALISSGGSYTYYTDSVKGR |
| | | | FTISRDNSKNTLYLQMSSLRAEDTAVYYCSRHG |
| | | | GNFFDYWGQGTTVTVSS |
| SEQ ID NO: 3263 | VH | D-VH-H.39 | QVQLVESGGGVVQPGRSLRLSCAVSGFTFRSYG |
| | | | MSWVRQAPGKGLEWVALISSGGSYTYYTDSVK |
| | | | GRFTISRDNSTNTLFLQMNSLRAEDTAVYYCSRH |
| | | | GGNFFDYWGQGTTVTVSS |
| SEQ ID NO: 3264 | VH | D-VH-H.40 | QVQLLESGGGLVKPGGSLRLSCAVSGFTFRSYG |
| | | | MSWVRQAPGKGLEWVALISSGGSYTYYTDSVK |
| | | | GRFTISRDNAKNSLYLQMNSLRAEDTAVYYCSR |
| | | | HGGNFFDYWGQGTTVTVSS |
| SEQ ID NO: 3265 | VH | D-VH-H.41 | EVQLVESGEGLVQPGGSLRLSCAVSGFTFRSYG |
| | | | MSWVRQAPGKGLEWVALISSGGSYTYYTDSVK |
| | | | GRFTISRDNSKNTLYLQMGSLRAEDMAVYYCSR |
| | | | HGGNFFDYWGQGTTVTVSS |
| SEQ ID NO: 3266 | VH | D-VH-H.42 | EVQLVESGGGLVQPGGSLRLSCAVSGFTFRSYG |
| | | | MSWVRQAPGKGLEWVALISSGGSYTYYTDSVK |
| | | | GRFTISRDNSKNTLYLQMGSLRAEDMAVYYCSR |
| | | | HGGNFFDYWGQGTTVTVSS |
| SEQ ID NO: 3267 | VH | D-VH-H.43 | EVQLVESGGGLVQPGGSLRLSCSVSGFTFRSYGM |
| | | | SWVRQAPGKGLEWVALISSGGSYTYYTDSVKGR |
| | | | FTISRDNSKNTLYVQMSSLRAEDTAVYYCSRHG |
| | | | GNFFDYWGQGTTVTVSS |
| SEQ ID NO: 3268 | VH | D-VH-H.44 | EVQLVESGGGLVQPGGSLRLSCAVSGFTFRSYG |
| | | | MSWVRQAPGKGLEWVALISSGGSYTYYTDSVK |
| | | | GRFIISRDNSRNSLYLQKNRRRAEDMAVYYCSR |
| | | | HGGNFFDYWGQGTTVTVSS |
| SEQ ID NO: 3269 | VH | D-VH-H.45 | EVQLVESGGGLVQPGGSLRLSCAVSGFTFRSYG |
| | | | MSWVHQAPGKGLEWVALISSGGSYTYYTDSVK |
| | | | GRFIISRDNSRNTLYLQTNSLRAEDTAVYYCSRH |
| | | | GGNFFDYWGQGTTVTVSS |
| SEQ ID NO: 3270 | VH | D-VH-H.46 | EVHLVESGGGLVQPGGALRLSCAVSGFTFRSYG |
| | | | MSWVRQATGKGLEWVALISSGGSYTYYTDSVK |
| | | | GRFTISRENAKNSLYLQMNSLRAGDTAVYYCSR |
| | | | HGGNFFDYWGQGTTVTVSS |
| SEQ ID NO: 3271 | VH | D-VH-H.47 | EVQLVESGGGLVQPRGSLRLSCAVSGFTFRSYG |
| | | | MSWVRQAPGKGLEWVALISSGGSYTYYTDSVK |
| | | | GRFTISRDNSKNTLYLQMNNLRAEGTAVYYCSR |
| | | | HGGNFFDYWGQGTTVTVSS |
| SEQ ID NO: 3272 | VH | D-VH-H.48 | EVQLVESGGGLVQPRGSLRLSCAVSGFTFRSYG |
| | | | MSWVRQAPGKGLEWVALISSGGSYTYYTDSVK |
| | | | GRFTISRDNSKNTLYLQMNNLRAEGTAAYYCSR |
| | | | HGGNFFDYWGQGTTVTVSS |
| SEQ ID NO: 3273 | VH | D-VH-H.49 | QVQLVQSGAEVKKPGASVKVSCKVSGFTFRSYG |
| | | | MSWVRQAPGKGLEWVALISSGGSYTYYTDSVK |
| | | | GRFTITRDNSTNTLYMELSSLRSEDTAVYYCSRH |
| | | | GGNFFDYWGQGTTVTVSS |
| SEQ ID NO: 3274 | VH | D-VH-H.50 | QVQLVQSGSELKKPGASVKVSCKVSGFTFRSYG |
| | | | MSWVRQAPGQGLEWVALISSGGSYTYYTDSVK |
| | | | GRFVISRDNSVNTLYLQISSLKAEDTAVYYCSRH |
| | | | GGNFFDYWGQGTTVTVSS |

In some embodiments, the anti-TCRβV10 antibody molecule comprises a VH or a VL of an antibody described in Table 8, or a sequence with at least 80%, 85%, 90%, 95%, 96%, 97%, 98%, 99% or more identity thereto.

In some embodiments, the anti-TCRβV10 antibody molecule comprises a VH and a VL of an antibody described in Table 8, or a sequence with at least 80%, 85%, 90%, 95%, 96%, 97%, 98%, 99% or more identity thereto.

### Additional Anti-TCRVβ Antibodies

Additional exemplary anti-TCRβV antibodies of the disclosure are provided in Table 9. In some embodiments, the anti-TCRβV antibody is a humanized antibody, e.g., as provided in Table 9. In some embodiments, the anti-TCRβV antibody comprises one or more (e.g., all three) of a LC CDR1, LC CDR2, and LC CDR3 provided in Table 9; and/or one or more (e.g., all three) of a HC CDR1, HC CDR2, and HC CDR3 provided in Table 9, or a sequence with at least 95% identity thereto. In some embodiments, the anti-TCRβV antibody comprises a variable heavy chain (VH) and/or a variable light chain (VL) provided in Table 9, or a sequence with at least 95% identity thereto.

TABLE 9
Amino acid sequences for additional anti-TCRβ V antibodies
Antibody G (murine) binds to human TCRVβ 20-1

| | | |
|---|---|---|
| SEQ ID NO: 1102 | HC CDR1 (Kabat) | SAYMH |
| SEQ ID NO: 1103 | HC CDR2 (Kabat) | RIDPATGKTKYAPKFQA |
| SEQ ID NO: 1104 | HC CDR3 (Kabat) | SLNWDYGLDY |
| SEQ ID NO: 1105 | HC CDR1 (Chothia) | GFNIKSA |
| SEQ ID NO: 1106 | HC CDR2 (Chothia) | DPATGK |
| SEQ ID NO: 1104 | HC CDR3 (Chothia) | SLNWDYGLDY |
| SEQ ID NO: 3474 | HC CDR1 (Combined) | GFNIKSAYMH |
| SEQ ID NO: 1103 | HC CDR2 (Combined) | RIDPATGKTKYAPKFQA |
| SEQ ID NO: 1104 | HC CDR3 (Combined) | SLNWDYGLDY |
| SEQ ID NO: 1107 | LC CDR1 (Kabat) | RASKSVSILGTHLIH |
| SEQ ID NO: 1108 | LC CDR2 (Kabat) | AASNLES |
| SEQ ID NO: 1109 | LC CDR3 (Kabat) | QQSIEDPWT |
| SEQ ID NO: 1110 | LC CDR1 (Chothia) | SKSVSILGTHL |
| SEQ ID NO: 1108 | LC CDR2 (Chothia) | AASNLES |
| SEQ ID NO: 1109 | LC CDR3 (Chothia) | QQSIEDPWT |
| SEQ ID NO: 1107 | LC CDR1 (Combined) | RASKSVSILGTHLIH |
| SEQ ID NO: 1108 | LC CDR2 (Combined) | AASNLES |
| SEQ ID NO: 1109 | LC CDR3(Combined) | QQSIEDPWT |
| SEQ ID NO: 1111 | VL | DIVLTQSPASLAVSLGQRATISCRASKSVSILGTHL |
| | | IHWYQQKPGQPPKLLIYAASNLESGVPARFSGSGS |
| | | ETVFTLNIHPVEEEDAATYFCQQSIEDPWTFGGGT |
| | | KLGIK |
| SEQ ID NO: 1112 | VH | EVQLQQSVADLVRPGASLKLSCTASGFNIKSAYM |
| | | HWVIQRPDQGPECLGRIDPATGKTKYAPKFQAKA |
| | | TITADTSSNTAYLQLSSLTSEDTAIYYCTRSLNWD |
| | | YGLDYWGQGTSVTVSS |

Antibody G-H (humanized) VHs binds to human TCRVβ 20-1

| | | |
|---|---|---|
| SEQ ID NO: 1113 | VH - 1 | QVQLVQSGAEVKKPGASVKVSCKASGFNIKSAY |
| | | MHWVRQAPGQGLEWMGRIDPATGKTKYAPKFQ |
| | | ARVTMTADTSTNTAYMELSSLRSEDTAVYYCAR |
| | | SLNWDYGLDYWGQGTLVTVSS |
| SEQ ID NO: 1114 | VH - 2 | QVQLVQSGAEVKKPGASVKVSCKASGFNIKSAY |
| | | MHWVRQAPGQEPGCMGRIDPATGKTKYAPKFQA |
| | | RVTMTADTSINTAYTELSSLRSEDTATYYCARSLN |
| | | WDYGLDYWGQGTLVTVSS |
| SEQ ID NO: 1115 | VH - 3 | QVQLVQSGAEVKKPGSSVKVSCKASGFNIKSAY |
| | | MHWVRQAPGQGLEWMGRIDPATGKTKYAPKFQ |
| | | ARVTITADTSTNTAYMELSSLRSEDTAVYYCARS |
| | | LNWDYGLDYWGQGTLVTVSS |
| SEQ ID NO: 1116 | VH - 4 | QVQLVQSGAEVKKPGASVKVSCKASGFNIKSAY |
| | | MHWVRQAPGQRLEWMGRIDPATGKTKYAPKFQ |
| | | ARVTITADTSANTAYMELSSLRSEDTAVYYCARS |
| | | LNWDYGLDYWGQGTLVTVSS |

Antibody G-H (humanized) VLs binds to human TCRVβ 20-1

| | | |
|---|---|---|
| SEQ ID NO: 1117 | VL - 1 | EIVLTQSPATLSLSPGERATLSCRASKSVSILGTHLI |
| | | HWYQQKPGQAPRLLIYAASNLESGIPARFSGSGSE |
| | | TDFTLTISSLEPEDFAVYFCQQSIEDPFGGGTKVEI |
| | | K |
| SEQ ID NO: 1118 | VL - 2 | EIVLTQSPATLSLSPGERATLSCRASKSVSILGTHLI |
| | | HWYQQKPGLAPRLLIYAASNLESGIPDRFSGSGSE |
| | | TDFTLTISRLEPEDFAVYFCQQSIEDPFGGGTKVEI |
| | | K |
| SEQ ID NO: 1119 | VL - 3 | EIVLTQSPGTLSLSPGERATLSCRASKSVSILGTHLI |
| | | HWYQQKPGQAPRLLIYAASNLESGIPDRFSGSGSE |
| | | TDFTLTISRLEPEDFAVYFCQQSIEDPFGGGTKVEI |
| | | K |

Antibody H (murine) binds to human TCRVβ 27

| | | |
|---|---|---|
| SEQ ID NO: 1120 | HC CDR1 (Kabat) | DTYMY |
| SEQ ID NO: 1121 | HC CDR2 (Kabat) | RIDPANGNTKYDPKFQD |
| SEQ ID NO: 1122 | HC CDR3 (Kabat) | GSYYYAMDY |
| SEQ ID NO: 1123 | HC CDR1 (Chothia) | GFKTEDT |
| SEQ ID NO: 1124 | HC CDR2 (Chothia) | DPANGN |
| SEQ ID NO: 1122 | HC CDR3 (Chothia) | GSYYYAMDY |
| SEQ ID NO: 1125 | HC CDR1 (Combined) | GFKTEDTYMY |
| SEQ ID NO: 1121 | HC CDR2 (Combined) | RIDPANGNTKYDPKFQD |
| SEQ ID NO: 1122 | HC CDR3(Combined) | GSYYYAMDY |
| SEQ ID NO: 1126 | LC CDR1 (Kabat) | RA SESVD SYGNSFMH |
| SEQ ID NO: 1127 | LC CDR2 (Kabat) | RASNLES |
| SEQ ID NO: 1128 | LC CDR3 (Kabat) | QQSNEDPYT |
| SEQ ID NO: 3475 | LC CDR1 (Chothia) | SESVDSYGNSF |
| SEQ ID NO: 1127 | LC CDR2 (Chothia) | RASNLES |
| SEQ ID NO: 1128 | LC CDR3 (Chothia) | QQSNEDPYT |
| SEQ ID NO: 1126 | LC CDR1 (Combined) | RASESVDSYGNSFMH |
| SEQ ID NO: 1127 | LC CDR2 (Combined) | RASNLES |
| SEQ ID NO: 1128 | LC CDR3(Combined) | QQSNEDPYT |
| SEQ ID NO: 1129 | VL | DIVLTQSPASLAVSLGQRATISCRASESVDSYGNS |
| | | FMHWYQQKPGQPPKLLIYRASNLESGIPARFSGSG |
| | | SRTDFTLTINPVEADDVATYYCQQSNEDPYTFGG |
| | | GTKLEIK |
| SEQ ID NO: 1130 | VH | EVQLQQSGAELVKPGASVKLSCTASGFKTEDTYM |
| | | YWVKQRPEQGLEWIGRIDPANGNTKYDPKFQDK |
| | | ATITADSSSNTAYLQLSSLPSEDTAVYYCARGSYY |
| | | YAMDYWGQGTSVTVSS |

Antibody H-H (humanized) VHs binds to human TCRVβ 27

| | | |
|---|---|---|
| SEQ ID NO: 1131 | VH - 1 | QVQLVQSGAEVKKPGSSVKVSCKASGFKTEDTY |
| | | MYWVRQAPGQGLEWIGRIDPANGNTKYDPKFQD |
| | | RATITADSSTNTAYMELSSLRSEDTAVYYCARGS |
| | | YYYAMDYWGQGTLVTVSS |
| SEQ ID NO: 1132 | VH - 2 | QVQLVQSGAEVKKPGASVKVSCKASGFKTEDTY |
| | | MYWVRQAPGQRLEWIGRIDPANGNTKYDPKFQD |
| | | RATITADSSANTAYMELSSLRSEDTAVYYCARGS |
| | | YYYAMDYWGQGTLVTVSS |
| SEQ ID NO: 1133 | VH - 3 | EVQLVESGGGLVQPGGSLKLSCAASGFKTEDTYM |
| | | YWVRQASGKGLEWIGRIDPANGNTKYDPKFQDR |
| | | ATISADSSKNTAYLQMNSLKTEDTAVYYCARGSY |
| | | YYAMDYWGQGTLVTVSS |
| SEQ ID NO: 1134 | VH - 4 | EVQLVQSGAEVKKPGESLRISCKASGFKTEDTYM |
| | | YWVRQMPGKGLEWIGRIDPANGNTKYDPKFQDQ |
| | | ATISADSSINTAYLQWSSLKASDTAMYYCARGSY |
| | | YYAMDYWGQGTLVTVSS |
| SEQ ID NO: 1135 | VH - 5 | QVQLVQSGSELKKPGASVKVSCKASGFKTEDTY |
| | | MYWVRQAPGQGLEWIGRIDPANGNTKYDPKFQD |
| | | RAVISADSSVNTAYLQISSLKAEDTAVYYCARGS |
| | | YYYAMDYWGQGTLVTVSS |

Antibody H-H (humanized) VLs Binds to human TCRVβ 27

| | | |
|---|---|---|
| SEQ ID NO: 1136 | VL - 1 | DIVLTQSPDSLAVSLGERATINCRASESVDSYGNS |
| | | FMHWYQQKPGQPPKLLIYRASNLESGVPDRFSGS |
| | | GSRTDFTLTISSLQAEDVAVYYCQQSNEDPYTFG |
| | | QGTKLEIK |
| SEQ ID NO: 1137 | VL - 2 | EIVLTQSPATLSLSPGERATLSCRASESVDSYGNSF |
| | | MHWYQQKPGQAPKLLIYRASNLESGIPARFSGSG |
| | | SRTDFTLTISRLEPEDFAVYYCQQSNEDPYTFGQG |
| | | TKLEIK |
| SEQ ID NO: 1138 | VL - 3 | DIQLTQSPSSLSASVGDRVTITCRASESVDSYGNSF |
| | | MHWYQQKPGQAPKLLIYRASNLESGVPSRFSGSG |
| | | SRTDFTLTISSLQPEDVATYYCQQSNEDPYTFGQG |
| | | TKLEIK |
| SEQ ID NO: 1139 | VL - 4 | AIQLTQSPSSLSASVGDRVTITCRASESVDSYGNSF |
| | | MHWYQQKPGKAPKLLIYRASNLESGVPSRFSGSG |
| | | SRTDFTLTISSLQPEDFATYYCQQSNEDPYTFGQG |
| | | TKLEIK |
| SEQ ID NO: 1140 | VL - 5 | EIVLTQSPDFQSVTPKEKVTITCRASESVDSYGNSF |
| | | MHWYQQKPDQSPKLLIYRASNLESGVPSRFSGSG |
| | | SRTDFTLTINSLEAEDAATYYCQQSNEDPYTFGQG |
| | | TKLEIK |

Antibody I(murine) binds to human TCRVβ 6-5 ,6-6, 6-9

| | | |
|---|---|---|
| SEQ ID NO: 1141 | HC CDR1 (Kabat) | SYAMS |
| SEQ ID NO: 1142 | HC CDR2 (Kabat) | HISNGGDYIYYADTVKG |
| SEQ ID NO: 1143 | HC CDR3 (Kabat) | PSYYSDPWFFDV |
| SEQ ID NO: 1144 | HC CDR1 (Chothia | GFTFRSY |
| SEQ ID NO: 1145 | HC CDR2 (Chothia) | SNGGDY |
| SEQ ID NO: 1143 | HC CDR3 (Chothia) | PSYYSDPWFFDV |
| SEQ ID NO: 1146 | HC CDR1 (Combined) | GFTFRSYAMS |
| SEQ ID NO: 1142 | HC CDR2 (Combined) | HISNGGDYIYYADTVKG |
| SEQ ID NO: 1143 | HC CDR3(Combined) | PSYYSDPWFFDV |
| SEQ ID NO: 1147 | LC CDR1 (Kabat) | SAGSSVSFMH |
| SEQ ID NO: 1148 | LC CDR2 (Kabat) | DTSKLAS |
| SEQ ID NO: 1149 | LC CDR3 (Kabat) | LQGSGFPLT |
| SEQ ID NO: 1150 | LC CDR1 (Chothia) | GSSVSF |
| SEQ ID NO: 1148 | LC CDR2 (Chothia) | DTSKLAS |
| SEQ ID NO: 1149 | LC CDR3 (Chothia) | LQGSGFPLT |
| SEQ ID NO: 1147 | LC CDR1 (Combined) | SAGSSVSFMH |
| SEQ ID NO: 1148 | LC CDR2 (Combined) | DTSKLAS |
| SEQ ID NO: 1149 | LC CDR3(Combined) | LQGSGFPLT |
| SEQ ID NO: 1151 | VL | ENVLTQSPAIMSASPGEKVTMTCSAGSSVSFMHW |
| | | YQQKSSTSPKLWIYDTSKLASGVPGRFSGSGSGNS |
| | | FSLTISSMEAEDVAIYYCLQGSGFPLTFGSGTKLEI |
| SEQ ID NO: 1152 | VH | DVKLVESGEGLVKPGGSLKLSCAASGFTFRSYAM |
| | | SWVRQTPEKRLEWVAHISNGGDYIYYADTVKGR |
| | | FTISRDNARNTLYLQMSSLKSEDTAMYYCTRPSY |
| | | YSDPWFFDVWGTGTTVTVSS |

Antibody I-H (humanized) VHs
Binds to human TCRVβ 6-5, 6-6, 6-9

| | | |
|---|---|---|
| SEQ ID NO: 1153 | VH - 1 | EVQLVESGGGLVQPGGSLRLSCAASGFTFRSYAM |
| | | SWVRQAPGKGLEWVAHISNGGDYIYYADTVKGR |
| | | FTISRDNAKNSLYLQMNSLRAEDTAVYYCTRPSY |
| | | YSDPWFFDVWGQGTTVTVSS |
| SEQ ID NO: 1154 | VH - 2 | QVQLVESGGGVVQPGRSLRLSCAASGFTFRSYAM |
| | | SWVRQAPGKGLEWVAHISNGGDYIYYADTVKGR |
| | | FTISRDNSKNTLYLQMSSLRAEDTAVYYCTRPSY |
| | | YSDPWFFDVWGQGTTVTVSS |
| SEQ ID NO: 1155 | VH - 3 | EVQLVESGGGLVQPGGSLRLSCAASGFTFRSYAM |
| | | SWVRQAPGKGLEWVAHISNGGDYIYYADTVKGR |
| | | FTISRDNSKNTLYLQMNSLRAEDTAVYYCTRPSY |
| | | YSDPWFFDVWGQGTTVTVSS |
| SEQ ID NO: 1156 | VH - 4 | QVQLVQSGSELKKPGASVKVSCKASGFTFRSYAM |
| | | SWVRQAPGQGLEWVAHISNGGDYIYYADTVKGR |
| | | FVISRDNSVNTLYLQISSLKAEDTAVYYCTRPSYY |
| | | SDPWFFDVWGQGTTVTVSS |
| SEQ ID NO: 1157 | VH - 5 | QVQLVQSGAEVKKPGASVKVSCKASGFTFRSYA |
| | | MSWVRQAPGQRLEWVAHISNGGDYIYYADTVKG |
| | | RFTITRDNSANTLYMELSSLRSEDTAVYYCTRPSY |
| | | YSDPWFFDVWGQGTTVTVSS |

Antibody I-H (humanized) VLs
Binds to human TCRVβ 6-5, 6-6, 6-9

| | | |
|---|---|---|
| SEQ ID NO: 1158 | VL - 1 | ENVLTQSPATLSLSPGERATLSCSAGSSVSFMHW |
| | | YQQKPGQAPKLLIYDTSKLASGIPARFSGSGSGND |
| | | FTLTISSLEPEDFAVYYCLQGSGFPLTFGQGTKLEI |
| | | K |
| SEQ ID NO: 1159 | VL - 2 | ENVLTQSPDFQSVTPKEKVTITCSAGSSVSFMHW |
| | | YQQKPDQSPKLLIYDTSKLASGVPSRFSGSGSGND |
| | | FTLTINSLEAEDAATYYCLQGSGFPLTFGQGTKLEI |
| | | K |
| SEQ ID NO: 1160 | VL - 3 | DNQLTQSPSSLSASVGDRVTITCSAGSSVSFMHW |
| | | YQQKPGKVPKLLIYDTSKLASGVPSRFSGSGSGN |
| | | DFTLTISSLQPEDVATYYCLQGSGFPLTFGQGTKL |
| | | EIK |
| SEQ ID NO: 1161 | VL - 4 | ANQLTQSPSSLSASVGDRVTITCSAGSSVSFMHW |
| | | YQQKPGKAPKLLIYDTSKLASGVPSRFSGSGSGN |
| | | DFTLTISSLQPEDFATYYCLQGSGFPLTFGQGTKL |
| | | EIK |
| SEQ ID NO: 1162 | VL - 5 | DNVLTQSPDSLAVSLGERATINCSAGSSVSFMHW |
| | | YQQKPGQPPKLLIYDTSKLASGVPDRFSGSGSGN |
| | | DFTLTISSLQAEDVAVYYCLQGSGFPLTFGQGTKL |
| | | EIK |

Antibody IJ (murine), Binds to human TCRVβ 5-1

| | | |
|---|---|---|
| SEQ ID NO: 1163 | HC CDR1 (Kabat) | DYNIH |
| SEQ ID NO: 1164 | HC CDR2 (Kabat) | YINPYNGRTGYNQKFKA |
| SEQ ID NO: 1165 | HC CDR3 (Kabat) | WDGS SYFDY |
| SEQ ID NO: 1166 | HC CDR1 (Chothia) | GYTFTDYNIH |
| SEQ ID NO: 1167 | HC CDR2 (Chothia) | NPYNGR |
| SEQ ID NO: 1165 | HC CDR3 (Chothia) | WDGSSYFDY |
| SEQ ID NO: 1166 | HC CDR1 (Combined) | GYTFTDYNIH |
| SEQ ID NO: 1164 | HC CDR2 (Combined) | YINPYNGRTGYNQKFKA |
| SEQ ID NO: 1165 | HC CDR3(Combined) | WDGSSYFDY |
| SEQ ID NO: 1168 | LC CDR1 (Kabat) | SASS SVSYMH |
| SEQ ID NO: 1169 | LC CDR2 (Kabat) | EISKLAS |
| SEQ ID NO: 1170 | LC CDR3 (Kabat) | QQWNYPLLT |
| SEQ ID NO: 3476 | LC CDR1 (Chothia) | SSSVSY |
| SEQ ID NO: 1169 | LC CDR2 (Chothia) | EISKLAS |
| SEQ ID NO: 1170 | LC CDR3 (Chothia) | QQWNYPLLT |
| SEQ ID NO: 1168 | LC CDR1 (Combined) | SASSSVSYMH |
| SEQ ID NO: 1169 | LC CDR2 (Combined) | EISKLAS |
| SEQ ID NO: 1170 | LC CDR3(Combined) | QQWNYPLLT |
| SEQ ID NO: 1171 | VL | EIVLTQSPAITAASLGQKVTITCSASSSVSYMHWY |
| | | QQKSGTSPKPWIYEISKLASGVPARFSGSGSGTSY |
| | | SLTISSMEAEDAAIYYCQQWNYPLLTFGAGTKLE |
| | | LK |
| SEQ ID NO: 1172 | VH | EVQLQQSGPVLVKPGASVRMSCKASGYTFTDYNI |
| | | HWVKQSHGRSLEWVGYINPYNGRTGYNQKFKA |
| | | KATLTVDKSSSTAYMDLRSLTSEDSAVYYCARW |
| | | DGSSYFDYWGQGTTLTVSS |

Antibody J-H(humanized) VHs
Binds to human TCRVβ 5-1

| | | |
|---|---|---|
| SEQ ID NO: 1173 | VH - 1 | QVQLVQSGAEVKKPGSSVKVSCKASGYTFTDYNI |
| | | HWVRQAPGQGLEWVGYINPYNGRTGYNQKFKA |
| | | RATLTVDKSTSTAYMELSSLRSEDTAVYYCARW |
| | | DGSSYFDYWGQGTTVTVSS |
| SEQ ID NO: 1174 | VH - 2 | QVQLVQSGAEVKKPGASVKVSCKASGYTFTDYNI |
| | | HWVRQAPGQGLEWVGYINPYNGRTGYNQKFKA |
| | | RATLTVDKSTSTAYMELRSLRSDDMAVYYCARW |
| | | DGSSYFDYWGQGTTVTVSS |
| SEQ ID NO: 1175 | VH - 3 | QVQLVQSGAEVKKPGASVKVSCKASGYTFTDYNI |
| | | HWVRQATGQGLEWVGYINPYNGRTGYNQKFKA |
| | | RATLTVNKSISTAYMELSSLRSEDTAVYYCARWD |
| | | GSSYFDYWGQGTTVTVSS |
| SEQ ID NO: 1176 | VH - 4 | EVQLVESGGGLVQPGRSLRLSCTASGYTFTDYNI |
| | | HWVRQAPGKGLEWVGYINPYNGRTGYNQKFKA |
| | | RATLSVDKSKSIAYLQMNSLKTEDTAVYYCARW |
| | | DGSSYFDYWGQGTTVTVSS |
| SEQ ID NO: 1177 | VH - 5 | QVQLVQSGSELKKPGASVKVSCKASGYTFTDYNI |
| | | HWVRQAPGQGLEWVGYINPYNGRTGYNQKFKA |
| | | RAVLSVDKSVSTAYLQISSLKAEDTAVYYCARWD |
| | | GSSYFDYWGQGTTVTVSS |

Antibody J-H (humanized) VLs
Binds to human TCRVβ 5-1

| | | |
|---|---|---|
| SEQ ID NO: 1178 | VL - 1 | EIVLTQSPATLSLSPGERATLSCSASSSVSYMHWY |
| | | QQKPGQAPKLLIYEISKLASGIPARFSGSGSGTDYT |
| | | LTISSLEPEDFAVYYCQQWNYPLLTFGQGTKLEIK |
| SEQ ID NO: 1179 | VL - 2 | EIVLTQSPATLSLSPGERATLSCSASSSVSYMHWY |
| | | QQKPGQAPKLLIYEISKLASGIPARFSGSGSGTDYT |
| | | LTISRLEPEDFAVYYCQQWNYPLLTFGQGTKLEIK |
| SEQ ID NO: 1180 | VL - 3 | EIVLTQSPDFQSVTPKEKVTITCSASSSVSYMHWY |
| | | QQKPDQSPKLLIYEISKLASGVPSRFSGSGSGTDYT |
| | | LTTNSLEAEDAATYYCQQWNYPLLTFGQGTKLEI |
| | | K |
| SEQ ID NO: 1181 | VL - 4 | DIQLTQSPSFLSASVGDRVTITCSASSSVSYMHWY |
| | | QQKPGKAPKLLIYEISKLASGVPSRFSGSGSGTEY |
| | | TLTISSLQPEDFATYYCQQWNYPLLTFGQGTKLEI |
| | | K |
| SEQ ID NO: 1182 | VL - 5 | AIQLTQSPSSLSASVGDRVTITCSASSSVSYMHWY |
| | | QQKPGKAPKLLIYEISKLASGVPSRFSGSGSGTDY |
| | | TLTISSLQPEDFATYYCQQWNYPLLTFGQGTKLEI |
| | | K |
| SEQ ID NO: 1183 | VL - 6 | AIRLTQSPFSLSASVGDRVTITCSASSSVSYMHWY |
| | | QQKPAKAPKLFIYEISKLASGVPSRFSGSGSGTDY |
| | | TLTISSLQPEDFATYYCQQWNYPLLTFGQGTKLEI |
| | | K |
| SEQ ID NO: 1184 | VL - 7 | DIVLTQSPDSLAVSLGERATINCSASSSVSYMHWY |
| | | QQKPGQPPKLLIYEISKLASGVPDRFSGSGSGTDY |
| | | TLTISSLQAEDVAVYYCQQWNYPLLTFGQGTKLE |
| | | IK |

Antibody K (murine), binds to human TCRVβ 28

| | | |
|---|---|---|
| SEQ ID NO: 1185 | HC CDR1 (Kabat) | GSWMN |
| SEQ ID NO: 1186 | HC CDR2 (Kabat) | RIYPGDGDTDYSGKFKG |
| SEQ ID NO: 1187 | HC CDR3 (Kabat) | SGYFNYVPVFDY |
| SEQ ID NO: 1188 | HC CDR1 (Chothia) | GYTFSGS |
| SEQ ID NO: 1189 | HC CDR2 (Chothia) | YPGDGD |
| SEQ ID NO: 1187 | HC CDR3 (Chothia) | SGYFNYVPVFDY |
| SEQ ID NO: 1190 | HC CDR1 (Combined) | GYTFSGSWMN |
| SEQ ID NO: 1186 | HC CDR2 (Combined) | RIYPGDGDTDYSGKFKG |
| SEQ ID NO: 1187 | HC CDR3(Combined) | SGYFNYVPVFDY |
| SEQ ID NO: 1191 | LC CDR1 (Kabat) | SANSTVGYIH |
| SEQ ID NO: 1192 | LC CDR2 (Kabat) | TTSNLAS |
| SEQ ID NO: 1193 | LC CDR3 (Kabat) | HQWSFYPT |
| SEQ ID NO: 1194 | LC CDR1 (Chothia) | NSTVGY |
| SEQ ID NO: 1192 | LC CDR2 (Chothia) | TTSNLAS |
| SEQ ID NO: 1193 | LC CDR3 (Chothia) | HQWSFYPT |
| SEQ ID NO: 1191 | LC CDR1 (Combined) | SANSTVGYIH |
| SEQ ID NO: 1192 | LC CDR2 (Combined) | TTSNLAS |
| SEQ ID NO: 1193 | LC CDR3(Combined) | HQWSFYPT |
| SEQ ID NO: 1195 | VL | QIVLTQSPAIMSASLGEEIALTCSANSTVGYIHWY |
| | | QQKSGTSPKLLIYTTSNLASGVPSRFSGSGSGTFYS |
| | | LTISSVEAEDAADYFCHQWSFYPTFGGGTKLEIK |
| SEQ ID NO: 1196 | VH | QIQLQQSGPEVVKPGASVQISCKASGYTFSGSWM |
| | | NWVKQRPGKGLEWIGRIYPGDGDTDYSGKFKGR |
| | | ATLTADKSSSTAYMRLSSLTSEDSAVYFCARSGY |
| | | FNYVPVFDYWGQGTTLSVSS |

Antibody K-H(humanized) VHs
Binds to human TCRVβ 28

| | | |
|---|---|---|
| SEQ ID NO: 1197 | VH - 1 | QIQLVQSGAEVKKPGASVKVSCKASGYTFSGSW |
| | | MNWVRQAPGQGLEWIGRIYPGDGDTDYSGKFKG |
| | | RATLTADKSTSTAYMELSSLRSEDTAVYYCARSG |
| | | YFNYVPVFDYWGQGTTVTVSS |
| SEQ ID NO: 1198 | VH - 2 | QIQLVQSGAEVKKPGSSVKVSCKASGYTFSGSWM |
| | | NWVRQAPGQGLEWIGRIYPGDGDTDYSGKFKGR |
| | | ATLTADKSTSTAYMELSSLRSEDTAVYYCARSGY |
| | | FNYVPVFDYWGQGTTVTVSS |
| SEQ ID NO: 1199 | VH - 3 | EIQLVQSGAEVKKPGESLKISCKASGYTFSGSWM |
| | | NWVRQMPGKGLEWIGRIYPGDGDTDYSGKFKGQ |
| | | ATLSADKSISTAYLQWSSLKASDTAMYYCARSGY |
| | | FNYVPVFDYWGQGTTVTVSS |
| SEQ ID NO: 1200 | VH - 4 | QIQLVQSGSELKKPGASVKVSCKASGYTFSGSWM |
| | | NWVRQAPGQGLEWIGRIYPGDGDTDYSGKFKGR |
| | | AVLSADKSVSTAYLQISSLKAEDTAVYYCARSGY |
| | | FNYVPVFDYWGQGTTVTVSS |
| SEQ ID NO: 1201 | VH - 5 | QIQLVQSGSELKKPGASVKVSCKASGYTFSGSWM |
| | | NWVRQAPGQGLEWIGRIYPGDGDTDYSGKFKGR |
| | | AVLSADKSVSMAYLQISSLKAEDTAVYYCARSGY |
| | | FNYVPVFDYWGQGTTVTVSS |
| SEQ ID NO: 1202 | VH - 6 | EIQLVESGGGLVQPGRSLRLSCTASGYTFSGSWM |
| | | NWVRQAPGKGLEWIGRIYPGDGDTDYSGKFKGR |
| | | ATLSADKSKSIAYLQMNSLKTEDTAVYYCARSGY |
| | | FNYVPVFDYWGQGTTVTVSS |

Antibody K-H (humanized) VLs
Binds to human TCRVβ 28

| | | |
|---|---|---|
| SEQ ID NO: 1203 | VL - 1 | EIVLTQSPATLSLSPGERATLSCSANSTVGYIHWY |
| | | QQKPGQAPKLLIYTTSNLASGIPARFSGSGSGTDY |
| | | TLTISSLEPEDFAVYFCHQWSFYPTFGQGTKLEIK |
| SEQ ID NO: 1204 | VL - 2 | DIQLTQSPSFLSASVGDRVTITCSANSTVGYIHWY |
| | | QQKPGKAPKLLIYTTSNLASGVPSRFSGSGSGTEY |
| | | TLTISSLQPEDFATYFCHQWSFYPTFGQGTKLEIK |
| SEQ ID NO: 1205 | VL - 3 | EIVLTQSPATLSLSPGERATLSCSANSTVGYIHWY |
| | | QQKPGQAPKLLIYTTSNLASGIPARFSGSGPGTDY |
| | | TLTISSLEPEDFAVYFCHQWSFYPTFGQGTKLEIK |
| SEQ ID NO: 1206 | VL - 4 | DIVLTQSPDSLAVSLGERATINCSANSTVGYIHWY |
| | | QQKPGQPPKLLIYTTSNLASGVPDRFSGSGSGTDY |
| | | TLTISSLQAEDVAVYFCHQWSFYPTFGQGTKLEIK |
| SEQ ID NO: 1207 | VL - 5 | EIVLTQSPDFQSVTPKEKVTITCSANSTVGYIHWY |
| | | QQKPDQSPKLLIYTTSNLASGVPSRFSGSGSGTDY |
| | | TLTINSLEAEDAATYFCHQWSFYPTFGQGTKLEIK |

Antibody L (murine), binds to human TCRVβ 4-1, 4-2, 4-3

| | | |
|---|---|---|
| SEQ ID NO: 1208 | HC CDR1 (Kabat) | DYYMY |
| SEQ ID NO: 1209 | HC CDR2 (Kabat) | TISGGGSYTYSPD SVKG |
| SEQ ID NO: 1210 | HC CDR3 (Kabat) | ERDIYYGNFNAMVY |
| SEQ ID NO: 1211 | HC CDR1 (Chothia) | GFTFSDY |
| SEQ ID NO: 1212 | HC CDR2 (Chothia) | SGGGSY |
| SEQ ID NO: 1210 | HC CDR3 (Chothia) | ERDIYYGNFNAMVY |
| SEQ ID NO: 1213 | HC CDR1 (Combined) | GFTFSDYYMY |
| SEQ ID NO: 1209 | HC CDR2 (Combined) | TISGGGSYTYSPDSVKG |
| SEQ ID NO: 1210 | HC CDR3(Combined) | ERDIYYGNFNAMVY |
| SEQ ID NO: 1214 | LC CDR1 (Kabat) | RASKSVSTSGYSYMH |
| SEQ ID NO: 1215 | LC CDR2 (Kabat) | LASNLES |
| SEQ ID NO: 1216 | LC CDR3 (Kabat) | QHSRDLPWT |
| SEQ ID NO: 1217 | LC CDR1 (Chothia) | SKSVSTSGYSY |
| SEQ ID NO: 1215 | LC CDR2 (Chothia) | LASNLES |
| SEQ ID NO: 1216 | LC CDR3 (Chothia) | QHSRDLPWT |
| SEQ ID NO: 1214 | LC CDR1 (Combined) | RASKSVSTSGYSYMH |
| SEQ ID NO: 1215 | LC CDR2 (Combined) | LASNLES |
| SEQ ID NO: 1216 | LC CDR3(Combined) | QHSRDLPWT |
| SEQ ID NO: 1218 | VL | DIVLTQSPVSLTVSLGQRATISCRASKSVSTSGYSY |
| | | MHWYQQKPGQPPKLLIYLASNLESGVPARFSGSG |
| | | SGTDFTLNIHPVEEEDAATYYCQHSRDLPWTFGG |
| | | GTKLEIK |
| SEQ ID NO: 1219 | VH | EVQLVESGGGLVKPGGSLKLSCAASGFTFSDYYM |
| | | YWVRQTPEKRLEWVATISGGGSYTYSPDSVKGRF |
| | | TISRDNAKNNLYLQMSSLRSEDTAMYFCARERDI |
| | | YYGNFNAMVYWGRGTSVTVSS |

Antibody L-H (humanized) VHs
Binds to human TCRVβ 4-1, 4-2, 4-3

| | | |
|---|---|---|
| SEQ ID NO: 1220 | VH - 1 | EVQLLESGGGLVQPGGSLRLSCAASGFTFSDYYM |
| | | YWVRQAPGKGLEWVATISGGGSYTYSPDSVKGR |
| | | FTISRDNSKNTLYLQMNSLRAEDTAVYYCARERD |
| | | IYYGNFNAMVYWGRGTLVTVSS |
| SEQ ID NO: 1221 | VH - 2 | EVQLVESGGGLVQPGGSLRLSCAASGFTFSDYYM |
| | | YWVRQAPGKGLEWVATISGGGSYTYSPDSVKGR |
| | | FTISRDNAKNSLYLQMNSLRAEDTAVYYCARERD |
| | | IYYGNFNAMVYWGRGTLVTVSS |
| SEQ ID NO: 1222 | VH - 3 | QVQLVESGGGVVQPGRSLRLSCAASGFTFSDYY |
| | | MYWVRQAPGKGLEWVATISGGGSYTYSPDSVK |
| | | GRFTISRDNSKNTLYLQMNSLRAEDTAVYYCAR |
| | | ERDIYYGNFNAMVYWGRGTLVTVSS |
| SEQ ID NO: 1223 | VH - 4 | QVQLVESGGGLVKPGGSLRLSCAASGFTFSDYYM |
| | | YWIRQAPGKGLEWVATISGGGSYTYSPDSVKGRF |
| | | TISRDNAKNSLYLQMNSLRAEDTAVYYCARERDI |
| | | YYGNFNAMVYWGRGTLVTVSS |

Antibody L-H(humanized) VLs
Binds to human TCRVβ 4-1, 4-2, 4-3

| | | |
|---|---|---|
| SEQ ID NO: 1224 | VL - 1 | EIVLTQSPGTLSLSPGERATLSCRASKSVSTSGYSY |
| | | MHWYQQKPGQAPRLLIYLASNLESGIPDRFSGSG |
| | | SGTDFTLTISRLEPEDFAVYYCQHSRDLPWTFGGG |
| | | TKVEIK |
| SEQ ID NO: 1225 | VL - 2 | EIVLTQSPATLSLSPGERATLSCRASKSVSTSGYSY |
| | | MHWYQQKPGQAPRLLIYLASNLESGIPARFSGSG |
| | | SGTDFTLTISSLEPEDFAVYYCQHSRDLPWTFGGG |
| | | TKVEIK |
| SEQ ID NO: 1226 | VL - 3 | DIQLTQSPSTLSASVGDRVTITCRASKSVSTSGYSY |
| | | MHWYQQKPGKAPKLLIYLASNLESGVPSRFSGSG |
| | | SGTEFTLTISSLQPDDFATYYCQHSRDLPWTFGGG |
| | | TKVEIK |
| SEQ ID NO: 1227 | VL - 4 | AIQLTQSPSSLSASVGDRVTITCRASKSVSTSGYSY |
| | | MHWYQQKPGKAPKWYLASNLESGVPSRFSGSG |
| | | SGTDFTLTISSLQPEDFATYYCQHSRDLPWTFGGG |
| | | TKVEIK |

Antibody M (murine), binds to human TCRVβ 19

| | | |
|---|---|---|
| SEQ ID NO: 1229 | HC CDR1 (Kabat) | GYFWN |
| SEQ ID NO: 1230 | HC CDR2 (Kabat) | YISYDGSNNYNPSLKN |
| SEQ ID NO: 1231 | HC CDR3 (Kabat) | PSPGTGYAVDY |
| SEQ ID NO: 1232 | HC CDR1 (Chothia) | GYSITSGY |
| SEQ ID NO: 1233 | HC CDR2 (Chothia) | SYDGSN |
| SEQ ID NO: 1231 | HC CDR3 (Chothia) | PSPGTGYAVDY |
| SEQ ID NO: 1234 | HC CDR1 (Combined) | GYSITSGYFWN |
| SEQ ID NO: 1230 | HC CDR2 (Combined) | YISYDGSNNYNPSLKN |
| SEQ ID NO: 1231 | HC CDR3(Combined) | PSPGTGYAVDY |
| SEQ ID NO: 1235 | LC CDR1 (Kabat) | RSSQSLVHSNGNTYLH |
| SEQ ID NO: 1236 | LC CDR2 (Kabat) | KVSNRFS |
| SEQ ID NO: 1237 | LC CDR3 (Kabat) | SQSTHVPFT |
| SEQ ID NO: 1238 | LC CDR1 (Chothia) | SQSLVHSNGNTY |
| SEQ ID NO: 1236 | LC CDR2 (Chothia) | KVSNRFS |
| SEQ ID NO: 1237 | LC CDR3 (Chothia) | SQSTHVPFT |
| SEQ ID NO: 1235 | LC CDR1 (Combined) | RSSQSLVHSNGNTYLH |
| SEQ ID NO: 1236 | LC CDR2 (Combined) | KVSNRFS |
| SEQ ID NO: 1237 | LC CDR3(Combined) | SQSTHVPFT |
| SEQ ID NO: 1239 | VL | NVVMTQTPLSLPVSLGDQASISCRSSQSLVHSNGN |
| | | TYLHWYLQKPGQSPKFLIYKVSNRFSGVPDRFSG |
| | | GGSGTEFTLKISRVEAEDLGVYFCSQSTHVPFTFG |
| | | SGTKLEIK |
| SEQ ID NO: 1240 | VH | NVQLQESGPGLVKPSQSLSLTCSVAGYSITSGYFW |
| | | NWIRQFPGNKLEWMGYISYDGSNNYNPSLKNRIS |
| | | ITRDTSKNQFFLKLNSVTTEDTATYYCASPSPGTG |
| | | YAVDYWGQGTSVTVSS |

Antibody M-H (humanized) VHs
Binds to human TCRVβ 19

| | | |
|---|---|---|
| SEQ ID NO: 1241 | VH - 1 | QVQLQESGPGLVKPSETLSLTCTVSGYSITSGYFW |
| | | NWIRQPPGKGLEWIGYISYDGSNNYNPSLKNRVTI |
| | | SRDTSKNQFSLKLSSVTAADTAVYYCASPSPGTG |
| | | YAVDYWGQGTLVTVSS |
| SEQ ID NO: 1242 | VH - 2 | QVQLQESGPGLVKPSETLSLTCTVSGYSITSGYFW |
| | | NWIRQPPGKGLEWIGYISYDGSNNYNPSLKNRVTI |
| | | SRDTSKNQFSLKLSSVTAADTAVYYCASPSPGTG |
| | | YAVDYWGQGTLVTVSS |
| SEQ ID NO: 1243 | VH - 3 | QVQLVESGGGLVQPGGSLRLSCSVSGYSITSGYF |
| | | WNWVRQAPGKGLEWVGYISYDGSNNYNPSLKN |
| | | RFTISRDTSKNTFYLQMNSLRAEDTAVYYCASPSP |
| | | GTGYAVDYWGQGTLVTVSS |

Antibody M - H (humanized) VLs
Binds to human TCRVβ 19

| | | |
|---|---|---|
| SEQ ID NO: 1244 | VL - 1 | VVMTQSPGTLSLSPGERATLSCRSSQSLVHSNGNT |
| | | YLHWYQQKPGQAPRFLIYKVSNRFSGIPDRFSGSG |
| | | SGTDFTLTISRLEPEDFAVYFCSQSTHVPFTFGQGT |
| | | KLEIK |
| SEQ ID NO: 1245 | VL - 2 | EVVMTQSPATLSLSPGERATLSCRSSQSLVHSNGN |
| | | TYLHWYQQKPGQAPRFLIYKVSNRFSGIPARFSGS |
| | | GSGTDFTLTISSLEPEDFAVYFCSQSTHVPFTFGQG |
| | | TKLEIK |
| SEQ ID NO: 1246 | VL - 3 | EVVMTQSPATLSVSPGERATLSCRSSQSLVHSNG |
| | | NTYLHWYQQKPGQAPRFLIYKVSNRFSGIPARFS |
| | | GSGSGTEFTLTISSLQSEDFAVYFCSQSTHVPFTFG |
| | | QGTKLEIK |
| SEQ ID NO: 1247 | VL - 4 | DVQMTQSPSSLSASVGDRVTITCRSSQSLVHSNGN |
| | | TYLHWYQQKPGKAPKFLIYKVSNRFSGVPSRFSG |
| | | SGSGTDFTFTISSLQPEDIATYFCSQSTHVPFTFGQ |
| | | GTKLEIK |

Antibody N(murine) binds to human TCRVβ 9

| | | |
|---|---|---|
| SEQ ID NO: 1248 | HC CDR1 (Kabat) | DYIVH |
| SEQ ID NO: 1249 | HC CDR2 (Kabat) | WINTYTGTPTYADDFEG |
| SEQ ID NO: 1250 | HC CDR3 (Kabat) | SWRRGIRGIGFDY |
| SEQ ID NO: 1251 | HC CDR1 (Chothia) | GYTFTDY |
| SEQ ID NO: 1252 | HC CDR2 (Chothia) | NTYTGT |
| SEQ ID NO: 1250 | HC CDR3 (Chothia) | SWRRGIRGIGFDY |
| SEQ ID NO: 1253 | HC CDR1 (Combined) | GYTFTDYIVH |
| SEQ ID NO: 1249 | HC CDR2 (Combined) | WINTYTGTPTYADDFEG |
| SEQ ID NO: 1250 | HC CDR3(Combined) | SWRRGIRGIGFDY |
| SEQ ID NO: 1254 | LC CDR1 (Kabat) | KASKSINKYLA |
| SEQ ID NO: 1255 | LC CDR2 (Kabat) | DGSTLQS |
| SEQ ID NO: 1256 | LC CDR3 (Kabat) | QQHNEYPPT |
| SEQ ID NO: 1257 | LC CDR1 (Chothia) | SKSINKY |
| SEQ ID NO: 1255 | LC CDR2 (Chothia) | DGSTLQS |
| SEQ ID NO: 1256 | LC CDR3 (Chothia) | QQHNEYPPT |
| SEQ ID NO: 1254 | LC CDR1 (Combined) | KASKSINKYLA |
| SEQ ID NO: 1255 | LC CDR2 (Combined) | DGSTLQS |
| SEQ ID NO: 1256 | LC CDR3(Combined) | QQHNEYPPT |
| SEQ ID NO: 1258 | VL | DVQMTQSPYNLAASPGESVSINCKASKSINKYLA |
| | | WYQQKPGKPNKLLIYDGSTLQSGIPSRFSGSGSGT |
| | | DFTLTIRGLEPEDFGLYYCQQHNEYPPTFGAGTKL |
| | | ELK |
| SEQ ID NO: 1259 | VH | QLQLVQSGPELREPGESVKISCKASGYTFTDYIVH |
| | | WVKQAPGKGLKWMGWINTYTGTPTYADDFEGR |
| | | FVFSLEASASTANLQISNLKNEDTATYFCARSWRR |
| | | GIRGIGFDYWGQGVMVTVSS |

Antibody N-H (humanized) VH's
Binds to human TCRVβ 9

| | | |
|---|---|---|
| SEQ ID NO: 1260 | VH - 1 | QLQLVQSGAEVKKPGASVKVSCKASGYTFTDYIV |
| | | HWVRQAPGQGLEWMGWINTYTGTPTYADDFEG |
| | | WVTMTLDASISTAYMELSRLRSDDTAVYYCARS |
| | | WRRGIRGIGFDYWGQGTMVTVSS |
| SEQ ID NO: 1261 | VH - 2 | QLQLVQSGAEVKKPGASVKVSCKASGYTFTDYIV |
| | | HWVRQAPGQGLEWMGWINTYTGTPTYADDFEG |
| | | RVTMTLDASTSTAYMELSSLRSEDTAVYYCARS |
| | | WRRGIRGIGFDYWGQGTMVTVSS |
| SEQ ID NO: 1262 | VH - 3 | QLQLVQSGAEVKKPGASVKVSCKASGYTFTDYIV |
| | | HWVRQAPGQRLEWMGWINTYTGTPTYADDFEG |
| | | RVTITLDASASTAYMELSSLRSEDMAVYYCARSW |
| | | RRGIRGIGFDYWGQGTMVTVSS |
| SEQ ID NO: 1263 | VH - 4 | QLQLVQSGAEVKKPGASVKVSCKASGYTFTDYIV |
| | | HWVRQATGQGLEWMGWINTYTGTPTYADDFEG |
| | | RVTMTLNASISTAYMELSSLRSEDTAVYYCARSW |
| | | RRGIRGIGFDYWGQGTMVTVSS |

Antibody N-H (humanized) VL's
Binds to human TCRVβ 9

| | | |
|---|---|---|
| SEQ ID NO: 1264 | VL - 1 | EVVMTQSPGTLSLSPGERATLSCKASKSNKYLA |
| | | WYQQKPGQAPRLLIYDGSTLQSGIPDRFSGSGSGT |
| | | DFTLTISRLEPEDFAVYYCQQHNEYPPTFGQGTKL |
| | | EIK |
| SEQ ID NO: 1265 | VL - 2 | EVVMTQSPATLSLSPGERATLSCKASKSINKYLA |
| | | WYQQKPGQAPRLLIYDGSTLQSGIPARFSGSGSGT |
| | | DFTLTISSLEPEDFAVYYCQQHNEYPPTFGQGTKL |
| | | EIK |
| SEQ ID NO: 1266 | VL - 3 | DVQMTQSPSSLSASVGDRVTITCKASKSINKYLA |
| | | WYQQKPGKAPKLLIYDGSTLQSGVPSRFSGSGSG |
| | | TDFTLTISSLQPEDFATYYCQQHNEYPPTFGQGTK |
| | | LEIK |
| SEQ ID NO: 1267 | VL - 4 | AVRMTQSPSSFSASTGDRVTITCKASKSINKYLAW |
| | | YQQKPGKAPKLLIYDGSTLQSGVPSRFSGSGSGTD |
| | | FTLTISCLQSEDFATYYCQQHNEYPPTFGQGTKLE |
| | | IK |

Antibody O (murine) binds to TRVβ 11-2

| | | |
|---|---|---|
| SEQ ID NO: 1268 | HC CDR1 (Kabat) | NYGVH |
| SEQ ID NO: 1269 | HC CDR2 (Kabat) | VIWSDGSTDYDTAFIS |
| SEQ ID NO: 1270 | HC CDR3 (Kabat) | RAVVADFDY |
| SEQ ID NO: 1271 | HC CDR1 (Chothia) | GFSLTN |
| SEQ ID NO: 1272 | HC CDR2 (Chothia) | VIWSDGSTD |
| SEQ ID NO: 1270 | HC CDR3 (Chothia) | RAVVADFDY |
| SEQ ID NO: 1273 | HC CDR1 (combined) | GFSLTNYGVH |
| SEQ ID NO: 1269 | HC CDR2 (combined) | VIWSDGSTDYDTAFIS |
| SEQ ID NO: 1270 | HC CDR3 (combined) | RAVVADFDY |
| SEQ ID NO: 1274 | VH | QVQLKQSGPGLLQPSQSLSITCTVSGFSLTNYGVH |
| | | WVRQSPGKGLEWLGVIWSDGSTDYDTAFISRLSIS |
| | | KDNSKSQVFFKLNSLQADDTAIYYCARRAVVADF |
| | | DYWGQGTTLTVSS |
| SEQ ID NO: 1275 | LC CDR1 (Kabat) | KASKEVTIFGSISALH |
| SEQ ID NO: 1276 | LC CDR2 (Kabat) | NGAKLES |
| SEQ ID NO: 1277 | LC CDR3 (Kabat) | LQNKEVPFT |
| SEQ ID NO: 1275 | LC CDR1 (Chothia) | KASKEVTIFGSISALH |
| SEQ ID NO: 1276 | LC CDR2 (Chothia) | NGAKLES |
| SEQ ID NO: 1277 | LC CDR3 (Chothia) | LQNKEVPFT |
| SEQ ID NO: 1275 | LC CDR1 (combined) | KASKEVTIFGSISALH |
| SEQ ID NO: 1276 | LC CDR2 (combined) | NGAKLES |
| SEQ ID NO: 1277 | LC CDR3 (combined) | LQNKEVPFT |
| SEQ ID NO: 1278 | VL | DIVLTQSPASLAVSLGQKATISCKASKEVTIFGSIS |
| | | ALHWYQQKPGQPPKLIYNGAKLESGVSARFSDS |
| | | GSQNRSPFGNQLSFTLTIAPVEADDAATYYCLQN |
| | | KEVPFTFGSGTKLEIK |

Antibody O-H (humanized) VL binds to TRVβ 11-2

| | | |
|---|---|---|
| SEQ ID NO: 1279 | VL-1 | DIVLTQSPDSLAVSLGERATINCKASKEVTIFGSIS |
| | | ALHWYQQKPGQPPKLLYNGAKLESGVSARFGVP |
| | | DRFSRSGSGLDFTLTISSLQAEDVAVYYCLQNKE |
| | | VPFTFGQGTKLEIK |
| SEQ ID NO: 1280 | VL-2 | EIVLTQSPDFQSVTPKEKVTITCKASKEVTIFGSIS |
| | | ALHWYQQKPDQSPKLLYNGAKLESGVSARFGVP |
| | | SRF SRSGSGLDFTLTINSLEAEDAATYYCLQNKE |
| | | VPFTFGQGTKLEIK |
| SEQ ID NO: 1281 | VL-3 | AIQLTQSPSSLSASVGDRVTITCKASKEVTIFGSIS |
| | | ALHWYQQKPGKAPKLLYNGAKLESGVSARFGV |
| | | PSRFSRSGSGLDFTLTISSLQPEDFATYYCLQNKE |
| | | VPFTFGQGTKLEIK |
| SEQ ID NO: 1282 | VL-4 | DIVLTQTPLSLSVTPGQPASISCKASKEVTIFGSIS |
| | | ALHWYLQKPGQPPKLLYNGAKLESGVSARFGVP |
| | | DRFSRSGSGLDFTLKISRVEAEDVGVYYCLQNKE |
| | | VPFTFGQGTKLEIK |

Antibody O-H (humanized) VH, binds to TRVβ 11-2

| | | |
|---|---|---|
| SEQ ID NO: 1283 | VH-1 | QVTLKESGPVLVKPTETLTLTCTVSGFSLTNYGV |
| | | HWVRQPPGKALEWLGVIWSDGSTDYDTAFISRL |
| | | TISKDNSKSQVVLTMTNMDPVDTATYYCARRAV |
| | | VADFDYWGQGTTVTVSS |
| SEQ ID NO: 1284 | VH-2 | QVQLQESGPGLVKPSGTLSLTCAVSGFSLTNYGV |
| | | HWVRQPPGKGLEWLGVIWSDGSTDYDTAFISRL |
| | | TISKDNSKSQVSLKLSSVTAADTAVYYCARRAV |
| | | VADFDYWGQGTTVTVSS |
| SEQ ID NO: 1285 | VH-3 | QVQLQQSGPGLVKPSQTLSLTCAVSGFSLTNYGV |
| | | HWVRQSPSRGLEWLGVIWSDGSTDYDTAFISRL |
| | | TINKDNSKSQVSLQLNSVTPEDTAVYYCARRAV |
| | | VADFDYWGQGTTVTVSS |
| SEQ ID NO: 1286 | VH-4 | EVQLVESGGGLVQPGPSLRLSCTVSGFSLTNYGV |
| | | HWVRQAPGKGLEWLGVIWSDGSTDYDTAFISRL |
| | | TISKDNSKSIVYLQMNSLKTEDTAVYYCARRAV |
| | | VADFDYWGQGTTVTVSS |
| SEQ ID NO: 1287 | VH-5 | EVQLVQSGAEVKKPGESLRISCKVSGFSLTNYGV |
| | | HWVRQMPGKGLEWLGVIWSDGSTDYDTAFISQ |
| | | LTISKDNSISTVYLQWSSLKASDTAMYYCARRA |
| | | VVADFDYWGQGTTVTVSS |

Amino acid and nucleotide sequences for murine and humanized antibody molecules which bind to various TCRVB families are disclosed. The amino acid the heavy and light chain CDRs, and the amino acid and nucleotide sequences of the heavy and light chain variable regions, and the heavy and light chains are shown. Antibodies disclosed in the table include, MPB2D5, CAS1.1.3, IMMU222, REA1062, and JOVI-3. MPB2D5 binds human TCRβV 20-1 (TCRβV2 per old nomenclature). CAS1.1.3 binds human TCRβV 27 (TCRβV14 per old nomenclature). IMMU 222 binds human TCRβV 6-5, TCRβV 6-6, or TCRβV 6-9 (TCRβV13.1 per old nomenclature). REA1062 binds human TCRβV 5-1). JOVI-3 binds human TCRβV 28 (TCRβV3.1 per old nomenclature).

### Anti-TCRVβ Antibody Effector Function and Fc Variants

In some embodiments, an anti-TCRVβ antibody disclosed herein comprises an Fc region, e.g., as described herein. In some embodiments, the Fc region is a wildtype Fc region, e.g., a wildtype human Fc region. In some embodiments, the Fc region comprises a variant, e.g., an Fc region comprising an addition, substitution, or deletion of at least one amino acid residue in the Fc region which results in, e.g., reduced or ablated affinity for at least one Fc receptor.

The Fc region of an antibody interacts with a number of receptors or ligands including Fc Receptors (e.g., FcyRI, FcyRIIA, FcyRIIIA), the complement protein C1q, and other molecules such as proteins A and G. These interactions are essential for a variety of effector functions and downstream signaling events including: antibody dependent cell-mediated cytotoxicity (ADCC), Antibody-dependent cellular phagocytosis (ADCP) and complement dependent cytotoxicity (CDC).

In some embodiments, an anti-TCRVβ antibody comprising a variant Fc region has reduced, e.g., ablated, affinity for an Fc receptor, e.g., an Fc receptor described herein. In some embodiments, the reduced affinity is compared to an otherwise similar antibody with a wildtype Fc region.

In some embodiments, an anti-TCRVβ antibody comprising a variant Fc region has one or more of the following properties: (1) reduced effector function (e.g., reduced ADCC, ADCP and/or CDC); (2) reduced binding to one or more Fc receptors; and/or (3) reduced binding to C1q complement. In some embodiments, the reduction in any one, or all of properties (1)-(3) is compared to an otherwise similar antibody with a wildtype Fc region.

In some embodiments, an anti-TCRVβ antibody comprising a variant Fc region has reduced affinity to a human Fc receptor, e.g., FcyR I, FcyR II and/or FcyR III. In some embodiments, the anti-TCRVβ antibody comprising a variant Fc region comprises a human IgG1 region or a human IgG4 region.

In some embodiments, an anti-TCRVβ antibody comprising a variant Fc region activates and/or expands T cells, e.g., as described herein. In some embodiments, an anti-TCRVβ antibody comprising a variant Fc region has a cytokine profile described herein, e.g., a cytokine profile that differs from a cytokine profile of a T cell engager that binds to a receptor or molecule other than a TCRβV region (“a non-TCRβV-binding T cell engager”). In some embodiments, the non-TCRβV-binding T cell engager comprises an antibody that binds to a CD3 molecule (e.g., CD3 epsilon (CD3e) molecule); or a TCR alpha (TCRα) molecule.

Exemplary Fc region variants are provided in Table 10 and also disclosed in Saunders O, (2019) Frontiers in Immunology; vol 10, article1296, the entire contents of which is hereby incorporated by reference.

In some embodiments, an anti-TCRVβ antibody disclosed herein comprises any one or all, or any combination of Fc region variants, e.g., mutations, disclosed in Table 10. In some embodiments, an anti-TCRVβ antibody disclosed herein comprise an Asn297Ala (N297A) mutation. In some embodiments, an anti-TCRVβ antibody disclosed herein comprise a Leu234A1a/Leu235Ala (LALA) mutation.

TABLE 10
Exemplary Fc modifications

| Modification or mutation | Altered effector function |
|---|---|
| Leu235Glu | ADCC; |
| Leu234Ala/Leu235A1a (LALA) | ADCC; ADCP; CDC |
| Ser228Pro/Leu235Glu | |
| Leu234Ala/Leu235Ala/Pro329G1y | ADCP |
| Pro331Ser/Leu234Glu/Leu235Phe | CDC |
| Asp265Ala | ADCC, ADCP |
| Gly237Ala | ADCP |
| Glu318Ala | ADCP |
| Glu233Pro | |
| Gly236Arg/Leu328Arg | ADCC |
| His268GlnNa1309Leu/Ala330Ser/Pro331Ser | ADCC; ADCP; CDC |
| Val234A1a/Gly237Ala/Pro238Ser/ | ADCC; ADCP; CDC |
| His268Ala/Va1309Leu/Ala330Ser/Pro331Ser | |
| Leu234Ala/L235Ala/Gly237Ala/P238Ser/ | ADCC; CDC |
| His268A1a/Ala330 Ser/Pro331 Ser | |
| Ala330Leu | CDC |
| Asp270Ala | CDC |
| Lys322Ala | CDC |
| Pro329Ala | CDC |
| Pro331Ala | CDC |
| Val264Ala | CDC |
| High mannose glycosylation | CDC |
| Phe241Ala | CDC |
| Asn297Ala or Gly or Gln | ADCC; ADCP; CDC |
| S228P/Phe234Ala/Leu235Ala | ADCC; CDC |

### Antibody Molecules

In one embodiment, the antibody molecule binds to an infectious disease antigen, e.g., as described herein. In some embodiments, the antigen is, e.g., a bacterial, viral, fungal, or malarial antigen. In other embodiments, the antibody molecule binds to an immune cell antigen, e.g., a mammalian, e.g., a human, immune cell antigen. For example, the antibody molecule binds specifically to an epitope, e.g., linear or conformational epitope, on the infectious disease antigen or the immune cell antigen.

In an embodiment, an antibody molecule is a monospecific antibody molecule and binds a single epitope. E.g., a monospecific antibody molecule having a plurality of immunoglobulin variable domain sequences, each of which binds the same epitope.

In an embodiment an antibody molecule is a multispecific or multifunctional antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a multispecific antibody molecule comprises a third, fourth or fifth immunoglobulin variable domain. In an embodiment, a multispecific antibody molecule is a bispecific antibody molecule, a trispecific antibody molecule, or a tetraspecific antibody molecule.

In an embodiment a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv or a Fab, or fragment thereof, have binding specificity for a first epitope and a scFv or a Fab, or fragment thereof, have binding specificity for a second epitope.

In an embodiment, an antibody molecule comprises a diabody, and a single-chain molecule, as well as an antigen-binding fragment of an antibody (e.g., Fab, F(ab′)₂, and Fv). For example, an antibody molecule can include a heavy (H) chain variable domain sequence (abbreviated herein as VH), and a light (L) chain variable domain sequence (abbreviated herein as VL). In an embodiment an antibody molecule comprises or consists of a heavy chain and a light chain (referred to herein as a half antibody. In another example, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The a preparation of antibody molecules can be monoclonal or polyclonal. An antibody molecule can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. The term “immunoglobulin” (Ig) is used interchangeably with the term “antibody” herein.

Examples of antigen-binding fragments of an antibody molecule include: (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments are obtained using conventional techniques known to those with skill in the art, and the fragments are screened for utility in the same manner as are intact antibodies.

Antibody molecules include intact molecules as well as functional fragments thereof. Constant regions of the antibody molecules can be altered, e.g., mutated, to modify the properties of the antibody (e.g., to increase or decrease one or more of: Fc receptor binding, antibody glycosylation, the number of cysteine residues, effector cell function, or complement function).

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg).

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be determined using any of a number of known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273,927-948 (“Chothia” numbering scheme). As used herein, the CDRs defined according the “Chothia” number scheme are also sometimes referred to as “hypervariable loops.”

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia, the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3).

Each VH and VL typically includes three CDRs and four FRs, arranged from amino-terminus to carboxy-terminus in the following order: FR1, CDR1, FR2, CDR2, FR3, CDR3, FR4.

The antibody molecule can be a polyclonal or a monoclonal antibody.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

The antibody can be recombinantly produced, e.g., produced by phage display or by combinatorial methods.

Phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In one embodiment, the antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immnol 21:1323-1326).

An antibody molecule can be one in which the variable region, or a portion thereof, e.g., the CDRs, are generated in a non-human organism, e.g., a rat or mouse. Chimeric, CDR-grafted, and humanized antibodies are within the invention. Antibody molecules generated in a non-human organism, e.g., a rat or mouse, and then modified, e.g., in the variable framework or constant region, to decrease antigenicity in a human are within the invention.

An “effectively human” protein is a protein that does substantially not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

Chimeric antibodies can be produced by recombinant DNA techniques known in the art (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U .S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immuoglobulin chains) replaced with a donor CDR. The antibody may be replaced with at least a portion of a non-human CDR or only some of the CDRs may be replaced with non-human CDRs. It is only necessary to replace the number of CDRs required for binding to the antigen. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody molecule can be humanized by methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference).

Humanized or CDR-grafted antibody molecules can be produced by CDR-grafting or CDR substitution, wherein one, two, or all CDRs of an immunoglobulin chain can be replaced. See e.g., U .S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibody molecules in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

The antibody molecule can be a single chain antibody. A single-chain antibody (scFv) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target protein.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has: effector function; and can fix complement. In other embodiments the antibody does not; recruit effector cells; or fix complement. In another embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g. altered affinity for an effector ligand, such as FcR on a cell, or the Cl component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Similar type of alterations could be described which if applied to the murine, or other species immunoglobulin would reduce or eliminate these functions.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules of the invention are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

### Multispecific or Multifunctional Antibody Molecules

Exemplary structures of multispecific and multifunctional molecules defined herein are described throughout. Exemplary structures are further described in: Weidle U et al. (2013) The Intriguing Options of Multispecific Antibody Formats for Treatment of Cancer. Cancer Genomics & Proteomics 10: 1-18 (2013); and Spiess C et al. (2015) Alternative molecular formats and therapeutic applications for bispecific antibodies. Molecular Immunology 67: 95-106; the full contents of each of which is incorporated by reference herein).

In embodiments, multispecific antibody molecules can comprise more than one antigen-binding site, where different sites are specific for different antigens. In embodiments, multispecific antibody molecules can bind more than one (e.g., two or more) epitopes on the same antigen. In embodiments, multispecific antibody molecules comprise an antigen-binding site specific for a target cell (e.g., an infectious agent) and a different antigen-binding site specific for an immune effector cell. In one embodiment, the multispecific antibody molecule is a bispecific antibody molecule. Bispecific antibody molecules can be classified into five different structural groups: (i) bispecific immunoglobulin G (BsIgG); (ii) IgG appended with an additional antigen-binding moiety; (iii) bispecific antibody fragments; (iv) bispecific fusion proteins; and (v) bispecific antibody conjugates.

BsIgG is a format that is monovalent for each antigen. Exemplary BsIgG formats include but are not limited to crossMab, DAF (two-in-one), DAF (four-in-one), DutaMab, DT-IgG, knobs-in-holes common LC, knobs-in-holes assembly, charge pair, Fab-arm exchange, SEEDbody, triomab, LUZ-Y, Fcab, κλ-body, orthogonal Fab. See Spiess et al. Mol. Immunol. 67(2015):95-106. Exemplary BsIgGs include catumaxomab (Fresenius Biotech, Trion Pharma, Neopharm), which contains an anti-CD3 arm and an anti-EpCAM arm; and ertumaxomab (Neovii Biotech, Fresenius Biotech), which targets CD3 and HER2. In some embodiments, BsIgG comprises heavy chains that are engineered for heterodimerization. For example, heavy chains can be engineered for heterodimerization using a “knobs-into-holes” strategy, a SEED platform, a common heavy chain (e.g., in κλ-bodies), and use of heterodimeric Fc regions. See Spiess et al. Mol. Immunol. 67(2015):95-106. Strategies that have been used to avoid heavy chain pairing of homodimers in BsIgG include knobs-in-holes, duobody, azymetric, charge pair, HA-TF, SEEDbody, and differential protein A affinity. See Id. BsIgG can be produced by separate expression of the component antibodies in different host cells and subsequent purification/assembly into a BsIgG. BsIgG can also be produced by expression of the component antibodies in a single host cell. BsIgG can be purified using affinity chromatography, e.g., using protein A and sequential pH elution.

IgG appended with an additional antigen-binding moiety is another format of bispecific antibody molecules. For example, monospecific IgG can be engineered to have bispecificity by appending an additional antigen-binding unit onto the monospecific IgG, e.g., at the N- or C-terminus of either the heavy or light chain. Exemplary additional antigen-binding units include single domain antibodies (e.g., variable heavy chain or variable light chain), engineered protein scaffolds, and paired antibody variable domains (e.g., single chain variable fragments or variable fragments). See Id. Examples of appended IgG formats include dual variable domain IgG (DVD-Ig), IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, zybody, and DVI-IgG (four-in-one). See Spiess et al. Mol. Immunol. 67(2015):95-106. An example of an IgG-scFv is MM-141 (Merrimack Pharmaceuticals), which binds IGF-1R and HER3. Examples of DVD-Ig include ABT-981 (AbbVie), which binds IL-1α and IL-1β; and ABT-122 (AbbVie), which binds TNF and IL-17A.

Bispecific antibody fragments (BsAb) are a format of bispecific antibody molecules that lack some or all of the antibody constant domains. For example, some BsAb lack an Fc region. In embodiments, bispecific antibody fragments include heavy and light chain regions that are connected by a peptide linker that permits efficient expression of the BsAb in a single host cell. Exemplary bispecific antibody fragments include but are not limited to nanobody, nanobody-HAS, BiTE, Diabody, DART, TandAb, scDiabody, scDiabody-CH3, Diabody-CH3, triple body, miniantibody, minibody, TriBi minibody, scFv-CH3 KIH, Fab-scFv, scFv-CH-CL-scFv, F(ab′)₂, F(ab')2-scFv2, scFv-KIH, Fab-scFv-Fc, tetravalent HCAb, scDiabody-Fc, Diabody-Fc, tandem scFv-Fc, and intrabody. See Id. For example, the BiTE format comprises tandem scFvs, where the component scFvs bind to CD3 on T cells and an antigen of an infectious agent or portion thereof.

Bispecific fusion proteins include antibody fragments linked to other proteins, e.g., to add additional specificity and/or functionality. An example of a bispecific fusion protein is an immTAC, which comprises an anti-CD3 scFv linked to an affinity-matured T-cell receptor that recognizes HLA-presented peptides. In embodiments, the dock-and-lock (DNL) method can be used to generate bispecific antibody molecules with higher valency. Also, fusions to albumin binding proteins or human serum albumin can be extend the serum half-life of antibody fragments. See Id.

In embodiments, chemical conjugation, e.g., chemical conjugation of antibodies and/or antibody fragments, can be used to create BsAb molecules. See Id. An exemplary bispecific antibody conjugate includes the CovX-body format, in which a low molecular weight drug is conjugated site-specifically to a single reactive lysine in each Fab arm or an antibody or fragment thereof. In embodiments, the conjugation improves the serum half-life of the low molecular weight drug. An exemplary CovX-body is CVX-241 (NCT01004822), which comprises an antibody conjugated to two short peptides inhibiting either VEGF or Ang2. See Id.

The antibody molecules can be produced by recombinant expression, e.g., of at least one or more component, in a host system. Exemplary host systems include eukaryotic cells (e.g., mammalian cells, e.g., CHO cells, or insect cells, e.g., SF9 or S2 cells) and prokaryotic cells (e.g., E. coli). Bispecific antibody molecules can be produced by separate expression of the components in different host cells and subsequent purification/assembly. Alternatively, the antibody molecules can be produced by expression of the components in a single host cell. Purification of bispecific antibody molecules can be performed by various methods such as affinity chromatography, e.g., using protein A and sequential pH elution. In other embodiments, affinity tags can be used for purification, e.g., histidine-containing tag, myc tag, or streptavidin tag.

### Infectious Disease Antigen Moiety

In an aspect, provided herein is a multispecific molecule, e.g., a bispecific molecule, comprising:
(i) a first moiety (e.g., a first immune cell engager) comprising the anti-TCRβV antibody molecule described herein; and
(ii) a second moiety comprising one or more of: an infectious disease-targeting moiety; a second immune cell engager; a cytokine molecule or a stromal modifying moiety.

In some embodiments of any of the compositions or methods disclosed herein, the infectious disease -targeting moiety is an antigen, e.g., an infectious disease antigen, e.g., as described herein.

In some embodiments of any of the compositions or methods disclosed herein, the infectious disease-targeting moiety, e.g., antigen from an infectious agent, is chosen from: EBNA3 (e.g., 339-347), EBNA1 (e.g., 407-417), BZLF1 (e.g., 52-64), matrix protein (e.g., influenza virus matrix protein, e.g., 58-66), HIV Gag (e.g., HIV Gag p17, e.g., 77-85), HIV Env, HIV p24 capsid, SIV Tat (e.g., 28-35), SIV Gag (e.g., 181-189), or HCMV pp65 (e.g., 495-503).

### CDR-Grafted Scaffolds

In embodiments, the antibody molecule is a CDR-grafted scaffold domain. In embodiments, the scaffold domain is based on a fibronectin domain, e.g., fibronectin type III domain. The overall fold of the fibronectin type III (Fn3) domain is closely related to that of the smallest functional antibody fragment, the variable domain of the antibody heavy chain. There are three loops at the end of Fn3; the positions of BC, DE and FG loops approximately correspond to those of CDR1, 2 and 3 of the VH domain of an antibody. Fn3 does not have disulfide bonds; and therefore Fn3 is stable under reducing conditions, unlike antibodies and their fragments (see, e.g., WO 98/56915; WO 01/64942; WO 00/34784). An Fn3 domain can be modified (e.g., using CDRs or hypervariable loops described herein) or varied, e.g., to select domains that bind to an antigen/marker/cell described herein.

In embodiments, a scaffold domain, e.g., a folded domain, is based on an antibody, e.g., a “minibody” scaffold created by deleting three beta strands from a heavy chain variable domain of a monoclonal antibody (see, e.g., Tramontano et al., 1994, J Mol. Recognit. 7:9; and Martin et al., 1994, EMBO J. 13:5303-5309). The “minibody” can be used to present two hypervariable loops. In embodiments, the scaffold domain is a V-like domain (see, e.g., Coia et al. WO 99/45110) or a domain derived from tendamistatin, which is a 74 residue, six-strand beta sheet sandwich held together by two disulfide bonds (see, e.g., McConnell and Hoess, 1995, J Mol. Biol. 250:460). For example, the loops of tendamistatin can be modified (e.g., using CDRs or hypervariable loops) or varied, e.g., to select domains that bind to a marker/antigen/cell described herein. Another exemplary scaffold domain is a beta-sandwich structure derived from the extracellular domain of CTLA-4 (see, e.g., WO 00/60070).

Other exemplary scaffold domains include but are not limited to T-cell receptors; MHC proteins; extracellular domains (e.g., fibronectin Type III repeats, EGF repeats); protease inhibitors (e.g., Kunitz domains, ecotin, BPTI, and so forth); TPR repeats; trifoil structures; zinc finger domains; DNA-binding proteins; particularly monomeric DNA binding proteins; RNA binding proteins; enzymes, e.g., proteases (particularly inactivated proteases), RNase; chaperones, e.g., thioredoxin, and heat shock proteins; and intracellular signaling domains (such as SH2 and SH3 domains). See, e.g., US 20040009530 and U.S. Pat. No. 7,501,121, incorporated herein by reference.

In embodiments, a scaffold domain is evaluated and chosen, e.g., by one or more of the following criteria: (1) amino acid sequence, (2) sequences of several homologous domains, (3) 3-dimensional structure, and/or (4) stability data over a range of pH, temperature, salinity, organic solvent, oxidant concentration. In embodiments, the scaffold domain is a small, stable protein domain, e.g., a protein of less than 100, 70, 50, 40 or 30 amino acids. The domain may include one or more disulfide bonds or may chelate a metal, e.g., zinc.

### Antibody-Based Fusions

A variety of formats can be generated which contain additional binding entities attached to the N or C terminus of antibodies. These fusions with single chain or disulfide stabilized Fvs or Fabs result in the generation of tetravalent molecules with bivalent binding specificity for each antigen. Combinations of scFvs and scFabs with IgGs enable the production of molecules which can recognize three or more different antigens.

### Antibody-Fab Fusion

Antibody-Fab fusions are bispecific antibodies comprising a traditional antibody to a first target and a Fab to a second target fused to the C terminus of the antibody heavy chain. Commonly the antibody and the Fab will have a common light chain. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J. et al. (1997) Nature Biotech 15:159.

### Antibody-scFv Fusion

Antibody-scFv Fusions are bispecific antibodies comprising a traditional antibody and a scFv of unique specificity fused to the C terminus of the antibody heavy chain. The scFv can be fused to the C terminus through the Heavy Chain of the scFv either directly or through a linker peptide. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J. et al. (1997) Nature Biotech 15:159.

### Variable Domain Immunoglobulin DVD

A related format is the dual variable domain immunoglobulin (DVD), which are composed of VH and VL domains of a second specificity place upon the N termini of the V domains by shorter linker sequences.

Other exemplary multispecific antibody formats include, e.g., those described in the following US20160114057A1, US20130243775A1, US20140051833, US20130022601, US20150017187A1, US20120201746A1, US20150133638A1, US20130266568A1, US20160145340A1, WO2015127158A1, US20150203591A1, US20140322221A1, US20130303396A1, US20110293613, US20130017200A1, US20160102135A1, WO2015197598A2, WO2015197582A1, US9359437, US20150018529, WO2016115274A1, WO2016087416A1, US20080069820A1, US9145588B, US7919257, and US20150232560A1. Exemplary multispecific molecules utilizing a full antibody-Fab/scFab format include those described in the following, U.S. Pat. No. 9,382,323B2, US20140072581A1, US20140308285A1, US20130165638A1, US20130267686A1, US20140377269A1, US7741446B2, and WO1995009917A1. Exemplary multispecific molecules utilizing a domain exchange format include those described in the following, US20150315296A1, WO2016087650A1, US20160075785A1, WO2016016299A1, US20160130347A1, US20150166670, U.S. Pat. No. 8,703,132B2, US20100316645, U.S. Pat. No. 8,227,577B2, US20130078249.

### Fc-Containing Entities (Mini-Antibodies)

Fc-containing entities, also known as mini-antibodies, can be generated by fusing scFv to the C-termini of constant heavy region domain 3 (CH3-scFv) and/or to the hinge region (scFv-hinge-Fc) of an antibody with a different specificity. Trivalent entities can also be made which have disulfide stabilized variable domains (without peptide linker) fused to the C-terminus of CH3 domains of IgGs.

### Fc-Containing Multispecific Molecules

In some embodiments, the multispecific molecules disclosed herein includes an immunoglobulin constant region (e.g., an Fc region). Exemplary Fc regions can be chosen from the heavy chain constant regions of IgG1, IgG2, IgG3 or IgG4; more particularly, the heavy chain constant region of human IgG1, IgG2, IgG3, or IgG4.

In some embodiments, the immunoglobulin chain constant region (e.g., the Fc region) is altered, e.g., mutated, to increase or decrease one or more of: Fc receptor binding, antibody glycosylation, the number of cysteine residues, effector cell function, or complement function.

In other embodiments, an interface of a first and second immunoglobulin chain constant regions (e.g., a first and a second Fc region) is altered, e.g., mutated, to increase or decrease dimerization, e.g., relative to a non-engineered interface, e.g., a naturally-occurring interface. For example, dimerization of the immunoglobulin chain constant region (e.g., the Fc region) can be enhanced by providing an Fc interface of a first and a second Fc region with one or more of: a paired protuberance-cavity (“knob-in-a hole”), an electrostatic interaction, or a strand-exchange, such that a greater ratio of heteromultimer to homomultimer forms, e.g., relative to a non-engineered interface.

In some embodiments, the multispecific molecules include a paired amino acid substitution at a position chosen from one or more of 347, 349, 350, 351, 366, 368, 370, 392, 394, 395, 397, 398, 399, 405, 407, or 409, e.g., of the Fc region of human IgG1 For example, the immunoglobulin chain constant region (e.g., Fc region) can include a paired an amino acid substitution chosen from: T366S, L368A, or Y407V (e.g., corresponding to a cavity or hole), and T366W (e.g., corresponding to a protuberance or knob).

In other embodiments, the multifunctional molecule includes a half-life extender, e.g., a human serum albumin or an antibody molecule to human serum albumin.

### Heterodimerized Antibody Molecules & Methods of Making

Various methods of producing multispecific antibodies have been disclosed to address the problem of incorrect heavy chain pairing. Exemplary methods are described below. Exemplary multispecific antibody formats and methods of making said multispecific antibodies are also disclosed in e.g., Speiss et al. Molecular Immunology 67 (2015) 95-106; and Klein et al mAbs 4:6, 653-663; November/December 2012; the entire contents of each of which are incorporated by reference herein.

Heterodimerized bispecific antibodies are based on the natural IgG structure, wherein the two binding arms recognize different antigens. IgG derived formats that enable defined monovalent (and simultaneous) antigen binding are generated by forced heavy chain heterodimerization, combined with technologies that minimize light chain mispairing (e.g., common light chain). Forced heavy chain heterodimerization can be obtained using, e.g., knob-in-hole OR strand exchange engineered domains (SEED).

Knob-in-Hole

Knob-in-Hole as described in U.S. Pat. Nos. 5,731,116, 7,476,724 and Ridgway, J. et al. (1996) Prot. Engineering 9(7): 617-621, broadly involves: (1) mutating the CH3 domain of one or both antibodies to promote heterodimerization; and (2) combining the mutated antibodies under conditions that promote heterodimerization. “Knobs” or “protuberances” are typically created by replacing a small amino acid in a parental antibody with a larger amino acid (e.g., T366Y or T366W); “Holes” or “cavities” are created by replacing a larger residue in a parental antibody with a smaller amino acid (e.g., Y407T, T366S, L368A and/or Y407V).

For bispecific antibodies including an Fc domain, introduction of specific mutations into the constant region of the heavy chains to promote the correct heterodimerization of the Fc portion can be utilized. Several such techniques are reviewed in Klein et al. (mAbs (2012) 4:6, 1-11), the contents of which are incorporated herein by reference in their entirety. These techniques include the “knobs-into-holes” (KiH) approach which involves the introduction of a bulky residue into one of the CH3 domains of one of the antibody heavy chains. This bulky residue fits into a complementary “hole” in the other CH3 domain of the paired heavy chain so as to promote correct pairing of heavy chains (see e.g., U.S. Pat. No. 7,642,228).

Exemplary KiH mutations include S354C, T366W in the “knob” heavy chain and Y349C, T366S, L368A, Y407V in the “hole” heavy chain. Other exemplary KiH mutations are provided in Table 11, with additional optional stabilizing Fc cysteine mutations.

TABLE 11
Exemplary Fc KiH mutations and optional Cysteine mutations

| | Knob | Hole |
|---|---|---|
| Position | Mutation | Mutation |
| 1366 | 1366W | 1366S |
| L368 | — | L368A |
| Y407 | — | Y407V |

Additional Cysteine Mutations to form a stabilizing disulfide bridge

| Position | Knob CH3 | Hole CH3 |
|---|---|---|
| S354 | S354C | — |
| Y349 | — | Y349C |

Other Fc mutations are provided by Igawa and Tsunoda who identified 3 negatively charged residues in the CH3 domain of one chain that pair with three positively charged residues in the CH3 domain of the other chain. These specific charged residue pairs are: E356-K439, E357-K370, D399-K409 and vice versa. By introducing at least two of the following three mutations in chain A: E356K, E357K and D399K, as well as K370E, K409D, K439E in chain B, alone or in combination with newly identified disulfide bridges, they were able to favor very efficient heterodimerization while suppressing homodimerization at the same time (Martens T et al. A novel one-armed antic- Met antibody inhibits glioblastoma growth in vivo. Clin Cancer Res 2006; 12:6144-52; PMID:17062691). Xencor defined 41 variant pairs based on combining structural calculations and sequence information that were subsequently screened for maximal heterodimerization, defining the combination of S364H, F405A (HA) on chain A and Y349T, T394F on chain B (TF) (Moore GL et al. A novel bispecific antibody format enables simultaneous bivalent and monovalent co-engagement of distinct target antigens. MAbs 2011; 3:546-57; PMID: 22123055).

Other exemplary Fc mutations to promote heterodimerization of multispecific antibodies include those described in the following references, the contents of each of which is incorporated by reference herein, WO2016071377A1, US20140079689A1, US20160194389A1, US20160257763, WO2016071376A2, WO2015107026A1, WO2015107025A1, WO2015107015A1, US20150353636A1, US20140199294A1, U.S. Pat. No. 7,750,128B2, US20160229915A1, US20150344570A1, U.S. Pat. No. 8,003,774A1, US20150337049A1, US20150175707A1, US20140242075A1, US20130195849A1, US20120149876A1, US20140200331A1, U.S. Pat. Nos. 9,309,311B2, 8,586,713, US20140037621A1, US20130178605A1, US20140363426A1, US20140051835A1 and US20110054151A1.

Stabilizing cysteine mutations have also been used in combination with KiH and other Fc heterodimerization promoting variants, see e.g., U.S. Pat. No. 7,183,076. Other exemplary cysteine modifications include, e.g., those disclosed in US20140348839A1, U.S. Pat. No. 7,855,275B2, and US9000130B2.

Strand Exchange Engineered Domains (SEED)

Heterodimeric Fc platform that support the design of bispecific and asymmetric fusion proteins by devising strand-exchange engineered domain (SEED) C(H)3 heterodimers are known. These derivatives of human IgG and IgA C(H)3 domains create complementary human SEED C(H)3 heterodimers that are composed of alternating segments of human IgA and IgG C(H)3 sequences. The resulting pair of SEED C(H)3 domains preferentially associates to form heterodimers when expressed in mammalian cells. SEEDbody (Sb) fusion proteins consist of [IgG1 hinge]-C(H)2-[SEED C(H)3], that may be genetically linked to one or more fusion partners (see e.g., Davis JH et al. SEEDbodies: fusion proteins based on strand exchange engineered domain (SEED) CH3 heterodimers in an Fc analogue platform for asymmetric binders or immunofusions and bispecific antibodies. Protein Eng Des Sel 2010; 23:195-202; PMID:20299542 and US8871912. The contents of each of which are incorporated by reference herein).

Duobody

“Duobody” technology to produce bispecific antibodies with correct heavy chain pairing are known. The DuoBody technology involves three basic steps to generate stable bispecific human IgGlantibodies in a post-production exchange reaction. In a first step, two IgGls, each containing single matched mutations in the third constant (CH3) domain, are produced separately using standard mammalian recombinant cell lines. Subsequently, these IgG1 antibodies are purified according to standard processes for recovery and purification. After production and purification (post-production), the two antibodies are recombined under tailored laboratory conditions resulting in a bispecific antibody product with a very high yield (typically >95%) (see e.g., Labrijn et al, PNAS 2013; 110(13):5145-5150 and Labrijn et al. Nature Protocols 2014; 9(10):2450-63, the contents of each of which are incorporated by reference herein).

Electrostatic Interactions

Methods of making multispecific antibodies using CH3 amino acid changes with charged amino acids such that homodimer formation is electrostatically unfavorable are disclosed. EP1870459 and WO 2009089004 describe other strategies for favoring heterodimer formation upon co-expression of different antibody domains in a host cell. In these methods, one or more residues that make up the heavy chain constant domain 3 (CH3), CH3-CH3 interfaces in both CH3 domains are replaced with a charged amino acid such that homodimer formation is electrostatically unfavorable and heterodimerization is electrostatically favorable. Additional methods of making multispecific molecules using electrostatic interactions are described in the following references, the contents of each of which is incorporated by reference herein, include US20100015133, US8592562B2, US9200060B2, US20140154254A1, and U.S. Pat. No. 9,358,286A1.

Common Light Chain

Light chain mispairing needs to be avoided to generate homogenous preparations of bispecific IgGs. One way to achieve this is through the use of the common light chain principle, i.e. combining two binders that share one light chain but still have separate specificities. An exemplary method of enhancing the formation of a desired bispecific antibody from a mixture of monomers is by providing a common variable light chain to interact with each of the heteromeric variable heavy chain regions of the bispecific antibody. Compositions and methods of producing bispecific antibodies with a common light chain as disclosed in, e.g., US7183076B2, US20110177073A1, EP2847231A1, WO2016079081A1, and EP3055329A1, the contents of each of which is incorporated by reference herein.

CrossMab

Another option to reduce light chain mispairing is the CrossMab technology which avoids non-specific L chain mispairing by exchanging CH1 and CL domains in the Fab of one half of the bispecific antibody. Such crossover variants retain binding specificity and affinity, but make the two arms so different that L chain mispairing is prevented. The CrossMab technology (as reviewed in Klein et al. Supra) involves domain swapping between heavy and light chains so as to promote the formation of the correct pairings. Briefly, to construct a bispecific IgG-like CrossMab antibody that could bind to two antigens by using two distinct light chain-heavy chain pairs, a two-step modification process is applied. First, a dimerization interface is engineered into the C-terminus of each heavy chain using a heterodimerization approach, e.g., Knob-into-hole (KiH) technology, to ensure that only a heterodimer of two distinct heavy chains from one antibody (e.g., Antibody A) and a second antibody (e.g., Antibody B) is efficiently formed. Next, the constant heavy 1 (CH1) and constant light (CL) domains of one antibody are exchanged (Antibody A), keeping the variable heavy (VH) and variable light (VL) domains consistent. The exchange of the CH1 and CL domains ensured that the modified antibody (Antibody A) light chain would only efficiently dimerize with the modified antibody (antibody A) heavy chain, while the unmodified antibody (Antibody B) light chain would only efficiently dimerize with the unmodified antibody (Antibody B) heavy chain; and thus only the desired bispecific CrossMab would be efficiently formed (see e.g., Cain, C. SciBX 4(28); doi:10.1038/scibx.2011.783, the contents of which are incorporated by reference herein).

Common Heavy Chain

An exemplary method of enhancing the formation of a desired bispecific antibody from a mixture of monomers is by providing a common variable heavy chain to interact with each of the heteromeric variable light chain regions of the bispecific antibody. Compositions and methods of producing bispecific antibodies with a common heavy chain are disclosed in, e.g., US20120184716, US20130317200, and US20160264685A1, the contents of each of which is incorporated by reference herein.

Amino Acid Modifications

Alternative compositions and methods of producing multispecific antibodies with correct light chain pairing include various amino acid modifications. For example, Zymeworks describes heterodimers with one or more amino acid modifications in the CH1 and/or CL domains, one or more amino acid modifications in the VH and/or VL domains, or a combination thereof, which are part of the interface between the light chain and heavy chain and create preferential pairing between each heavy chain and a desired light chain such that when the two heavy chains and two light chains of the heterodimer pair are co-expressed in a cell, the heavy chain of the first heterodimer preferentially pairs with one of the light chains rather than the other (see e.g., WO2015181805). Other exemplary methods are described in WO2016026943 (Argen-X), US20150211001, US20140072581A1, US20160039947A1, and US20150368352.

Lambda/Kappa Formats

Multispecific molecules (e.g., multispecific antibody molecules) that include the lambda light chain polypeptide and a kappa light chain polypeptides, can be used to allow for heterodimerization. Methods for generating bispecific antibody molecules comprising the lambda light chain polypeptide and a kappa light chain polypeptides are disclosed in PCT/US17/53053 filed on Sep. 22, 2017 and designated publication number WO 2018/057955, incorporated herein by reference in its entirety.

In embodiments, the multispecific molecule includes a multispecific antibody molecule, e.g., an antibody molecule comprising two binding specificities, e.g., a bispecific antibody molecule. The multispecific antibody molecule includes: a lambda light chain polypeptide 1 (LLCP1) specific for a first epitope; a heavy chain polypeptide 1 (HCP1) specific for the first epitope; a kappa light chain polypeptide 2 (KLCP2) specific for a second epitope; and a heavy chain polypeptide 2 (HCP2) specific for the second epitope.

“Lambda light chain polypeptide 1 (LLCP1)”, as that term is used herein, refers to a polypeptide comprising sufficient light chain (LC) sequence, such that when combined with a cognate heavy chain variable region, can mediate specific binding to its epitope and complex with an HCP1. In an embodiment it comprises all or a fragment of a CH1 region. In an embodiment, an LLCP1 comprises LC-CDR1, LC-CDR2, LC-CDR3, FR1, FR2, FR3, FR4, and CH1, or sufficient sequence therefrom to mediate specific binding of its epitope and complex with an HCP1. LLCP1, together with its HCP1, provide specificity for a first epitope (while KLCP2, together with its HCP2, provide specificity for a second epitope). As described elsewhere herein, LLCP1 has a higher affinity for HCP1 than for HCP2.

“Kappa light chain polypeptide 2 (KLCP2)”, as that term is used herein, refers to a polypeptide comprising sufficient light chain (LC) sequence, such that when combined with a cognate heavy chain variable region, can mediate specific binding to its epitope and complex with an HCP2. In an embodiments it comprises all or a fragment of a CH1 region. In an embodiment, a KLCP2 comprises LC-CDR1, LC-CDR2, LC-CDR3, FR1, FR2, FR3, FR4, and CH1, or sufficient sequence therefrom to mediate specific binding of its epitope and complex with an HCP2. KLCP2, together with its HCP2, provide specificity for a second epitope (while LLCP1, together with its HCP1, provide specificity for a first epitope).

“Heavy chain polypeptide 1 (HCP1)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In an embodiments it comprises all or a fragment of a CHlregion. In an embodiment, it comprises all or a fragment of a CH2 and/or CH3 region. In an embodiment an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an LLCP1, (ii) to complex preferentially, as described herein to LLCP1 as opposed to KLCP2; and (iii) to complex preferentially, as described herein, to an HCP2, as opposed to another molecule of HCP1. HCP1, together with its LLCP1, provide specificity for a first epitope (while KLCP2, together with its HCP2, provide specificity for a second epitope).

“Heavy chain polypeptide 2 (HCP2)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In an embodiments it comprises all or a fragment of a CHlregion. In an embodiments it comprises all or a fragment of a CH2 and/or CH3 region. In an embodiment an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an KLCP2, (ii) to complex preferentially, as described herein to KLCP2 as opposed to LLCP1; and (iii) to complex preferentially, as described herein, to an HCP1, as opposed to another molecule of HCP2. HCP2, together with its KLCP2, provide specificity for a second epitope (while LLCP1, together with its HCP1, provide specificity for a first epitope).

In some embodiments of the multispecific antibody molecule disclosed herein:
LLCP1 has a higher affinity for HCP1 than for HCP2; and/or
KLCP2 has a higher affinity for HCP2 than for HCP1.

In embodiments, the affinity of LLCP1 for HCP1 is sufficiently greater than its affinity for HCP2, such that under preselected conditions, e.g., in aqueous buffer, e.g., at pH 7, in saline, e.g., at pH 7, or under physiological conditions, at least 75, 80, 90, 95, 98, 99, 99.5, or 99.9% of the multispecific antibody molecule molecules have a LLCPlcomplexed, or interfaced with, a HCP .

In some embodiments of the multispecific antibody molecule disclosed herein: the HCP1 has a greater affinity for HCP2, than for a second molecule of HCP1; and/or the HCP2 has a greater affinity for HCP1, than for a second molecule of HCP2.

In embodiments, the affinity of HCP1 for HCP2 is sufficiently greater than its affinity for a second molecule of HCP1, such that under preselected conditions, e.g., in aqueous buffer, e.g., at pH 7, in saline, e.g., at pH 7, or under physiological conditions, at least 75%, 80, 90, 95, 98, 99 99.5 or 99.9% of the multispecific antibody molecule molecules have a HCPlcomplexed, or interfaced with, a HCP2.

In another aspect, disclosed herein is a method for making, or producing, a multispecific antibody molecule. The method includes:
(i) providing a first heavy chain polypeptide (e.g., a heavy chain polypeptide comprising one, two, three or all of a first heavy chain variable region (first VH), a first CH1, a first heavy chain constant region (e.g., a first CH2, a first CH3, or both));
(ii) providing a second heavy chain polypeptide (e.g., a heavy chain polypeptide comprising one, two, three or all of a second heavy chain variable region (second VH), a second CH1, a second heavy chain constant region (e.g., a second CH2, a second CH3, or both));
(iii) providing a lambda chain polypeptide (e.g., a lambda light variable region (VLX), a lambda light constant chain (VLX), or both) that preferentially associates with the first heavy chain polypeptide (e.g., the first VH); and
(iv) providing a kappa chain polypeptide (e.g., a lambda light variable region (VLK), a lambda light constant chain (VL⁻k), or both) that preferentially associates with the second heavy chain polypeptide (e.g., the second VH), under conditions where (i)-(iv) associate.

In embodiments, the first and second heavy chain polypeptides form an Fc interface that enhances heterodimerization.

In embodiments, (i)-(iv) (e.g., nucleic acid encoding (i)-(iv)) are introduced in a single cell, e.g., a single mammalian cell, e.g., a CHO cell. In embodiments, (i)-(iv) are expressed in the cell.

In embodiments, (i)-(iv) (e.g., nucleic acid encoding (i)-(iv)) are introduced in different cells, e.g., different mammalian cells, e.g., two or more CHO cell. In embodiments, (i)-(iv) are expressed in the cells.

In one embodiments, the method further comprises purifying a cell-expressed antibody molecule, e.g., using a lambda- and/or- kappa-specific purification, e.g., affinity chromatography.

In embodiments, the method further comprises evaluating the cell-expressed multispecific antibody molecule. For example, the purified cell-expressed multispecific antibody molecule can be analyzed by techniques known in the art, include mass spectrometry. In one embodiment, the purified cell-expressed antibody molecule is cleaved, e.g., digested with papain to yield the Fab moieties and evaluated using mass spectrometry.

In embodiments, the method produces correctly paired kappa/lambda multispecific, e.g., bispecific, antibody molecules in a high yield, e.g., at least 75%, 80, 90, 95, 98, 99 99.5 or 99.9%.

In other embodiments, the multispecific, e.g., a bispecific, antibody molecule that includes:
(i) a first heavy chain polypeptide (HCP1) (e.g., a heavy chain polypeptide comprising one, two, three or all of a first heavy chain variable region (first VH), a first CH1, a first heavy chain constant region (e.g., a first CH2, a first CH3, or both)), e.g., wherein the HCP1 binds to a first epitope;
(ii) a second heavy chain polypeptide (HCP2) (e.g., a heavy chain polypeptide comprising one, two, three or all of a second heavy chain variable region (second VH), a second CH1, a second heavy chain constant region (e.g., a second CH2, a second CH3, or both)), e.g., wherein the HCP2 binds to a second epitope;
(iii) a lambda light chain polypeptide (LLCP1) (e.g., a lambda light variable region (VL1), a lambda light constant chain (VL1), or both) that preferentially associates with the first heavy chain polypeptide (e.g., the first VH), e.g., wherein the LLCP1 binds to a first epitope; and
(iv) a kappa light chain polypeptide (KLCP2) (e.g., a lambda light variable region (VLk), a lambda light constant chain (VLk), or both) that preferentially associates with the second heavy chain polypeptide (e.g., the second VH), e.g., wherein the KLCP2 binds to a second epitope.

In embodiments, the first and second heavy chain polypeptides form an Fc interface that enhances heterodimerization. In embodiments, the multispecific antibody molecule has a first binding specificity that includes a hybrid VL1-CL1 heterodimerized to a first heavy chain variable region connected to the Fc constant, CH2-CH3 domain (having a knob modification) and a second binding specificity that includes a hybrid VLk-CLk heterodimerized to a second heavy chain variable region connected to the Fc constant, CH2-CH3 domain (having a hole modification).

### Cytokine Molecules

Cytokines are generally polypeptides that influence cellular activity, for example, through signal transduction pathways. Accordingly, a cytokine of the multispecific or multifunctional polypeptide is useful and can be associated with receptor-mediated signaling that transmits a signal from outside the cell membrane to modulate a response within the cell. Cytokines are proteinaceous signaling compounds that are mediators of the immune response. They control many different cellular functions including proliferation, differentiation and cell survival/apoptosis; cytokines are also involved in several pathophysiological processes including viral infections and autoimmune diseases. Cytokines are synthesized under various stimuli by a variety of cells of both the innate (monocytes, macrophages, dendritic cells) and adaptive (T- and B-cells) immune systems. Cytokines can be classified into two groups: pro- and anti-inflammatory. Pro-inflammatory cytokines, including IFNγ, IL-1, IL-6 and TNF-alpha, are predominantly derived from the innate immune cells and Thl cells. Anti-inflammatory cytokines, including IL-10, IL-4, IL-13 and IL-5, are synthesized from Th2 immune cells.

The present disclosure provides, inter alia, multispecific (e.g., bi-, tri-, quad- specific) or multifunctional molecules, that include, e.g., are engineered to contain, one or more cytokine molecules, e.g., immunomodulatory (e.g., proinflammatory) cytokines and variants, e.g., functional variants, thereof. Accordingly, in some embodiments, the cytokine molecule is an interleukin or a variant, e.g., a functional variant thereof. In some embodiments the interleukin is a proinflammatory interleukin. In some embodiments the interleukin is chosen from interleukin-2 (IL-2), interleukin-12 (IL-12), interleukin-15 (IL-15), interleukin-18 (IL-18), interleukin-21 (IL-21), interleukin-7 (IL-7), or interferon gamma. In some embodiments, the cytokine molecule is a proinflammatory cytokine.

In certain embodiments, the cytokine is a single chain cytokine. In certain embodiments, the cytokine is a multichain cytokine (e.g., the cytokine comprises 2 or more (e.g., 2) polypeptide chains. An exemplary multichain cytokine is IL-12.

Examples of useful cytokines include, but are not limited to, GM-CSF, IL-1α, IL-1β, IL-2, IL-3, IL-4, IL-5, IL-6, IL-7, IL-8, IL-10, IL-12, IL-21, IFN-α, IFN-β, IFN-γ, MIP-1α, MIP-1β, TGF-β, TNF-α, and TNFβ. In one embodiment the cytokine of the multispecific or multifunctional polypeptide is a cytokine selected from the group of GM-CSF, IL-2, IL-7, IL-8, IL-10, IL-12, IL-15, IL-21, IFN-α, IFN-γ, MIP-1α, MIP-1β and TGF-β. In one embodiment the cytokine of the i the multispecific or multifunctional polypeptide is a cytokine selected from the group of IL-2, IL-7, IL-10, IL-12, IL-15, IFN-α, and IFN-γ. In certain embodiments the cytokine is mutated to remove N- and/or O-glycosylation sites. Elimination of glycosylation increases homogeneity of the product obtainable in recombinant production.

In one embodiment, the cytokine of the multispecific or multifunctional polypeptide is IL-2. In a specific embodiment, the IL-2 cytokine can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) cytotoxicity. In another particular embodiment the IL-2 cytokine is a mutant IL-2 cytokine having reduced binding affinity to the .alpha.-subunit of the IL-2 receptor. Together with the .beta.- and .gamma.-subunits (also known as CD122 and CD132, respectively), the .alpha.-subunit (also known as CD25) forms the heterotrimeric high-affinity IL-2 receptor, while the dimeric receptor consisting only of the β- and γ-subunits is termed the intermediate-affinity IL-2 receptor. As described in PCT patent application number PCT/EP2012/051991, which is incorporated herein by reference in its entirety, a mutant IL-2 polypeptide with reduced binding to the .alpha.-subunit of the IL-2 receptor has a reduced ability to induce IL-2 signaling in regulatory T cells, induces less activation-induced cell death (AICD) in T cells, and has a reduced toxicity profile in vivo, compared to a wild-type IL-2 polypeptide. The use of such an cytokine with reduced toxicity is particularly advantageous in a multispecific or multifunctional polypeptide according to the invention, having a long serum half-life due to the presence of an Fc domain. In one embodiment, the mutant IL-2 cytokine of the multispecific or multifunctional polypeptide according to the invention comprises at least one amino acid mutation that reduces or abolishes the affinity of the mutant IL-2 cytokine to the .alpha.-subunit of the IL-2 receptor (CD25) but preserves the affinity of the mutant IL-2 cytokine to the intermediate-affinity IL-2 receptor (consisting of the 0 and y subunits of the IL-2 receptor), compared to the non-mutated IL-2 cytokine. In one embodiment the one or more amino acid mutations are amino acid substitutions. In a specific embodiment, the mutant IL-2 cytokine comprises one, two or three amino acid substitutions at one, two or three position(s) selected from the positions corresponding to residue 42, 45, and 72 of human IL-2. In a more specific embodiment, the mutant IL-2 cytokine comprises three amino acid substitutions at the positions corresponding to residue 42, 45 and 72 of human IL-2. In an even more specific embodiment, the mutant IL-2 cytokine is human IL-2 comprising the amino acid substitutions F42A, Y45A and L72G. In one embodiment the mutant IL-2 cytokine additionally comprises an amino acid mutation at a position corresponding to position 3 of human IL-2, which eliminates the O-glycosylation site of IL-2. Particularly, said additional amino acid mutation is an amino acid substitution replacing a threonine residue by an alanine residue. A particular mutant IL-2 cytokine useful in the invention comprises four amino acid substitutions at positions corresponding to residues 3, 42, 45 and 72 of human IL-2. Specific amino acid substitutions are T3A, F42A, Y45A and L72G. As demonstrated in PCT patent application number PCT/EP2012/051991 and in the appended Examples, said quadruple mutant IL-2 polypeptide (IL-2 qm) exhibits no detectable binding to CD25, reduced ability to induce apoptosis in T cells, reduced ability to induce IL-2 signaling in T.sub.reg cells, and a reduced toxicity profile in vivo. However, it retains ability to activate IL-2 signaling in effector cells, to induce proliferation of effector cells, and to generate IFN-y as a secondary cytokine by NK cells.

The IL-2 or mutant IL-2 cytokine according to any of the above embodiments may comprise additional mutations that provide further advantages such as increased expression or stability. For example, the cysteine at position 125 may be replaced with a neutral amino acid such as alanine, to avoid the formation of disulfide-bridged IL-2 dimers. Thus, in certain embodiments the IL-2 or mutant IL-2 cytokine of the multispecific or multifunctional polypeptide according to the invention comprises an additional amino acid mutation at a position corresponding to residue 125 of human IL-2. In one embodiment said additional amino acid mutation is the amino acid substitution C125A.

In a specific embodiment the IL-2 cytokine of the multispecific or multifunctional polypeptide comprises the polypeptide sequence of SEQ ID NO: 227

[APTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSET
TFMCEYADETATIVEFLNRWITFAQSIISTLT].

In another specific embodiment the IL-2 cytokine of the multispecific or multifunctional polypeptide comprises the polypeptide sequence of SEQ ID NO: 228

[APASSSTKKTQLQLEHLLLDLQMILNGINNYKNPKLTRMLTAKFAMPKKA
TELKHLQCLEEELKPLEEVLNGAQSKNFHLRPRDLISNINVIVLELKGSET
TFMCEYADETATIVEFLNRWITFAQSIISTLT].

In another embodiment the cytokine of the multispecific or multifunctional polypeptide is IL-12. In a specific embodiment said IL-12 cytokine is a single chain IL-12 cytokine. In an even more specific embodiment the single chain IL-12 cytokine comprises the polypeptide sequence of SEQ ID NO: 229 [IWELKKDVYVVELDWYPDAPGEMVVLTCDTPEEDGITWTLDQSSEVLGSGKTLTIQVK EFGDAGQYTCHKGGEVLSHSLLLLHKKEDGIWSTDILKDQKEPKNKTFLRCEAKNYSGR FTCWWLTTISTDLTFSVKSSRGSSDPQGVTCGAATLSAERVRGDNKEYEYSVECQEDSA CPAAEESLPIEVMVDAVHKLKYENYTSSFFIRDIIKPDPPKNLQLKPLKNSRQVEVSWEY PDTWSTPHSYFSLTFCVQVQGKSKREKKDRVFTDKTSATVICRKNASISVRAQDRYYSS SWSEWASVPCSGGGGSGGGGSGGGGSRNLPVATPDPGMFPCLHESQNLLRAVSNMLQ KARQTLEFYPCTSEEIDHEDITKDKTSTVEACLPLELTKNESCLNSRETSFITNGSCLASRK TSFMMALCLSSIYEDLKMYQVEFKTMNAKLLMDPKRQIFLDQNMLAVIDELMQALNFN SETVPQKSSLEEPDFYKTKIKLCILLHAFRIRAVTIDRVMSYLNAS]. In one embodiment, the IL-12 cytokine can elicit one or more of the cellular responses selected from the group consisting of: proliferation in a NK cell, differentiation in a NK cell, proliferation in a T cell, and differentiation in a T cell.

In another embodiment the cytokine of the multispecific or multifunctional polypeptide is IL-10. In a specific embodiment said IL-10 cytokine is a single chain IL-10 cytokine. In an even more specific embodiment the single chain IL-10 cytokine comprises the polypeptide sequence of SEQ ID NO: 3471

[SPGQGTQSENSCTHFPGNLPNMLRDLRDAFSRVKTFFQMKDQLDNLLLKE
SLLEDFKGYLGCQALSEMIQFYLEEVMPQAENQDPDIKAHVNSLGENLKTL
RLRLRRCHRFLPCENKSKAVEQVKNAFNKLQEKGIYKAMSEFDIFINYIEA
YMTMKIRNGGGGSGGGGSGGGGSGGGGSSPGQGTQSENSCTHFPGNLPNML
RDLRDAFSRVKTFFQMKDQLDNLLLKESLLEDFKGYLGCQALSEMIQFYLE
EVMPQAENQDPDIKAHVNSLGENLKTLRLRLRRCHRFLPCENKSKAVEQVK
NAFNKLQEKGIYKAMSEFDIFINYIEAYMTMKIRN].

In another specific embodiment the IL-10 cytokine is a monomeric IL-10 cytokine. In a more specific embodiment the monomeric IL-10 cytokine comprises the polypeptide sequence of SEQ ID NO: 3472 [SPGQGTQSENSCTHFPGNLPNMLRDLRDAFSRVKTFFQMKDQLDNLLLKESLLEDFKG YLGCQALSEMIQFYLEEVMPQAENQDPDIKAHVNSLGENLKTLRLRLRRCHRFLPCENG GGSGGKSKAVEQVKNAFNKLQEKGIYKAMSEFDIFINYIEAYMTMKIRN]. In one embodiment, the IL-10 cytokine can elicit one or more of the cellular responses selected from the group consisting of: inhibition of cytokine secretion, inhibition of antigen presentation by antigen presenting cells, reduction of oxygen radical release, and inhibition of T cell proliferation. A multispecific or multifunctional polypeptide according to the invention wherein the cytokine is IL-10 is particularly useful for downregulation of inflammation, e.g. in the treatment of an inflammatory disorder.

In another embodiment, the cytokine of the multispecific or multifunctional polypeptide is IL-15. In a specific embodiment said IL-15 cytokine is a mutant IL-15 cytokine having reduced binding affinity to the a-subunit of the IL-15 receptor. Without wishing to be bound by theory, a mutant IL-15 polypeptide with reduced binding to the .alpha.-subunit of the IL-15 receptor has a reduced ability to bind to fibroblasts throughout the body, resulting in improved pharmacokinetics and toxicity profile, compared to a wild-type IL-15 polypeptide. The use of an cytokine with reduced toxicity, such as the described mutant IL-2 and mutant IL-15 effector moieties, is particularly advantageous in a multispecific or multifunctional polypeptide according to the invention, having a long serum half-life due to the presence of an Fc domain. In one embodiment the mutant IL-15 cytokine of the multispecific or multifunctional polypeptide according to the invention comprises at least one amino acid mutation that reduces or abolishes the affinity of the mutant IL-15 cytokine to the .alpha.-subunit of the IL-15 receptor but preserves the affinity of the mutant IL-15 cytokine to the intermediate-affinity IL-15/IL-2 receptor (consisting of the .beta.- and .gamma.-subunits of the IL-15/IL-2 receptor), compared to the non-mutated IL-15 cytokine. In one embodiment the amino acid mutation is an amino acid substitution. In a specific embodiment, the mutant IL-15 cytokine comprises an amino acid substitution at the position corresponding to residue 53 of human IL-15. In a more specific embodiment, the mutant IL-15 cytokine is human IL-15 comprising the amino acid substitution E53A. In one embodiment the mutant IL-15 cytokine additionally comprises an amino acid mutation at a position corresponding to position 79 of human IL-15, which eliminates the N-glycosylation site of IL-15. Particularly, said additional amino acid mutation is an amino acid substitution replacing an asparagine residue by an alanine residue. In an even more specific embodiment the IL-15 cytokine comprises the polypeptide sequence of SEQ ID NO: 3473 [NWVNVISDLKKIEDLIQSMHIDATLYTESDVHPSCKVTAMKCFLLELQVISLASGDASIH DTVENLIILANNSLSSNGAVTESGCKECEELEEKNIKEFLQSFVHIVQMFINT S]. In one embodiment, the IL-15 cytokine can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) cytotoxicity.

Mutant cytokine molecules useful as effector moieties in the multispecific or multifunctional polypeptide can be prepared by deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing. Substitution or insertion may involve natural as well as non-natural amino acid residues. Amino acid modification includes well known methods of chemical modification such as the addition or removal of glycosylation sites or carbohydrate attachments, and the like.

In one embodiment, the cytokine, particularly a single-chain cytokine, of the multispecific or multifunctional polypeptide is GM-CSF. In a specific embodiment, the GM-CSF cytokine can elicit proliferation and/or differentiation in a granulocyte, a monocyte or a dendritic cell. In one embodiment, the cytokine, particularly a single-chain cytokine, of the multispecific or multifunctional polypeptide is IFN-α. In a specific embodiment, the IFN-α cytokine can elicit one or more of the cellular responses selected from the group consisting of: inhibiting viral replication in a virus-infected cell, and upregulating the expression of major histocompatibility complex I (MHC I). In another specific embodiment, the IFN-α cytokine can inhibit proliferation in a cell. In one embodiment the cytokine, particularly a single-chain cytokine, of the multispecific or multifunctional polypeptide is IFNγ. In a specific embodiment, the IFN-γ cytokine can elicit one or more of the cellular responses selected from the group of: increased macrophage activity, increased expression of MHC molecules, and increased NK cell activity. In one embodiment the cytokine, particularly a single-chain cytokine, of the multispecific or multifunctional polypeptide is IL-7. In a specific embodiment, the IL-7 cytokine can elicit proliferation of T and/or B lymphocytes. In one embodiment, the cytokine, particularly a single-chain cytokine, of the multispecific or multifunctional polypeptide is IL-8. In a specific embodiment, the IL-8 cytokine can elicit chemotaxis in neutrophils. In one embodiment, the cytokine, particularly a single-chain cytokine, of the multispecific or multifunctional polypeptide, is MIP-1α. In a specific embodiment, the MIP-la cytokine can elicit chemotaxis in monocytes and T lymphocyte cells. In one embodiment, the cytokine, particularly a single-chain cytokine, of the multispecific or multifunctional polypeptide is MIP-1β. In a specific embodiment, the MIP-1β cytokine can elicit chemotaxis in monocytes and T lymphocyte cells. In one embodiment, the cytokine, particularly a single-chain cytokine, of the multispecific or multifunctional polypeptide is TGF-β. In a specific embodiment, the TGF-β cytokine can elicit one or more of the cellular responses selected from the group consisting of: chemotaxis in monocytes, chemotaxis in macrophages, upregulation of IL-1 expression in activated macrophages, and upregulation of IgA expression in activated B cells.

In one embodiment, the multispecific or multifunctional polypeptide of the invention binds to an cytokine receptor with a dissociation constant (K_{D}) that is at least about 1, 1.5, 2, 2.5, 3, 3.5, 4, 4.5, 5, 5.5, 6, 6.5, 7, 7.5, 8, 8.5, 9, 9.5 or 10 times greater than that for a control cytokine. In another embodiment, the multispecific or multifunctional polypeptide binds to an cytokine receptor with a K_{D} that is at least 2, 3, 4, 5, 6, 7, 8, 9, or 10 times greater than that for a corresponding multispecific or multifunctional polypeptide comprising two or more effector moieties. In another embodiment, the multispecific or multifunctional polypeptide binds to an cytokine receptor with a dissociation constant K_{D} that is about 10 times greater than that for a corresponding the multispecific or multifunctional polypeptide comprising two or more cytokines.

In some embodiments, the multispecific molecules disclosed herein include a cytokine molecule. In embodiments, the cytokine molecule includes a full length, a fragment or a variant of a cytokine; a cytokine receptor domain, e.g., a cytokine receptor dimerizing domain; or an agonist of a cytokine receptor, e.g., an antibody molecule (e.g., an agonistic antibody) to a cytokine receptor.

In some embodiments the cytokine molecule is chosen from IL-2, IL-12, IL-15, IL-18, IL-7, IL-21, or interferon gamma, or a fragment or variant thereof, or a combination of any of the aforesaid cytokines. The cytokine molecule can be a monomer or a dimer. In embodiments, the cytokine molecule can further include a cytokine receptor dimerizing domain.

In other embodiments, the cytokine molecule is an agonist of a cytokine receptor, e.g., an antibody molecule (e.g., an agonistic antibody) to a cytokine receptor chosen from an IL-15Ra or IL-21R.

In one embodiment, the cytokine molecule is IL-15, e.g., human IL-15 (e.g., comprising the amino acid sequence: NWVNVISDLKKIEDLIQSMHIDATLYTESDVHPSCKVTAMKCFLLELQVISLESGDASIH DTVENLIILANNSLSSNGNVTESGCKECEELEEKNIKEFLQSFVHIVQMFINTS (SEQ ID NO: 3437), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3437.

In some embodiments, the cytokine molecule comprises a receptor dimerizing domain, e.g., an IL15Ralpha dimerizing domain. In one embodiment, the IL15Ralpha dimerizing domain comprises the amino acid sequence: MAPRRARGCRTLGLPALLLLLLLRPPATRGITCPPPMSVEHADIWVKSYSLYSRERYICN SGFKRKAGTSSLTECVL (SEQ ID NO: 3438), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3438. In some embodiments, the cytokine molecule (e.g., IL-15) and the receptor dimerizing domain (e.g., an IL15Ralpha dimerizing domain) of the multispecific molecule are covalently linked, e.g., via a linker (e.g., a Gly-Ser linker, e.g., a linker comprising the amino acid sequence SGGSGGGGSGGGSGGGGSLQ (SEQ ID NO: 3439). In other embodiments, the cytokine molecule (e.g., IL-15) and the receptor dimerizing domain (e.g., an IL15Ralpha dimerizing domain) of the multispecific molecule are not covalently linked, e.g., are non-covalently associated.

In other embodiments, the cytokine molecule is IL-2, e.g., human IL-2 (e.g., comprising the amino acid sequence: APTS S STKKTQLQLEHLLLDLQMILNGINNYKNPKLTRMLTFKFYMPKKATELKHLQCL EEELKPLEEVLNLAQSKNFHLRPRDLISNINVIVLELKGSETTFMCEYADETATIVEFLNR WITFCQSIISTLT (SEQ ID NO: 3440), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3440).

In other embodiments, the cytokine molecule is IL-18, e.g., human IL-18 (e.g., comprising the amino acid sequence: YFGKLESKLSVIRNLNDQVLFIDQGNRPLFEDMTDSDCRDNAPRTIFIISMYKDSQPRGM AVTISVKCEKISTLSCENKIISFKEMNPPDNIKDTKSDIIFFQRSVPGHDNKMQFESSSYEG YFLACEKERDLFKLILKKEDELGDRSIMFTVQNED (SEQ ID NO: 121), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3441).

In other embodiments, the cytokine molecule is IL-21, e.g., human IL-21 (e.g., comprising the amino acid sequence: QGQDRHMIRMRQLIDIVDQLKNYVNDLVPEFLPAPEDVETNCEWSAFSCFQKAQLKSA NTGNNERIINVSIKKLKRKPPSTNAGRRQKHRLTCPSCDSYEKKPPKEFLERFKSLLQKMI HQHLSSRTHGSEDS (SEQ ID NO: 3442), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3442).

In yet other embodiments, the cytokine molecule is interferon gamma, e.g., human interferon gamma (e.g., comprising the amino acid sequence: QDPYVKEAENLKKYFNAGHSDVADNGTLFLGILKNWKEESDRKINIQSQIVSFYFKLFK NFKDDQSIQKSVETIKEDMNVKFFNSNKKKRDDFEKLTNYSVTDLNVQRKAIHELIQVM AELSPAAKTGKRKRSQMLFRG (SEQ ID NO: 3443), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3443).

### Immune Cell Engagers

The immune cell engagers, e.g., first and/or second immune cell engager, of the multispecific or multifunctional molecules disclosed herein can mediate binding to, and/or activation of, an immune cell, e.g., an immune effector cell. In some embodiments, the immune cell is chosen from a T cell, an NK cell, a B cell, a dendritic cell, or a macrophage cell engager, or a combination thereof. In some embodiments, the immune cell engager is chosen from one, two, three, or all of a T cell engager, NK cell engager, a B cell engager, a dendritic cell engager, or a macrophage cell engager, or a combination thereof. The immune cell engager can be an agonist of the immune system. In some embodiments, the immune cell engager can be an antibody molecule, a ligand molecule (e.g., a ligand that further comprises an immunoglobulin constant region, e.g., an Fc region), a small molecule, a nucleotide molecule.

### Natural Killer Cell Engagers

Natural Killer (NK) cells recognize and destroy infected cells in an antibody-independent manner. The regulation of NK cells is mediated by activating and inhibiting receptors on the NK cell surface. One family of activating receptors is the natural cytotoxicity receptors (NCRs) which include NKp30, NKp44 and NKp46. The NCRs initiate targeting by recognition of heparan sulfate on cells. NKG2D is a receptor that provides both stimulatory and costimulatory innate immune responses on activated killer (NK) cells, leading to cytotoxic activity. DNAM1 is a receptor involved in intercellular adhesion, lymphocyte signaling, cytotoxicity and lymphokine secretion mediated by cytotoxic T-lymphocyte (CTL) and NK cell. DAP10 (also known as HCST) is a transmembrane adapter protein which associates with KLRK1 to form an activation receptor KLRK1-HCST in lymphoid and myeloid cells; this receptor plays a major role in triggering cytotoxicity against target cells expressing cell surface ligands such as MHC class I chain-related MICA and MICB, and U(optionally L1)6-binding proteins (ULBPs); it KLRK1-HCST receptor plays a role in immune surveillance is involved cytolysis of cells; indeed, melanoma cells that do not express KLRK1 ligands escape from immune surveillance mediated by NK cells. CD16 is a receptor for the Fc region of IgG, which binds complexed or aggregated IgG and also monomeric IgG and thereby mediates antibody-dependent cellular cytotoxicity (ADCC) and other antibody-dependent responses, such as phagocytosis.

In some embodiments, the NK cell engager is a viral hemagglutinin (HA), HA is a glycoprotein found on the surface of influenza viruses. It is responsible for binding the virus to cells with sialic acid on the membranes, such as cells in the upper respiratory tract or erythrocytes. HA has at least 18 different antigens. These subtypes are named H1 through H18. NCRs can recognize viral proteins. NKp46 has been shown to be able to interact with the HA of influenza and the HA-NA of Paramyxovirus, including Sendai virus and Newcastle disease virus. Besides NKp46, NKp44 can also functionally interact with HA of different influenza subtypes.

The present disclosure provides, inter alfa, multispecific (e.g., bi-, tri-, quad- specific) or multifunctional molecules, that are engineered to contain one or more NK cell engagers that mediate binding to and/or activation of an NK cell. Accordingly, in some embodiments, the NK cell engager is selected from an antigen binding domain or ligand that binds to (e.g., activates): NKp30, NKp40, NKp44, NKp46, NKG2D, DNAM1, DAP10, CD16 (e.g., CD16a, CD16b, or both), CRTAM, CD27, PSGL1, CD96, CD100 (SEMA4D), NKp80, CD244 (also known as SLAMF4 or 2B4), SLAMF6, SLAMF7, KIR2DS2, KIR2DS4, KIR3DS1, KIR2DS3, KIR2DS5, KIR2DS1, CD94, NKG2C, NKG2E, or CD160.

In one embodiment, the NK cell engager is a ligand of NKp30 is a B7-6, e.g., comprises the amino acid sequence of: DLKVEMMAGGTQITPLNDNVTIFCNIFYSQPLNITSMGITWFWKSLTFDKEVKVFEFFGD HQEAFRPGAIVSPWRLKSGDASLRLPGIQLEEAGEYRCEVVVTPLKAQGTVQLEVVASP ASRLLLDQVGMKENEDKYMCES SGFYPEAINITWEKQTQKFPHPIEISEDVITGPTIKNM DGTFNVTSCLKLNSSQEDPGTVYQCVVRHASLHTPLRSNFTLTAARHSLSETEKTDNFS (SEQ ID NO: 3444), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3444.

In other embodiments, the NK cell engager is a ligand of NKp44 or NKp46, which is a viral HA. Viral hemagglutinins (HA) are glyco proteins which are on the surface of viruses. HA proteins allow viruses to bind to the membrane of cells via sialic acid sugar moieties which contributes to the fusion of viral membranes with the cell membranes (see e.g., Eur J Immunol. 2001 Sep;31(9):2680-9 “Recognition of viral hemagglutinins by NKp44 but not by NKp30”; and Nature. 2001 Feb 22;409(6823):1055-60 “Recognition of haemagglutinins on virus-infected cells by NKp46 activates lysis by human NK cells” the contents of each of which are incorporated by reference herein).

In other embodiments, the NK cell engager is a ligand of NKG2D chosen from MICA, MICB, or ULBP1, e.g., wherein:
(i) MICA comprises the amino acid sequence: EPHSLRYNLTVLSWDGSVQSGFLTEVHLDGQPFLRCDRQKCRAKPQGQWAEDVLGNK TWDRETRDLTGNGKDLRMTLAHIKDQKEGLHSLQEIRVCEIHEDNSTRSSQHFYYDGEL FLSQNLETKEWTMPQSSRAQTLAMNVRNFLKEDAMKTKTHYHAMHADCLQELRRYLK SGVVLRRTVPPMVNVTRSEASEGNITVTCRASGFYPWNITLSWRQDGVSLSHDTQQWG DVLPDGNGTYQTWVATRICQGEEQRFTCYMEHSGNHSTHPVPSGKVLVLQSHW (SEQ ID NO: 3445), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3445;
(ii) MICB comprises the amino acid sequence: AEPHSLRYNLMVLSQDESVQSGFLAEGHLDGQPFLRYDRQKRRAKPQGQWAEDVLGA KTWDTETEDLTENGQDLRRTLTHIKDQKGGLHSLQEIRVCEIHEDSSTRGSRHFYYDGEL FLSQNLETQESTVPQSSRAQTLAMNVTNFWKEDAMKTKTHYRAMQADCLQKLQRYLK SGVAIRRTVPPMVNVTCSEVSEGNITVTCRASSFYPRNITLTWRQDGVSLSHNTQQWGD VLPDGNGTYQTWVATRIRQGEEQRFTCYMEHSGNHGTHPVPSGKVLVLQSQRTD (SEQ ID NO: 3446), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3446; or (iii) ULBP1 comprises the amino acid sequence: GWVDTHCLCYDFIITPKSRPEPQWCEVQGLVDERPFLHYDCVNHKAKAFASLGKKVNV TKTWEEQTETLRDVVDFLKGQLLDIQVENLIPIEPLTLQARMSCEHEAHGHGRGSWQFL FNGQKFLLFD SNNRKWTALHPGAKKMTEKWEKNRDVTMFFQKISLGDCKMWLEEFL MYWEQMLDPTKPPSLAPG (SEQ ID NO: 3447), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3447.

In other embodiments, the NK cell engager is a ligand of DNAM1 chosen from NECTIN2 or NECL5, e.g., wherein:
(i) NECTIN2 comprises the amino acid sequence: QDVRVQVLPEVRGQLGGTVELPCHLLPPVPGLYISLVTWQRPDAPANHQNVAAFHPKM GPSFPSPKPGSERLSFVSAKQ STGQDTEAELQDATLALHGLTVEDEGNYTCEFATFPKGS VRGMTWLRVIAKPKNQAEAQKVTFSQDPTTVALCISKEGRPPARISWLSSLDWEAKETQ VSGTLAGTVTVTSRFTLVPSGRADGVTVTCKVEHESFEEPALIPVTLSVRYPPEVSISGYD DNWYLGRTDATLSCDVRSNPEPTGYDWSTTSGTFPTSAVAQGSQLVIHAVD SLFNTTF V CTVTNAVGMGRAEQVIFVRETPNTAGAGATGG (SEQ ID NO: 3448), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3448; or
(ii) NECLS comprises the amino acid sequence: WPPPGTGDVVVQAPTQVPGFLGDSVTLPCYLQVPNMEVTHVSQLTWARHGESGSMAV FHQTQGPSYSESKRLEFVAARLGAELRNASLRMFGLRVEDEGNYTCLFVTFPQGSRSVD IWLRVLAKPQNTAEVQKVQLTGEPVPMARCVSTGGRPPAQITWHSDLGGMPNTSQVPG FLSGTVTVTSLWILVPSSQVDGKNVTCKVEHESFEKPQLLTVNLTVYYPPEVSISGYDNN WYLGQNEATLTCDARSNPEPTGYNWSTTMGPLPPFAVAQGAQLLIRPVDKPINTTLICN VTNALGARQAELTVQVKEGPPSEHSGISRN (SEQ ID NO: 3449), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3449.

In yet other embodiments, the NK cell engager is a ligand of DAP10, which is an adapter for NKG2D (see e.g., Proc Natl Acad Sci U S A. 2005 May 24; 102(21): 7641-7646; and Blood, 15 September 2011 Volume 118, Number 11, the full contents of each of which is incorporated by reference herein).

In other embodiments, the NK cell engager is a ligand of CD16, which is a CD16a/b ligand, e.g., a CD16a/b ligand further comprising an antibody Fc region (see e.g., Front Immunol. 2013; 4: 76 discusses how antibodies use the Fc to trigger NK cells through CD16,the full contents of which are incorporated herein).

In other embodiments, the NK cell engager is a ligand of CRTAM, which is NECL2, e.g., wherein NECL2 comprises the amino acid sequence: QNLFTKDVTVIEGEVATISCQVNKSDDSVIQLLNPNRQTIYFRDFRPLKDSRFQLLNFSSS ELKVSLTNVSISDEGRYFCQLYTDPPQESYTTITVLVPPRNLMIDIQKDTAVEGEEIEVNC TAMASKPATTIRWFKGNTELKGKSEVEEWSDMYTVTSQLMLKVHKEDDGVPVICQVE HPAVTGNLQTQRYLEVQYKPQVHIQMTYPLQGLTREGDALELTCEAIGKPQPVMVTWV RVDDEMPQHAVLSGPNLFINNLNKTDNGTYRCEASNIVGKAHSDYMLYVYDPPTTIPPP TTTTTTTTTTTTTILTIITDSRAGEEGSIRAVDH (SEQ ID NO: 3450), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3450.

In other embodiments, the NK cell engager is a ligand of CD27, which is CD70, e.g., wherein CD70 comprises the amino acid sequence: QRFAQAQQQLPLESLGWDVAELQLNHTGPQQDPRLYWQGGPALGRSFLHGPELDKGQ LRIHRDGIYMVHIQVTLAICSSTTASRHHPTTLAVGICSPASRSISLLRLSFHQGCTIASQR LTPLARGDTLCTNLTGTLLPSRNTDETFFGVQWVRP (SEQ ID NO: 3451), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3451.

In other embodiments, the NK cell engager is a ligand of PSGL1, which is L-selectin (CD62L), e.g., wherein L-selectin comprises the amino acid sequence: WTYHYSEKPMNWQRARRFCRDNYTDLVAIQNKAEIEYLEKTLPF SRSYYWIGIRKIGGI WTWVGTNKSLTEEAENWGDGEPNNKKNKEDCVEIYIKRNKDAGKWNDDACHKLKAA LCYTASCQPWSCSGHGECVEIINNYTCNCDVGYYGPQCQFVIQCEPLEAPELGTMDCTH PLGNFSFSSQCAFSCSEGTNLTGIEETTCGPFGNWSSPEPTCQVIQCEPLSAPDLGIMNCSH PLASFSFTSACTFICSEGTELIGKKKTICESSGIWSNPSPICQKLDKSFSMIKEGDYN (SEQ ID NO: 3452), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3452.

In other embodiments, the NK cell engager is a ligand of CD96, which is NECLS, e.g., wherein NECLS comprises the amino acid sequence: WPPPGTGDVVVQAPTQVPGFLGDSVTLPCYLQVPNMEVTHVSQLTWARHGESGSMAV FHQTQGPSYSESKRLEFVAARLGAELRNASLRMFGLRVEDEGNYTCLFVTFPQGSRSVD IWLRVLAKPQNTAEVQKVQLTGEPVPMARCVSTGGRPPAQITWHSDLGGMPNTSQVPG FLSGTVTVTSLWILVPSSQVDGKNVTCKVEHESFEKPQLLTVNLTVYYPPEVSISGYDNN WYLGQNEATLTCDARSNPEPTGYNWSTTMGPLPPFAVAQGAQLLIRPVDKPINTTLICN VTNALGARQAELTVQVKEGPPSEHSGISRN (SEQ ID NO: 3449), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3449.

In other embodiments, the NK cell engager is a ligand of CD100 (SEMA4D), which is CD72, e.g., wherein CD72 comprises the amino acid sequence: RYLQVSQQLQQTNRVLEVTNSSLRQQLRLKITQLGQSAEDLQGSRRELAQSQEALQVEQ RAHQAAEGQLQACQADRQKTKETLQSEEQQRRALEQKLSNMENRLKPFFTCGSADTCC PSGWIMHQKSCFYISLTSKNWQESQKQCETLSSKLATFSEIYPQSHSYYFLNSLLPNGGS GNSYWTGLSSNKDWKLTDDTQRTRTYAQSSKCNKVHKTWSWWTLESESCRSSLPYICE MTAFRFPD (SEQ ID NO: 3453), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3453.

In other embodiments, the NK cell engager is a ligand of NKp80, which is CLEC2B (AICL), e.g., wherein CLEC2B (AICL) comprises the amino acid sequence: KLTRDSQSLCPYDWIGFQNKCYYFSKEEGDWNSSKYNCSTQHADLTIIDNIEEMNFLRR YKCSSDHWIGLKMAKNRTGQWVDGATFTKSFGMRGSEGCAYLSDDGAATARCYTER KWICRKRIH (SEQ ID NO: 3454), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3454.

In other embodiments, the NK cell engager is a ligand of CD244, which is CD48, e.g., wherein CD48 comprises the amino acid sequence: QGHLVHMTVVSGSNVTLNISESLPENYKQLTWFYTFDQKIVEWDSRKSKYFESKFKGR VRLDPQSGALYISKVQKEDNSTYIMRVLKKTGNEQEWKIKLQVLDPVPKPVIKIEKIEDM DDNCYLKLSCVIPGESVNYTWYGDKRPFPKELQNSVLETTLMPHNYSRCYTCQVSNSVS SKNGTVCLSPPCTLARS (SEQ ID NO: 3455), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3455.

### T Cell Engagers

The present disclosure provides, inter alia, multispecific (e.g., bi-, tri-, quad- specific) or multifunctional molecules, that are engineered to contain one or more T cell engager that mediate binding to and/or activation of a T cell. In some embodiments, the T cell engager is an antigen binding domain that binds to, e.g., activates TCRβ, e.g., a TCRβ V region, as described herein. In some embodiments, the T cell engager is selected from an antigen binding domain or ligand that binds to (e.g., and in some embodiments activates) one or more of CD3, TCRα, TCRγ, TCRζ, ICOS, CD28, CD27, HVEM, LIGHT, CD40, 4-1BB, OX40, DR3, GITR, CD30, TIM1, SLAM, CD2, or CD226. In other embodiments, the T cell engager is selected from an antigen binding domain or ligand that binds to and does not activate one or more of CD3, TCRα, TCRγ, TCRζ, ICOS, CD28, CD27, HVEM, LIGHT, CD40, 4-1BB, OX40, DR3, GITR, CD30, TIM1, SLAM, CD2, or CD226.

### B Cell, Macrophage & Dendritic Cell Engagers

Broadly, B cells, also known as B lymphocytes, are a type of white blood cell of the lymphocyte subtype. They function in the humoral immunity component of the adaptive immune system by secreting antibodies. Additionally, B cells present antigen (they are also classified as professional antigen-presenting cells (APCs)) and secrete cytokines. Macrophages are a type of white blood cell that engulfs and digests cellular debris, foreign substances, microbes, and other infectious agents via phagocytosis. Besides phagocytosis, they play important roles in nonspecific defense (innate immunity) and also help initiate specific defense mechanisms (adaptive immunity) by recruiting other immune cells such as lymphocytes. For example, they are important as antigen presenters to T cells. Beyond increasing inflammation and stimulating the immune system, macrophages also play an important anti-inflammatory role and can decrease immune reactions through the release of cytokines. Dendritic cells (DCs) are antigen-presenting cells that function in processing antigen material and present it on the cell surface to the T cells of the immune system.

The present disclosure provides, inter alfa, multispecific (e.g., bi-, tri-, quad- specific) or multifunctional molecules, that include, e.g., are engineered to contain, one or more B cell, macrophage, and/or dendritic cell engager that mediate binding to and/or activation of a B cell, macrophage, and/or dendritic cell.

Accordingly, in some embodiments, the immune cell engager comprises a B cell, macrophage, and/or dendritic cell engager chosen from one or more of CD40 ligand (CD4OL) or a CD70 ligand; an antibody molecule that binds to CD40 or CD70; an antibody molecule to OX40; an OX40 ligand (OX4OL); an agonist of a Toll-like receptor (e.g., as described herein, e.g., a TLR4, e.g., a constitutively active TLR4 (caTLR4), or a TLR9 agonists); a 41BB; a CD2; a CD47; or a STING agonist, or a combination thereof.

In some embodiments, the B cell engager is a CD40L, an OX40L, or a CD70 ligand, or an antibody molecule that binds to OX40, CD40 or CD70.

In some embodiments, the macrophage engager is a CD2 agonist. In some embodiments, the macrophage engager is an antigen binding domain that binds to: CD40L or antigen binding domain or ligand that binds CD40, a Toll like receptor (TLR) agonist (e.g., as described herein), e.g., a TLR9 or TLR4 (e.g., caTLR4 (constitutively active TLR4), CD47, or a STING agonist. In some embodiments, the STING agonist is a cyclic dinucleotide, e.g., cyclic di-GMP (cdGMP) or cyclic di-AMP (cdAMP). In some embodiments, the STING agonist is biotinylated.

In some embodiments, the dendritic cell engager is a CD2 agonist. In some embodiments, the dendritic cell engager is a ligand, a receptor agonist, or an antibody molecule that binds to one or more of: OX40L, 41BB, a TLR agonist (e.g., as described herein) (e.g., TLR9 agonist, TLR4 (e.g., caTLR4 (constitutively active TLR4)), CD47, or and a STING agonist. In some embodiments, the STING agonist is a cyclic dinucleotide, e.g., cyclic di-GMP (cdGMP) or cyclic di-AMP (cdAMP). In some embodiments, the STING agonist is biotinylated.

In other embodiments, the immune cell engager mediates binding to, or activation of, one or more of a B cell, a macrophage, and/or a dendritic cell. Exemplary B cell, macrophage, and/or dendritic cell engagers can be chosen from one or more of CD40 ligand (CD40L) or a CD70 ligand; an antibody molecule that binds to CD40 or CD70; an antibody molecule to OX40; an OX40 ligand (OX40L); a Toll-like receptor agonist (e.g., a TLR4, e.g., a constitutively active TLR4 (caTLR4) or a TLR9 agonist); a 41BB agonist; a CD2; a CD47; or a STING agonist, or a combination thereof.

In some embodiments, the B cell engager is chosen from one or more of a CD40L, an OX40L, or a CD70 ligand, or an antibody molecule that binds to OX40, CD40 or CD70.

In other embodiments, the macrophage cell engager is chosen from one or more of a CD2 agonist; a CD40L; an OX40L; an antibody molecule that binds to OX40, CD40 or CD70; a Toll-like receptor agonist or a fragment thereof (e.g., a TLR4, e.g., a constitutively active TLR4

In other embodiments, the dendritic cell engager is chosen from one or more of a CD2 agonist, an OX40 antibody, an OX40L, 41BB agonist, a Toll-like receptor agonist or a fragment thereof (e.g., a TLR4, e.g., a constitutively active TLR4 (caTLR4)), CD47 agonist, or a STING agonist.

In one embodiment, the OX40L comprises the amino acid sequence: QVSHRYPRIQSIKVQFTEYKKEKGFILTSQKEDEIMKVQNNSVIINCDGFYLISLKGYFSQ EVNISLHYQKDEEPLFQLKKVRSVNSLMVASLTYKDKVYLNVTTDNTSLDDFHVNGGE LILIHQNPGEFCVL (SEQ ID NO: 3456), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3456.

In another embodiment, the CD40L comprises the amino acid sequence: MQKGDQNPQIAAHVISEASSKTTSVLQWAEKGYYTMSNNLVTLENGKQLTVKRQGLY YIYAQVTFCSNREASSQAPFIASLCLKSPGRFERILLRAANTHSSAKPCGQQSIHLGGVFE LQPGASVFVNVTDPSQVSHGTGFTSFGLLKL (SEQ ID NO: 3457), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3457.

In yet other embodiments, the STING agonist comprises a cyclic dinucleotide, e.g., a cyclic di-GMP (cdGMP), a cyclic di-AMP (cdAMP), or a combination thereof, optionally with 2′,5′ or 3′,5′ phosphate linkages.

In one embodiment, the immune cell engager includes 41BB ligand, e.g., comprising the amino acid sequence: ACPWAVSGARASPGSAASPRLREGPELSPDDPAGLLDLRQGMFAQLVAQNVLLIDGPLS WYSDPGLAGVSLTGGLSYKEDTKELVVAKAGVYYVFFQLELRRVVAGEGSGSVSLALH LQPLRSAAGAAALALTVDLPPAS SEARNSAFGFQGRLLHL SAGQRLGVHLHTEARARH AWQLTQGATVLGLFRVTPEIPAGLPSPRSE (SEQ ID NO: 3458), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3458.

### Toll-Like Receptors

Toll-Like Receptors (TLRs) are evolutionarily conserved receptors are homologues of the Drosophila Toll protein, and recognize highly conserved structural motifs known as pathogen-associated microbial patterns (PAMPs), which are exclusively expressed by microbial pathogens, or danger-associated molecular patterns (DAMPs) that are endogenous molecules released from necrotic or dying cells. PAMPs include various bacterial cell wall components such as lipopolysaccharide (LPS), peptidoglycan (PGN) and lipopeptides, as well as flagellin, bacterial DNA and viral double-stranded RNA. DAMPs include intracellular proteins such as heat shock proteins as well as protein fragments from the extracellular matrix. Stimulation of TLRs by the corresponding PAMPs or DAMPs initiates signaling cascades leading to the activation of transcription factors, such as AP-1, NF-κB and interferon regulatory factors (IRFs). Signaling by TLRs results in a variety of cellular responses, including the production of interferons (IFNs), pro-inflammatory cytokines and effector cytokines that direct the adaptive immune response. TLRs are implicated in a number of inflammatory and immune disorders .

TLRs are type I transmembrane proteins characterized by an extracellular domain containing leucine-rich repeats (LRRs) and a cytoplasmic tail that contains a conserved region called the Toll/IL-1 receptor (TIR) domain. Ten human and twelve murine TLRs have been characterized, TLR1 to TLR10 in humans, and TLR1 to TLR9, TLR11, TLR12 and TLR13 in mice, the homolog of TLR10 being a pseudogene. TLR2 is essential for the recognition of a variety of PAMPs from Gram-positive bacteria, including bacterial lipoproteins, lipomannans and lipoteichoic acids. TLR3 is implicated in virus-derived double-stranded RNA. TLR4 is predominantly activated by lipopolysaccharide. TLRS detects bacterial flagellin and TLR9 is required for response to unmethylated CpG DNA. Finally, TLR7 and TLR8 recognize small synthetic antiviral molecules, and single-stranded RNA was reported to be their natural ligand. TLR11 has been reported to recognize uropathogenic E.coli and a profilin-like protein from Toxoplasma gondii. The repertoire of specificities of the TLRs is apparently extended by the ability of TLRs to heterodimerize with one another. For example, dimers of TLR2 and TLR6 are required for responses to diacylated lipoproteins while TLR2 and TLR1 interact to recognize triacylated lipoproteins. Specificities of the TLRs are also influenced by various adapter and accessory molecules, such as MD-2 and CD14 that form a complex with TLR4 in response to LPS.

TLR signaling consists of at least two distinct pathways: a MyD88-dependent pathway that leads to the production of inflammatory cytokines, and a MyD88-independent pathway associated with the stimulation of IFN-β and the maturation of dendritic cells. The MyD88-dependent pathway is common to all TLRs, except TLR3 (Adachi O. et al., 1998. Targeted disruption of the MyD88 gene results in loss of IL-1- and IL-18-mediated function. Immunity. 9(1):143-50). Upon activation by PAMPs or DAMPs, TLRs hetero- or homodimerize inducing the recruitment of adaptor proteins via the cytoplasmic TIR domain. Individual TLRs induce different signaling responses by usage of the different adaptor molecules. TLR4 and TLR2 signaling requires the adaptor TIRAP/Mal, which is involved in the MyD88-dependent pathway. TLR3 triggers the production of IFN-β in response to double-stranded RNA, in a MyD88-independent manner, through the adaptor TRIF/TICAM-1. TRAM/TICAM-2 is another adaptor molecule involved in the MyD88-independent pathway which function is restricted to the TLR4 pathway.

TLR3, TLR7, TLR8 and TLR9 recognize viral nucleic acids and induce type I IFNs. The signaling mechanisms leading to the induction of type I IFNs differ depending on the TLR activated. They involve the interferon regulatory factors, IRFs, a family of transcription factors known to play a critical role in antiviral defense, cell growth and immune regulation. Three IRFs (IRF3, IRF5 and IRF7) function as direct transducers of virus-mediated TLR signaling. TLR3 and TLR4 activate IRF3 and IRF7, while TLR7 and TLR8 activate IRF5 and IRF7 (Doyle S. et al., 2002. IRF3 mediates a TLR3/TLR4-specific antiviral gene program. Immunity. 17(3):251-63). Furthermore, type I IFN production stimulated by TLR9 ligand CpG-A has been shown to be mediated by PI(3)K and mTOR (Costa-Mattioli M. & Sonenberg N. 2008. RAPping production of type I interferon in pDCs through mTOR. Nature Immunol. 9: 1097-1099).

TLR-9

TLR9 recognizes unmethylated CpG sequences in DNA molecules. CpG sites are relatively rare (-1%) on vertebrate genomes in comparison to bacterial genomes or viral DNA. TLR9 is expressed by numerous cells of the immune system such as B lymphocytes, monocytes, natural killer (NK) cells, and plasmacytoid dendritic cells. TLR9 is expressed intracellularly, within the endosomal compartments and functions to alert the immune system of viral and bacterial infections by binding to DNA rich in CpG motifs. TLR9 signals leads to activation of the cells initiating pro-inflammatory reactions that result in the production of cytokines such as type-I interferon and IL-12.

TLR Agonists

A TLR agonist can agonize one or more TLR, e.g., one or more of human TLR- 1, 2, 3, 4, 5, 6, 7, 8, 9, or 10. In some embodiments, an adjunctive agent described herein is a TLR agonist. In some embodiments, the TLR agonist specifically agonizes human TLR-9. In some embodiments, the TLR-9 agonist is a CpG moiety. As used herein, a CpG moiety, is a linear dinucleotide having the sequence: 5′-C-phosphate-G-3′, that is, cytosine and guanine separated by only one phosphate.

In some embodiments, the CpG moiety comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more CpG dinucleotides. In some embodiments, the CpG moiety consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 CpG dinucleotides. In some embodiments, the CpG moiety has 1-5, 1-10, 1-20, 1-30, 1-40, 1-50, 5-10, 5-20, 5-30, 10-20, 10-30, 10-40, or 10-50 CpG dinucleotides.

In some embodiments, the TLR-9 agonist is a synthetic ODN (oligodeoxynucleotides). CpG ODNs are short synthetic single-stranded DNA molecules containing unmethylated CpG dinucleotides in particular sequence contexts (CpG motifs). CpG ODNs possess a partially or completely phosphorothioated (PS) backbone, as opposed to the natural phosphodiester (PO) backbone found in genomic bacterial DNA. There are three major classes of CpG ODNs: classes A, B and C, which differ in their immunostimulatory activities. CpG-A ODNs are characterized by a PO central CpG-containing palindromic motif and a PS-modified 3′ poly-G string. They induce high IFN-α production from pDCs but are weak stimulators of TLR9-dependent NF-κB signaling and pro-inflammatory cytokine (e.g. IL-6) production. CpG-B ODNs contain a full PS backbone with one or more CpG dinucleotides. They strongly activate B cells and TLR9-dependent NF-κB signaling but weakly stimulate IFN-a secretion. CpG-C ODNs combine features of both classes A and B. They contain a complete PS backbone and a CpG-containing palindromic motif. C-Class CpG ODNs induce strong IFN-a production from pDC as well as B cell stimulation.

### Infectious Disease-Targeting Moieties

The present disclosure provides, inter alfa, multispecific (e.g., bi-, tri-, tetra- specific) molecules, that include, e.g., are engineered to contain, one or more infectious disease-targeting moieties that direct the molecule to an infectious agent, or a portion thereof, e.g., as described herein.

In certain embodiments, the multispecific molecules disclosed herein include an infectious disease-targeting moiety. The infectious disease-targeting moiety can be chosen from an antibody molecule (e.g., an antigen binding domain as described herein), a receptor or a receptor fragment, or a ligand or a ligand fragment, or a combination thereof. In some embodiments, the infectious disease-targeting moiety associates with, e.g., binds to, an infectious agent or a portion thereof (e.g., a molecule, e.g., antigen, present on, derived from, or comprised in the infectious agent). In certain embodiments, the infectious disease-targeting moiety targets, e.g., directs the multispecific molecules disclosed herein to an infectious agent or portion thereof, e.g., as described herein.

### Stromal Modifying Moieties

Stromal modifying moieties described herein include moieties (e.g., proteins, e.g., enzymes) capable of degrading a component of the stroma, e.g., an ECM component, e.g., a glycosaminoglycan, e.g., hyaluronan (also known as hyaluronic acid or HA), chondroitin sulfate, chondroitin, dermatan sulfate, heparin sulfate, heparin, entactin, tenascin, aggrecan and keratin sulfate; or an extracellular protein, e.g., collagen, laminin, elastin, fibrinogen, fibronectin, and vitronectin.

### Stromal Modifying Enzymes

In some embodiments, the stromal modifying moiety is an enzyme. For example, the stromal modifying moiety can include, but is not limited to a hyaluronidase, a collagenase, a chondroitinase, a matrix metalloproteinase (e.g., macrophage metalloelastase).

Hyaluronidases

Hyaluronidases are a group of neutral- and acid-active enzymes found throughout the animal kingdom. Hyaluronidases vary with respect to substrate specificity, and mechanism of action. There are three general classes of hyaluronidases: (1) Mammalian-type hyaluronidases, (EC 3.2.1.35) which are endo-beta-N-acetylhexosaminidases with tetrasaccharides and hexasaccharides as the major end products. They have both hydrolytic and transglycosidase activities, and can degrade hyaluronan and chondroitin sulfates; (2) Bacterial hyaluronidases (EC 4.2.99.1) degrade hyaluronan and, and to various extents, chondroitin sulfate and dermatan sulfate. They are endo-beta-N-acetylhexosaminidases that operate by a beta elimination reaction that yields primarily disaccharide end products; (3) Hyaluronidases (EC 3.2.1.36) from leeches, other parasites, and crustaceans are endo-beta-glucuronidases that generate tetrasaccharide and hexasaccharide end products through hydrolysis of the beta 1-3 linkage.

Mammalian hyaluronidases can be further divided into two groups: (1) neutral active and (2) acid active enzymes. There are six hyaluronidase-like genes in the human genome, HYAL1, HYAL2, HYAL3 HYAL4 HYALP1 and PH20/SPAM1. HYALP1 is a pseudogene, and HYAL3 has not been shown to possess enzyme activity toward any known substrates. HYAL4 is a chondroitinase and lacks activity towards hyaluronan. HYAL1 is the prototypical acid-active enzyme and PH20 is the prototypical neutral-active enzyme. Acid active hyaluronidases, such as HYAL1 and HYAL2 lack catalytic activity at neutral pH. For example, HYAL1 has no catalytic activity in vitro over pH 4.5 (Frost and Stern, “A Microtiter-Based Assay for Hyaluronidase Activity Not Requiring Specialized Reagents”, Analytical Biochemistry, vol. 251, pp. 263-269 (1997). HYAL2 is an acid active enzyme with a very low specific activity in vitro.

In some embodiments the hyaluronidase is a mammalian hyaluronidase. In some embodiments the hyaluronidase is a recombinant human hyaluronidase. In some embodiments, the hyaluronidase is a neutral active hyaluronidase. In some embodiments, the hyaluronidase is a neutral active soluble hyaluronidase. In some embodiments, the hyaluronidase is a recombinant PH20 neutral-active enzyme. In some embodiments, the hyaluronidase is a recombinant PH20 neutral-active soluble enzyme. In some embodiments the hyaluronidase is glycosylated. In some embodiments, the hyaluronidase possesses at least one N-linked glycan. A recombinant hyaluronidase can be produced using conventional methods known to those of skill in the art, e.g., U.S. Pat. No. 7,767,429, the entire contents of which are incorporated by reference herein.

In some embodiments the hyaluronidase is rHuPH20 (also referred to as Hylenex®; presently manufactured by Halozyme; approved by the FDA in 2005 (see e.g., Scodeller P (2014) Hyaluronidase and other Extracellular Matrix Degrading Enzymes for Cancer Therapy: New Uses and Nano- Formulations. J Carcinog Mutage 5:178; U.S. Pat. Nos. 7,767,429; 8,202,517; 7,431,380; 8,450,470; 8,772,246; 8,580,252, the entire contents of each of which is incorporated by reference herein). rHuPH20 is produced by genetically engineered CHO cells containing a DNA plasmid encoding for a soluble fragment of human hyaluronidase PH20. In some embodiments the hyaluronidase is glycosylated. In some embodiments, the hyaluronidase possesses at least one N-linked glycan. A recombinant hyaluronidase can be produced using conventional methods known to those of skill in the art, e.g., U.S. Pat. No. 7,767,429, the entire contents of which are incorporated by reference herein. In some embodiments, rHuPH20 has a sequence at least 95% (e.g., at least 96%, 97%, 98%, 99%, 100%) identical to the amino acid sequence of

(SEQ ID NO: 3459)
LNFRAPPVIPNVPFLWAWNAPSEFCLGKFDEPLDMSLFSFIGSPRINATGQ
GVTIFYVDRLGYYPYIDSITGVTVNGGIPQKISLQDHLDKAKKDITFYMPV
DNLGMAVIDWEEWRPTWARNWKPKDVYKNRSIELVQQQNVQLSLTEATEKA
KQEFEKAGKDFLVETIKLGKLLRPNHLWGYYLFPDCYNHHYKKPGYNGSCF
NVEIKRNDDLSWLWNESTALYPSIYLNTQQSPVAATLYVRNRVREAIRVSK
IPDAKSPLPVFAYTRIVFTDQVLKFLSQDELVYTFGETVALGASGIVIWGT
LSIMRSMKSCLLLDNYMETILNPYIINVTLAAKMCSQVLCQEQGVCIRKNW
NSSDYLHLNPDNFAIQLEKGGKFTVRGKPTLEDLEQFSEKFYCSCYSTLSC
KEKADVKDTDAVDVCIADGVCIDAFLKPPMETEEPQIFYNASPSTLS.

In any of the methods provided herein, the anti-hyaluronan agent can be an agent that degrades hyaluronan or can be an agent that inhibits the synthesis of hyaluronan. For example, the anti-hyaluronan agent can be a hyaluronan degrading enzyme. In another example, the anti-hyaluronan agent or is an agent that inhibits hyaluronan synthesis. For example, the anti-hyaluronan agent is an agent that inhibits hyaluronan synthesis such as a sense or antisense nucleic acid molecule against an HA synthase or is a small molecule drug. For example, an anti-hyaluronan agent is 4-methylumbelliferone (MU) or a derivative thereof, or leflunomide or a derivative thereof. Such derivatives include, for example, a derivative of 4-methylumbelliferone (MU) that is 6,7-dihydroxy-4-methyl coumarin or 5,7-dihydroxy-4-methyl coumarin.

In further examples of the methods provided herein, the hyaluronan degrading enzyme is a hyaluronidase. In some examples, the hyaluronan-degrading enzyme is a PH20 hyaluronidase or truncated form thereof to lacking a C-terminal glycosylphosphatidylinositol (GPI) attachment site or a portion of the GPI attachment site. In specific examples, the hyaluronidase is a PH20 selected from a human, monkey, bovine, ovine, rat, mouse or guinea pig PH20. For example, the hyaluronan- degrading enzyme is a human PH20 hyaluronidase that is neutral active and N- glycosylated and is selected from among (a) a hyaluronidase polypeptide that is a full- length PH20 or is a C-terminal truncated form of the PH20, wherein the truncated form includes at least amino acid residues 36-464 of SEQ ID NO: 3459, such as 36-481 , 36-482, 36-483, where the full-length PH20 has the sequence of amino acids set forth in SEQ ID NO: 3459; or (b) a hyaluronidase polypeptide comprising a sequence of amino acids having at least 85%, 86%, 87%, 88%, 89%, 90%, 91%, 92%, 93%, 94%, 95%, 96%, 97%, 98%, 99% or more sequence identity with the polypeptide or truncated form of sequence of amino acids set forth in SEQ ID NO: 3459; or (c) a hyaluronidase polypeptide of (a) or (b) comprising amino acid substitutions, whereby the hyaluronidase polypeptide has a sequence of amino acids having at least 85%, 86 %, 87 %, 88 %, 89 %, 90 %, 91 %, 92 %, 93 %, 94 %, 95 %, 96 %, 97 %, 98%, 99% or more sequence identity with the polypeptide set forth in SEQ ID NO: 3459or the with the corresponding truncated forms thereof. In exemplary examples, the hyaluronan- degrading enzyme is a PH20 that comprises a composition designated rHuPH20.

In other examples, the anti-hyaluronan agent is a hyaluronan degrading enzyme that is modified by conjugation to a polymer. The polymer can be a PEG and the anti-hyaluronan agent a PEGylated hyaluronan degrading enzyme. Hence, in some examples of the methods provided herein the hyaluronan-degrading enzyme is modified by conjugation to a polymer. For example, the hyaluronan-degrading enzyme is conjugated to a PEG, thus the hyaluronan degrading enzyme is PEGylated. In an exemplary example, the hyaluronan-degrading enzyme is a PEGylated PH20 enzyme (PEGPH20). In the methods provided herein, the corticosteroid can be a glucocorticoid that is selected from among cortisones, dexamethasones, hydrocortisones, methylprednisolones, prednisolones and prednisones.

Chondroitinases

Chondroitinases are enzymes found throughout the animal kingdom which degrade glycosaminoglycans, specifically chondroitins and chondroitin sulfates, through an endoglycosidase reaction. In some embodiments the chondroitinase is a mammalian chondroitinase. In some embodiments the chondroitinase is a recombinant human chondroitinase. In some embodiments the chondroitinase is HYAL4. Other exemplary chondroitinases include chondroitinase ABC (derived from Proteus vulgaris; Japanese Patent Application Laid-open No 6-153947, T. Yamagata et al. J. Biol. Chem., 243, 1523 (1968), S. Suzuki et al, J. Biol. Chem., 243, 1543 (1968)), chondroitinase AC (derived from Flavobacterium heparinum; T. Yamagata et al., J. Biol. Chem., 243, 1523 (1968)), chondroitinase AC II (derived from Arthrobacter aurescens; K. Hiyama, and S. Okada, J. Biol. Chem., 250, 1824 (1975), K. Hiyama and S. Okada, J. Biochem. (Tokyo), 80, 1201 (1976)), Hyaluronidase ACIII (derived from Flavobacterium sp. Hp102; Hirofumi Miyazono et al., Seikagaku, 61, 1023 (1989)), chondroitinase B (derived from Flavobacterium heparinum; Y. M. Michelacci and C. P. Dietrich, Biochem. Biophys. Res. Commun., 56, 973 (1974), Y. M. Michelacci and C. P. Dietrich, Biochem. J., 151, 121 (1975), Kenichi Maeyama et al, Seikagaku, 57, 1189 (1985)), chondroitinase C (derived from Flavobacterium sp. Hp102; Hirofumi Miyazono et al, Seikagaku, 61, 1023 (1939)), and the like.

Matrix Metalloproteinases

Matrix metalloproteases (MMPs) are zinc-dependent endopeptidases that are the major proteases involved in extracellular matrix (ECM) degradation. MMPs are capable of degrading a wide range of extracellular molecules and a number of bioactive molecules. Twenty-four MMP genes have been identified in humans, which can be organized into six groups based on domain organization and substrate preference: Collagenases (MMP-1,-8 and -13), Gelatinases (MMP-2 and MMP-9), Stromelysins (MMP-3,-10 and -11), Matrilysin (MMP-7 and MMP-26), Membrane-type (MT)-MMPs (MMP-14,-15,-16,-17,-24 and -25) and others (MMP-12,-19,-20,-21,-23,-27 and -28). In some embodiments, the stromal modifying moiety is a human recombinant MMP (e.g., MMP-1,-2,-3,-4,-5,-6,-7,-8,-9, 10,-11,-12,-13,-14, 15,-15,-17, -18,-19, 20,-21,-22,-23, or -24).

Collagenases

The three mammalian collagenases (MMP-1,-8, and -13) are the principal secreted endopeptidases capable of cleaving collagenous extracellular matrix. In addition to fibrillar collagens, collagenases can cleave several other matrix and non-matrix proteins including growth factors. Collagenases are synthesized as inactive pro-forms, and once activated, their activity is inhibited by specific tissue inhibitors of metalloproteinases, TIMPs, as well as by non-specific proteinase inhibitors (Ala-aho R et al. Biochimie. Collagenases in cancer. 2005 March-April; 87(3-4):273-86). In some embodiments, the stromal modifying moiety is a collagenase. In some embodiments, the collagenase is a human recombinant collagenase. In some embodiments, the collagenase is MMP-1. In some embodiments, the collagenase is MMP-8. In some embodiments, the collagenase is MMP-13.

Macrophage Metalloelastase

Macrophage metalloelastase (MME), also known as MMP-12, is a member of the stromelysin subgroup of MMPs and catalyzes the hydrolysis of soluble and insoluble elastin and a broad selection of matrix and nonmatrix substrates including type IV collagen, fibronectin, laminin, vitronectin, entactin, heparan, and chondroitin sulfates (Erj a Kerkela et al. Journal of Investigative Dermatology (2000) 114, 1113-1119; doi:10.1046/j.1523-1747.2000.00993). In some embodiments, the stromal modifying moiety is a MME. In some embodiments, the MME is a human recombinant MME. In some embodiments, the MME is MMP-12.

### Additional Stromal Modifying Moieties

In some embodiments, the stromal modifying moiety decreases the level or production of a stromal or extracellular matrix (ECM) component.

In some embodiments, the stromal or ECM component decreased is chosen from a glycosaminoglycan or an extracellular protein, or a combination thereof. In some embodiments, the glycosaminoglycan is chosen from hyaluronan (also known as hyaluronic acid or HA), chondroitin sulfate, chondroitin, dermatan sulfate, heparin, heparin sulfate, entactin, tenascin, aggrecan and keratin sulfate. In some embodiments, the extracellular protein is chosen from collagen, laminin, elastin, fibrinogen, fibronectin, or vitronectin. In some embodiments, the stromal modifying moiety includes an enzyme molecule that degrades a stroma or extracellular matrix (ECM). In some embodiments, the enzyme molecule is chosen from a hyaluronidase molecule, a collagenase molecule, a chondroitinase molecule, a matrix metalloproteinase molecule (e.g., macrophage metalloelastase), or a variant (e.g., a fragment) of any of the aforesaid. The term “enzyme molecule” includes a full length, a fragment or a variant of the enzyme, e.g., an enzyme variant that retains at least one functional property of the naturally-occurring enzyme.

In some embodiments, the stromal modifying moiety decreases the level or production of hyaluronic acid. In other embodiments, the stromal modifying moiety comprises a hyaluronan degrading enzyme, an agent that inhibits hyaluronan synthesis, or an antibody molecule against hyaluronic acid.

In some embodiments, the hyaluronan degrading enzyme is a hyaluronidase molecule, e.g., a full length or a variant (e.g., fragment thereof) thereof. In some embodiments, the hyaluronan degrading enzyme is active in neutral or acidic pH, e.g., pH of about 4-5. In some embodiments, the hyaluronidase molecule is a mammalian hyaluronidase molecule, e.g., a recombinant human hyaluronidase molecule, e.g., a full length or a variant (e.g., fragment thereof, e.g., a truncated form) thereof. In some embodiments, the hyaluronidase molecule is chosen from HYAL1, HYAL2, or PH-20/SPAM1, or a variant thereof (e.g., a truncated form thereof). In some embodiments, the truncated form lacks a C-terminal glycosylphosphatidylinositol (GPI) attachment site or a portion of the GPI attachment site. In some embodiments, the hyaluronidase molecule is glycosylated, e.g., comprises at least one N-linked glycan.

In some embodiments, the hyaluronidase molecule comprises the amino acid sequence: LNFRAPPVIPNVPFLWAWNAPSEFCLGKFDEPLDMSLFSFIGSPRINATGQGVTIFYVDRL GYYPYID SITGVTVNGGIPQKISLQDHLDKAKKDITFYMPVDNLGMAVIDWEEWRPTW ARNWKPKDVYKNRSIELVQQQNVQLSLTEATEKAKQEFEKAGKDFLVETIKLGKLLRP NHLWGYYLFPDCYNHHYKKPGYNGSCFNVEIKRNDDLSWLWNESTALYPSIYLNTQQS PVAATLYVRNRVREAIRVSKIPDAKSPLPVFAYTRIVFTDQVLKFLSQDELVYTFGETVA LGASGIVIWGTLSIMRSMKSCLLLDNYMETILNPYIINVTLAAKMCSQVLCQEQGVCIRK NWNSSDYLHLNPDNFAIQLEKGGKFTVRGKPTLEDLEQFSEKFYCSCYSTLSCKEKADV KDTDAVDVCIADGVCIDAFLKPPMETEEPQIFYNASPSTLS (SEQ ID NO: 3464), or a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3464.

In some embodiments, the hyaluronidase molecule comprises:
(i) the amino acid sequence of 36-464 of SEQ ID NO: 3464;
(ii) the amino acid sequence of 36-481, 36-482, or 36-483 of PH20, wherein PH20 has the sequence of amino acids set forth in SEQ ID NO: 3464; or
(iii) an amino acid sequence having at least 95% to 100% sequence identity to the polypeptide or truncated form of sequence of amino acids set forth in SEQ ID NO: 3464; or
(iv) an amino acid sequence having 30, 20, 10, 5 or fewer amino acid substitutions to the amino acid sequence set forth in SEQ ID NO: 3464. In some embodiments, the hyaluronidase molecule comprises an amino acid sequence at least 95% (e.g., at least 95%, 96%, 97%, 98%, 99%, 100%) identical to the amino acid sequence of SEQ ID NO: 3464. In some embodiments, the hyaluronidase molecule is encoded by a nucleotide sequence at least 95% (e.g., at least 96%, 97%, 98%, 99%, 100%) identical to the nucleotide sequence of SEQ ID NO: 3464.

In some embodiments, the hyaluronidase molecule is PH20, e.g., rHuPH20. In some embodiments, the hyaluronidase molecule is HYAL1 and comprises the amino acid sequence: FRGPLLPNRPF TTVWNANTQWCLERHGVDVDVSVFDVVANPGQTFRGPDMTIFYS SQG TYPYYTPTGEPVFGGLPQNASLIAHLARTFQDILAAIPAPDF SGLAVIDWEAWRPRWAFN WDTKDIYRQRSRALVQAQHPDWPAPQVEAVAQDQFQGAARAWMAGTLQLGRALRPR GLWGFYGFPDCYNYDFLSPNYTGQCPSGIRAQNDQLGWLWGQSRALYPSIYMPAVLEG TGKSQMYVQHRVAEAFRVAVAAGDPNLPVLPYVQIFYDTTNHFLPLDELEHSLGESAA QGAAGVVLWVSWENTRTKESCQAIKEYMDTTLGPFILNVTSGALLCSQALCSGHGRCV RRTSHPKALLLLNPASFSIQLTPGGGPLSLRGALSLEDQAQMAVEFKCRCYPGWQAPWC ERKSMW (SEQ ID NO: 3465), or a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3465.

In some embodiments, the hyaluronan degrading enzyme, e.g., the hyaluronidase molecule, further comprises a polymer, e.g., is conjugated to a polymer, e.g., PEG. In some embodiments, the hyaluronan-degrading enzyme is a PEGylated PH20 enzyme (PEGPH20). In some embodiments, the hyaluronan degrading enzyme, e.g., the hyaluronidase molecule, further comprises an immunoglobulin chain constant region (e.g., Fc region) chosen from, e.g., the heavy chain constant regions of IgG1, IgG2, IgG3, and IgG4, more particularly, the heavy chain constant region of human IgG1, IgG2, IgG3, or IgG4. In some embodiments, the immunoglobulin constant region (e.g., the Fc region) is linked, e.g., covalently linked to, the hyaluronan degrading enzyme, e.g., the hyaluronidase molecule. In some embodiments, the immunoglobulin chain constant region (e.g., Fc region) is altered, e.g., mutated, to increase or decrease one or more of: Fc receptor binding, antibody glycosylation, the number of cysteine residues, effector cell function, or complement function. In some embodiments, the hyaluronan degrading enzyme, e.g., the hyaluronidase molecule forms a dimer.

In some embodiments, the stromal modifying moiety comprises an inhibitor of the synthesis of hyaluronan, e.g., an HA synthase. In some embodiments, the inhibitor comprises a sense or an antisense nucleic acid molecule against an HA synthase or is a small molecule drug. In some embodiments, the inhibitor is 4-methylumbelliferone (MU) or a derivative thereof (e.g., 6,7-dihydroxy-4-methyl coumarin or 5,7-dihydroxy-4-methyl coumarin), or leflunomide or a derivative thereof.

In some embodiments, the stromal modifying moiety comprises antibody molecule against hyaluronic acid.

In some embodiments, the stromal modifying moiety comprises a collagenase molecule, e.g., a mammalian collagenase molecule, or a variant (e.g., fragment) thereof. In some embodiments, the collagenase molecule is collagenase molecule IV, e.g., comprising the amino acid sequence of: YNFFPRKPKWDKNQITYRIIGYTPDLDPETVDDAFARAFQVWSDVTPLRFSRIHDGEADI MINFGRWEHGDGYPFDGKDGLLAHAFAPGTGVGGDSHFDDDELWTLGEGQVVRVKY GNADGEYCKFPFLFNGKEYNSCTDTGRSDGFLWCSTTYNFEKDGKYGFCPHEALFTMG GNAEGQPCKFPFRFQGTSYDSCTTEGRTDGYRWCGTTEDYDRDKKYGFCPETAMSTVG GNSEGAPCVFPFTFLGNKYESCTSAGRSDGKMWCATTANYDDDRKWGFCPDQGYSLF LVAAHEFGHAMGLEHSQDPGALMAPIYTYTKNFRLSQDDIKGIQELYGASPDIDLGTGP TPTLGPVTPEICKQDIVFDGIAQIRGEIFFFKDRFIWRTVTPRDKPMGPLLVATFWPELPEK IDAVYEAPQEEKAVFFAGNEYWIYSASTLERGYPKPLTSLGLPPDVQRVDAAFNWSKNK KTYIFAGDKFWRYNEVKKKMDPGFPKLIADAWNAIPDNLDAVVDLQGGGHSYFFKGA YYLKLENQSLKSVKFGSIKSDWLGC (SEQ ID NO: 3466), or a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3466.

### Linkers

The multispecific or multifunctional molecule disclosed herein can further include a linker, e.g., a linker between one or more of: the antigen binding domain and the cytokine molecule, the antigen binding domain and the immune cell engager, the antigen binding domain and the stromal modifying moiety, the cytokine molecule and the immune cell engager, the cytokine molecule and the stromal modifying moiety, the immune cell engager and the stromal modifying moiety, the antigen binding domain and the immunoglobulin chain constant region, the cytokine molecule and the immunoglobulin chain constant region, the immune cell engager and the immunoglobulin chain constant region, or the stromal modifying moiety and the immunoglobulin chain constant region. In embodiments, the linker is chosen from: a cleavable linker, a non-cleavable linker, a peptide linker, a flexible linker, a rigid linker, a helical linker, or a non-helical linker, or a combination thereof.

In one embodiment, the multispecific molecule can include one, two, three or four linkers, e.g., a peptide linker. In one embodiment, the peptide linker includes Gly and Ser. In some embodiments, the peptide linker is selected from GGGGS (SEQ ID NO: 3460); GGGGSGGGGS (SEQ ID NO: 3461); GGGGSGGGGSGGGGS (SEQ ID NO: 3462); and DVPSGPGGGGGSGGGGS (SEQ ID NO: 3463). In some embodiments, the peptide linker is a A(EAAAK)nA (SEQ ID NO: 3477) family of linkers (e.g., as described in Protein Eng. (2001) 14 (8): 529-532). These are stiff helical linkers with n ranging from 2-5. In some embodiments, the peptide linker is selected from AEAAAKEAAAKAAA (SEQ ID NO: 3467); AEAAAKEAAAKEAAAKAAA (SEQ ID NO: 3468); AEAAAKEAAAKEAAAKEAAAKAAA (SEQ ID NO: 3469); and AEAAAKEAAAKEAAAKEAAAKEAAAKAAA(SEQ ID NO: 3470).

### Nucleic Acids

Nucleic acids encoding the aforementioned antibody molecules, e.g., anti-TCRβV antibody molecules, multispecific or multifunctional molecules are also disclosed.

In certain embodiments, the invention features nucleic acids comprising nucleotide sequences that encode heavy and light chain variable regions and CDRs or hypervariable loops of the antibody molecules, as described herein. For example, the invention features a first and second nucleic acid encoding heavy and light chain variable regions, respectively, of an antibody molecule chosen from one or more of the antibody molecules disclosed herein. The nucleic acid can comprise a nucleotide sequence as set forth in the tables herein, or a sequence substantially identical thereto (e.g., a sequence at least about 85%, 90%, 95%, 99% or more identical thereto, or which differs by no more than 3, 6, 15, 30, or 45 nucleotides from the sequences shown in the tables herein.

In certain embodiments, the nucleic acid can comprise a nucleotide sequence encoding at least one, two, or three CDRs or hypervariable loops from a heavy chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In other embodiments, the nucleic acid can comprise a nucleotide sequence encoding at least one, two, or three CDRs or hypervariable loops from a light chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In yet another embodiment, the nucleic acid can comprise a nucleotide sequence encoding at least one, two, three, four, five, or six CDRs or hypervariable loops from heavy and light chain variable regions having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions).

In certain embodiments, the nucleic acid can comprise a nucleotide sequence encoding at least one, two, or three CDRs or hypervariable loops from a heavy chain variable region having the nucleotide sequence as set forth in the tables herein, a sequence substantially homologous thereto (e.g., a sequence at least about 85%, 90%, 95%, 99% or more identical thereto, and/or capable of hybridizing under the stringency conditions described herein). In another embodiment, the nucleic acid can comprise a nucleotide sequence encoding at least one, two, or three CDRs or hypervariable loops from a light chain variable region having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein). In yet another embodiment, the nucleic acid can comprise a nucleotide sequence encoding at least one, two, three, four, five, or six CDRs or hypervariable loops from heavy and light chain variable regions having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein).

In certain embodiments, the nucleic acid can comprise a nucleotide sequence encoding a cytokine molecule, an immune cell engager, or a stromal modifying moiety disclosed herein.

In another aspect, the application features host cells and vectors containing the nucleic acids described herein. The nucleic acids may be present in a single vector or separate vectors present in the same host cell or separate host cell, as described in more detail hereinbelow.

### Vectors

Further provided herein are vectors comprising the nucleotide sequences encoding antibody molecules, e.g., anti-TCRβV antibody molecules, or a multispecific or multifunctional molecule described herein. In one embodiment, the vectors comprise nucleic acid sequences encoding antibody molecules, e.g., anti-TCRβV antibody molecules, or multispecific or multifunctional molecule described herein. In one embodiment, the vectors comprise the nucleotide sequences described herein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity.

Methods and conditions for culturing the resulting transfected cells and for recovering the antibody molecule produced are known to those skilled in the art, and may be varied or optimized depending upon the specific expression vector and mammalian host cell employed, based upon the present description.

### Cells

In another aspect, the application features host cells and vectors containing the nucleic acids described herein. The nucleic acids may be present in a single vector or separate vectors present in the same host cell or separate host cell. The host cell can be a eukaryotic cell, e.g., a mammalian cell, an insect cell, a yeast cell, or a prokaryotic cell, e.g., E. coli. For example, the mammalian cell can be a cultured cell or a cell line. Exemplary mammalian cells include lymphocytic cell lines (e.g., NSO), Chinese hamster ovary cells (CHO), COS cells, oocyte cells, and cells from a transgenic animal, e.g., mammary epithelial cell.

The invention also provides host cells comprising a nucleic acid encoding an antibody molecule as described herein.

In one embodiment, the host cells are genetically engineered to comprise nucleic acids encoding the antibody molecule.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The invention also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

Method of Expanding Cells with Anti-TCRVB Antibodies

Any of the compositions and methods described herein can be used to expand an immune cell population. An immune cell provided herein includes an immune cell derived from a hematopoietic stem cell or an immune cell derived from a non-hematopoietic stem cell, e.g., by differentiation or de-differentiation.

An immune cell includes a hematopoietic stem cell, progeny thereof and/or cells that have differentiated from said HSC, e.g., lymphoid cells or myeloid cells. An immune cell can be an adaptive immune cell or an innate immune cell. Examples of immune cells include T cells, B cells, Natural Killer cells, Natural Killer T cells, neutrophils, dendritic cells, monocytes, macrophages, and granulocytes.

In some embodiments of any of the methods of compositions disclosed herein, an immune cell is a T cell. In some embodiments, a T cell includes a CD4+T cell, a CD8+T cell, a TCR alpha-beta T cell, a TCR gamma-delta T cell. In some embodiments, a T cell comprises a memory T cell (e.g., a central memory T cell, or an effector memory T cell (e.g., a T_{EMRA}) or an effector T cell.

In some embodiments of any of the methods of compositions disclosed herein, an immune cell is an NK cell.

In certain aspects of the present disclosure, immune cells, e.g., T cells, can be obtained from a unit of blood collected from a subject using any number of techniques known to the skilled artisan, such as FicollTM separation. In one aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and, optionally, to place the cells in an appropriate buffer or media for subsequent processing steps. In one embodiment, the cells are washed with phosphate buffered saline (PBS). In an alternative embodiment, the wash solution lacks calcium and may lack magnesium or may lack many if not all divalent cations. The methods described herein can include more than one selection step, e.g., more than one depletion step.

In one embodiment, the methods of the application can utilize culture media conditions comprising DMEM, DMEM F12, RPMI 1640, and/or AIM V media. The media can be supplemented with glutamine, HEPES buffer (e.g., 10 mM), serum (e.g., heat-inactivated serum, e.g., 10%), and/or beta mercaptoethanol (e.g., 55 uM). IN some embodiments, the culture conditions disclosed herein comprise one or more supplements, cytokines, growth factors, or hormones. In some embodiments, the culture condition comprises one or more of IL-2, IL-15 or IL-7, or a combination thereof.

Immune effector cells such as T cells may be activated and expanded generally using methods as described, for example, in U.S. Pat. Nos. 6,352,694; 6,534,055; or 6,905,680. Generally, a population of immune cells, may be expanded by contact with an agent that stimulates a CD3/TCR complex associated signal and a ligand that stimulates a costimulatory molecule on the surface of the T cells; and/or by contact with a cytokine, e.g., IL-2, IL-15 or IL-7. T cell expansion protocols can also include stimulation, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For example, a population of T cells can be contacted with an anti-CD3 antibody and an anti-CD28 antibody, under conditions appropriate for stimulating proliferation of the T cells. To stimulate proliferation of either CD4+T cells or CD8+T cells, an anti-CD3 antibody and an anti-CD28 antibody can be used. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

A TIL population can also be expanded by methods known in the art. For example, a population of TILs can be expanded as described in Hall et al., Journal for ImmunoTherapy of Cancer (2016) 4:61, the entire contents of which are hereby incorporated by reference. Briefly, TILs can be isolated from a sample by mechanical and/or physical digestion. The resultant TIL population can be stimulated with an anti-CD3 antibody in the presence of non-dividing feeder cells. In some embodiments, the TIL population can be cultured, e.g., expanded, in the presence of IL-2, e.g., human IL-2. In some embodiments, the TIL cells can be cultured, e.g., expanded fora period of at least 1-21 days, e.g., at least 1, 2, 3, 4, 5, 6, 7, 8, 9, 10, 11, 12, 13, 14, 15, 16, 17, 18, 19, 20 or 21 days.

As disclosed herein, in some embodiments, an immune cell population (e.g., a T cell (e.g., a T_{EMRA} cell or a TIL population) can be expanded by contacting the immune cell population with an anti-TCRVB antibody, e.g., as described herein.

In some embodiments, the expansion occurs in vivo, e.g., in a subject. In some embodiments, a subject is administered an anti-TCRβV antibody molecule disclosed herein resulting in expansion of immune cells in vivo.

In some embodiments, the expansion occurs ex vivo, e.g., in vitro. In some embodiments, cells from a subject, e.g., T cells, e.g., TIL cells, are expanded in vitro with an anti-TCRβV antibody molecule disclosed herein. In some embodiments, the expanded TILs are administered to the subject to treat a disease or a symptom of a disease.

In some embodiments, a method of expansion disclosed herein results in an expansion of at least 1.1-10 fold, 10-20 fold, or 20-50 fold expansion. In some embodiments, the expansion is at least 1.1, 1.2, 1.3, 1.4, 1.5, 2, 3, 4, 5, 6, 7, 8, 9, 10, 15, 20, 25, 30, 35, 40, 45 or 50 fold expansion.

In some embodiments, a method of expansion disclosed herein comprises culturing, e.g., expanding, the cells for at least about 4 hours, 6 hours, 10 hours, 12 hours, 15 hours, 18 hours, 20 hours, or 22 hours. In some embodiments, a method of expansion disclosed herein comprises culturing, e.g., expanding, the cells for at least 1, 2, 3, 4, 5, 6, 7, 8, 9, 10, 11, 12, 13, 14, 15, 1,6 17, 18, 19, 20 or 21 days. In some embodiments, a method of expansion disclosed herein comprises culturing, e.g., expanding, the cells for at least about 1 week, 2 weeks, 3 weeks, 4 weeks, 5 weeks, 6 weeks, 7 weeks or 8 weeks.

In some embodiments, a method of expansion disclosed herein is performed on immune cells obtained from a healthy subject.

In some embodiments, a method of expansion disclosed herein is performed on immune cells (e.g., TILs) obtained from a subject having a disease, e.g., an infectious disease as disclosed herein.

In some embodiments, a method of expansion disclosed herein further comprises contacting the population of cells with an agent, that promotes, e.g., increases, immune cell expansion. In some embodiments, the agent comprises an immune checkpoint inhibitor, e.g., a PD-1 inhibitor, a LAG-3 inhibitor, a CTLA4 inhibitor, or a TIM-3 inhibitor. In some embodiments, the agent comprises a 4-1BB agonist, e.g., an anti-4-1BB antibody.

Without wishing to be bound by theory, it is believed that an anti-TCRβV antibody molecule disclosed herein can expand, e.g., selectively or preferentially expand, T cells expressing a T cell receptor (TCR) comprising a TCR alpha and/or TCR beta molecule, e.g., TCR alpha-beta T cells (αβ T cells). In some embodiments, an anti-TCRβV antibody molecule disclosed herein does not expand, or induce proliferation of T cells expressing a TCR comprising a TCR gamma and/or TCR delta molecule, e.g., TCR gamma-delta T cells (γδ T cells). In some embodiments, an anti-TCRβV antibody molecule disclosed herein, selectively or preferentially expands cx13 T cells over ya T cells.

Without wishing to be bound by theory, it is believed that, in some embodiments, γδ T cells are associated with cytokine release syndrome (CRS). In some embodiments, an anti-TCRβV antibody molecule disclosed herein results in selective expansion of non-γδ T cells, e.g., expansion of αβ T cels, thus reducing CRS.

In some embodiments, any of the compositions or methods disclosed herein result in an immune cell population having a reduction of, e.g., depletion of, γδ T cells. In some embodiments, the immune cell population is contacted with an agent that reduces, e.g., inhibits or depletes, γδ T cells, e.g., an anti-IL-17 antibody or an agent that binds to a TCR gamma and/or TCR delta molecule.

### Uses and Combination Therapies

Methods described herein include treating an infectious disease in a subject by using an anti-TCRβV antibody molecule, a multispecific or multifunctional molecule described herein, e.g., using a pharmaceutical composition described herein. Also provided are methods for reducing or ameliorating a symptom of an infectious disease in a subject, as well as methods for inhibiting the growth of an infectious disease and/or killing one or more infectious agents.

In embodiments, the infectious disease is selected from Epstein-Barr virus (EBV), influenza, human immunodeficiency virus (HIV), simian immunodeficiency virus (SIV), tuberculosis, malaria, or human cytomegalovirus (HCMV), or a combination thereof.

In embodiments, the anti-TCRβV antibody molecule, multispecific or multifunctional molecules (or pharmaceutical composition) are administered in a manner appropriate to the disease to be treated or prevented. The quantity and frequency of administration will be determined by such factors as the condition of the patient, and the type and severity of the patient's disease. Appropriate dosages may be determined by clinical trials. For example, when “an effective amount” or “a therapeutic amount” is indicated, the precise amount of the pharmaceutical composition (or multispecific or multifunctional molecules) to be administered can be determined by a physician with consideration of individual differences in symptoms (or severity thereof), extent of infection, age, weight, and condition of the subject. In embodiments, the pharmaceutical composition described herein can be administered at a dosage of 10⁴ to 10⁹ cells/kg body weight, e.g., 10⁵ to 10⁶ cells/kg body weight, including all integer values within those ranges. In embodiments, the pharmaceutical composition described herein can be administered multiple times at these dosages. In embodiments, the pharmaceutical composition described herein can be administered using infusion techniques described in immunotherapy (see, e.g., Rosenberg et al., New Eng. J. of Med. 319:1676, 1988).

In embodiments, the anti-TCRβV antibody molecule, multispecific or multifunctional molecules or pharmaceutical composition is administered to the subject parentally. In embodiments, the cells are administered to the subject intravenously, subcutaneously, intranodally, intramuscularly, intradermally, or intraperitoneally. In embodiments, the cells are administered, e.g., injected, directly into a lymph node. In embodiments, the cells are administered as an infusion (e.g., as described in Rosenberg et al., New Eng. J. of Med. 319:1676, 1988) or an intravenous push. In embodiments, the cells are administered as an injectable depot formulation.

In embodiments, the subject is a mammal. In embodiments, the subject is a human, monkey, pig, dog, cat, cow, sheep, goat, rabbit, rat, or mouse. In embodiments, the subject is a human. In embodiments, the subject is a pediatric subject, e.g., less than 18 years of age, e.g., less than 17, 16, 15, 14, 13, 12, 11, 10, 9, 8, 7, 6, 5, 4, 3, 2, 1 or less years of age. In embodiments, the subject is an adult, e.g., at least 18 years of age, e.g., at least 19, 20, 21, 22, 23, 24, 25, 25-30, 30-35, 35-40, 40-50, 50-60, 60-70, 70-80, or 80-90 years of age.

### Combination Therapies

The anti-TCRβV antibody molecule, multispecific or multifunctional molecules disclosed herein can be used in combination with a second therapeutic agent or procedure.

In embodiments, the anti-TCRβV antibody molecule, multispecific or multifunctional molecule and the second therapeutic agent or procedure are administered/performed after a subject has been diagnosed with an infectious disease, e.g., before the infectious disease has been eliminated from the subject. In embodiments, the anti-TCRβV antibody molecule, multispecific or multifunctional molecule and the second therapeutic agent or procedure are administered/performed simultaneously or concurrently. For example, the delivery of one treatment is still occurring when the delivery of the second commences, e.g., there is an overlap in administration of the treatments. In other embodiments, the anti-TCRβV antibody molecule, multispecific or multifunctional molecule and the second therapeutic agent or procedure are administered/performed sequentially. For example, the delivery of one treatment ceases before the delivery of the other treatment begins.

In embodiments, combination therapy can lead to more effective treatment than monotherapy with either agent alone. In embodiments, the combination of the first and second treatment is more effective (e.g., leads to a greater reduction in symptoms and/or infectious agents) than the first or second treatment alone. In embodiments, the combination therapy permits use of a lower dose of the first or the second treatment compared to the dose of the first or second treatment normally required to achieve similar effects when administered as a monotherapy. In embodiments, the combination therapy has a partially additive effect, wholly additive effect, or greater than additive effect.

In one embodiment, the anti-TCRBV antibody, multispecific or multifunctional molecule is administered in combination with a therapy, e.g., a therapy for treating the infectious disease. The administration of the multispecific or multifunctional molecule and the therapy can be sequential (with or without overlap) or simultaneous. Administration of the anti-TCRBV antibody, multispecific or multifunctional molecule can be continuous or intermittent during the course of therapy.

Infectious Diseases

In some embodiments, the anti-TCRβV antibody molecules, e.g., the multispecific antibody molecules, disclosed herein can be used to treat infectious diseases. In some embodiments, the antibody molecules, e.g., the multispecific antibody molecules, disclosed herein deplete cells expressing a viral or bacterial antigen. In some embodiments, the anti-TCRβ V antibody molecule further comprises a binding specificity that binds to an antigen present on the surface of an infected cell, e.g., a viral infected cell.

Some examples of pathogenic viruses causing infections treatable by methods include HIV, hepatitis (A, B, or C), herpes virus (e.g., VZV, HSV-1, HAV-6, HSV-II, and CMV, Epstein Barr virus), adenovirus, influenza virus, flaviviruses, echovirus, rhinovirus, coxsackie virus, cornovirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus. In one embodiment, the infection is an influenza infection.

In another embodiment, the infection is a hepatitis infection, e.g., a Hepatitis B or C infection.

Exemplary viral disorders that can be treated include, but are not limited to, Epstein Bar Virus (EBV), influenza virus, HIV, SIV, tuberculosis, malaria and HCMV.

Some examples of pathogenic bacteria causing infections treatable by methods of the invention include syphilis, chlamydia, rickettsial bacteria, mycobacteria, staphylococci, streptococci, pneumonococci, meningococci and conococci, klebsiella, proteus, serratia, pseudomonas, legionella, diphtheria, salmonella, bacilli, cholera, tetanus, botulism, anthrax, plague, leptospirosis, and Lymes disease bacteria. The anti-TCRβV antibody molecules can be used in combination with existing treatment modalities for the aforesaid infections. For example, treatments for syphilis include penicillin (e.g., penicillin G.), tetracycline, doxycycline, ceftriaxone and azithromycin.

### CRS Grading

In some embodiments, CRS can be graded in severity from 1-5 as follows. Grades 1-3 are less than severe CRS. Grades 4-5 are severe CRS. For Grade 1 CRS, only symptomatic treatment is needed (e.g., nausea, fever, fatigue, myalgias, malaise, headache) and symptoms are not life threatening. For Grade 2 CRS, the symptoms require moderate intervention and generally respond to moderate intervention. Subjects having Grade 2 CRS develop hypotension that is responsive to either fluids or one low-dose vasopressor; or they develop grade 2 organ toxicity or mild respiratory symptoms that are responsive to low flow oxygen (<40% oxygen). In Grade 3 CRS subjects, hypotension generally cannot be reversed by fluid therapy or one low-dose vasopressor. These subjects generally require more than low flow oxygen and have grade 3 organ toxicity (e.g., renal or cardiac dysfunction or coagulopathy) and/or grade 4 transaminitis. Grade 3 CRS subjects require more aggressive intervention, e.g., oxygen of 40% or higher, high dose vasopressor(s), and/or multiple vasopressors. Grade 4 CRS subjects suffer from immediately life-threatening symptoms, including grade 4 organ toxicity or a need for mechanical ventilation. Grade 4 CRS subjects generally do not have transaminitis. In Grade 5 CRS subjects, the toxicity causes death. Sets of criteria for grading CRS are provided herein as Table 12, Table 13, and Table 14. Unless otherwise specified, CRS as used herein refers to CRS according to the criteria of Table 13.

In embodiments, CRS is graded according to Table 12:

TABLE 12
CRS grading

| | |
|---|---|
| Gr1 | Supportive care only |
| Gr2 | IV therapies +/− hospitalization. |
| Gr3 | Hypotension requiring IV fluids or low-dose vasoactives or |
| | hypoxemia requiring oxygen, |
| | CPAP, or BIPAP. |
| Gr4 | Hypotension requiring high-dose vasoactives or hypoxemia |
| | requiring mechanical ventilation. |
| Gr 5 | Death |

TABLE 13
CTCAE v 4.0 CRS grading scale

| CRS grade | Characteristics |
|---|---|
| Grade 1 | Mild; No infusion interruption; No intervention |
| Grade 2 | Infusion interruption indicated but responds promptly to |
| | symptomatic treatment (e.g., |
| | antihistamines, NSAIDS, narcotics, IV fluids); |
| | prophylactic medications indicated for |
| | <= 24 hrs |
| Grade 3 | Prolonged (e.g., not rapidly responsive to symptomatic |
| | medications and/or brief |
| | interruption of infusion); recurrence of symptoms |
| | following initial improvement; |
| | hospitalization indicated for clinical sequelae |
| | (e.g., renal impairment, pulmonary infiltrates) |
| Grade 4 | Life threatening consequences; |
| | pressor or ventilator support |

TABLE 14

| NCI CRS | grading scale |
|---|---|
| CRS grade | Characteristics |
| Grade 1 | Symptoms are not life threatening and require symptomatic |
| | treatment only; e.g., fever, |
| | nausea, fatigue, headache, myalgias, malaise |
| Grade 2 | Symptoms require and respond to moderate intervention; |
| | Oxygen requirement <40% |
| | or hypotension responsive to fluids or low dose pressors or |
| | Grade 2 organ toxicity |
| Grade 3 | Symptoms require and respond to aggressive intervention; |
| | Oxygen requirement |
| | >= 40% or Hypotension requiring high dose or multiple |
| | pressors or grade 3 organ |
| | toxicity or grade 4 transaminitis |
| Grade 4 | Life threatening symptoms Requirement for ventilator |
| | support or Grade 4; organ |
| | toxicity (excluding transaminitis) |

### EXAMPLES

### Example 1

### Humanization of α-TRBV6-5 Antibody Clone Antibody A

The germline for the mouse a-TCRI3 antibody clone Antibody A VH and VL were assigned using IMGT nomenclature, with CDR regions defined by a combined Kabat and Chothia classification. SEQ ID NO: 1 and SEQ ID NO: 2 are the Antibody A VH and VL sequences respectively where the VH germline is mouse IGHV1S12*01 and the VL germline is mouse IGKV6-15*01. SEQ ID NOs: 3-5 are the Antibody A VH CDR regions 1-3 respectively and SEQ ID NOs: 6-8 correspond to the VL CDR regions 1-3 (as described in Table 3).

Humanization of the Antibody A VH and VL sequences was done separately using similar methodology. Amino acids positions were identified in the framework regions which were important for the success of CDR grafting. Human germline sequences were identified which preserved the necessary residues and contained a high amount of overall identity. When the human germline framework sequence did not contain a matching important amino acid, it was back mutated to match the mouse sequence. CDR regions were grafted onto the human germline unchanged. The Antibody A VH was humanized into human IGHV1-69*01 and the Antibody A VL was humanized into IGKV1-17*01 and IGKV1-27*01. All 3 humanized sequences were confirmed to contain no introduced potential negative post translational modification sites such as NG, DG, NS, NN, DS, NT, NXS, or NXT as a result of the humanization process. SEQ ID NO: 9 is the humanized Antibody A-H.1 VH and SEQ ID NOs: 10 and 11 are the humanized VL IGKV1-17*01 and IGKV1-27*01 germlines respectively (as described in Table 3). FIGS. 1A and 1B show the murine and humanized sequences with annotations depicting the CDR and framework regions (FR).

### Example 2

### Humanization of α-TRBV12-3 and TRBV12-4 Antibody Clone Antibody B

The germline for the mouse a-TCRI3 antibody clone Antibody B VH and VL were assigned using IMGT nomenclature, with CDR regions defined by a combined Kabat and Chothia classification. SEQ ID NO: 15 and SEQ ID NO: 16 are the Antibody B VH and VL sequences respectively where the VH germline is mouse IGHV5-17*02 and the VL germline is mouse IGKV4-50*01. SEQ ID NOs: 17-19 are the B-H VH CDR regions 1-3 respectively and SEQ ID NOs: 20-22 are the B-H VL CDR regions 1-3 (as described in Table 4).

The method applied to humanize Antibody A described in Example 1 was used to humanize Antibody B. The Antibody B VH was humanized into human IGHV3-30*01, IGHV3-48*01, and IGHV3-66*01 and the Antibody B VL was humanized into human IGKV1-9*01, IGKV1-39*01, IGKV3-15*01, IGLV1-47*01 and IGLV3-10*01. SEQ ID NOs: 23-25 are the B-H.1A, B-H.1B, and B-H.1C humanized heavy chains and SEQ ID NOs: 26-30 are the B-H.1D, B-H.1E, B-H.1F, B-H.1G and B-H.1H humanized light chains (as described in Table 4). FIGS. 2A and 2B show the murine and humanized sequences with annotations depicting the CDR and framework regions (FR).

### Example 3

### Characteristics of Anti-TCRβV antibodies

Introduction

Current bispecific constructs designed to redirect T cells to promote tumor cell lysis for cancer immunotherapy typically utilize single chain variable fragments (scFvs) that are derived from monoclonal antibodies (mAb) directed against the CD3e subunit of the T cell receptor (TCR). However, there are limitations to this approach which may prevent the full realization of the therapeutic potential for such bispecific constructs. Previous studies have shown that, e.g., low “activating” doses of anti-CD3e mAb can cause long-term T cell dysfunction and exert immunosuppressive effects. In addition, anti-CD3e mAbs bind to all T cells and thus activate equally all T cells, which has been associated with the first dose side effects of anti-CD3e mAbs that result from massive T cell activation. These large number of activated T cells secrete substantial amounts of cytokines, the most important of which is Interferon gamma (IFNγ). This excess amount of IFNγ in turn, e.g., activates macrophages which then can overproduce proinflammatory cytokines such as IL-1, IL-6 and TNF-alpha, causing a “cytokine storm” known as the cytokine release syndrome (CRS). Thus, it might be advantageous to develop antibodies that are capable of binding and activating only a subset of necessary effector T cells to reduce the CRS.

Results

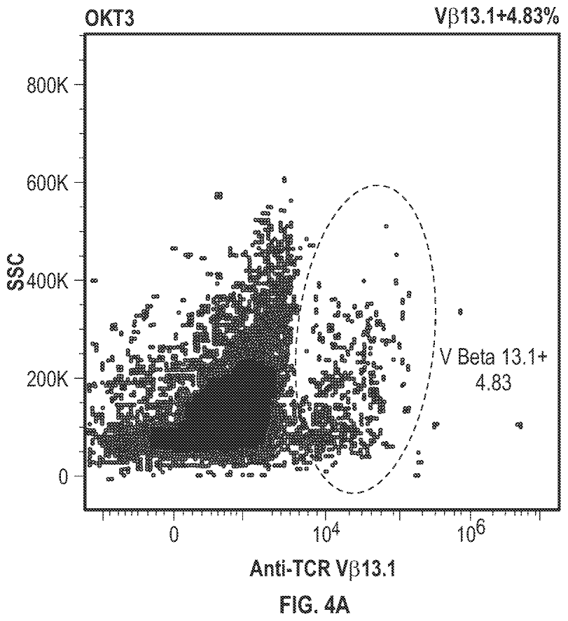

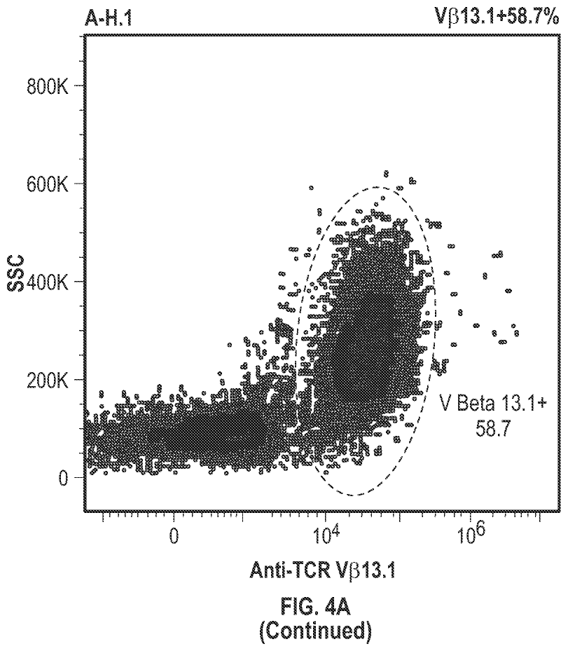

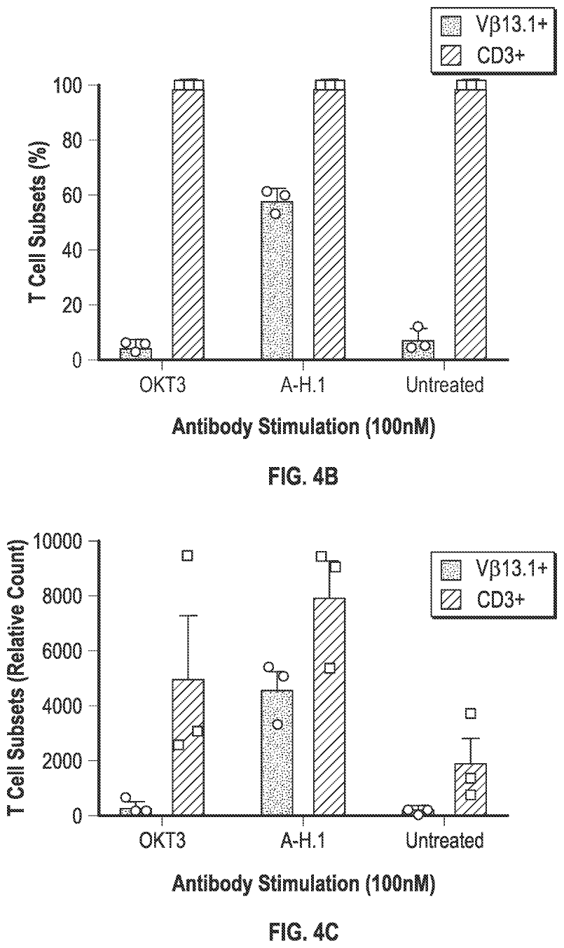

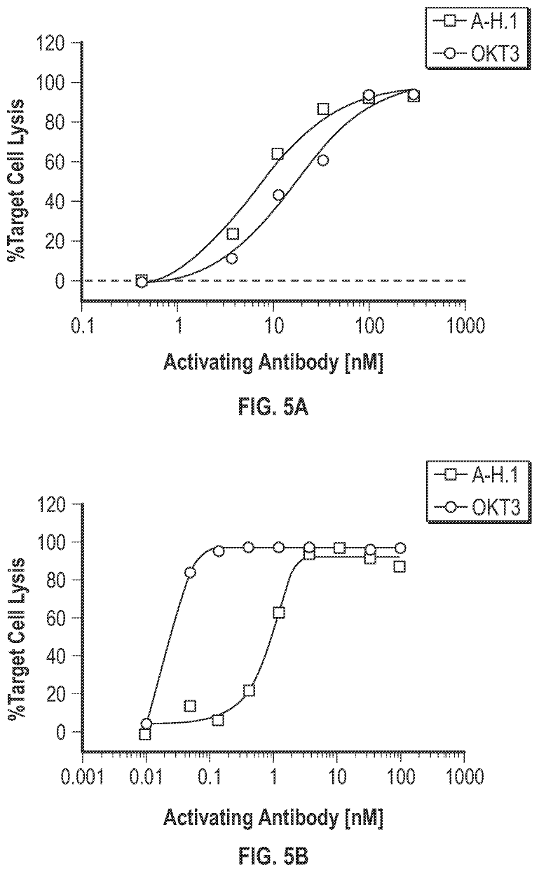

To that end, antibodies directed to the variable chain of the beta subunit of TCR (TCR Vb) were identified. These anti-TCR Vb antibodies bind and activate a subset of T cells, but with, e.g., no or markedly reduced CRS. Using plate-bound anti-TCR Vb13.1 mAbs (A-H.1 and A-H.2) it was shown that a population of T cells, defined by positive staining with A-H.1, can be expanded (from ˜5% of T cells on day 0 to almost 60% of total T cells on day 6 of cell culture) (FIGS. 4A-4C). For this experiment, human CD3+T cells were isolated using magnetic-bead separation (negative selection) and activated with immobilized (plate-coated) A-H.1 or OKT3 (anti-CD3e) antibodies at 100nM for 6 days. The expanded Vb13.1+T cells display cytolytic activity against transformed cell line RPMI-8226 when co-cultured with purified CD3+T cells (FIGS. 5A-5B).

Next, the ability of PBMCs activated by anti-TCR VB antibodies to produce cytokines was assessed. The cytokine production of PBMCs activated with anti-TCR VB antibodies was compared to the cytokine production of PBMCs activated with: (i) anti-CD3e antibodies (OKT3 or SP34-2); (ii) anti-TCR V alpha (TCR VA) antibodies including anti-TCR VA 12.1 antibody 6D6.6, anti-TCR VA24JA18 antibody 6B11; (iii) anti-TCR alpha beta antibody T10B9; and/or (iv) isotype control (BGM0109). The anti-TCR VB antibodies tested include: humanized anti-TCRVB 13.1 antibodies (A-H.1, or A-H.2), murine anti-TCR VB5 antibody Antibody E, murine anti-TCR VB8.1 antibody Antibody B, and murine anti-TCR VB12 antibody Antibody D. BGM0109 comprises the amino acid sequence of

(SEQ ID NO: 3282)
METDTLLLWVLLLWVPGSTGGLNDIFEAQKIEWHEGGGGSEPRTDTDTCPN
PPDPCPTCPTPDLLGGPSVFIFPPKPKDVLMISLTPKITCVVVDVSEEEPD
VQFNWYVNNVEDKTAQTETRQRQYNSTYRVVSVLPIKHQDWMSGKVFKCKV
NNNALPSPIEKTISKPRGQVRVPQIYTFPPPIEQTVKKDVSVTCLVTGFLP
QDIHVEWESNGQPQPEQNYKNTQPVLDSDGSYFLYSKLNVPKSRWDQGDSF
TCSVIHEALHNEIRMTKTISRSLGNGGGGS.

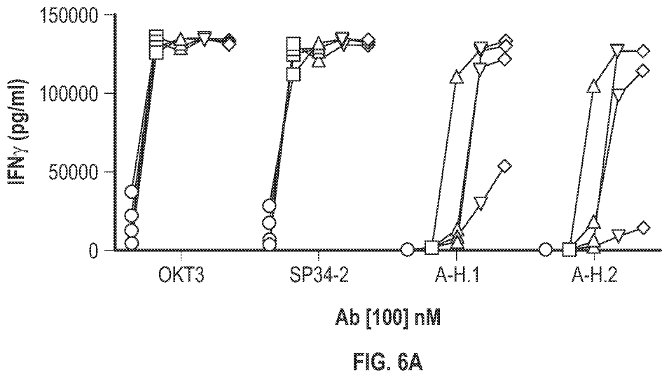

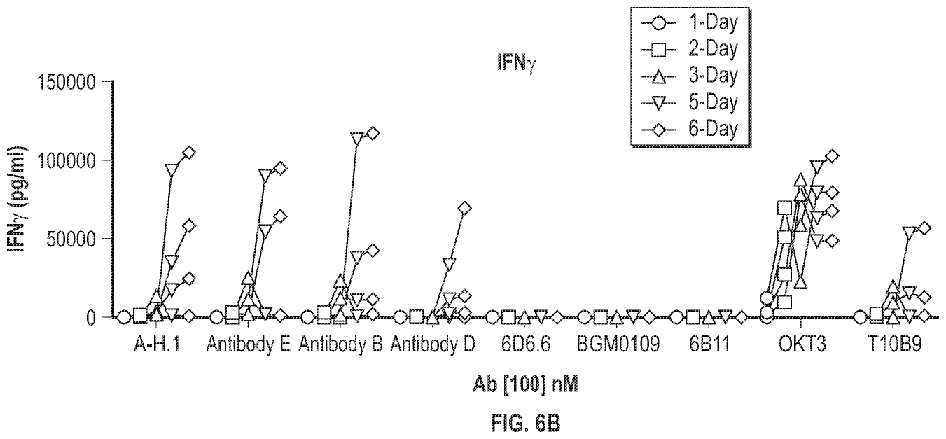

As shown in FIG. 6A, when plate-bound A-H.1 or A-H.2, or anti-CD3e antibodies (OKT3 or SP34-2) were used to activate human PBMCs, the T cell cytokine IFNγ was induced (FIG. 6A). All anti-TCR VB antibodies tested had a similar effect on the production of IFNγ(FIG. 6B). The anti-TCR VA antibodies did not induce similar IFNγ production.

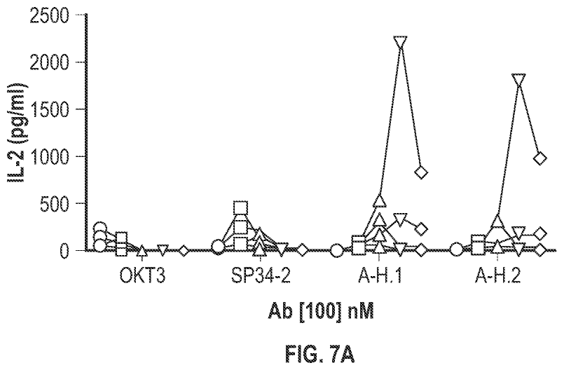

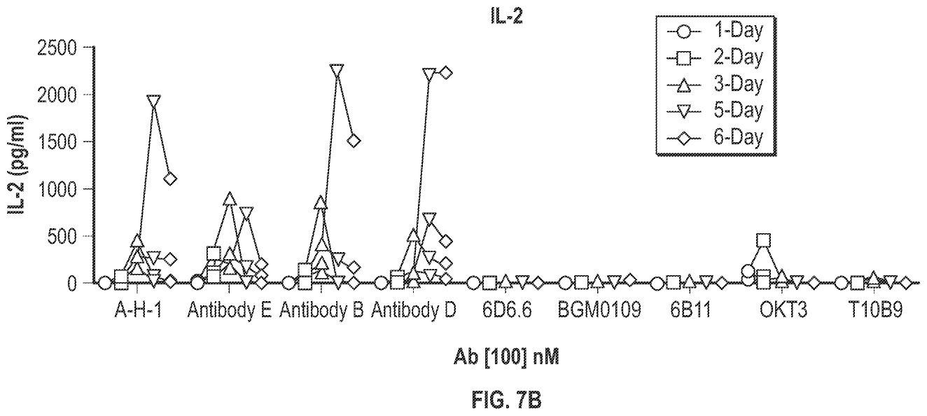

With respect to IL-2 production, PBMCs activated with A-H.1 and A-H.2 resulted in increased IL-2 production (FIG. 7A) with delayed kinetics (FIG. 7B) as compared to PBMCs activated with anti-CD3e antibodies (OKT3 or SP34-2). FIG. 7B shows that anti-TCR VB antibody activated PBMCs demonstrate peak production of IL-2 at Day 5 or Day 6 post-activation (incubation with plate-coated antibodies). In contrast, IL-2 production in PBMCs activated with OKT3 peaked at day 2 post-activation. As with IFNG, the IL-2 effect (e.g., enhanced production of IL-2 and delayed kinetics) was similar across all anti-TCR VB antibodies tested (FIG. 7B).

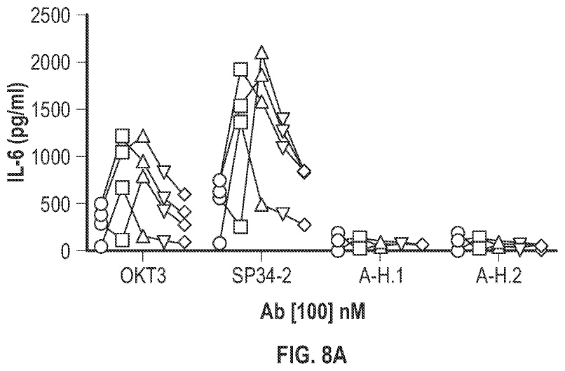

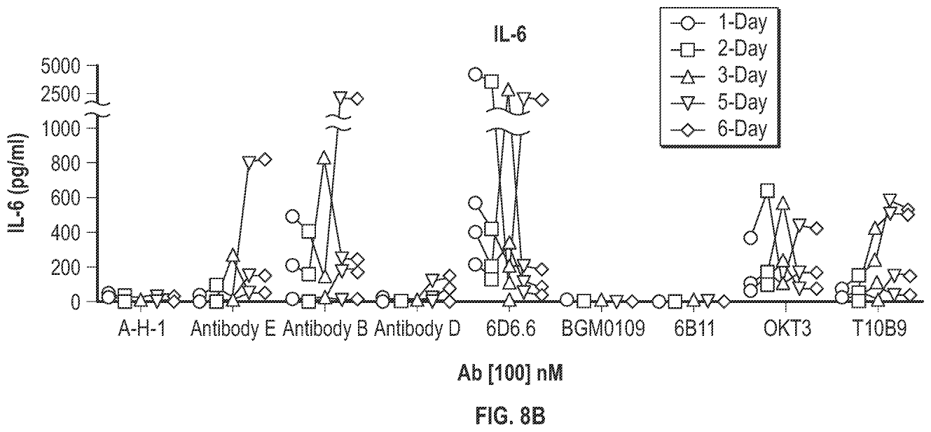

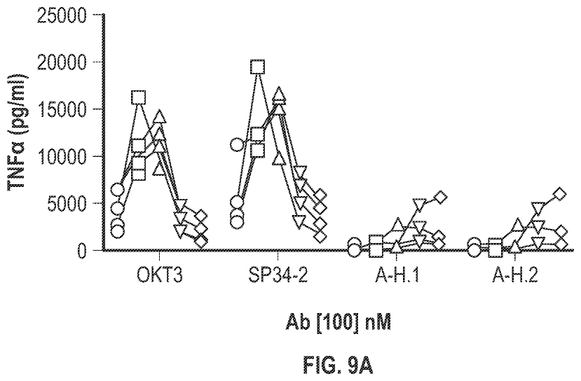

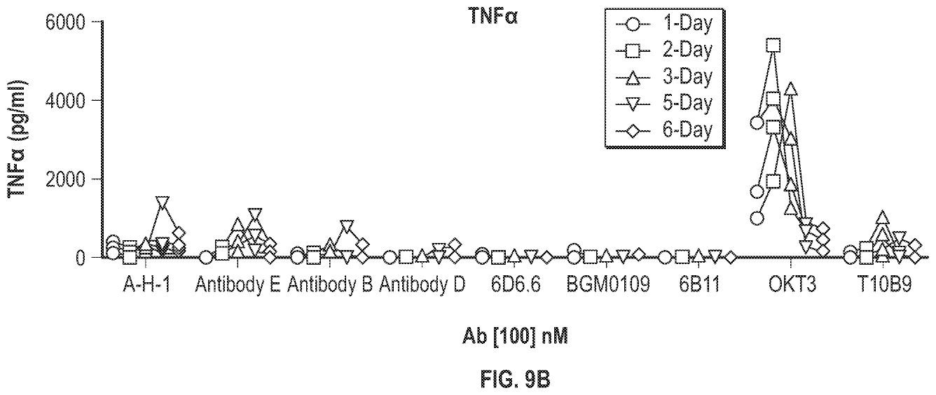

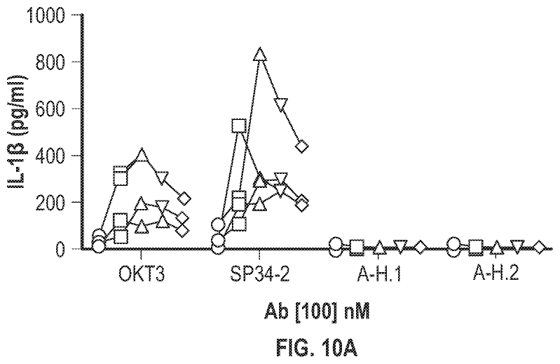

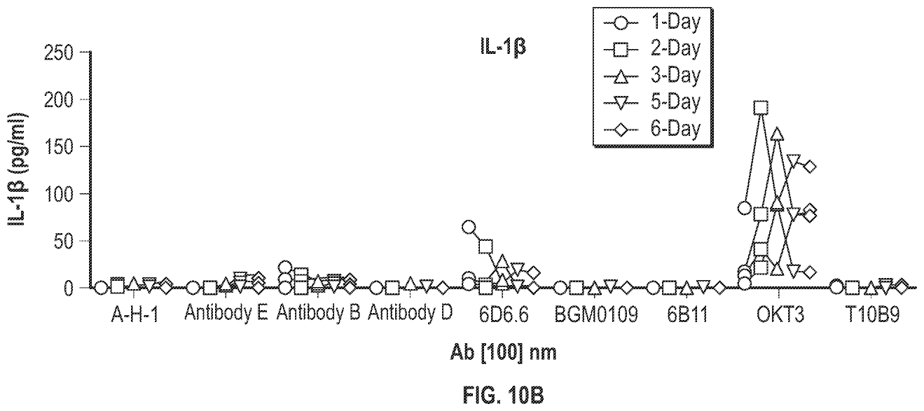

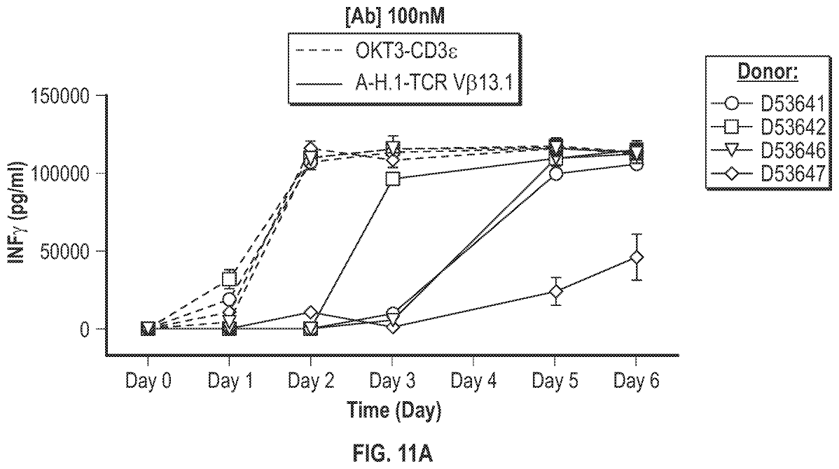

The production of cytokines IL-6, IL-10 and TNF-alpha which are associated with “cytokine storms” (and accordingly CRS) was also assessed under similar conditions. FIGS. 8A, 9A and 10A shows that while PBMCs activated with anti-CD3e antibodies demonstrate production of IL-6 (FIG. 8A), TNF-alpha (FIG. 9A) and IL-10 (FIG. 10A), no or little induction of these cytokines was observed with PBMCs activated with A-H.1 or A-H.2. As shown in FIGS. 9B and 10B, TNF-alpha and IL-10 production was not induced by activation of PBMCs with any of the anti-TCR VB antibodies.

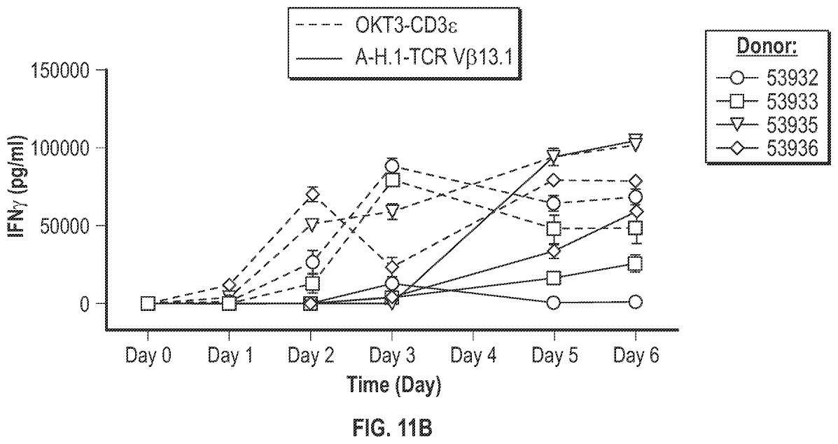

It was further noted that the kinetics of IFNγ production by A-H.1-activated CD3+T cells was delayed relative to those produced by CD3+T cells activated by anti-CD3e mAbs (OKT3 and SP34-2) (FIGS. 11A and 11B).

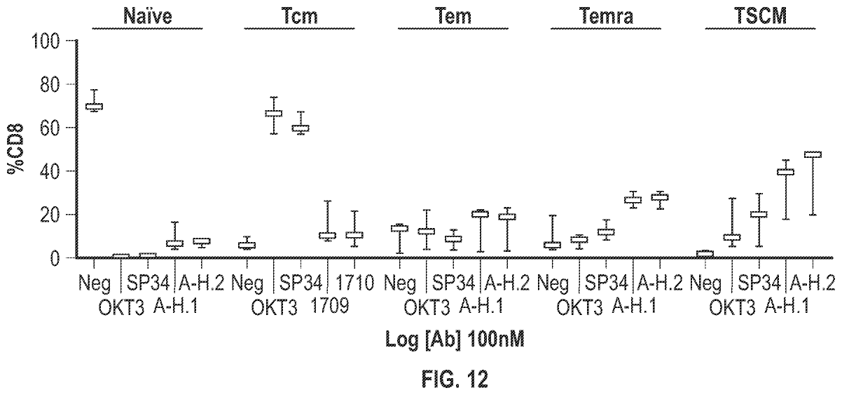

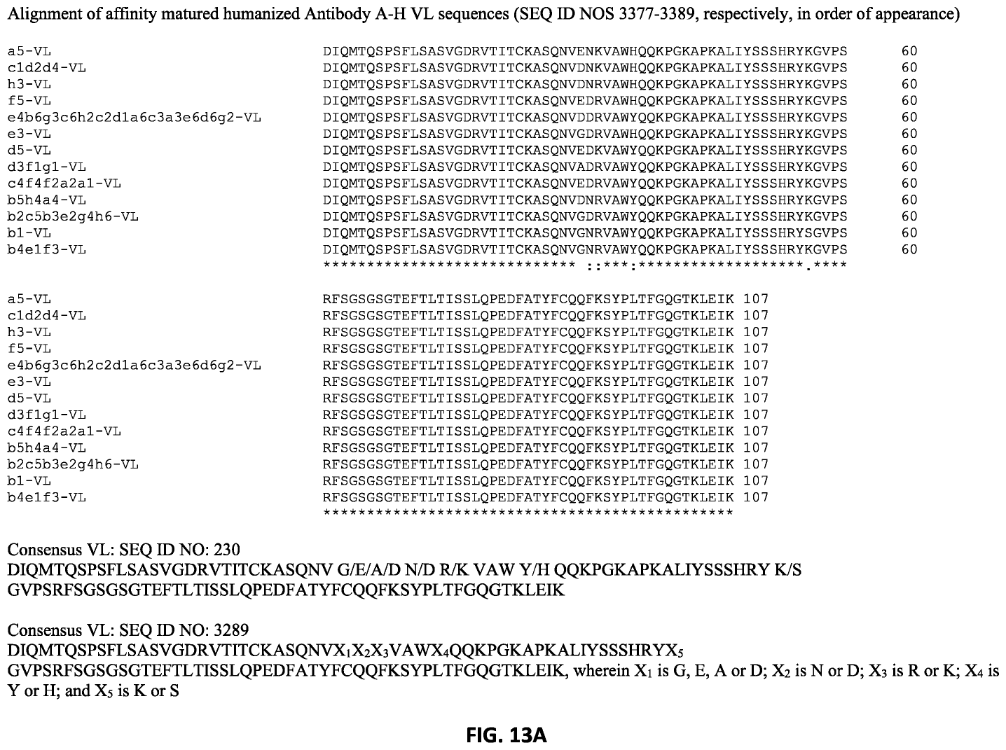

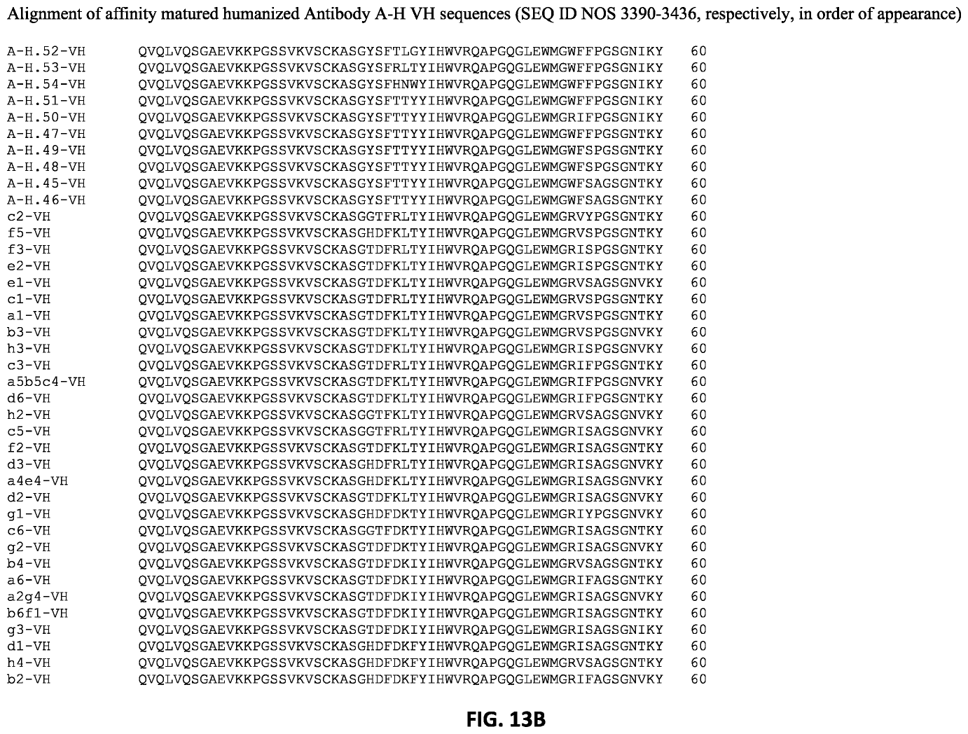

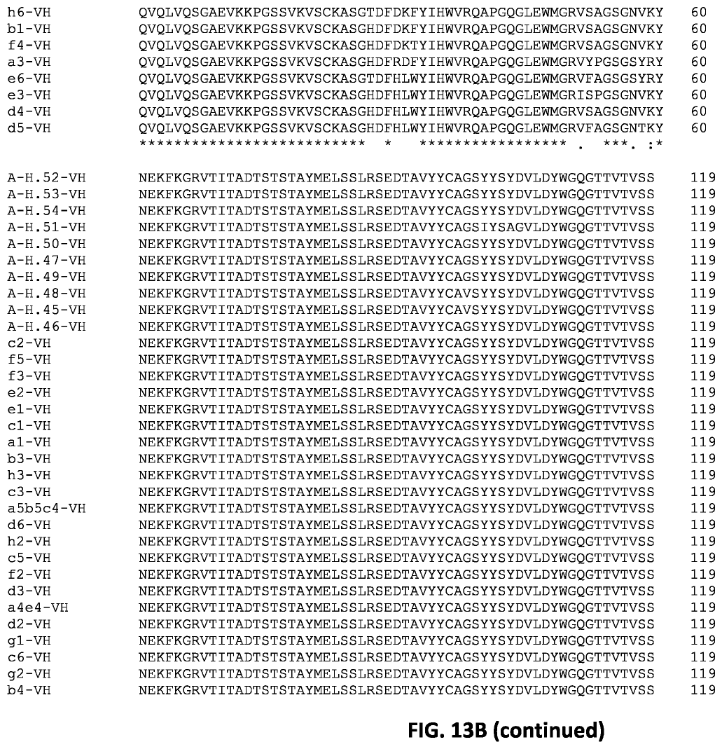

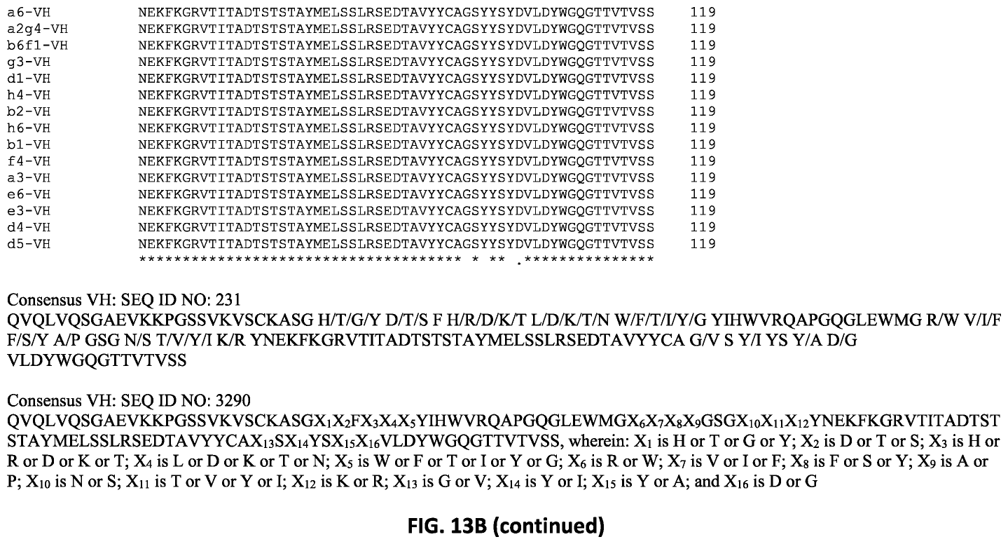

Finally, it was observed that the subset of memory effector T cells known as T_{EMRA} was preferentially expanded in CD8+T cells activated by A-H.1 or A-H.2 (FIG. 12). Isolated human PBMCs were activated with immobilized (plate-coated) anti-CD3e or anti-TCR Vβ13.1 at 100 nM for 6-days. After a 6-day incubation, T-cell subsets were identified by FACS staining for surface markers for Naive T cell (CD8+, CD95-, CD45RA+, CCR7+), T stem cell memory (TSCM; CD8+, CD95+, CD45RA+, CCR7+), T central memory (Tcm; CD8+, CD95+, CD45RA-, CCR7+), T effector memory (Tem; CD8+, CD95+, CD45RA-, CCR7-), and T effector memory re-expressing CD45RA (Temra; CD8+, CD95+, CD45RA+, CCR7-). Human PBMCs activated by anti-TCR Vβ13.1 antibodies (A-H.1 or A-H.2) increased CD8+TSCM and Temra T cell subsets when compared to PBMCs activated by anti-CD3e antibodies (OKT3 or SP34-2). Similar expansion was observed with CD4+T cells.

Conclusion

The data provided in this Example show that antibodies directed against TCR Vb can, e.g., preferentially activate a subset of T cells, leading to an expansion of T_{EMRA}, which can, e.g., promote tumor cell lysis but not CRS. Thus, bispecific constructs utilizing either a Fab or scFv or a peptide directed to the TCR Vb can, e.g., be used to activate and redirect T cells to promote tumor cell lysis for cancer immunotherapy, without, e.g., the harmful side-effects of CRS associated with anti-CD3e targeting.

### INCORPORATION BY REFERENCE

All publications and patents mentioned herein are hereby incorporated by reference in their entirety as if each individual publication or patent was specifically and individually indicated to be incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of expanding, e.g., increasing the number of, a T cell population comprising a TCRβV molecule (e.g., as described herein), the method comprising: contacting the T cell population with an antibody molecule, e.g., humanized antibody molecule, which binds, e.g., specifically binds, to a T cell receptor beta variable chain (TCRβV) region, thereby expanding the T cell population, wherein the T cell population is obtained from or comprised in a subject having an infectious disease.

2. A method of treating a subject having an infectious disease, the method comprising administering an effective amount of an anti-TCRβV antibody molecule (e.g., a TCRβV agonist) to the subject, thereby treating the infectious disease.

3. A method of evaluating, e.g., identifying the level or activity of a TCRβV molecule in a subject having an infectious disease, the method comprising acquiring a status for the TCRβV molecule in the subject;
wherein the level or activity of the TCRβV molecule is higher (e.g., at least about 1.1, 1.2, 1.3, 1.4, 1.5, 2, 3, 4, 5, 6, 7, 8, 9, 10, 20, 30, 40, 50, 60, 70, 80, 90, 100, 200, 300, 400, 500, 600, 700, 800, 900, 1000, 5000, 10,000, or 100,000-fold higher) relative to the level or activity of the TCRβV molecule in a healthy subject (e.g., a subject that does not have the infectious disease).

4. A method of treating a subject having an infectious disease, the method comprising:
(i) acquiring a status for the TCRβV molecule in the subject; and
(ii) administering an effective amount of an anti-TCRβV antibody molecule (e.g., a TCRβV agonist) to the subject, thereby treating the infectious disease;
wherein the level or activity of the TCRβV molecule is higher (e.g., at least about 1.1, 1.2, 1.3, 1.4, 1.5, 2, 3, 4, 5, 6, 7, 8, 9, 10, 20, 30, 40, 50, 60, 70, 80, 90, 100, 200, 300, 400, 500, 600, 700, 800, 900, 1000, 5000, 10,000, or 100,000-fold higher) relative to the level or activity of the TCRβV molecule in a healthy subject (e.g., a subject that does not have the infectious disease).

5. A method of evaluating a subject for the presence of an infectious disease, the method comprising:
(i) acquiring a status for one or more TCRβV molecules in a biological sample from the subject and in a biological sample from a healthy subject (e.g., a subject that does not have the infectious disease); and
(ii) determining whether one or more of the TCRβV molecules exhibits an elevated level or activity in the subject relative to the healthy subject;
wherein an elevated level or activity in the subject relative to in the healthy subject is indicative of the presence of the infectious disease.

6. A method of treating a subject having an infectious disease, the method comprising:
(i) acquiring a status for one or more TCRβV molecules in a biological sample from the subject and in a biological sample from a healthy subject (e.g., a subject that does not have the infectious disease);
(ii) determining whether one or more of the TCRβV molecules exhibits an elevated level or activity in the subject relative to the healthy subject; and
(iii) if an elevated level or activity in the subject relative to in the healthy subject is determined, administering an effective amount of an anti-TCRβV antibody molecule (e.g., a TCRβV agonist) to the subject.

7. The method of any of the preceding claims, wherein the status is indicative of the subject having the infectious disease or a symptom thereof.

8. The method of any of the preceding claims, wherein the status is indicative of responsiveness to a therapy, e.g., a TCRβV molecule.

9. The method of any of the preceding claims, wherein the status is determined, e.g., measured, by an assay described herein.

10. The method of any of the preceding claims, wherein the acquiring comprises: isolating a biological sample from the subject, contacting the biological sample with an anti-TCRβV antibody molecule (e.g., the same anti-TCRβV antibody molecule or a different anti-TCRβV antibody molecule), and determining a level of T cell expansion in the biological sample, e.g., relative to the level of T cell expansion in a biological sample obtained from a healthy subject (e.g., a subject that does not have the infectious disease).

11. The method of claim 10, further comprising administering expanded T cells from the biological sample to the subject.

12. The method of any of the preceding claims, wherein the acquiring comprises: isolating a biological sample from the subject, contacting the biological sample with an anti-TCRβV antibody molecule (e.g., the same anti-TCRβV antibody molecule or a different anti-TCRβV antibody molecule), and determining a level of T cell function (e.g., cytotoxic activity) in the biological sample, e.g., relative to the level of T cell expansion in a biological sample obtained from a healthy subject (e.g., a subject that does not have the infectious disease).

13. A method of identifying one or more TCRβV molecules associated with a disease, the method comprising:
(i) acquiring a status for a plurality of TCRβV molecules in a biological sample from a first subject having the disease and in a biological sample from a second subject not having the disease; and
(ii) determining whether one or more of the TCRβV molecules exhibits an elevated level or activity in the first subject relative to the second subject;
thereby identifying one or more TCRβV molecules associated with the disease.

14. The method of any of the preceding claims, wherein the infectious disease is selected from Epstein-Barr virus (EBV), influenza, human immunodeficiency virus (HIV), simian immunodeficiency virus (SIV), tuberculosis, malaria, or human cytomegalovirus (HCMV).

15. The method of any of the preceding claims, wherein the TCRβV is selected from TCRβV V5-6, TCRβV V6-5, TCRβV V7, TCRβV V9, TCRβV V10, TCRβV V12 (e.g., TCRβV V12-4), TCRβV V13, TCRβV V14, TCRβV V19, TCRβV V23-1, or a subfamily member thereof (e.g., as listed in Table 1 or Table 2).

16. The method of any of the preceding claims, wherein the anti-TCRβV antibody molecule induces expansion, e.g., increasing the number of, a T cell population comprising a TCRβV molecule (e.g., the TCRβV bound by the anti-TCRβV antibody molecule).

17. The method of claim 16, wherein the T cell population comprises CD4 T cells, CD8 T cells, or CD3 T cells.

18. The method of claim 16, wherein the T cell population derived from peripheral blood.

19. The method of any of the preceding claims, wherein the anti-TCRβV antibody molecule comprises:
(1) a heavy chain complementarity determining region (HC CDR1), a HC CDR2 and/or a HC CDR3 of SEQ ID NO: 3, SEQ ID NO: 4, and/or SEQ ID NO: 5; and/or
(2) a light chain complementarity determining region 1 (LC CDR1), a LC CDR2, and/or a LC CDR3 of SEQ ID NO: 6, SEQ ID NO: 7, and/or SEQ ID NO: 8.

20. The method of any of the preceding claims, wherein the anti-TCRβV antibody molecule comprises:
(1) a heavy chain complementarity determining region (HC CDR1), a HC CDR2 and/or a HC CDR3 of SEQ ID NO: 45, SEQ ID NO: 46, and/or SEQ ID NO: 47; and/or
(2) a light chain complementarity determining region 1 (LC CDR1), a LC CDR2, and/or a LC CDR3 of SEQ ID NO: 51, SEQ ID NO: 52, and/or SEQ ID NO: 53.

21. The method of any of the preceding claims, wherein the anti-TCRβV antibody molecule comprises:
(1) a heavy chain complementarity determining region (HC CDR1), a HC CDR2 and/or a HC CDR3 of SEQ ID NO: 48, SEQ ID NO: 49, and/or SEQ ID NO: 50; and/or
(2) a light chain complementarity determining region 1 (LC CDR1), a LC CDR2, and/or a LC CDR3 of SEQ ID NO: 54, SEQ ID NO: 55, and/or SEQ ID NO: 56.

22. The method of any of the preceding claims, wherein the anti-TCRβV antibody molecule comprises:
(1) a heavy chain complementarity determining region (HC CDR1), a HC CDR2 and/or a HC CDR3 of SEQ ID NO: 17, SEQ ID NO: 18, and/or SEQ ID NO: 19; and/or
(2) a light chain complementarity determining region 1 (LC CDR1), a LC CDR2, and/or a LC CDR3 of SEQ ID NO: 20, SEQ ID NO: 21, and/or SEQ ID NO: 22.

23. The method of any of the preceding claims, wherein the anti-TCRβV antibody molecule comprises:
(1) a heavy chain complementarity determining region (HC CDR1), a HC CDR2 and/or a HC CDR3 of SEQ ID NO: 57, SEQ ID NO: 58, and/or SEQ ID NO: 59; and/or
(2) a light chain complementarity determining region 1 (LC CDR1), a LC CDR2, and/or a LC CDR3 of SEQ ID NO: 63, SEQ ID NO: 64, and/or SEQ ID NO: 65.

24. The method of any of the preceding claims, wherein the anti-TCRβV antibody molecule comprises:
(1) a heavy chain complementarity determining region (HC CDR1), a HC CDR2 and/or a HC CDR3 of SEQ ID NO: 60, SEQ ID NO: 61, and/or SEQ ID NO: 62; and/or (2) a light chain complementarity determining region 1 (LC CDR1), a LC CDR2, and/or a LC CDR3 of SEQ ID NO: 66, SEQ ID NO: 67, and/or SEQ ID NO: 68.

25. The method of any of the preceding claims, wherein the anti-TCRβV antibody molecule comprises a VH having at least 85% (e.g., at least 85%, 90%, 95%, 96%, 97%, 98%, 99%, or 100%) sequence identity to SEQ ID NO: 9.

26. The method of any of the preceding claims, wherein the anti-TCRβV antibody molecule comprises a VL having at least 85% (e.g., at least 85%, 90%, 95%, 96%, 97%, 98%, 99%, or 100%) sequence identity to SEQ ID NO: 10.

27. The method of any of the preceding claims, wherein the anti-TCRβV antibody molecule comprises a VH having at least 85% (e.g., at least 85%, 90%, 95%, 96%, 97%, 98%, 99%, or 100%) sequence identity to SEQ ID NO: 9 and a VL having at least X % sequence identity to SEQ ID NO: 10.

28. The method of any of the preceding claims, wherein the anti-TCRβV antibody molecule comprises a heavy chain having at least 85% (e.g., at least 85%, 90%, 95%, 96%, 97%, 98%, 99%, or 100%) sequence identity to SEQ ID NO: 69.

29. The method of any of the preceding claims, wherein the anti-TCRβV antibody molecule comprises a heavy chain having at least 85% (e.g., at least 85%, 90%, 95%, 96%, 97%, 98%, 99%, or 100%) sequence identity to SEQ ID NO: 70.

30. The method of any of the preceding claims, wherein the anti-TCRβV antibody molecule comprises a heavy chain having at least 85% (e.g., at least 85%, 90%, 95%, 96%, 97%, 98%, 99%, or 100%) sequence identity to SEQ ID NO: 71.

31. The method of any of the preceding claims, wherein the anti-TCRβV antibody molecule comprises a light chain having at least 85% (e.g., at least 85%, 90%, 95%, 96%, 97%, 98%, 99%, or 100%) sequence identity to SEQ ID NO: 72.

32. The method of any of the preceding claims, wherein the anti-TCRβV antibody molecule comprises a heavy chain having at least 85% (e.g., at least 85%, 90%, 95%, 96%, 97%, 98%, 99%, or 100%) sequence identity to SEQ ID NO: 69 and a light chain having at least 85% (e.g., at least 85%, 90%, 95%, 96%, 97%, 98%, 99%, or 100%) sequence identity to SEQ ID NO: 72.

33. The method of any of the preceding claims, wherein the anti-TCRβV antibody molecule comprises a heavy chain having at least 85% (e.g., at least 85%, 90%, 95%, 96%, 97%, 98%, 99%, or 100%) sequence identity to SEQ ID NO: 70 and a light chain having at least 85% (e.g., at least 85%, 90%, 95%, 96%, 97%, 98%, 99%, or 100%) sequence identity to SEQ ID NO: 72.

34. The method of any of the preceding claims, wherein the anti-TCRβV antibody molecule comprises a heavy chain having at least 85% (e.g., at least 85%, 90%, 95%, 96%, 97%, 98%, 99%, or 100%) sequence identity to SEQ ID NO: 71 and a light chain having at least 85% (e.g., at least 85%, 90%, 95%, 96%, 97%, 98%, 99%, or 100%) sequence identity to SEQ ID NO: 72.

35. The method of any of the preceding claims, wherein the anti-TCRβV antibody molecule is co-expressed with an IgJ chain (e.g., an IgJ chain comprising at least 85%, 90%, 95%, 96%, 97%, 98%, 99%, or 100% sequence identity to SEQ ID NO: 76).

36. The method of any of the preceding claims, wherein the anti-TCRβV antibody molecule comprises a heavy chain having at least 85% (e.g., at least 85%, 90%, 95%, 96%, 97%, 98%, 99%, or 100%) sequence identity to SEQ ID NO: 69 and a light chain having at least 85% (e.g., at least 85%, 90%, 95%, 96%, 97%, 98%, 99%, or 100%) sequence identity to SEQ ID NO: 72;
and wherein the anti-TCRβV antibody molecule is co-expressed with an IgJ chain (e.g., an IgJ chain comprising at least 85%, 90%, 95%, 96%, 97%, 98%, 99%, or 100% sequence identity to SEQ ID NO: 76).

37. The method of any of the preceding claims, wherein the anti-TCRβV antibody molecule comprises a VH having at least 85% (e.g., at least 85%, 90%, 95%, 96%, 97%, 98%, 99%, or 100%) sequence identity to SEQ ID NO: 15.

38. The method of any of the preceding claims, wherein the anti-TCRβV antibody molecule comprises a VL having at least 85% (e.g., at least 85%, 90%, 95%, 96%, 97%, 98%, 99%, or 100%) sequence identity to SEQ ID NO: 16.

39. The method of any of the preceding claims, wherein the anti-TCRβV antibody molecule comprises a VH having at least 85% (e.g., at least 85%, 90%, 95%, 96%, 97%, 98%, 99%, or 100%) sequence identity to SEQ ID NO: 23.

40. The method of any of the preceding claims, wherein the anti-TCRβV antibody molecule comprises a VH having at least 85% (e.g., at least 85%, 90%, 95%, 96%, 97%, 98%, 99%, or 100%) sequence identity to SEQ ID NO: 24.

41. The method of any of the preceding claims, wherein the anti-TCRβV antibody molecule comprises a VH having at least 85% (e.g., at least 85%, 90%, 95%, 96%, 97%, 98%, 99%, or 100%) sequence identity to SEQ ID NO: 25.

42. The method of any of the preceding claims, wherein the anti-TCRβV antibody molecule comprises a VL having at least 85% (e.g., at least 85%, 90%, 95%, 96%, 97%, 98%, 99%, or 100%) sequence identity to SEQ ID NO: 26.

43. The method of any of the preceding claims, wherein the anti-TCRβV antibody molecule comprises a VL having at least 85% (e.g., at least 85%, 90%, 95%, 96%, 97%, 98%, 99%, or 100%) sequence identity to SEQ ID NO: 27.

44. The method of any of the preceding claims, wherein the anti-TCRβV antibody molecule comprises a VL having at least 85% (e.g., at least 85%, 90%, 95%, 96%, 97%, 98%, 99%, or 100%) sequence identity to SEQ ID NO: 28.

45. The method of any of the preceding claims, wherein the anti-TCRβV antibody molecule comprises a VL having at least 85% (e.g., at least 85%, 90%, 95%, 96%, 97%, 98%, 99%, or 100%) sequence identity to SEQ ID NO: 29.

46. The method of any of the preceding claims, wherein the anti-TCRβV antibody molecule comprises a VL having at least 85% (e.g., at least 85%, 90%, 95%, 96%, 97%, 98%, 99%, or 100%) sequence identity to SEQ ID NO: 30.

47. The method of any of the preceding claims, wherein the anti-TCRβV antibody molecule comprises a VH amino acid sequence as listed in Table 3 or Table 4, and/or a VL amino acid sequence as listed in Table 3 or Table 4.

48. The method of any of the preceding claims, wherein the anti-TCRβV antibody molecule selectively or preferentially expands αβ T cels over y6 T cells.

49. The method of any of the preceding claims, wherein the anti-TCRβV antibody molecule does not induce cytokine release syndrome (CRS).

50. The method of any of the preceding claims, wherein binding of the anti-TCRβV antibody molecule to the TCRβV region results in one, two, three, four, five, six, seven, eight, nine, ten or more (e.g., all) of the following:
(i) reduced level, e.g., expression level, and/or activity of IL-10;
(ii) reduced level, e.g., expression level, and/or activity of IL-6;
(iii) reduced level, e.g., expression level, and/or activity of TNFα;
(iv) increased level, e.g., expression level, and/or activity of IL-2;
(v) a delay, e.g., at least 1, 2, 3, 4, 5, 6, 7, 8, 9, 10 or more hours delay, in increased level, e.g., expression level, and/or activity of IL-2;
(vi) a delay, e.g., at least 1, 2, 3, 4, 5, 6, 7, 8, 9, 10 hours delay, in increased level, e.g., expression level, and/or activity of IFNγ;
(vii) reduced T cell proliferation kinetics; or
(viii) reduced cytokine storm, e.g., cytokine release syndrome (CRS), e.g., as measured by an assay of Example 3;
(ix) cell killing, e.g., target cell killing,
(x) increased level, e.g., expression level, and/or activity of IL-15; or
(xi) increased Natural Killer (NK) cell proliferation, e.g., expansion, compared to an antibody that binds to: a CD3 molecule, e.g., CD3 epsilon (CD3e) molecule; or a TCR alpha (TCRα) molecule, wherein the anti-TCRβV antibody molecule:
(1) does not bind to TCRβ V12, TCRβ V5-5*01 or TCRβ V5-1*01;
(2) binds to TCRβ V12 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of the murine mAb Antibody B; and/or
(3) binds to TCRβ V5-5*01 TCRβ V5-1*01or with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of murine Antibody C.

51. The method of any of the preceding claims, wherein binding of the anti-TCRβV antibody molecule to the TCRβV region results in expansion, e.g., at least about 1.1-10 fold expansion (e.g., at least about 1.1, 1.2, 1.3, 1.4, 1.5, 2, 3, 4, 5, 6, 7, 8, 9, or 10 fold expansion), of a population of memory T cells, e.g., T effector memory (T_{EM}) cells, e.g., T_{EM} cells expressing CD45RA (T_{EMRA}) cells, wherein the anti-TCRβV antibody molecule:
(1) does not bind to TCRβ V12, TCRβ V5-5*01 or TCRβ V5-1*01;
(2) binds to TCRβ V12 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of the murine mAb Antibody B; and/or
(3) binds to TCRβ V5-5*01 TCRβ V5-1*01 or with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of murine Antibody C.

52. The method of any of the preceding claims, wherein binding of the anti-TCRβV antibody molecule to a TCRβV region results in a reduction of at least 2, 5, 10, 20, 50, 100, or 200 fold, or at least 2-200 fold (e.g., 5-150, 10-100, 20-50 fold) in the expression level and or activity of IL-1β as measured by an assay of Example 3.

53. The method of any of the preceding claims, wherein binding of the anti-TCRβV antibody molecule to a TCRβV region results in a reduction of at least 2, 5, 10, 20, 50, 100, 200, 300, 400, 500, 600, 700, 800, 900, or 1000 fold, or at least 2-1000 fold (e.g., 5-900, 10-800, 20-700, 50-600, 100-500, or 200-400 fold) in the expression level and or activity of IL-6 as measured by an assay of Example 3.

54. The method of any of the preceding claims, wherein binding of the anti-TCRβV antibody molecule to a TCRβV region results in a reduction of at least 2, 5, 10, 20, 50, 100, 200, 300, 400, 500, 600, 700, 800, 900, 1000, or 2000 fold, or at least 2-2000 fold (e.g., 5-1000, 10-900, 20-800, 50-700, 100-600, 200-500, or 300-400 fold) in the expression level and or activity of TNFα as measured by an assay of Example 3.

55. The method of any of the preceding claims, wherein binding of the anti-TCRβV antibody molecule to a TCRβV region results in an increase of at least 2, 5, 10, 20, 50, 100, 200, 300, 400, 500, 600, 700, 800, 900, 1000, or 2000 fold, or at least 2-2000 fold (e.g., 5-1000, 10-900, 20-800, 50-700, 100-600, 200-500, or 300-400 fold) in the expression level and or activity of IL-2 as measured by an assay of Example 3.

56. The method of any of the preceding claims, wherein the anti-TCRβV antibody molecule binds to one or more (e.g., all) of the following TCRβV subfamilies:
(i) TCRβ V6 subfamily comprising, e.g., TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01;
(ii) TCRβ V10 subfamily comprising, e.g., TCRβ V10-1*01, TCRβ V10-1*02, TCRβ V10-3*01 or TCRβ V10-2*01;
(iii) TCRβ V12 subfamily comprising, e.g., TCRβ V12-4*01, TCRβ V12-3*01, or TCRβ V12-5*01;
(iv) TCRβ V5 subfamily comprising, e.g., TCRβ V5-5*01, TCRβ V5-6*01, TCRβ V5-4*01, TCRβ V5-8*01, or TCRβ V5-1*01;
(v) TCRβ V7 subfamily comprising, e.g., TCRβ V7-7*01, TCRβ V7-6*01, TCRβ V7-8*02, TCRβ V7-4*01 TCRβ V7-2*02, TCRβ V7-2*03, TCRβ V7-2*01, TCRβ V7-3*01, TCRβ V7-9*03, or TCRβ V7-9*01;
(vi) TCRβ V11 subfamily comprising, e.g., TCRβ V11-1*01, TCRβ V11-2*01 or TCRβ V11-3*01;
(vii) TCRβ V14 subfamily comprising, e.g., TCRβ V14*01;
(viii) TCRβ V16 subfamily comprising, e.g., TCRβ V16*01;
(ix) TCRβ V18 subfamily comprising, e.g., TCRβ V18*01;
(x) TCRβ V9 subfamily comprising, e.g., TCRβ V9*01 or TCRβ V9*02;
(xi) TCRβ V13 subfamily comprising, e.g., TCRβ V13*01;
(xii) TCRβ V4 subfamily comprising, e.g., TCRβ V4-2*01, TCRβ V4-3*01, or TCRβ V4-1*01;
(xiii) TCRβ V3 subfamily comprising, e.g., TCRβ V3-1*01;
(xiv) TCRβ V2 subfamily comprising, e.g., TCRβ V2*01;
(xv) TCRβ V15 subfamily comprising, e.g., TCRβ V15*01;
(xvi) TCRβ V30 subfamily comprising, e.g., TCRβ V30*01, or TCRβ V30*02;
(xvii) TCRβ V19 subfamily comprising, e.g., TCRβ V19*01, or TCRβ V19*02;
(xviii) TCRβ V27 subfamily comprising, e.g., TCRβ V27*01;
(xix) TCRβ V28 subfamily comprising, e.g., TCRβ V28*01;
(xx) TCRβ V24 subfamily comprising, e.g., TCRβ V24-1*01;
(xxi) TCRβ V20 subfamily comprising, e.g., TCRβ V20-1*01, or TCRβ V20-1*02;
(xxii) TCRβ V25 subfamily comprising, e.g., TCRβ V25-1*01;
(xxiii) TCRβ V29 subfamily comprising, e.g., TCRβ V29-1*01; or
(xxiv) TCRβ V23 subfamily comprising, e.g., TCRβ V23-1.

57. The method of any of the preceding claims, wherein the anti-TCRβV antibody molecule binds to one or more (e.g., all) of the following TCRβV subfamilies:
(i) TCRβ V6 subfamily comprising, e.g., TCRβ V6-5*01;
(ii) TCRβ V10 subfamily comprising, e.g., TCRβ V10-1*01, TCRβ V10-1*02, TCRβ V10-3*01 or TCRβ V10-2*01;
(iii) TCRβ V12 subfamily comprising, e.g., TCRβ V12-4*01, TCRβ V12-3*01, or TCRβ V12-5*01;
(iv) TCRβ V5 subfamily comprising, e.g., TCRβ V5-6*01;
(v) TCRβ V7 subfamily comprising, e.g., TCRβ V7-7*01, TCRβ V7-6*01, TCRβ V7-8*02, TCRβ V7-4*01 TCRβ V7-2*02, TCRβ V7-2*03, TCRβ V7-2*01, TCRβ V7-3*01, TCRβ V7-9*03, or TCRβ V7-9*01;
(vi) TCRβ V14 subfamily comprising, e.g., TCRβ V14*01;
(vii) TCRβ V9 subfamily comprising, e.g., TCRβ V9*01 or TCRβ V9*02;
(viii) TCRβ V13 subfamily comprising, e.g., TCRβ V13*01;
(ix) TCRβ V19 subfamily comprising, e.g., TCRβ V19*01, or TCRβ V19*02; or (x) TCRβ V23 subfamily comprising, e.g., TCRβ V23-1.

58. The method of any of the preceding claims, wherein the infectious disease is SIV and the anti-TCRβV antibody molecule binds to the TCRβ V6 subfamily, e.g., comprising TCRβ V6-5*01.

59. The method of claim 58, wherein the subject has a higher, e.g., increased, level or activity of a TCRβ V6 subfamily, e.g., comprising TCRβ V6-5*01.

60. The method of any of the preceding claims, wherein the infectious disease is HCMV and the anti-TCRβV antibody molecule binds to the TCRβ V6 subfamily, e.g., comprising TCRβ V6-5*01.

61. The method of claim 60, wherein the subject has a higher, e.g., increased, level or activity of a TCRβ V6 subfamily, e.g., comprising TCRβ V6-5*01.

62. The method of any of claims 58-61, wherein the anti-TCRβV antibody molecule comprises:
(1) a heavy chain complementarity determining region (HC CDR1), a HC CDR2 and/or a HC CDR3 of SEQ ID NO: 3, SEQ ID NO: 4, and/or SEQ ID NO: 5; and/or
(2) a light chain complementarity determining region 1 (LC CDR1), a LC CDR2, and/or a LC CDR3 of SEQ ID NO: 6, SEQ ID NO: 7, and/or SEQ ID NO: 8.

63. The method of any of claims 58-61, wherein the anti-TCRβV antibody molecule comprises:
(1) a heavy chain complementarity determining region (HC CDR1), a HC CDR2 and/or a HC CDR3 of SEQ ID NO: 45, SEQ ID NO: 46, and/or SEQ ID NO: 47; and/or
(2) a light chain complementarity determining region 1 (LC CDR1), a LC CDR2, and/or a LC CDR3 of SEQ ID NO: 51, SEQ ID NO: 52, and/or SEQ ID NO: 53.

64. The method of any of claims 58-61, wherein the anti-TCRβV antibody molecule comprises:
(1) a heavy chain complementarity determining region (HC CDR1), a HC CDR2 and/or a HC CDR3 of SEQ ID NO: 48, SEQ ID NO: 49, and/or SEQ ID NO: 50; and/or
(2) a light chain complementarity determining region 1 (LC CDR1), a LC CDR2, and/or a LC CDR3 of SEQ ID NO: 54, SEQ ID NO: 55, and/or SEQ ID NO: 56.

65. The method of any of claims 58-64, wherein the anti-TCRβV antibody molecule comprises a VH having at least 85% (e.g., at least 85%, 90%, 95%, 96%, 97%, 98%, 99%, or 100%) sequence identity to SEQ ID NO: 9.

66. The method of any of claims 58-65, wherein the anti-TCRβV antibody molecule comprises a VL having at least 85% (e.g., at least 85%, 90%, 95%, 96%, 97%, 98%, 99%, or 100%) sequence identity to SEQ ID NO: 10.

67. The method of any of claims 58-64, wherein the anti-TCRβV antibody molecule comprises a VH having at least 85% (e.g., at least 85%, 90%, 95%, 96%, 97%, 98%, 99%, or 100%) sequence identity to SEQ ID NO: 9 and a VL having at least X % sequence identity to SEQ ID NO: 10.

68. The method of any of claims 58-67, wherein the anti-TCRβV antibody molecule comprises a heavy chain having at least 85% (e.g., at least 85%, 90%, 95%, 96%, 97%, 98%, 99%, or 100%) sequence identity to SEQ ID NO: 69 and a light chain having at least 85% (e.g., at least 85%, 90%, 95%, 96%, 97%, 98%, 99%, or 100%) sequence identity to SEQ ID NO: 72.

69. The method of any of the preceding claims, wherein the infectious disease is EBV and the anti-TCRβV antibody molecule binds to the TCRβ V10 subfamily, e.g., comprising TCRβ V10-1*01, TCRβ V10-1*02, TCRβ V10-3*01 or TCRβ V10-2*01.

70. The method of claim 69, wherein the antigen is BZLF1(52-64).

71. The method of claim 69 or 70, wherein the MHC restriction is HLA-B*3508.

72. The method of any of claims 69-71, wherein the subject has a higher, e.g., increased, level or activity of a TCRβ V10 subfamily, e.g., comprising TCRβ V10-1*01, TCRβ V10-1*02, TCRβ V10-3*01 or TCRβ V10-2*01.

73. The method of any of the preceding claims, wherein the infectious disease is malaria and the anti-TCRβV antibody molecule binds to the TCRβ V12 subfamily, e.g., comprising TCRβ V12-4*01, TCRβ V12-3*01, or TCRβ V12-5*01.

74. The method of claim 73, wherein the subject has a higher, e.g., increased, level or activity of a TCRβ V12 subfamily, e.g., comprising TCRβ V12-4*01, TCRβ V12-3*01, or TCRβ V12-5*01.

75. The method of any of the preceding claims, wherein the infectious disease is tuberculosis and the anti-TCRβV antibody molecule binds to the TCRβ V12 subfamily, e.g., comprising TCRβ V12-4*01, TCRβ V12-3*01, or TCRβ V12-5*01.

76. The method of claim 75, wherein the subject has a higher, e.g., increased, level or activity of a TCRβ V12 subfamily, e.g., comprising TCRβ V12-4*01, TCRβ V12-3*01, or TCRβ V12-5*01.

77. The method of any of the preceding claims, wherein the infectious disease is HCMV and the anti-TCRβV antibody molecule binds to the TCRβ V12 subfamily, e.g., comprising TCRβ V12-4*01.

78. The method of claim 77, wherein the subject has a higher, e.g., increased, level or activity of a TCRβ V12 subfamily, e.g., comprising TCRβ V12-4*01.

79. The method of any of claims 73-78, wherein the anti-TCRβV antibody molecule comprises:
(1) a heavy chain complementarity determining region (HC CDR1), a HC CDR2 and/or a HC CDR3 of SEQ ID NO: 17, SEQ ID NO: 18, and/or SEQ ID NO: 19; and/or
(2) a light chain complementarity determining region 1 (LC CDR1), a LC CDR2, and/or a LC CDR3 of SEQ ID NO: 20, SEQ ID NO: 21, and/or SEQ ID NO: 22.

80. The method of any of claims 73-78, wherein the anti-TCRβV antibody molecule comprises:
(1) a heavy chain complementarity determining region (HC CDR1), a HC CDR2 and/or a HC CDR3 of SEQ ID NO: 57, SEQ ID NO: 58, and/or SEQ ID NO: 59; and/or (2) a light chain complementarity determining region 1 (LC CDR1), a LC CDR2, and/or a LC CDR3 of SEQ ID NO: 63, SEQ ID NO: 64, and/or SEQ ID NO: 65.

81. The method of any of claims 73-78, wherein the anti-TCRβV antibody molecule comprises:
(1) a heavy chain complementarity determining region (HC CDR1), a HC CDR2 and/or a HC CDR3 of SEQ ID NO: 60, SEQ ID NO: 61, and/or SEQ ID NO: 62; and/or (2) a light chain complementarity determining region 1 (LC CDR1), a LC CDR2, and/or a LC CDR3 of SEQ ID NO: 66, SEQ ID NO: 67, and/or SEQ ID NO: 68.

82. The method of any of claims 73-81, wherein the anti-TCRβV antibody molecule comprises a VH having at least 85% (e.g., at least 85%, 90%, 95%, 96%, 97%, 98%, 99%, or 100%) sequence identity to SEQ ID NO: 15.

83. The method of any of claims 73-82, wherein the anti-TCRβV antibody molecule comprises a VL having at least 85% (e.g., at least 85%, 90%, 95%, 96%, 97%, 98%, 99%, or 100%) sequence identity to SEQ ID NO: 16, optionally wherein the anti-TCRβV antibody molecule comprises a VH having at least 85% (e.g., at least 85%, 90%, 95%, 96%, 97%, 98%, 99%, or 100%) sequence identity to SEQ ID NO: 15 and a VL having at least 85% (e.g., at least 85%, 90%, 95%, 96%, 97%, 98%, 99%, or 100%) sequence identity to SEQ ID NO: 16.

84. The method of any of claims 73-81, wherein the anti-TCRβV antibody molecule comprises a VH having at least 85% (e.g., at least 85%, 90%, 95%, 96%, 97%, 98%, 99%, or 100%) sequence identity to SEQ ID NO: 23.

85. The method of any of claims 73-81, wherein the anti-TCRβV antibody molecule comprises a VH having at least 85% (e.g., at least 85%, 90%, 95%, 96%, 97%, 98%, 99%, or 100%) sequence identity to SEQ ID NO: 24.

86. The method of any of claims 73-81, wherein the anti-TCRβV antibody molecule comprises a VH having at least 85% (e.g., at least 85%, 90%, 95%, 96%, 97%, 98%, 99%, or 100%) sequence identity to SEQ ID NO: 25.

87. The method of any of claims 73-86, wherein the anti-TCRβV antibody molecule comprises a VL having at least 85% (e.g., at least 85%, 90%, 95%, 96%, 97%, 98%, 99%, or 100%) sequence identity to SEQ ID NO: 26.

88. The method of any of claims 73-86, wherein the anti-TCRβV antibody molecule comprises a VL having at least 85% (e.g., at least 85%, 90%, 95%, 96%, 97%, 98%, 99%, or 100%) sequence identity to SEQ ID NO: 27.

89. The method of any of claims 73-86, wherein the anti-TCRβV antibody molecule comprises a VL having at least 85% (e.g., at least 85%, 90%, 95%, 96%, 97%, 98%, 99%, or 100%) sequence identity to SEQ ID NO: 28.

90. The method of any of claims 73-86, wherein the anti-TCRβV antibody molecule comprises a VL having at least 85% (e.g., at least 85%, 90%, 95%, 96%, 97%, 98%, 99%, or 100%) sequence identity to SEQ ID NO: 29.

91. The method of any of claims 73-86, wherein the anti-TCRβV antibody molecule comprises a VL having at least 85% (e.g., at least 85%, 90%, 95%, 96%, 97%, 98%, 99%, or 100%) sequence identity to SEQ ID NO: 30.

92. The method of any of the preceding claims, wherein the infectious disease is HIV and the anti-TCRβV antibody molecule binds to the TCRβ V5 subfamily, e.g., comprising TCRβ V5-6*01.

93. The method of claim 92, wherein the antigen is Gag p17 (77-85).

94. The method of claim 92 or 93, wherein the MHC restriction is HLA-B*0801.

95. The method of any of claims 92-94, wherein the subject has a higher, e.g., increased, level or activity of a TCRβ V5 subfamily, e.g., comprising TCRβ V5-6*01.

96. The method of any of the preceding claims, wherein the infectious disease is EBV and the anti-TCRβV antibody molecule binds to the TCRβ V7 subfamily, e.g., comprising TCRβ V7-7*01, TCRβ V7-6*01, TCRβ V7-8*02, TCRβ V7-4*01, TCRβ V7-2*02, TCRβ V7-2*03, TCRβ V7-2*01, TCRβ V7-3*01, TCRβ V7-9*03, or TCRβ V7-9*01.

97. The method of claim 96, wherein the antigen is EBNA3(339-347).

98. The method of claim 96 or 97, wherein the MHC restriction is HLA-B*0801.

99. The method of any of claims 96-98, wherein the subject has a higher, e.g., increased, level or activity of a TCRβ V7 subfamily, e.g., comprising TCRβ V7-7*01, TCRβ V7-6*01, TCRβ V7 -8*02, TCRβ V7-4*01, TCRβ V7-2*02, TCRβ V7-2*03, TCRβ V7-2*01, TCRβ V7-3*01, TCRβ V7-9*03, or TCRβ V7-9*01.

100. The method of any of the preceding claims, wherein the infectious disease is SIV and the anti-TCRβV antibody molecule binds to the TCRβ V14 subfamily, e.g., comprising TCRβ V14*01.

101. The method of claim 100, wherein the subject has a higher, e.g., increased, level or activity of a TCRβ V14 subfamily, e.g., comprising TCRβ V14*01.

102. The method of any of the preceding claims, wherein the infectious disease is EBV and the anti-TCRβV antibody molecule binds to the TCRβ V9 subfamily, e.g., comprising TCRβ V9*01 or TCRβ V9*02.

103. The method of claim 102, wherein the antigen is EBNA1(407-417).

104. The method of claim 102 or 103, wherein the MHC restriction is HLA-B*3508 or HLA-B*3501.

105. The method of any of claims 102-104, wherein the subject has a higher, e.g., increased, level or activity of a TCRβ V9 subfamily, e.g., comprising TCRβ V9*01 or TCRβ V9*02.

106. The method of any of the preceding claims, wherein the infectious disease is SIV and the anti-TCRβV antibody molecule binds to the TCRβ V13 subfamily, e.g., comprising TCRβ V13*01.

107. The method of claim 106, wherein the subject has a higher, e.g., increased, level or activity of a TCRβ V13 subfamily, e.g., comprising TCRβ V13*01.

108. The method of any of the preceding claims, wherein the infectious disease is influenza and the anti-TCRβ V antibody molecule binds to the TCRβ V19 subfamily, e.g., comprising TCRβ V19*01, or TCRβ V19*02.

109. The method of claim 108, wherein the antigen is Matrix protein (58-66).

110. The method of claim 108 or 109, wherein the MHC restriction is HLA-A2.

111. The method of any of claims 108-110, wherein the subject has a higher, e.g., increased, level or activity of a TCRβ V19 subfamily, e.g., comprising TCRβ V19*01, or TCRβ V19*02.

112. The method of any of the preceding claims, wherein the infectious disease is HIV and the anti-TCRβ V antibody molecule binds to the TCRα V19 subfamily, e.g., comprising TCRβ V19*01, or TCRβ V19*02.

113. The method of claim 112, wherein the subject has a higher, e.g., increased, level or activity of a TCRβ V19 subfamily, e.g., comprising TCRβ V19*01, or TCRβ V19*02.

114. The method of any of the preceding claims, wherein the infectious disease is HIV and the anti-TCRβ V antibody molecule binds to the TCRβ V23 subfamily, e.g., comprising TCRβ V23-1.

115. The method of claim 114, wherein the subject has a higher, e.g., increased, level or activity of a TCRβ V23 subfamily, e.g., comprising TCRβ V23-1.

116. The method of any of the preceding claims, wherein the anti-TCRβ V antibody molecule:
(i) binds specifically to an epitope on TCRβ V, e.g., the same or similar epitope as the epitope recognized by an anti-TCRβ V antibody molecule as described herein, e.g., a second anti-TCRβ V antibody molecule;
(ii) shows the same or similar binding affinity or specificity, or both, as an anti-TCRβ V antibody molecule as described herein, e.g., a second anti-TCRβ V antibody molecule;
(iii) inhibits, e.g., competitively inhibits, the binding of an anti-TCRβ V antibody molecule as described herein, e.g., a second anti-TCRβ V antibody molecule;
(iv) binds the same or an overlapping epitope with an anti-TCRβ V antibody molecule as described herein, e.g., a second anti-TCRβ V antibody molecule; or
(v) competes for binding, and/or binds the same epitope, with an anti-TCRβ V antibody molecule as described herein, e.g., a second anti-TCRβ V antibody molecule.

117. The method of any of the preceding claims, wherein the anti-TCRβV antibody molecule comprises an antigen binding domain comprising:
(i) a heavy chain complementarity determining region 1 (HC CDR1), a heavy chain complementarity determining region 2 (HC CDR2) and/or a heavy chain complementarity determining region 3 (HC CDR3) of SEQ ID NO: 1 or SEQ ID NO: 9; or
(ii) a light chain complementarity determining region 1 (LC CDR1), a light chain complementarity determining region 2 (LC CDR2), and/or a light chain complementarity determining region 3 (LC CDR3) of SEQ ID NO: 2, SEQ ID NO: 10, or SEQ ID NO: 11.

118. The method of any of the preceding claims, wherein the anti-TCRβV antibody molecule comprises an antigen binding domain comprising a light chain variable region (VL) comprising one, two or all (e.g., three) of a LC CDR1, a LC CDR2 and a LC CDR3 of SEQ ID NO: 2, SEQ ID NO: 10, or SEQ ID NO: 11.

119. The method of any of the preceding claims, wherein the anti-TCRβV antibody molecule comprises an antigen binding domain comprising a heavy chain variable region (VH) comprising one, two or all (e.g., three) of a HC CDR1, a HC CDR2 and a HC CDR3 of SEQ ID NO:1 or SEQ ID NO: 9.

120. The method of any of the preceding claims, wherein the anti-TCRβV antibody molecule comprises an antigen binding domain comprising:
(i) a VL comprising: a LC CDR1 amino acid sequence of SEQ ID NO: 6 (or an amino acid sequence with not more than 1, 2, 3 or 4 modifications, e.g., substitutions, additions or deletions thereof), a LC CDR2 amino acid sequence of SEQ ID NO:7 (or an amino acid sequence with not more than 1, 2, 3 or 4 modifications, e.g., substitutions, additions or deletions thereof), and/or a LC CDR3 amino acid sequence of SEQ ID NO:8 (or an amino acid sequence with not more than 1, 2, 3 or 4 modifications, e.g., substitutions, additions or deletions thereof); and/or
(ii) a VH comprising: a HC CDR1 amino acid sequence of SEQ ID NO: 3 (or an amino acid sequence with not more than 1, 2, 3 or 4 modifications, e.g., substitutions, additions or deletions thereof), a HC CDR2 amino acid sequence of SEQ ID NO:4 (or an amino acid sequence with not more than 1, 2, 3 or 4 modifications, e.g., substitutions, additions or deletions thereof), and/or a HC CDR3 amino acid sequence of SEQ ID NO:5 (or an amino acid sequence with not more than 1, 2, 3 or 4 modifications, e.g., substitutions, additions or deletions thereof).

121. The method of any of the preceding claims, wherein the anti-TCRβV antibody molecule comprises an antigen binding domain comprising:
a variable heavy chain (VH) of SEQ ID NO: 9, or a sequence having at least about 85%, 90%, 95%, or 99% sequence identity thereto; and/or
a variable light chain (VL) of SEQ ID NO: 10 or SEQ ID NO: 11, or a sequence having at least about 85%, 90%, 95%, or 99% sequence identity thereto.

122. The method of any of the preceding claims, wherein the anti-TCRβV antibody molecule comprises an antigen binding domain comprising the VH amino acid sequence of SEQ ID NO: 9 and the VL amino acid sequence of SEQ ID NO: 10.

123. The method of any of the preceding claims, wherein the anti-TCRβV antibody molecule comprises an antigen binding domain comprising the VH amino acid sequence of SEQ ID NO: 9 and the VL amino acid sequence of SEQ ID NO: 11.

124. The method of any of the preceding claims, wherein the anti-TCRβV antibody molecule comprises an antigen binding domain comprising a single chain Fv (scFv) or a Fab.

125. The method of any of the preceding claims, wherein the anti-TCRβV antibody molecule binds to a conformational or a linear epitope on the T cell.

126. The method of any of the preceding claims, wherein the anti-TCRβV antibody molecule is a full antibody (e.g., an antibody that includes at least one, and preferably two, complete heavy chains, and at least one, and preferably two, complete light chains), or an antigen-binding fragment (e.g., a Fab, F(ab′)₂, Fv, a single chain Fv fragment, a single domain antibody, a diabody (dAb), a bivalent antibody, or bispecific antibody or fragment thereof, a single domain variant thereof, or a camelid antibody).

127. The method of claim 126, wherein the anti-TCRβV antibody molecule comprises a heavy chain constant region chosen from IgG1, IgG2, IgG3, or IgG4, or a fragment thereof.

128. The method of claim 126 or 127, wherein the anti-TCRβV antibody molecule comprises a light chain constant region chosen from the light chain constant regions of kappa or lambda, or a fragment thereof.

129. A method of making, e.g., producing or manufacturing, the anti-TCRβV antibody molecule of the method of any of the preceding claims, comprising culturing a host cell comprising a nucleic acid encoding the anti-TCRβV antibody molecule, under suitable conditions, e.g., conditions suitable expression of the anti- TCRβV antibody molecule.

130. A pharmaceutical composition comprising the anti-TCRβV antibody molecule of the method of any of the preceding claims, and a pharmaceutically acceptable carrier, excipient, or stabilizer.

131. The method of any of claims 1-128, wherein the expansion occurs in vivo or ex vivo (e.g., in vitro).

132. The method of any of claim 1-128 or 131, wherein the T cell population comprises a T cell, a Natural Killer cell, a B cell, or a myeloid cell.

133. The method of any of claim 1-128, 131, or 132, wherein the T cell population comprises a CD4 T cell, a CD8 T cell, e.g., an effector T cell or a memory T cell (e.g., a memory effector T cell (e.g., T_{EM} cell, e.g., T_{EMRA} cell), or a combination thereof.

134. The method of any of claim 1-128 or 131-133, wherein the T cell population is obtained from a healthy subject.

135. The method of any of claim 1-128 or 131-134, wherein the T cell population is obtained from a subject (e.g., from an apheresis sample from the subject) having a disease, e.g., an infectious disease, e.g., as described herein.

136. The method of any of claim 1-128 or 131-135, wherein the method results in an expansion of at least 1.1-10 fold (e.g., at least 1.1, 1.2, 1.3, 1.4, 1.5, 2, 3, 4, 5, 6, 7, 8, 9, or 10 fold expansion).

137. The method of any of claim 1-128 or 131-136, further comprising contacting the population of cells with an agent that promotes, e.g., increases, immune cell (e.g., T cell) expansion.

138. The method of any of claim 1-128 or 131-137, further comprising contacting the population of cells with an additional therapeutic agent.

139. The method of claim 138, wherein the additional therapeutic agent targets the infectious disease.

140. The method of any of claim 1-128 or 131-139, further comprising contacting the population of cells with a non-dividing population of cells, e.g., feeder cells, e.g., irradiated allogenic human PBMCs.

141. The method of any of claim 1-128 or 131-140, wherein the population of cells is expanded in an appropriate media (e.g., media described herein) that includes one or more cytokines, e.g., IL-2, IL-7, IL-15, or a combination thereof.

142. The method of any of claim 1-128 or 131-141, wherein the population of cells is expanded for a period of at least about 4 hours, 6 hours, 10 hours, 12 hours, 15 hours, 18 hours, 20 hours, or 22 hours, or for at least 1, 2, 3, 4, 5, 6, 7, 8, 9, 10, 11, 12, 13, 14, 15, 1,6 17, 18, 19, 20 or 21 days, or for at least about 1 week, 2 weeks, 3 weeks, 4 weeks, 5 weeks, 6 weeks, 7 weeks or 8 weeks.

143. The method of any of claim 1-128 or 131-142, wherein expansion of the population of T cells is compared to expansion of a similar population of cells with an antibody that binds to: a CD3 molecule, e.g., CD3 epsilon (CD3e) molecule; or a TCR alpha (TCRα) molecule.

144. The method of any of claim 1-128 or 131-143, wherein expansion of the population of T cells is compared to expansion of a similar population of cells not contacted with the anti-TCRβV antibody molecule.

145. The method of any of claim 1-128 or 131-144, wherein expansion of the population of T cells, e.g., memory effector T cells, e.g., T_{EM} cells, e.g., T_{EMRA} cells, is compared to expansion of a similar population of cells with an antibody that binds to: a CD3 molecule, e.g., CD3 epsilon (CD3e) molecule; or a TCR alpha (TCRα) molecule.

146. The method of any of claim 1-128 or 131-145, wherein the population of expanded T cells, e.g., expanded T effector memory cells, comprises cells which:
(i) have a detectable level of CD45RA, e.g., express or re-express CD45RA;
(ii) have low or no expression of CCR7; and/or
(iii) have a detectable level of CD95, e.g., express CD95,
e.g., a population of CD45RA+, CCR7-, CD95+T cells, optionally wherein the T cells comprise CD3+, CD4+or CD8+T cells.

147. The method of any of claim 1-128 or 131-146, wherein the antibody molecule, e.g., humanized antibody molecule, which binds, e.g., specifically binds, to the TCRβV region (the anti-TCRβV antibody molecule) is chosen from:
(A) a humanized antibody molecule which binds, e.g., specifically binds, to a T cell receptor beta variable chain (TCRβV) region chosen from TCRβV V5-6, TCRβV V6-5, TCRβV V7, TCRβV V9, TCRβV V10, TCRβV V12 (e.g., TCRβV V12-4), TCRβV V13, TCRβV V14, TCRβV V19, TCRβV V23-1, or a subfamily member thereof (e.g., as listed in Table 1 or Table 2);
(B) a humanized antibody molecule which:
(i) binds specifically to an epitope on TCRβV, e.g., the same or similar epitope as the epitope recognized by a second anti-TCRβV antibody molecule;
(ii) shows the same or similar binding affinity or specificity, or both, as a second anti-TCRβV antibody molecule;
(iii) inhibits, e.g., competitively inhibits, the binding of a second anti-TCRβV antibody molecule;
(iv) binds the same or an overlapping epitope with an anti-TCRβV antibody molecule as a second anti-TCRβV antibody molecule; or
(v) competes for binding, and/or binds the same epitope, with a second anti-TCRβV antibody molecule,
wherein the second anti-TCRβV antibody molecule comprises an antigen binding domain comprising:
(1) a heavy chain complementarity determining region (HC CDR1), a HC CDR2 and/or a HC CDR3 of SEQ ID NO: 3, SEQ ID NO: 4, and/or SEQ ID NO: 5, and/or
(2) a light chain complementarity determining region 1 (LC CDR1), a LC CDR2, and/or a LC CDR3 of SEQ ID NO: 6, SEQ ID NO: 7, and/or SEQ ID NO: 8;
(1) a heavy chain complementarity determining region (HC CDR1), a HC CDR2 and/or a HC CDR3 of SEQ ID NO: 45, SEQ ID NO: 46, and/or SEQ ID NO: 47, and/or
(2) a light chain complementarity determining region 1 (LC CDR1), a LC CDR2, and/or a LC CDR3 of SEQ ID NO: 51, SEQ ID NO: 52, and/or SEQ ID NO: 53;
(1) a heavy chain complementarity determining region (HC CDR1), a HC CDR2 and/or a HC CDR3 of SEQ ID NO: 48, SEQ ID NO: 49, and/or SEQ ID NO: 50, and/or
(2) a light chain complementarity determining region 1 (LC CDR1), a LC CDR2, and/or a LC CDR3 of SEQ ID NO: 54, SEQ ID NO: 55, and/or SEQ ID NO: 56;
(1) a heavy chain complementarity determining region (HC CDR1), a HC CDR2 and/or a HC CDR3 of SEQ ID NO: 17, SEQ ID NO: 18, and/or SEQ ID NO: 19, and/or
(2) a light chain complementarity determining region 1 (LC CDR1), a LC CDR2, and/or a LC CDR3 of SEQ ID NO: 20, SEQ ID NO: 21, and/or SEQ ID NO: 22;
(1) a heavy chain complementarity determining region (HC CDR1), a HC CDR2 and/or a HC CDR3 of SEQ ID NO: 57, SEQ ID NO: 58, and/or SEQ ID NO: 59, and/or
(2) a light chain complementarity determining region 1 (LC CDR1), a LC CDR2, and/or a LC CDR3 of SEQ ID NO: 63, SEQ ID NO: 64, and/or SEQ ID NO: 65;
(1) a heavy chain complementarity determining region (HC CDR1), a HC CDR2 and/or a HC CDR3 of SEQ ID NO: 60, SEQ ID NO: 61, and/or SEQ ID NO: 62, and/or
(2) a light chain complementarity determining region 1 (LC CDR1), a LC CDR2, and/or a LC CDR3 of SEQ ID NO: 66, SEQ ID NO: 67, and/or SEQ ID NO: 68; or
(1) a heavy chain complementarity determining region (HC CDR1), a HC CDR2 and/or a HC CDR3 of SEQ ID NO: 15, SEQ ID NO: 23, SEQ ID NO: 24, or SEQ ID NO: 25, and/or
(2) a light chain complementarity determining region 1 (LC CDR1), a LC CDR2, and/or a LC CDR3 of SEQ ID NO: 16, SEQ ID NO: 26, SEQ ID NO: 27, SEQ ID NO: 28, SEQ ID NO: 29, or SEQ ID NO: 30; or
(C) a humanized antibody molecule which binds, e.g., specifically binds, to a T cell receptor beta variable chain (TCRβV) region, wherein the anti-TCRβV antibody molecule comprises an antigen binding domain comprising:
(1) a heavy chain complementarity determining region (HC CDR1), a HC CDR2 and/or a HC CDR3 of SEQ ID NO: 3, SEQ ID NO: 4, and/or SEQ ID NO: 5, and/or
(2) a light chain complementarity determining region 1 (LC CDR1), a LC CDR2, and/or a LC CDR3 of SEQ ID NO: 6, SEQ ID NO: 7, and/or SEQ ID NO: 8;
(1) a heavy chain complementarity determining region (HC CDR1), a HC CDR2 and/or a HC CDR3 of SEQ ID NO: 45, SEQ ID NO: 46, and/or SEQ ID NO: 47, and/or
(2) a light chain complementarity determining region 1 (LC CDR1), a LC CDR2, and/or a LC CDR3 of SEQ ID NO: 51, SEQ ID NO: 52, and/or SEQ ID NO: 53;
(1) a heavy chain complementarity determining region (HC CDR1), a HC CDR2 and/or a HC CDR3 of SEQ ID NO: 48, SEQ ID NO: 49, and/or SEQ ID NO: 50, and/or
(2) a light chain complementarity determining region 1 (LC CDR1), a LC CDR2, and/or a LC CDR3 of SEQ ID NO: 54, SEQ ID NO: 55, and/or SEQ ID NO: 56;
(1) a heavy chain complementarity determining region (HC CDR1), a HC CDR2 and/or a HC CDR3 of SEQ ID NO: 17, SEQ ID NO: 18, and/or SEQ ID NO: 19, and/or
(2) a light chain complementarity determining region 1 (LC CDR1), a LC CDR2, and/or a LC CDR3 of SEQ ID NO: 20, SEQ ID NO: 21, and/or SEQ ID NO: 22;
(1) a heavy chain complementarity determining region (HC CDR1), a HC CDR2 and/or a HC CDR3 of SEQ ID NO: 57, SEQ ID NO: 58, and/or SEQ ID NO: 59, and/or
(2) a light chain complementarity determining region 1 (LC CDR1), a LC CDR2, and/or a LC CDR3 of SEQ ID NO: 63, SEQ ID NO: 64, and/or SEQ ID NO: 65;
(1) a heavy chain complementarity determining region (HC CDR1), a HC CDR2 and/or a HC CDR3 of SEQ ID NO: 60, SEQ ID NO: 61, and/or SEQ ID NO: 62, and/or
(2) a light chain complementarity determining region 1 (LC CDR1), a LC CDR2, and/or a LC CDR3 of SEQ ID NO: 66, SEQ ID NO: 67, and/or SEQ ID NO: 68; or
(i) a heavy chain complementarity determining region (HC CDR1), a HC CDR2 and/or a HC CDR3 of SEQ ID NO: 15, SEQ ID NO: 23, SEQ ID NO: 24, or SEQ ID NO: 25; and/or
(ii) a light chain complementarity determining region 1 (LC CDR1), a LC CDR2, and/or a LC CDR3 of SEQ ID NO: 16, SEQ ID NO: 26, SEQ ID NO: 27, SEQ ID NO:
28, SEQ ID NO: 29, or SEQ ID NO: 30.

148. The method of any of claim 1-128 or 131-147, wherein the anti-TCRβV antibody molecule comprises an antigen binding domain comprising a light chain variable region (VL) comprising one, two or all of a LC CDR1, a LC CDR2 and a LC CDR3 of SEQ ID NO: 16, SEQ ID NO: 26, SEQ ID NO: 27, SEQ ID NO: 28, SEQ ID NO: 29, or SEQ ID NO: 30.

149. The method of any of claim 1-128 or 131-148, wherein the anti-TCRβV antibody molecule comprises an antigen binding domain comprising a heavy chain variable region (VH) comprising one, two or all of a HC CDR1, a HC CDR2 and a HC CDR3 of SEQ ID NO: 15, SEQ ID NO: 23, SEQ ID NO: 24, or SEQ ID NO: 25.

150. The method of any of claim 1-128 or 131-149, wherein the anti-TCRβV antibody molecule comprises an antigen binding domain comprising:
(i) a VL comprising: a LC CDR1 amino acid sequence of SEQ ID NO: 20 (or an amino acid sequence with not more than 1, 2, 3 or 4 modifications, e.g., substitutions, additions or deletions thereof), a LC CDR2 amino acid sequence of SEQ ID NO:21 (or an amino acid sequence with not more than 1, 2, 3 or 4 modifications, e.g., substitutions, additions or deletions thereof), and/or a LC CDR3 amino acid sequence of SEQ ID NO:22 (or an amino acid sequence with not more than 1, 2, 3 or 4 modifications, e.g., substitutions, additions or deletions thereof); and/or
(ii) a VH comprising: a HC CDR1 amino acid sequence of SEQ ID NO: 17 (or an amino acid sequence with not more than 1, 2, 3 or 4 modifications, e.g., substitutions, additions or deletions thereof), a HC CDR2 amino acid sequence of SEQ ID NO:18 (or an amino acid sequence with not more than 1, 2, 3 or 4 modifications, e.g., substitutions, additions or deletions thereof), and/or a HC CDR3 amino acid sequence of SEQ ID NO:19 (or an amino acid sequence with not more than 1, 2, 3 or 4 modifications, e.g., substitutions, additions or deletions thereof).

151. The method of any of claim 1-128 or 131-150, wherein the anti-TCRβV antibody molecule comprises an antigen binding domain comprising:
a variable heavy chain (VH) of SEQ ID NO: 23, SEQ ID NO: 24, or SEQ ID NO: 25, or a sequence having at least about 85%, 90%, 95%, or 99% sequence identity thereto; and/or
a variable light chain (VL) of SEQ ID NO: 26, SEQ ID NO: 27, SEQ ID NO: 28, SEQ ID NO: 29, or SEQ ID NO: 30, or a sequence having at least about 85%, 90%, 95%, or 99% sequence identity thereto.

152. The method of any of claim 1-128 or 131-151, wherein the anti-TCRβV antibody molecule comprises a light chain comprising a framework region, e.g., framework region 1 (FR1), comprising one, two or all (e.g., three) of:
(i) an Aspartic Acid at position 1, e.g., a substitution at position 1 according to Kabat numbering, e.g., a Alanine to Aspartic Acid substitution; or
(ii) an Asparagine at position 2, e.g., a substitution at position 2 according to Kabat numbering, e.g., a Isoleucine to Asparagine, a Serine to Asparagine, or a Tyrosine to Asparagine substitution; or
(iii) a Leucine at position 4, e.g., a substitution at position 4 according to Kabat numbering, e.g., a Methionine to Leucine substitution,
wherein the substitution is relative to a human germline light chain framework region sequence.

153. The method of any of claim 1-128 or 131-152, wherein the anti-TCRβV antibody molecule comprises a light chain comprising a framework region, e.g., framework region 3 (FR3), comprising one, two or all (e.g., three) of:
(i) a Glycine at position 66, e.g., a substitution at position 66 according to Kabat numbering, e.g., a Lysine to Glycine, or a Serine to Glycine substitution; or
(ii) an Asparagine at position 69, e.g., a substitution at position 69 according to Kabat numbering, e.g., a Threonine to Asparagine substitution; or
(iii) a Tyrosine at position 71, e.g., a substitution at position 71 according to Kabat numbering, e.g., a Phenylalanine to Tyrosine, or Alanine to Tyrosine substitution,
wherein the substitution is relative to a human germline light chain framework region sequence.

154. The method of any of claim 1-128 or 131-153, wherein the method results in expansion of, e.g., selective or preferential expansion of, T cells expressing a T cell receptor (TCR) comprising a TCR alpha and/or TCR beta molecule, e.g., TCR alpha-beta T cells (αβ T cells).

155. The method of any of claim 1-128 or 131-154, wherein the method results in expansion of αβT cells over expansion of T cells expressing a TCR comprising a TCR gamma and/or TCR delta molecule, e.g., TCR gamma-delta T cells (γδ T cells).
